(19) United States
(12) Patent Application Publication
(10) Pub. No.: US 2022/0206001 A1
(43) Pub. Date: Jun. 30, 2022

(54) METHODS AND SYSTEMS FOR DETECTING CORONAVIRUS

(71) Applicant: Twist Bioscience Corporation, South San Francisco, CA (US)

(72) Inventor: Aaron SATO, Burlingame, CA (US)

(21) Appl. No.: 17/507,703

(22) Filed: Oct. 21, 2021

Related U.S. Application Data
(60) Provisional application No. 63/104,368, filed on Oct. 22, 2020.

(51) Int. Cl. G01N 33/569; C07K 16/10

(52) U.S. Cl. CPC G01N 33/56983; C07K 16/10; A61K 2039/505; C07K 2317/92; C07K 2317/565; C07K 2317/76

(57) ABSTRACT

Provided herein are methods and systems for detecting coronavirus. Methods and systems as described herein comprise using antibodies with improved specificity and sensitivity for accurately detecting coronavirus.

### CROSS-REFERENCE

This application claims the benefit of U.S. Provisional Patent Application No. 63/104,368 filed on Oct. 22, 2020, which is incorporated by reference in its entirety.

### SEQUENCE LISTING

The instant application contains a Sequence Listing which has been submitted electronically in ASCII format and is hereby incorporated by reference in its entirety. Said ASCII copy, created on Nov. 30, 2021, is named 44854-810_201_SL.txt and is 2,287,083 bytes in size.

### BACKGROUND

Coronaviruses like severe acute respiratory coronavirus 2 (SARS-CoV-2) can cause severe respiratory problems. Accurate and timely detection of infection is important for diagnosis and identifying effective treatments. Antibodies possess the capability to bind with high specificity and affinity to biological targets and be incorporated in systems and devices for detecting coronavirus.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are devices for detecting a virus in a sample comprising: a) a sample application pad for receiving the sample; and b) a membrane substrate comprising a first test line, the first test line comprising an immobilized antibody or antibody fragment, wherein the immobilized antibody or antibody fragment comprises a predetermined number of variants within a complementarity determining region (CDR) relative to a reference antibody or antibody fragment, and wherein the immobilized antibody or antibody fragment comprises at least a 2.5× higher binding affinity than a binding affinity of the reference antibody or antibody fragment. Further provided herein are devices, wherein the device is a lateral flow immunoassay. Further provided herein are devices, wherein the immobilized antibody comprises a light chain variable domain comprising at least about 80% sequence identity to any one of SEQ ID NOs: 1, 3, 5, 7, 9, 11, or 13. Further provided herein are devices, wherein the immobilized antibody comprises a heavy chain variable domain comprising at least about 80% sequence identity to any one of SEQ ID NOs: 2, 4, 6, 8, 10, 12, 14, or 15. Further provided herein are devices, wherein the immobilized antibody comprises a heavy chain variable domain CDR comprising at least about 80% sequence identity to any one of SEQ ID NOs: 16-39. Further provided herein are devices, wherein the immobilized antibody comprises a light chain variable domain CDR comprising at least about 80% sequence identity to any one of SEQ ID NOs: 40-60. Further provided herein are devices, wherein the CDR comprises at least one variant relative to the reference antibody or antibody fragment. Further provided herein are devices, wherein the CDR comprises at least two variants relative to the reference antibody or antibody fragment. Further provided herein are devices, wherein the immobilized antibody or antibody fragment comprises at least 5× higher binding affinity than a binding affinity of the reference antibody or antibody fragment. Further provided herein are devices, wherein the immobilized antibody or antibody fragment comprises at least 25× higher binding affinity than a binding affinity of the reference antibody or antibody fragment. Further provided herein are devices, wherein the CDR is a CDR1, CDR2, and CDR3 on a heavy chain. Further provided herein are devices, wherein the CDR is a CDR1, CDR2, and CDR3 on a light chain. Further provided herein are devices, wherein the immobilized antibody comprises an EC50 of less than about 5 nM. Further provided herein are devices, wherein the immobilized antibody comprises an EC50 of less than about 1 nM. Further provided herein are devices, wherein the immobilized antibody comprises a binding affinity of less than about 100 nM. Further provided herein are devices, wherein the immobilized antibody comprises a binding affinity of less than about 25 nM. Further provided herein are devices, wherein the immobilized antibody comprises a binding affinity of less than about 1 nM. Further provided herein are devices, wherein the virus is a respiratory virus. Further provided herein are devices, wherein the respiratory virus is a coronavirus. Further provided herein are devices, wherein the coronavirus is SARS, MERS, COVID-19, bovine, norovirus, orthoreoviruses (reoviruses), human rotaviruses, human coronaviruses, herpesvirus, or adenoviruses. Further provided herein are devices, wherein the immobilized antibody detects SARS-CoV-2. Further provided herein are devices, wherein the sample comprises saliva, blood, semen, vaginal fluid, or urine. Further provided herein are devices, wherein the sample comprises saliva. Further provided herein are devices, wherein the membrane substrate further comprises at least one control line. Further provided herein are devices, wherein the device further comprises a backing. Further provided herein are devices, wherein the device further comprises a wicking pad. Further provided herein are devices, wherein the device comprises a sensitivity of at least about 70% for detecting the virus. Further provided herein are devices, wherein the device detects viral titers in a range of about 10³ to about 10⁴ viral particles. Further provided herein are devices, wherein the device comprises a specificity of at least about 70% for detecting the virus as compared to another virus. Further provided herein are devices, wherein the device is specific for detecting SARS-CoV-2. Further provided herein are devices, wherein the device comprises a limit of detection of at least about 10³ copies/mL.

Provided herein are methods of detecting a virus, the method comprising: a) contacting a sample comprising the virus with a device described herein; and b) detecting the virus if the first test line undergoes a color change. Further provided herein are methods, wherein the method detects the virus in at most about 20 minutes. Further provided herein are methods, wherein the method detects the virus in at most about 15 minutes.

Provided herein are kits comprising: a) a device described herein, and b) instructions for use thereof.

### BRIEF DESCRIPTION OF THE DRAWINGS

FIG. 1 depicts an exemplary lateral flow assay device.

FIG. 2 presents a diagram of steps demonstrating an exemplary process workflow for gene synthesis as disclosed herein.

FIG. 3 illustrates an example of a computer system.

FIG. 4 is a block diagram illustrating an architecture of a computer system.

FIG. 5 is a diagram demonstrating a network configured to incorporate a plurality of computer systems, a plurality of cell phones and personal data assistants, and Network Attached Storage (NAS).

FIG. 6 is a block diagram of a multiprocessor computer system using a shared virtual address memory space.

FIGS. 7A-7K are graphs showing antibody binding using surface plasmon resonance (SPR, Carterra LSA) to either recombinant SARS-CoV-2 S1 monomer, stabilized SARS-CoV-2 S Trimer, or SARS-CoV S1 monomer.

FIGS. 7L-7M show gel and electropherograms of Ab-1 analysis (FIG. 7L) and Ab-8 (FIG. 7M).

FIG. 8 are graphs of Ab-1 capturing either S trimer from Acro Biosystems (left panel) or S Spike Protein (right panel) using Ab-7, Ab-4, or CR3022 detection.

FIG. 9 shows images of a lateral flow antibody test.

FIG. 10A shows graphs of data from the lateral flow antibody test using a dry capture.

FIG. 10B is an image of test strips from the lateral flow antibody test.

FIG. 10C is an image of test strips from the lateral flow antibody test showing the spike trimer passing through the device.

FIG. 11A shows an image of test trips testing inactivated virus.

FIG. 11B shows data from live virus on swabs.

FIG. 12 is a schema for a rapid antigen detection (RAD) assay for detecting SARS-CoV-2.

FIGS. 13A-13B are images of test strips using the lateral flow device as compared to data from PCR.

FIGS. 13C-13E are images of test strips for detecting SARS-CoV-2 in SARS-CoV-2 positive saliva samples.

FIGS. 14A-14E are images of the lateral flow assays and detection cassettes.

FIGS. 15A-15B are a schema for detection of SARS-CoV-2 using the integrated cassette.

FIGS. 16A-16B are a schema for detection of SARS-CoV-2 using the open well cassette.

FIG. 17 depicts lateral flow assays used to detect SARS-CoV-2 at different concentrations.

FIGS. 18A-18D are lateral flow assay cassettes used to detect different concentrations of SARS-CoV-2.

FIG. 19 is a representative lateral flow assay cassette used in the clinical trial.

FIGS. 20A-20C depict the effects of single and double purification of saliva samples on nonspecific binding.

FIG. 21 depicts a lateral flow strip tested at a pH of 4 and a pH of 10.

FIG. 22 depicts a lateral flow assay using Ab-10 as a detector antibody Ab-10 and Ab-9 as a capture antibody.

FIG. 23A compares pairs of 7 conjugate detector antibodies with 5 capture antibodies targeting nucleocapsid.

FIG. 23B depicts a comparison of two candidate detector antibodies against 5 capture antibodies targeting nucleocapsid.

FIG. 23C-23D depicts the effects of an optimized buffer on nucleocapsid and spike binding and detection.

FIGS. 24A-24B depicts the results of different rations of capture and detector antibodies on nonspecific binding.

FIG. 25 depicts positive results of a clinical trial to detect SARS-CoV-2.

FIG. 26 depicts the results of buffer optimization on nonspecific binding.

FIG. 27 depicts the results of purification of saliva samples on nonspecific binding.

FIG. 28 depicts the ability of the nucleocapsid and spike assay to detect virus in diluted inactivated virus and nasopharyngeal samples.

FIG. 29 compares the ability to detect dilutions of inactivated virus in saliva of the nucleocapsid+spike lateral flow assay to the spike lateral flow assay.

FIG. 30 compares the ability to detect dilutions of virus in nasopharyngeal samples of the nucleocapsid+spike lateral flow assay to the spike lateral flow assay.

FIG. 31 depicts the effect of adding mucolytic agents to saliva samples on nonspecific binding.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein the terms “individual,” “patient,” or “subject” are used interchangeably and refer to individuals diagnosed with, suspected of being afflicted with, or at-risk of developing at least one disease for which the described systems and devices are useful for detecting. In embodiments the individual is a mammal. In embodiments, the mammal is a mouse, rat, rabbit, dog, cat, horse, cow, sheep, pig, goat, llama, alpaca, or yak. In embodiments, the individual is a human.

As used herein, the terms “polypeptide”, “protein” and “peptide” are used interchangeably and refer to a polymer of amino acid residues linked via peptide bonds and which may be composed of two or more polypeptide chains. The terms “polypeptide”, “protein” and “peptide” refer to a polymer of at least two amino acid monomers joined together through amide bonds. An amino acid may be the L-optical isomer or the D-optical isomer. More specifically, the terms “polypeptide”, “protein” and “peptide” refer to a molecule composed of two or more amino acids in a specific order; for example, the order as determined by the base sequence of nucleotides in the gene or RNA coding for the protein. In some cases, a protein is a portion of the protein, for example, a domain, a subdomain, or a motif of the protein. In some cases, a protein is a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant.

Unless specifically stated, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred herein may comprise at least one region encoding for exon sequences without an intervening intron sequence in the genomic equivalent sequence. cDNA described herein may be generated by de novo synthesis.

Systems and Devices for Detecting Coronavirus

Provided herein are methods, devices, and systems comprising antibodies that comprise high affinity and high specificity. In some embodiments, the antibodies detect SARS-CoV, MERS-CoV, CoV-229E, HCoV-NL63, HCoV-OC43, or HCoV-HKU1. In some embodiments, the antibodies detect SARS-CoV-2. In some embodiments, the antibodies detect a receptor that binds to the coronavirus. In some embodiments, the receptor of the coronavirus is ACE2 or dipeptidyl peptidase 4 (DPP4). In some embodiments, the antibodies detect angiotensin-converting enzyme 2 (ACE2). The antibodies as described herein may be optimized using methods as described herein to have improved specificity, sensitivity, accuracy, and reliability.

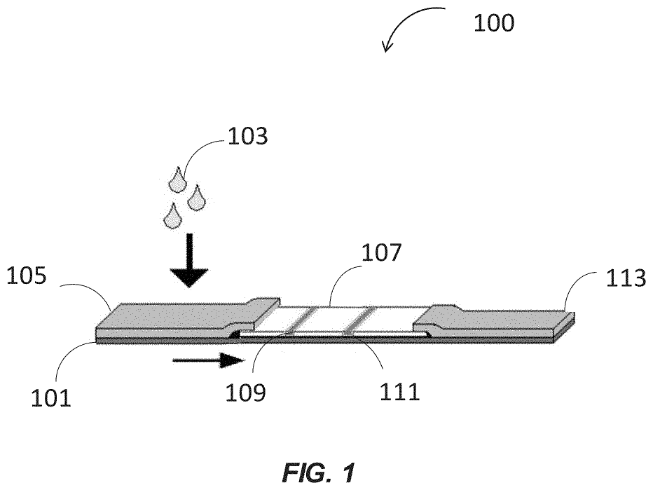

The antibodies as described herein may be used for a device for detecting SARS-CoV-2. FIG. 1 shows an example of a virus being captured and detected using a device with respect to some embodiments disclosed herein. The device 100 is a lateral flow device. In some instances the device comprises a backing 101. In some instances, the backing is a solid backing. The solid backing may comprise any suitable material including, but not limited to, plastic, fiber, and glass. In some instances, the backing comprises an adhesive. A sample 103 is applied to the sample application pad 105. In some instances, the sample is a biological sample. In some instances, the biological sample is a fluid (e.g., bodily fluid) sample. In some instances, the fluid is saliva, blood, semen, vaginal fluid, or urine. In some instances, the fluid is saliva. The sample then travels to the membrane substrate 107 and the capture and detection process occurs on a test line 109. The test line may comprise an immobilized antibody. In some instances, the immobilized antibody is an antibody to detect SARS-CoV, MERS-CoV, CoV-229E, HCoV-NL63, HCoV-OC43, or HCoV-HKU1. In some embodiments, the antibody detects SARS-CoV-2. In some embodiments, the antibody detects a receptor that binds to the coronavirus. In some embodiments, the receptor of the coronavirus is ACE2 or dipeptidyl peptidase 4 (DPP4). In some instances, the antibodies are conjugated with latex via Amide Beads. The membrane substrate also includes a control line 111. The device may further comprise a wicking pad 113. Wicking pads may prevent backflow. In some instances, the wicking pad comprises a cellulose filter. Results from the test line and the control line may be visible, for example, as a color or a color change. In some instances, the results from the test line and the control line are compared. Results from the device may then be transferred. In some instances, the results are transferred, wirelessly or through a cable, to a computerized device to process and display the information. In some embodiments, the result is transmitted to a software program on a computerized device, where the computerized device has a graphical user interface that displays the assay results. In some instances, the results are transferred to a database. In some instances, the results from the database are used for bioinformatics applications such as functional genomics and homology searching.

Described herein are methods and devices for detecting SARS-CoV-2, wherein the device can comprise a sample application pad for receiving the sample. In some instances, the sample application pad further comprises a buffer, or pH calibrator, a peptide, or an antibody. In some instances, the buffer is a running/chase buffer. The running buffer is a component of lateral flow assay and may depend upon the choice of the conjugate conditions, membrane selection criteria, sample matrix, and sample pad material. The running buffer may facilitate the flow of the fluid in the detection and diagnostic device. In some cases, the sample application pad comprises a phosphate-buffered saline, blocking buffer (e.g., casein or Tween reagent), a surfactant, additives, and other reagents to increase sensitivity of the assay. In some instances, the running buffer comprises phosphate buffer comprising casein, BSA, and Tween 20. In some instances, the running buffer comprises 1×PBS, 0.25% Casein, 0.5% BSA, and 2% Tween20. In some instances, the buffer is a citrate buffer.

Various types of samples can be used with the methods and devices described herein. In some instances, the sample is a biological sample. In some instances, the biological sample is a fluid (e.g., bodily fluid). In some instances, the fluid is saliva, blood, semen, vaginal fluid, or urine. In some instances, the fluid is saliva. In some instances, the sample is collected from a human subject or an animal subject. In some cases, the sample is collected by means including but not limited to spitting, wiping saliva, nasal swab, mouth swab, or urinating. The sample may then be transferred to the sample pad. In some cases, the sample is directly collected on the sample application pad such as by spitting on the sample application pad.

A small volume of sample is required to practice the systems and devices as described herein. In some embodiments, a suitable amount of sample applied to the sample pad is about 5 uL to about 50 uL. In some embodiments, a suitable amount of sample applied to the sample pad is at least about 5 uL. In some embodiments, a suitable amount of sample applied to the sample pad is at most about 500 uL. In some embodiments, a suitable amount of sample applied to the sample pad is about 500 uL, 1000 uL, 1500 uL, 2000 uL, 2500 uL, 3000 uL, 3500 uL, 4000 uL, 4500 uL, or 5000 uL. In some embodiments, a suitable amount of sample applied to the sample pad is about 500 uL to about 1000 uL, about 500 uL to about 1500 uL, about 500 uL to about 2000 uL, about 500 uL to about 2500 uL, about 500 uL to about 3000 uL, about 500 uL to about 2500 uL, about 500 uL to about 4000 uL, about 500 uL to about 4500 uL, about 500 uL to about 5000 uL, about 1000 uL to about 1500 uL, about 1000 uL to about 2000 uL, about 1000 uL to about 2500 uL, about 1000 uL to about 3000 uL, about 1000 uL to about 2500 uL, about 1000 uL to about 4000 uL, about 1000 uL to about 4500 uL, about 1000 uL to about 5000 uL, about 1500 uL to about 2000 uL, about 1500 uL to about 2500 uL, about 1500 uL to about 3000 uL, about 1500 uL to about 2500 uL, about 1500 uL to about 4000 uL, about 1500 uL to about 4500 uL, about 1500 uL to about 5000 uL, about 2000 uL to about 2500 uL, about 2000 uL to about 3000 uL, about 2000 uL to about 2500 uL, about 2000 uL to about 4000 uL, about 2000 uL to about 4500 uL, about 2000 uL to about 5000 uL, about 2500 uL to about 3000 uL, about 2500 uL to about 2500 uL, about 2500 uL to about 4000 uL, about 2500 uL to about 4500 uL, about 2500 uL to about 5000 uL, about 3000 uL to about 2500 uL, about 3000 uL to about 4000 uL, about 3000 uL to about 4500 uL, about 3000 uL to about 5000 uL, about 2500 uL to about 4000 uL, about 2500 uL to about 4500 uL, about 2500 uL to about 5000 uL, about 4000 uL to about 4500 uL, about 4000 uL to about 5000 uL, or about 4500 uL to about 5000 uL.

Following application of the sample to the sample application pad, the virus is detected on a membrane. In some instances, the membrane comprises woven mesh, cellulose filters, glass fiber, mixed glass fiber and cellulose, synthetic fiber, mixed fiber, surface modified plastic (polyester, polypropylene, or polyethylene), graded density polyethersulfone (PES), or combinations thereof. In some aspects, the membrane comprises nitrocellulose.

The membrane substrate can comprise any suitable form. In some instances, the membrane is in a form of a strip. In some instances, the membrane is in a form of a circle.

The membrane substrate may be modified into a predefined dimension to control the speed and accuracy of the test. In some instances, the membrane substrate is about 4 millimeters (mm) to about 100 mm. In some instances, the membrane substrate is at least about 4 mm, 5 mm, 6 mm, 7 mm, 8 mm, 9 mm, 10 mm, 11 mm, 12 mm, 16 mm, 18 mm, 20 mm, 24 mm, 26 mm, 28 mm, 30 mm, 40 mm, 50 mm, 60 mm, or more than 60 mm by about 4 mm, 5 mm, 6 mm, 7 mm, 8 mm, 9 mm, 10 mm, 11 mm, 12 mm, 16 mm, 18 mm, 20 mm, 24 mm, 26 mm, 28 mm, 30 mm, 40 mm, 50 mm, 60 mm, or more than 60 mm. In some instances, the membrane substrate is at least about 4 mm, 5 mm, 6 mm, 7 mm, 8 mm, 9 mm, 10 mm, 11 mm, 12 mm, 16 mm, 18 mm, 20 mm, 24 mm, 26 mm, 28 mm, 30 mm, 40 mm, 50 mm, 60 mm, or more than 60 mm in width. the membrane substrate is at least about 4 mm, 5 mm, 6 mm, 7 mm, 8 mm, 9 mm, 10 mm, 11 mm, 12 mm, 16 mm, 18 mm, 20 mm, 24 mm, 26 mm, 28 mm, 30 mm, 40 mm, 50 mm, 60 mm, or more than 60 mm in length. In some instances, the membrane substrate is at least about 4 mm, 5 mm, 6 mm, 7 mm, 8 mm, 9 mm, 10 mm, 11 mm, 12 mm, 16 mm, 18 mm, 20 mm, 24 mm, 26 mm, 28 mm, 30 mm, 40 mm, 50 mm, 60 mm, or more than 60 mm in thickness. In some aspects, the membrane substrate is about 8 mm in thickness.

In some instances, at least a portion of the membrane is supported by a solid backing. The solid backing may comprise any suitable material including, but not limited to, plastic, fiber, and glass. In some instances, the backing comprises an adhesive.

In some instances, the membrane comprises a first test location on the membrane. In some instances, the first test location is a surface that is amenable to antibody immobilization. In some instances, the first test location comprises a reagent that binds to an analyte in the sample. The analyte may be a virus. In some instances, the analyte is SARS-CoV-2. The reagent that binds to the analyte, in some instances, is an antibody.

As used herein, the term antibody will be understood to include proteins having the characteristic two-armed, Y-shape of a typical antibody molecule as well as one or more fragments of an antibody that retain the ability to specifically bind to an antigen. Exemplary antibodies include, but are not limited to, a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL, VH, CL, and CH1 domains), a F(ab′)2 fragment (including bivalent fragments comprising two Fab fragments linked by a disulfide bridge at the hinge region), a Fd fragment (including fragments comprising the VH and CH1 fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of only a single monomeric variable domain, disulfide-linked Fvs (sdFv), an intrabody, an anti-idiotypic (anti-Id) antibody, or ab antigen-binding fragments thereof. In some instances, the libraries disclosed herein comprise nucleic acids encoding for an antibody, wherein the antibody is a Fv antibody, including Fv antibodies comprised of the minimum antibody fragment which contains a complete antigen-recognition and antigen-binding site. In some embodiments, the Fv antibody consists of a dimer of one heavy chain and one light chain variable domain in tight, non-covalent association, and the three hypervariable regions of each variable domain interact to define an antigen-binding site on the surface of the VH-VL dimer. In some embodiments, the six hypervariable regions confer antigen-binding specificity to the antibody. In some embodiments, a single variable domain (or half of an Fv comprising only three hypervariable regions specific for an antigen, including single domain antibodies isolated from camelid animals comprising one heavy chain variable domain such as VHH antibodies or nanobodies) has the ability to recognize and bind antigen. In some instances, the libraries disclosed herein comprise nucleic acids encoding for an antibody, wherein the antibody is a single-chain Fv or scFv, including antibody fragments comprising a VH, a VL, or both a VH and VL domain, wherein both domains are present in a single polypeptide chain. In some embodiments, the Fv polypeptide further comprises a polypeptide linker between the VH and VL domains allowing the scFv to form the desired structure for antigen binding. In some instances, a scFv is linked to the Fc fragment or a VHH is linked to the Fc fragment (including minibodies). In some instances, the antibody comprises immunoglobulin molecules and immunologically active fragments of immunoglobulin molecules, e.g., molecules that contain an antigen binding site. Immunoglobulin molecules are of any type (e.g., IgG, IgE, IgM, IgD, IgA and IgY), class (e.g., IgG 1, IgG 2, IgG 3, IgG 4, IgA 1 and IgA 2) or subclass.

Methods and systems as described herein may comprise various test lines. In some instances, the first test line comprises at least one immobilized antibody coupled to the membrane. In some instances, the first test line comprises at least two immobilized antibodies coupled to the membrane. In some instances, an immobilized antibody targets or detects a coronavirus. In some instances, the immobilized antibody targets or detects a structural protein of a virus, or a fragment of a viral protein. In some instances, the immobilized antibody targets or detects a spike protein, a membrane protein, an envelope protein, a nucleocapsid protein, or combinations thereof. In some instances, the immobilized antibody targets or detects an angiotensin converting enzyme 2. In some instances, the at least two immobilized antibodies detect a spike protein and a nucleocapsid protein.

In some instances, the second test line is a control line. In some embodiments, the control line comprises an antibody or antibody fragment. In some instances, the antibody is a mouse, rat, rabbit, cat, dog, goat, chicken, bovine, horse, llama, camel, dromedary, shark, non-human primate, human, or humanized antibody. In some instances, the first test line is placed upstream of the control line. In some instances, the first line is placed downstream of the control line. In some instances, the control line is compared to the first test line.

Antibodies described herein for use with the test device for detecting SARS-CoV-2 may be optimized by the design of in-silico libraries comprising variant sequences of an input antibody sequence. Input sequences are in some instances modified in-silico with one or more mutations to generate libraries of optimized sequences. In some instances, such libraries are synthesized, cloned into expression vectors, and translation products (antibodies) evaluated for activity. In some instances, fragments of sequences are synthesized and subsequently assembled. In some instances, expression vectors are used to display and enrich desired antibodies, such as phage display. Selection pressures used during enrichment in some instances includes, but is not limited to, binding affinity, toxicity, immunological tolerance, stability, receptor-ligand competition, or developability. Such expression vectors allow antibodies with specific properties to be selected (“panning”), and subsequent propagation or amplification of such sequences enriches the library with these sequences. Panning rounds can be repeated any number of times, such as 1, 2, 3, 4, 5, 6, 7, or more than 7 rounds. Sequencing at one or more rounds is in some instances used to identify which sequences have been enriched in the library.

Described herein are methods and systems of in-silico library design. For example, an antibody or antibody fragment sequence is used as input. In some instances, the antibody sequence used as input is an antibody or antibody fragment sequence that binds SARS-CoV-2. In some instances, the input is an antibody or antibody fragment sequence that binds a protein of SARS-CoV-2. In some instances, the protein is a spike glycoprotein, a membrane protein, an envelope protein, a nucleocapsid protein, or combinations thereof. In some instances, the protein is a spike glycoprotein of SARS-CoV-2. In some instances, the protein is a receptor binding domain of SARS-CoV-2. In some instances, the input sequence is an antibody or antibody fragment sequence that binds angiotensin-converting enzyme 2 (ACE2). In some instances, the input sequence is an antibody or antibody fragment sequence that binds an extracellular domain of the angiotensin-converting enzyme 2 (ACE2).

In some instances, the antibodies described herein are optimized by assaying for functional activity, structural stability (e.g., thermal stable or pH stable), expression, specificity, or a combination thereof. In some instances, the antibodies are assayed for antibody capable of folding. In some instances, a region of the antibody is assayed for functional activity, structural stability, expression, specificity, folding, or a combination thereof.

Antibodies to be used with the methods and systems as described herein may comprise a sequence set forth in Table 1 or Tables 9-14. In some embodiments, the sequence comprises at least or about 70%, 80%, 85%, 90%, 91%, 92%, 93%, 94%, 95%, 96%, 97%, 98%, 99%, or 100% sequence identity to any one of SEQ ID NOs: 1, 2, 3, 4, 5, 6, 7, 8, 9, 10, 11, 12, 13, 14, 15, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 81, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, or 141. In some instances, the sequence comprises at least or about 95% homology to any one of SEQ ID NOs: 1, 2, 3, 4, 5, 6, 7, 8, 9, 10, 11, 12, 13, 14, 15, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 81, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, or 141. In some instances, the sequence comprises at least or about 97% homology to any one of SEQ ID NOs: 1, 2, 3, 4, 5, 6, 7, 8, 9, 10, 11, 12, 13, 14, 15, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 81, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, or 141. In some instances, the sequence comprises at least or about 99% homology to any one of SEQ ID NOs: 1, 2, 3, 4, 5, 6, 7, 8, 9, 10, 11, 12, 13, 14, 15, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 81, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, or 141. In some instances, the sequence comprises at least or about 100% homology to any one of SEQ ID NOs: 1, 2, 3, 4, 5, 6, 7, 8, 9, 10, 11, 12, 13, 14, 15, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 81, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, or 141. In some instances, the sequence comprises at least a portion having at least or about 10, 20, 30, 40, 50, 60, 70, 80, 90, 100, 110, or more than 110 amino acids of any one of SEQ ID NOs: 1, 2, 3, 4, 5, 6, 7, 8, 9, 10, 11, 12, 13, 14, 15, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 81, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, or 141.

TABLE 1
SARS-CoV-2 Variant Light Chain (LC) Variable Domain and Heavy
Chain (HC) Variable Domain Sequences

| Construct | | SEQ ID |
|---|---|---|
| Description | Amino Acid Sequence | NO: |
| Ab-1 LC | DIQMTQSPSSLSASVGDRVTITCRASQSIDTYLNWYQQKPGKA | 1 |
| | PKLLIYAASALASGVPSRFSGSGSGTDFTLTISSLQPEDFATYYC | |
| | QQSYSAPPYTFGQGTKVEIK | |
| Ab-1 HC | EVQLLESGGGLVQPGGSLRLSCAASGFTFSNYPMNWVRQAPG | 2 |
| | KGLEWVSTISGSGGNTFYADSVKGRFTISRDNSKNTLYLQMNS | |
| | LRAEDTAVYYCVRHDEYSFDYWGQGTLVTVSS | |
| Ab-2 LC | QSALTQPASVSGSPGQSITISCTGTSSDVGHYNLVSWYQQHPG | 3 |
| | KAPKLMIYEGTKRPSGVSNRFSGSKSGNTASLTISGLQAEDEA | |
| | DYYCCSYAGSSSFVVFGGGTKLTVL | |
| Ab-2 HC | EVQLLESGGGLVQPGGSLRLSCAASGITFSSYAMSWVRQAPG | 4 |
| | KGLEWVSGISGSGGSTYYADSVKGRFTISRDNSKNTLYLQMNS | |
| | LRAEDTAVYYCAKHGSGTIFGVVIAKYYFDYWGQGTLVTVSS | |
| Ab-3 LC | DIQMTQSPSSLSASVGDRVTITCRASQTINTFLNWYQQKPGKA | 5 |
| | PKLLIYSASTLQSGVPSRFSGSGSGTDFTLTISSLQPEDFATYYC | |
| | QQSYSTFTFGGGTKVEIK | |
| Ab-3 HC | EVQLLESGGGLVQPGGSLRLSCAASGFTFSRHAMNVVVRQAPG | 6 |
| | KGLEWVSGITGSGDETYYADSVKGRFTISRDNSKNTLYLQMN | |
| | SLKAEDTAVYYCARDLPASYYDSSGYYWHNGMDVWGQGTL | |
| | VTVSS | |
| Ab-4 LC | DIQMTQSPSSLSASVGDRVTITCRASQTINTYLNWYQQKPGKA | 7 |
| | PKLLIYSASTLQSGVPSRFSGSGSGTDFTLTISSLQPEDFATYYC | |
| | QQSYSTFTFGQGTKVEIK | |
| Ab-4 HC | EVQLLESGGGLVQPGGSLRLSCAASGFTFSRHAMNWVRQAPG | 8 |
| | KGLEWVSGISGSGDETYYADSVKGRFTISRDNSKNTLYLQMN | |
| | SLRAEDTAVYYCARDLPASYYDSSGYYWHNGMDVWGQGTL | |
| | VTVSS | |
| Ab-5 LC | QSALTQPASVSGSPGQSITISCTGTSSDVGSYYLVSWYQQHPG | 9 |
| | KAPKLMIYEGDKRPSGVSNRFSGSKSGNTASLTISGLQAEDEA | |
| | DYYCCSHAGRYPYVFGGGTKLTVL | |
| Ab-5 HC | EVQLLESGGGLVQPGGSLRLSCAASGFMFSSYAMSWVRQAPG | 10 |
| | KGLEWVSAISGSGGSTYYTDSVKGRFTISRDNSKNTLYLQMNS | |
| | LRAEDTAVYYCAKDGASGWPNWHFDLWGQGTLVTVSS | |
| Ab-6 LC | QSALTQPASVSGSPGQSITISCTGTSSDVGSYSLVSWYQQHPGK | 11 |
| | APKLMIYEGTKRPSGVSNRFSGSKSGNTASLTISGLQAEDEAD | |
| | YYCCSYAGSYSYVVFGGGTKLTVL | |
| Ab-6 HC | EVQLLESGGGLVQPGGSLRLSCAASGFTFSNYAMSWVRQAPG | 12 |
| | KGLEWVSDISGSGGSTYYADSVKGRFTISRDNSKNTLYLQMNS | |
| | LRAEDTAVYYCVKGTIPIFGVIRSAFDYWGQGTLVTVSS | |
| Ab-7 LC | DIQMTQSPSSLSASVGDRVTITCRASQSIHTYLNWYQQKPGKA | 13 |
| | PKLLIYAASALASGVPSRFSGSGSGTDFTLTISSLQPEDFATYYC | |
| | QQSYSAPPYTFGQGTKVEIK | |
| Ab-7 HC | EVQLLESGGGLVQPGGSLRLSCAASGFTFSDFAMAWVRQAPG | 14 |
| | KGLEWVSAISGSGDITYYADSVKGRFTISRDNSKNTLYLQMNS | |
| | LRAEDTAVYYCAREADGLHSPWHFDLWGQGTLVTVSS | |
| Ab-8 HC | EVQLVESGGGLVQPGGSLRLSCAASGFNVNDYAMGWFRQAP | 15 |
| | GKEREFVAGITSSVGVTNYADSVKGRFTISADNSKNTAYLQM | |
| | NSLKPEDTAVYYCAADIFFVNVVGRGTLVTVSS | |
| Ab-9 HC | EVQLVESGGGLVQPGGSLRLSCAASGFTLDYYAMGWFRQAP | 61 |
| | GKEREFVAAINWSGDNTHYADSVKGRFTISADNSKNTAYLQM | |
| | NSLKPEDTAVYYCARAPFYCTTTKCQDNYYYMDVWGQGTLV | |
| | TVSS | |
| Ab-10 HC | EVQLVESGGGLVQPGGSLRLSCAASGGTFSSIGMGWFRQAPG | 62 |
| | KEREFVAAISWDGGATAYADSVKGRFTISADNSKNTAYLQMN | |
| | SLKPEDTAVYYCAKEDVGKPFDWGQGTLVTVSS | |
| Ab-11 | EVQLVESGGGLVQPGGSLRLSCAASGFTLGDYVMGWFRQAP | 63 |
| | GKEREFVAAIHSGGSTYADSVKGRFTISADNSKNTAYLQMNSL | |
| | KPEDTAVYYCAAKEYGGTRRYDRAYNWGQGTLVTVSS | |
| Ab-12 | EVQLVESGGGLVQPGGSLRLSCAASGGGTFGSYAMGWFRQAP | 64 |
| | GKERELVAAISSGGSTNYADSVKGRFTISADNSKNTAYLQMNS | |
| | LKPEDTAVYYCARGDWRYGWGQGTLVTVSS | |
| Ab-13 | EVQLVESGGGLVQPGGSLRLSCAASGRTYSISAMGWFRQAPG | 65 |
| | KEREFVAAISMSGDDSAYADSVKGRFTISADNSKNTAYLQMN | |
| | SLKPEDTAVYYCAAQLGYESGYSLTYDYDWGQGTLVTVSS | |
| Ab-14 | EVQLVESGGGLVQPGGSLRLSCAASGGTFSTYPMGWFRQAPG | 66 |
| | KEREFVAAITSDGSTLYADSVKGRFTISADNSKNTAYLQMNSL | |
| | KPEDTAVYYCAATDYNKAYAREGRRYDWGQGTLVTVSS | |
| Ab-15 | EVQLVESGGGLVQPGGSLRLSCAASGSIFRINAMGWFRQAPGK | 67 |
| | EREFVAAIHWSGSSTRYADSVKGRFTISADNSKNTAYLQMNSL | |
| | KPEDTAVYYCAAQDRRRGDYYTFDYHWGQGTLVTVSS | |
| Ab-16 | EVQLVESGGGLVQPGGSLRLSCAASGGTFNNYAMGWFRQAP | 68 |
| | GKERELVAAITSGGSTDYADSVKGRFTISADNSKNTAYLQMNS | |
| | LKPEDTAVYYCARGDWRYGWGQGTLVTVSS | |
| Ab-17 | EVQLVESGGGLVQPGGSLRLSCAASGTIVNINVMGWFRQAPG | 69 |
| | KEREFVAAIHWSGGLKAYADSVKGRFTISADNSKNTAYLQMN | |
| | SLKPEDTAVYYCAMNRAGIYEWGQGTLVTVSS | |
| Ab-18 | EVQLVESGGGLVQPGGSLRLSCAASGSTFSNYAMGWFRQAPG | 70 |
| | KERELVAAITSGGSTSYADSVKGRFTISADNSKNTAYLQMNSL | |
| | KPEDTAVYYCARGDWRYGWGQGTLVTVSS | |
| Ab-19 | EVQLVESGGGLVQPGGSLRLSCAASGFSFDDYVMGWFRQAPG | 71 |
| | KEREFVAAISRSGNLKSYADSVKGRFTISADNSKNTAYLQMNS | |
| | LKPEDTAVYYCAAKEYGGTRRYDRAYNWGQGTLVTVSS | |
| Ab-20 | EVQLVESGGGLVQPGGSLRLSCAASGSAFRSTVMGWFRQAPG | 72 |
| | KEREFVAAVIGSSGITDYADSVKGRFTISADNSKNTAYLQMNS | |
| | LKPEDTAVYYCARGDWRYGWGQGTLVTVSS | |
| Ab-21 | EVQLVESGGGLVQPGGSLRLSCAASGRTFSDAGMGWFRQAPG | 73 |
| | KEREFVAAISRSGNLKAYADSVKGRFTISADNSKNTAYLQMNS | |
| | LKPEDTAVYYCAVQVNGTWAWGQGTLVTVSS | |
| Ab-22 | EVQLVESGGGLVQPGGSLRLSCAASGFTLDYYAMGWFRQAP | 74 |
| | GKERELVAAISWNGGSTSYADSVKGRFTISADNSKNTAYLQM | |
| | NSLKPEDTAVYYCARGDWRYGWGQGTLVTVSS | |
| Ab-23 | EVQLVESGGGLVQPGGSLRLSCAASGGTFSTYVMGWFRQAPG | 75 |
| | KEREFVAAISWSGESTLYADSVKGRFTISADNSKNTAYLQMNS | |
| | LKPEDTAVYYCAADLMYGVDRRYDWGQGTLVTVSS | |
| Ab-24 | EVQLVESGGGLVQPGGSLRLSCAASGISSSKRNMGWFRQAPG | 76 |
| | KEREFVAGISWTGGITYYADSVKGRFTISADNSKNTAYLQMNS | |
| | LKPEDTAVYYCAIAGRGRWGQGTLVTVSS | |
| Ab-25 | EVQLVESGGGLVQPGGSLRLSCAASGRRFSAYGMGWFRQAPG | 77 |
| | KEREFVAVISRSGTLTRYADSVKGRFTISADNSKNTAYLQMNS | |
| | LKPEDTAVYYCASSGPADARNGERWHWGQGTLVTVSS | |
| Ab-26 | EVQLVESGGGLVQPGGSLRLSCAASGLTFSSFVMGWFRQAPG | 78 |
| | KEREFVAAISSNGGSTRYADSVKGRFTISADNSKNTAYLQMNS | |
| | LKPEDTAVYYCAAKEYGGTRRYDRAYNWGQGTLVTVSS | |
| Ab-27 | EVQLVESGGGLVQPGGSLRLSCAASGTVFSISAMGWFRQAPG | 79 |
| | KEREFVAAISMSGDDTAYADSVKGRFTISADNSKNTAYLQMN | |
| | SLKPEDTAVYYCAAQLGYESGYSLTYDYDWGQGTLVTVSS | |
| Ab-28 | EVQLVESGGGLVQPGGSLRLSCAASGSIFSPNVMGWFRQAPG | 80 |
| | KEREFVAAITNGGSTKYADSVKGRFTISADNSKNTAYLQMNSL | |
| | KPEDTAVYYCAAQRWRGGSYEWGQGTLVTVSS | |
| Ab-29 | EVQLVESGGGLVQPGGSLRLSCAASGIPASIRVMGWFRQAPGK | 81 |
| | EREFVAAIHWSGSSTRYADSVKGRFTISADNSKNTAYLQMNSL | |
| | KPEDTAVYYCALSRAIVPGDSEYDYRWGQGTLVTVSS | |
| Ab-30 | EVQLVESGGGLVQPGGSLRLSCAASGRTFSMSAMGWFRQAPG | 82 |
| | KEREFVSAISWSGGSTLYADSVKGRFTISADNSKNTAYLQMNS | |
| | LKPEDTAVYYCAAQLGYESGYSLTYDYDWGQGTLVTVSS | |
| Ab-31 | EVQLVESGGGLVQPGGSLRLSCAASGRTFSNYAMGWFRQAPG | 83 |
| | KERELVAAITSGGSTDYADSVKGRFTISADNSKNTAYLQMNSL | |
| | KPEDTAVYYCARGDWRYGWGQGTLVTVSS | |
| Ab-32 | EVQLVESGGGLVQPGGSLRLSCAASGRTFSSYAMGWFRQAPG | 84 |
| | KERELVAAISTGGSTYYADSVKGRFTISADNSKNTAYLQMNSL | |
| | KPEDTAVYYCARGDWRYGWGQGTLVTVSS | |
| Ab-33 | EVQLVESGGGLVQPGGSLRLSCAASGRSFSSVGMGWFRQAPG | 85 |
| | KEREFVAVISRSGASTAYADSVKGRFTISADNSKNTAYLQMNS | |
| | LKPEDTAVYYCASAGPADARNGERWAWGQGTLVTVSS | |
| Ab-34 | EVQLVESGGGLVQPGGSLRLSCAASGRAFRRYTMGWFRQAPG | 86 |
| | KERELIAVINWSGDRRYYADSVKGRFTISADNSKNTAYLQMN | |
| | SLKPEDTAVYYCAATLAKGGGRWGQGTLVTVSS | |
| Ab-35 | EVQLVESGGGLVQPGGSLRLSCAAMAWAGFARRRAKNAKW | 87 |
| | WRALPRGGPTYADSVKGRFTISADNSKNTAYLQMNSLKPEDT | |
| | AVYYCAAGGMWYGSSLYVRFDLLEDGMDWGQGTLVTVSS | |
| Ab-36 | EVQLVESGGGLVQPGGSLRLSCAASGSISSINGMGWFRQAPGK | 88 |
| | ERELVALISRSGGTTYYADSVKGRFTISADNSKNTAYLQMNSL | |
| | KPEDTAVYYCASAGPADARNGERWAWGQGTLVTVSS | |
| Ab-37 | EVQLVESGGGLVQPGGSLRLSCAASGRTFSNNVMGWFRQAPG | 89 |
| | KERELVAAAISGGSTYYADSVKGRFTISADNSKNTAYLQMNSL | |
| | KPEDTAVYYCARGDWRYGWGQGTLVTVSS | |
| Ab-38 | EVQLVESGGGLVQPGGSLRLSCAASGRTFSISAMGWFRQAPG | 90 |
| | KEREFVAAISRSGTTMYADSVKGRFTISADNSKNTAYLQMNSL | |
| | KPEDTAVYYCAAQLGYESGYSLTYDYDWGQGTLVTVSS | |
| Ab-39 | EVQLVESGGGLVQPGGSLRLSCAASGGTFSYYDLAAMGWFR | 91 |
| | QAPGKEREFVAAISWSQYNTKYADSVKGRFTISADNSKNTAY | |
| | LQMNSLKPEDTAVYYCAARVVVRTAHGFEDNWGQGTLVTVSS | |
| Ab-40 | EVQLVESGGGLVQPGGSLRLSCAASGRTFNNYGMGWFRQAP | 92 |
| | GKEREFVAVISRSGSLKAYADSVKGRFTISADNSKNTAYLQMN | |
| | SLKPEDTAVYYCASDPTYGSGRWTWGQGTLVTVSS | |
| Ab-41 | EVQLVESGGGLVQPGGSLRLNCAASGFTLDDYVMGWFRQTP | 93 |
| | GKEREFVAAISSSGALTSYADSVKGRFTISADNSKNTAYLQMN | |
| | SLKPEDAAVYYCAAKEYGGTRRYDRAYNWGQGTLVTVSS | |
| Ab-42 | EVQLVESGGGLVQPGGSLRLSCAASGRTFNAMGWFRQAPGKE | 94 |
| | REFVAAIRWSGDMSVYADSVKGRFTISADNSKNTAYLQMNSL | |
| | KPEDTAVYYCAAQDRRRGDYYTFDYHWGQGTLVTVSS | |
| Ab-43 | EVQLVESGGGLVQPGGSLRLSCAASGLTFSTYAMGWFRQAPG | 95 |
| | KEREFVAAITSGGSTDYADSVKGRFTISADNSKNTAYLQMNSL | |
| | KPEDTAVYYCARGDWRYGWGQGTLVTVSS | |
| Ab-44 | EVQLVESGGGLVQPGGSLRLSCAASGSIFTINAMGWFRQAPGK | 96 |
| | EREGVAAIGSDGSTSYADSVKGRFTISADNSKNTAYLQMNSLK | |
| | PEDTAVYYCAVVRWGADWGQGTLVTVSS | |
| Ab-45 | EVQLVESGGGLVQPGGSLRLSCAASGLTFSSYAMGWFRQAPG | 97 |
| | KERELVAAITSSSGSTPAYADSVKGRFTISADNSKNTAYLQMN | |
| | SLKPEDTAVYYCARGDWRYGWGQGTLVTVSS | |
| Ab-46 | EVQLVESGGGLVQPGGSLRLSCAASGIPFSTRTMGWFRQAPGK | 98 |
| | EREFVAAISWSQYNTKYADSVKGRFTISADNSKNTAYLQMNS | |
| | LKPEDTAVYYCAARHWGMFSRSENDYNWGQGTLVTVSS | |
| Ab-47 | EVQLVESGGGLVQPGGSLRLSCAASGRSRFSTYVMGWFRQAP | 99 |
| | GKEREFVAAISWSQYNTKYADSVKGRFTISADNSKNTAYLQM | |
| | NSLKPEDTAVYYCAAGNGGRNYGHSRARYDWGQGTLVTVSS | |
| Ab-48 | EVQLVESGGGLVQPGGSLRLSCAASGLTLSSYGMGWFRQAPG | 100 |
| | KEREYVAVISRSGSLKAYADSVKGRFTISADNSKNTAYLQMNS | |
| | LKPEDTAVYYCATRADAEGWWDWGQGTLVTVSS | |
| Ab-49 | EVQLVESGGGLVQPGGSLRLSCAASGSIFRVNVMGWFRQAPG | 101 |
| | KEREFVAAINNFGTTKYADSVKGRFTISADNSKNTAYLQMNSL | |
| | KPEDTAVYYCAADLPSRWGQGTLVTVSS | |
| Ab-50 | EVQLVESGGGLVQPGGSLRLSCAASGRTFRNYAMGWFRQAP | 102 |
| | GKERELVAAISSGGSTDYADSVKGRFTISADNSKNTAYLQMNS | |
| | LKPEDTAVYYCARGDWRYGWGQGTLVTVSS | |
| Ab-51 | EVQLVESGGGLVQPGGSLRLSCAASGRTFSSFAMGWFRQAPG | 103 |
| | KERELVAAISSGGSTNYADSVKGRFTISADNSKNTAYLQMNSL | |
| | KPEDTAVYYCARGDWRYGWGQGTLVTVSS | |
| Ab-52 | EVQLVESGGGLVQPGGSLRLSCAASGTTFRINAMGWFRQAPG | 104 |
| | KEREFVAAMNWSGGSTKYADSVKGRFTISADNSKNTAYLQM | |
| | NSLKPEDTAVYYCAAQDRRRGDYYTFDYHWGQGTLVTVSS | |
| Ab-53 | EVQLVESGGGLVQPGGSLRLSCAASGFTLGDYVMGWFRQAP | 105 |
| | GKEREFVAAIHSGGSTLYADSVKGRFTISADNSKNTAYLQMNS | |
| | LKPEDTAVYYCAAKEYGGTRRYDRTYNWGQGTLVTVSS | |
| Ab-54 | EVQLVESGGGLVQPGGSLRLSCAASGFTFSRSAMGWFRQAPG | 106 |
| | KERELVAGILSSGATVYADSVKGRFTISADNSKNTAYLQMNSL | |
| | KPEDTAVYYCAKAPRDWGQGTLVTVSS | |
| Ab-55 | EVQLVESGGGLVQPGGSLRLSCAASGRTFNNYAMGWFRQAP | 107 |
| | GKERELVAAITSGGSTDYADSVKGRFTISADNSKNTAYLQMNS | |
| | LKPEDTAVYYCARGDWRYGWGQGTLVTVSS | |
| Ab-56 | EVQLVESGGGLVQPGGSLRLSCAASGFTFRSYPMGWFRQAPG | 108 |
| | KEREFVAAINNFGTTKYADSVKGRFTISADNSKNTAYLQMNSL | |
| | KPEDTAVYYCAAAKGIGVYGWGQGTLVTVSS | |
| Ab-57 | EVQLVESGGGLVQPGGSLRLSCAASGNIFTRNVMGWFRQAPG | 109 |
| | KEREFVAAIHWNGDSTKYADSVKGRFTISADNSKNTAYLQMN | |
| | SLKPEDTAVYYCAAGSNIGGSRWRYDWGQGTLVTVSS | |
| Ab-58 | EVQLVESGGGLVQPGGSLRLSCAASGRTISRYTMGWFRQAPG | 110 |
| | KERELVAAIKWSGASTVYADSVKGRFTISADNSKNTAYLQMN | |
| | SLKPEDTAVYYCAAKGIWDYLGRRDFGDWGQGTLVTVSS | |
| Ab-59 | EVQLVESGGGLVQPGGSLRLSCAASGFRFSSYGMGWFRQAPG | 111 |
| | KEREFVAIITSGGLTVYADSVKGRFTISADNSKNTAYLQMNSL | |
| | KPEDTAVYYCAARKTFYFGTSSYPNDYAWGQGTLVTVSS | |
| Ab-60 | EVQLVESGGGLVQPGGSLRLSCAASGRTFDNHAMGWFRQAP | 112 |
| | GKEREGVAAIGSDGSTSYADSVKGRFTISADNSKNTAYLQMN | |
| | SLKPEDTAVYYCAVVRWGVDWGQGTLVTVSS | |
| Ab-61 | EVQLVESGGGLVQPGGSLRLSCAASGFTFSSHAMGWFRQAPG | 113 |
| | KEREFVAGISWSGESTLTRYADSVKGRFTISADNSKNTAYLQM | |
| | NSLKPEDTAVYYCADVNGDWGQGTLVTVSS | |
| Ab-62 | EVQLVESGGGLVQPGGSLRLSCAASGMTFRLYAMGWFRQAP | 114 |
| | GKEREFVAAISWSQYNTKYADSVKGRFTISADNSKNTAYLQM | |
| | NSLKPEDTAVYYCAAQLGYESGYSLTYDYDWGQGTLVTVSS | |
| Ab-63 | EVQLVESGGGLVQPGGSLRLSCAASGGTFRKLAMGWFRQAPG | 115 |
| | KEREFVAVISWTGGSSYYADSVKGRFTISADNSKNTAYLQMN | |
| | SLKPEDTAVYYCARLTSFATWGQGTLVTVSS | |
| Ab-64 | EVQLVESGGGLVQPGGSLRLSCAASGRTFSANGMGWFRQAPG | 116 |
| | KEREFVAAISASGTLRAYADSVKGRFTISADNSKNTAYLQMNS | |
| | LKPEDTAVYYCAARSPMSPTWDWGQGTLVTVSS | |
| Ab-65 | EVQLVESGGGLVQPGGSLRLSCAASGSAFRSTVMGWFRQAPG | 117 |
| | KEREFVAAISWTGESTLYADSVKGRFTISADNSKNTAYLQMNS | |
| | LKPEDTAVYYCATGPYRSYFARSYLWGQGTLVTVSS | |
| Ab-66 | EVQLVESGGGLVQPGGSLRLSCAASGGTFDYSGMGWFRQAPG | 118 |
| | KEREFVAVVSQSGRTTYYADSVKGLFTITADNSKNTAYLQMN | |
| | LLKPEDTAVYYCPTATRPGEWDGGQGTLVTVSR | |
| Ab-67 | EVQLVESGGGLVQPGGSLRLSCAASGVFGPIRAMGWFRQAPG | 119 |
| | KERELVALMGNDGSTYADSVKGRFTISADNSKNTAYLQMNSL | |
| | KPEDTAVYYCAAIGWRWGQGTLVTVSS | |
| Ab-68 | EVQLVESGGGLVQPGGSLRLSCAASGFNFNWYPMGWFRQAP | 120 |
| | GKEREFVAAIRWSGGITYYADSVKGRFTISADNSKNTAYLQM | |
| | NSLKPEDTAVYYCATGPYRSYFARSYLWGQGTLVTVSS | |
| Ab-69 | EVQLVESGGGLVQPGGSLRLSCAASGMTFHRYVMGWFRQAP | 121 |
| | GKERELVASITTGGTPNYADSVKGRFTIITDNNKNTAYLLMINL | |
| | QPEDTAVYYCCKVPYIWGQGTLGTVGT | |
| Ab-70 | EVQLVESGGGLVQPGGSLRLSCAASGISTMGWFRQAPGKERE | 122 |
| | FVAAINNFGTTKYADSVKGRFTISADNSKNTAYLQMNSLKPED | |
| | TAVYYCAAASQSGSGYDWGQGTLVTVSS | |
| Ab-71 | EVQLVESGGGLVQPGGSLRLSCAASGRAFNTRAMGWFRQAP | 123 |
| | GKERELVALMGNDGSTYADSVKGRFTISADNSKNTAYLQMNS | |
| | LKPEDTAVYYCAAIGWRWGQGTLVTVSS | |
| Ab-72 | EVQLVESGGGLVQPGGSLRLSCAASGLTDRRYTMGWFRQAPG | 124 |
| | KEREFVAAINSGGSTLYADSVKGRFTISADNSKNTAYLQMNSL | |
| | KPEDTAVYYCAAGNGGRTYGHSRARYEWGQGTLVTVSS | |
| Ab-73 | EVQLVESGGGLVQPGGSLRLSCAASGRTFNVMGWFRQAPGKE | 125 |
| | RELVALMGNDGSTYADSVKGRFTISADNSKNTAYLQMNSLKP | |
| | EDTAVYYCAAVRWGVDWGQGTLVTVSS | |
| Ab-74 | EVQLVESGGGLVQPGGSLRLSCAASGRAFNTRAMGWFRQAP | 126 |
| | GKERELVALMGNDGSTNYADSVKGRFTISADNSKNTAYLQM | |
| | NSLKPEDTAVYYCAAIGWRWGQGTLVTVSS | |
| Ab-75 | EVQVVESGGGVVHPGGSVRMRCAASGVTVDYSGMGWFGQA | 127 |
| | PGKEREFVAVVSQSARTTYYADSVKGRFTISADNSKNTEYLQ | |
| | MNSMKPEDTAVYYCATATRPGEWDWGQGTLVTVSS | |
| Ab-76 | EVQLVESGGGLVQPGGSLRLSCAASGRTPRLGAMGWFRQAPG | 128 |
| | KEREFVAAISRSGGLTSYADSVKGRFTISADNSKNTAYLQMNS | |
| | LKPEDTAVYYCAAQLVGSNIGGSRWRYDWGQGTLVTVSS | |
| Ab-77 | EVQLVESGGGLVQPGGSLRLSCAASGLTFRNYAMGWFRQAPG | 129 |
| | KEREFVAAITSGGSTLYADSVKGRFTISADNSKNTAYLQMNSL | |
| | KPEDTAVYYCARGDWRYGWGHGTLVTESS | |
| Ab-78 | EVQLVESGGGLVQPGGSLRLSCAASGGRTFSDLAMGWFRQAP | 130 |
| | GKEREFVALITRSGGTTFYADSVKGRFTISADNSKNTAYLQMN | |
| | SLKPEDTAVYYCAIGRGSWGQGTLVTVSS | |
| Ab-79 | EVQLVESGGGLVQPGGSLRLSCAASGFTFGEYAMGWFRQAPG | 131 |
| | KEREFVAAVSSLGPFTRYADSVKGRFTISADNSKNTAYLQMNS | |
| | LKPEDTAVYYCAAVLDGYSGSWGQGTLVTVSS | |
| Ab-80 | EVQLVESGGGLVQPGGSLRLSCAASGFAFSSYGMGWFRQAPG | 132 |
| | KEREFVAAISWSGVRSGVSAIYADSVKGRFTISADNSKNTAYL | |
| | QMNSLKPEDTAVYYCTTDLTGDLWYFDLWGQGTLVTVSS | |
| Ab-81 | EVQLVESGGGLVQPGGSLRLSCAASGLTAGTYAMCWFRQAP | 133 |
| | GKEREGVACASSTDGSTAYADSVKGRFTISADNSKNTAYLQM | |
| | NSLKPEDTAVYYCAAVRTYGSATYDWGQGTLVTVSS | |
| Ab-82 | EVQLVESGGGLVQPGGSLRLSCAASGFTLDDYVMGWFRQAP | 134 |
| | GKERELVAAVSSLGPFTRYADSVKGRFTISADNSKNTAYLQM | |
| | NSLKPEDTAVYYCAAKEYGGTRRYDRAYNWGQGTLVTVSS | |
| Ab-83 | EVQLVESGGGLVQPGGSLRLSCAASGPTLGSYVMGWFRQAPG | 135 |
| | KEREFVAAISWSQYNTKYADSVKGRFTISADNSKNTAYLQMN | |
| | SLKPEDTAVYYCAAQRWRGGSYEWGQGTLVTVSS | |
| Ab-84 | EVQLVESGGGLVQPGGSLRLSCAASGPTFSSYVMGWFRQAPG | 136 |
| | KEREFVAAISWSQYNTKYADSVKGRFTISADNSKNTAYLQMN | |
| | SLKPEDTAVYYCAAASRSGSGYDWGQGTLVTVSS | |
| Ab-85 | EVQLVESGGGLVQPGGSLRLSCAASGYLYSKDCMGWFRQAP | 137 |
| | GKEREGVATICTGDGSTAYADSVKGRFTISADNSKNTAYLQM | |
| | NSLKPEDTAVYYCAVIAYEEGVYRWDWGQGTLVTVSS | |
| Ab-86 | EVQLVESGGGLVQPGGSLRLSCAASGFTIDDYAMGWFRQAPG | 138 |
| | KEREGVAAISGSGDDTYYADSVKGRFTISADNSKNTAYLQMN | |
| | SLKPEDTAVYYCAKLPYVSGDYWGQGTLVTVSS | |
| Ab-87 | EVQLVESGGGLVQPGGSLRLSCAASGGRFSDYGMGWFRQAP | 139 |
| | GKERELVALISRSGNLKSYADSVKGRFTISADNSKNTAYLQMN | |
| | SLKPEDTAVYYCAAKTGTSFVWGQGTLVTVSS | |
| Ab-88 | EVQLVESGGGLVQPGGSLRLSCAASGLSFSNYAMGWFRQAPG | 140 |
| | KERELVAAITSGGSTDYADSVKGRFTISADNSKNTAYLQMNSL | |
| | KPEDTAVYYCARGDWRYGWGQGTLVTVSS | |
| Ab-89 | EVQLVESGGGLVQPGGSLRLSCAASGIPSTLRAMGWFRQAPG | 141 |
| | KEREFVALINRSGGSQFYADSVKGRFTISADNSKNTAYLQMNS | |
| | LKPEDTAVYYCAIGRGSWGQGTLVTVSS | |

In some embodiments, the sequence comprises at least or about 70%, 80%, 85%, 90%, 91%, 92%, 93%, 94%, 95%, 96%, 97%, 98%, 99%, or 100% sequence identity to any one of SEQ ID NOs: 1-4212. In some instances, the sequence comprises at least or about 95% homology to any one of SEQ ID NOs: 1-4212. In some instances, the sequence comprises at least or about 97% homology to any one of SEQ ID NOs: 1-4212. In some instances, the sequence comprises at least or about 99% homology to any one of SEQ ID NOs: 1-4212. In some instances, the sequence comprises at least or about 100% homology to any one of SEQ ID NOs: 1-4212. In some instances, the sequence comprises at least a portion having at least or about 10, 20, 30, 40, 50, 60, 70, 80, 90, 100, 110, or more than 110 amino acids of any one of SEQ ID NOs: 1-4212.

Described herein, in some embodiments, are antibodies or antibody fragments comprising a variable domain, heavy chain region (VH) and a variable domain, light chain region (VL), wherein the VH comprises an amino acid sequence at least about 90% identical to a sequence as set forth in any one of SEQ ID NOs: 2927-3998, and wherein the VL comprises an amino acid sequence at least about 90% identical to a sequence as set forth in any one of SEQ ID NOs: 3999-4174. In some instances, the antibodies or antibody fragments comprise VH comprising at least or about 70%, 80%, 85%, 90%, 91%, 92%, 93%, 94%, 95%, 96%, 97%, 98%, 99%, or 100% sequence identity to any one of SEQ ID NOs: 2927-3998, and VL comprising at least or about 70%, 80%, 85%, 90%, 91%, 92%, 93%, 94%, 95%, 96%, 97%, 98%, 99%, or 100% sequence identity to any one of SEQ ID NOs: 3999-4174.

Described herein, in some embodiments, are antibodies or antibody fragments comprising a variable domain, heavy chain region (VH), wherein the VH comprises an amino acid sequence at least about 90% identical to a sequence as set forth in any one of SEQ ID NOs: 2927-3998. In some instances, the antibodies or antibody fragments comprise VH comprising at least or about 70%, 80%, 85%, 90%, 91%, 92%, 93%, 94%, 95%, 96%, 97%, 98%, 99%, or 100% sequence identity to any one of SEQ ID NOs: 2927-3998.

Described herein, in some embodiments, are antibodies or antibody fragments comprising a variable domain, light chain region (VL), wherein the VL comprises an amino acid sequence at least about 90% identical to a sequence as set forth in any one of SEQ ID NOs: 3999-4174. In some instances, the antibodies or antibody fragments comprise VL comprising at least or about 70%, 80%, 85%, 90%, 91%, 92%, 93%, 94%, 95%, 96%, 97%, 98%, 99%, or 100% sequence identity to any one of SEQ ID NOs: 3999-4174.

In some instances, an antibody described herein comprises a heavy chain variable domain complementarity determining region (CDRH) sequence as listed in Table 2. In some instances, an antibody described herein comprises a CDRH1 sequence of any one of SEQ ID NOs: 16, 19, 22, 25, 28, 31, 34, 37, 142 or 146. In some instances, an antibody described herein comprises a sequence that is at least 80% identical to a CDRH1 sequence of any one of SEQ ID NOs: 16, 19, 22, 25, 28, 31, 34, 37, 142 or 146. In some instances, an antibody described herein comprises a sequence that is at least 85% identical to a CDRH1 sequence of any one of SEQ ID NOs: 16, 19, 22, 25, 28, 31, 34, 37, 142 or 146. In some instances, an antibody described herein comprises a sequence that is at least 90% identical to a CDRH1 sequence of any one of SEQ ID NOs: 16, 19, 22, 25, 28, 31, 34, 37, 142 or 146. In some instances, an antibody described herein comprises a sequence that is at least 95% identical to a CDRH1 sequence of any one of SEQ ID NOs: 16, 19, 22, 25, 28, 31, 34, 37, 142 or 146. In some instances, an antibody described herein comprises a CDRH2 sequence of any one of SEQ ID NOs: 17, 20, 23, 26, 29, 32, 35, 38, 143 or 146. In some instances, an antibody described herein comprises a sequence that is at least 80% identical to a CDRH2 sequence of any one of SEQ ID NOs: 17, 20, 23, 26, 29, 32, 35, 38, 143 or 146. In some instances, an antibody described herein comprises a sequence that is at least 85% identical to a CDRH2 sequence of any one of SEQ ID NOs: 17, 20, 23, 26, 29, 32, 35, 38, 143 or 146. In some instances, an antibody described herein comprises a sequence that is at least 90% identical to a CDRH2 sequence of any one of SEQ ID NOs: 17, 20, 23, 26, 29, 32, 35, 38, 143 or 146. In some instances, an antibody described herein comprises a sequence that is at least 95% identical to a CDRH2 sequence of any one of SEQ ID NOs: 17, 20, 23, 26, 29, 32, 35, 38, 143 or 146. In some instances, an antibody described herein comprises a CDRH3 sequence of any one of SEQ ID NOs: 18, 21, 24, 27, 30, 33, 36, 39, 144 or 147. In some instances, an antibody described herein comprises a sequence that is at least 80% identical to a CDRH3 sequence of any one of SEQ ID NOs: 18, 21, 24, 27, 30, 33, 36, 39, 144 or 147. In some instances, an antibody described herein comprises a sequence that is at least 85% identical to a CDRH3 sequence of any one of SEQ ID NOs: 18, 21, 24, 27, 30, 33, 36, 39, 144 or 147. In some instances, an antibody described herein comprises a sequence that is at least 90% identical to a CDRH3 sequence of any one of SEQ ID NOs: 18, 21, 24, 27, 30, 33, 36, 39, 144 or 147. In some instances, an antibody described herein comprises a sequence that is at least 95% identical to a CDRH3 sequence of any one of SEQ ID NOs: 18, 21, 24, 27, 30, 33, 36, 39, 144 or 147.

TABLE 2
SARS-CoV-2 Variant Heavy Chain Variable Domain
Complementarity Determining Regions (CDR)

| Construct | Amino Acid | SEQ ID |
|---|---|---|
| Description | Sequence | NO: |
| Ab-1 CDRH1 | FTFSNYPMN | 16 |
| Ab-1 CDRH2 | STISGSGGNTFYA | 17 |
| Ab-1 CDRH3 | CVRHDEYSFDYW | 18 |
| Ab-2 CDRH1 | ITFSSYAMS | 19 |
| Ab-2 CDRH2 | SGISGSGGSTYYA | 20 |
| Ab-2 CDRH3 | CAKHGSGTIFGVVIAKYYFDYW | 21 |
| Ab-3 CDRH1 | FTFSRHAMN | 22 |
| Ab-3 CDRH2 | SGITGSGDETYYA | 23 |
| Ab-3 CDRH3 | CARDLPASYYDSSGYYWHNGMDVW | 24 |
| Ab-4 CDRH1 | FTFSRHAMN | 25 |
| Ab-4 CDRH2 | SGISGSGDETYYA | 26 |
| Ab-4 CDRH3 | CARDLPASYYDSSGYYWHNGMDVW | 27 |
| Ab-5 CDRH1 | FMFSSYAMS | 28 |
| Ab-5 CDRH2 | SAISGSGGSTYYT | 29 |
| Ab-5 CDRH3 | CAKDGASGWPNWHFDLW | 30 |
| Ab-6 CDRH1 | FTFSNYAMS | 31 |
| Ab-6 CDRH2 | SDISGSGGSTYYA | 32 |
| Ab-6 CDRH3 | CVKGTIPIFGVIRSAFDYW | 33 |
| Ab-7 CDRH1 | FTFSDFAMA | 34 |
| Ab-7 CDRH2 | SAISGSGDITYYA | 35 |
| Ab-7 CDRH3 | CAREADGLHSPWHFDLW | 36 |
| Ab-8 CDRH1 | FNVNDYAMG | 37 |
| Ab-8 CDRH2 | AGITSSVGVTNYA | 38 |
| Ab-8 CDRH3 | CAADIFFVNW | 39 |
| Ab-9 CDRH1 | FTLDYYAMG | 142 |
| Ab-9 CDRH2 | AAINWSGDNTHYA | 143 |
| Ab-9 CDRH3 | CARAPFYCTTTKCQDNYYYMDVW | 144 |
| Ab-10 CDRH1 | GTFSSIGMG | 145 |
| Ab-10 CDRH2 | AAISWDGGATAYA | 146 |
| Ab-10 CDR-H3 | CAKEDVGKPFDW | 147 |

In some instances, an antibody described herein comprises a heavy chain variable domain complementarity determining region (CDRH) sequence as listed in Table 2. In some instances, an antibody described herein comprises a CDRH1 sequence of any one of SEQ ID NOs: 148-882. In some instances, an antibody described herein comprises a sequence that is at least 80% identical to a CDRH1 sequence of any one of SEQ ID NOs: 148-882. In some instances, an antibody described herein comprises a sequence that is at least 85% identical to a CDRH1 sequence of any one of SEQ ID NOs: 148-882. In some instances, an antibody described herein comprises a sequence that is at least 90% identical to a CDRH1 sequence of any one of SEQ ID NOs: 148-882. In some instances, an antibody described herein comprises a sequence that is at least 95% identical to a CDRH1 sequence of any one of SEQ ID NOs: 148-882. In some instances, an antibody described herein comprises a CDRH2 sequence of any one of SEQ ID NOs: 883-1617. In some instances, an antibody described herein comprises a sequence that is at least 80% identical to a CDRH2 sequence of any one of SEQ ID NOs: 883-1617. In some instances, an antibody described herein comprises a sequence that is at least 85% identical to a CDRH2 sequence of any one of SEQ ID NOs: 883-1617. In some instances, an antibody described herein comprises a sequence that is at least 90% identical to a CDRH2 sequence of any one of SEQ ID NOs: 883-1617. In some instances, an antibody described herein comprises a sequence that is at least 95% identical to a CDRH2 sequence of any one of SEQ ID NOs: 883-1617. In some instances, an antibody described herein comprises a CDRH3 sequence of any one of SEQ ID NOs: 1618-2416 or 4177-4212. In some instances, an antibody described herein comprises a sequence that is at least 80% identical to a CDRH3 sequence of any one of SEQ ID NOs: 1618-2416 or 4177-4212. In some instances, an antibody described herein comprises a sequence that is at least 85% identical to a CDRH3 sequence of any one of SEQ ID NOs: 1618-2416 or 4177-4212. In some instances, an antibody described herein comprises a sequence that is at least 90% identical to a CDRH3 sequence of any one of SEQ ID NOs: 1618-2416 or 4177-4212. In some instances, an antibody described herein comprises a sequence that is at least 95% identical to a CDRH3 sequence of any one of SEQ ID NOs: 1618-2416 or 4177-4212.

In some instances, an antibody described herein comprises a light chain variable domain complementarily determining region (CDRL) sequence as listed in Table 3. In some instances, an antibody described herein comprises a CDRL1 sequence of any one of SEQ ID NOs: 40, 43, 46, 49, 52, 55, or 58. In some instances, an antibody described herein comprises a sequence that is at least 80% identical to a CDRL1 sequence of any one of SEQ ID NOs: 40, 43, 46, 49, 52, 55, or 58. In some instances, an antibody described herein comprises a sequence that is at least 85% identical to a CDRL1 sequence of any one of SEQ ID NOs: 40, 43, 46, 49, 52, 55, or 58. In some instances, an antibody described herein comprises a sequence that is at least 90% identical to a CDRL1 sequence of any one of SEQ ID NOs: 40, 43, 46, 49, 52, 55, or 58. In some instances, an antibody described herein comprises a sequence that is at least 95% identical to a CDRL1 sequence of any one of SEQ ID NOs: 40, 43, 46, 49, 52, 55, or 58. In some instances, an antibody described herein comprises a CDRL2 sequence of any one of SEQ ID NOs: 41, 44, 47, 50, 53, 56, or 59. In some instances, an antibody described herein comprises a sequence that is at least 80% identical to a CDRL2 sequence of any one of SEQ ID NOs: 41, 44, 47, 50, 53, 56, or 59. In some instances, an antibody described herein comprises a sequence that is at least 85% identical to a CDRL2 sequence of any one of SEQ ID NOs: 41, 44, 47, 50, 53, 56, or 59. In some instances, an antibody described herein comprises a sequence that is at least 90% identical to a CDRL2 sequence of any one of SEQ ID NOs: 41, 44, 47, 50, 53, 56, or 59. In some instances, an antibody described herein comprises a sequence that is at least 95% identical to a CDRL2 sequence of any one of SEQ ID NOs: 41, 44, 47, 50, 53, 56, or 59. In some instances, an antibody described herein comprises a CDRL3 sequence of any one of SEQ ID NOs: 42, 45, 48, 51, 54, 57, or 60. In some instances, an antibody described herein comprises a sequence that is at least 80% identical to a CDRL3 sequence of any one of SEQ ID NOs: 42, 45, 48, 51, 54, 57, or 60. In some instances, an antibody described herein comprises a sequence that is at least 85% identical to a CDRL3 sequence of any one of SEQ ID NOs: 42, 45, 48, 51, 54, 57, or 60. In some instances, an antibody described herein comprises a sequence that is at least 90% identical to a CDRL3 sequence of any one of SEQ ID NOs: 42, 45, 48, 51, 54, 57, or 60. In some instances, an antibody described herein comprises a sequence that is at least 95% identical to a CDRL3 sequence of any one of SEQ ID NOs: 42, 45, 48, 51, 54, 57, or 60.

TABLE 3
SARS-CoV-2 S1 Variant Light Chain Variable
Domain Complementarity Determining Regions (CDR)

| Construct | | SEQ ID |
|---|---|---|
| Description | Amino Acid Sequence | NO: |
| Ab-1 CDRL1 | RASQSIGNYLN | 40 |
| Ab-1 CDRL2 | GVSSLQS | 41 |
| Ab-1 CDRL3 | CQQSHSAPLTF | 42 |
| Ab-2 CDRL1 | TGTSSDVGHYNLVS | 43 |
| Ab-2 CDRL2 | EGTKRPS | 44 |
| Ab-2 CDRL3 | CCSYAGSSSFVVF | 45 |
| Ab-3 CDRL1 | RASQTINTFLN | 46 |
| Ab-3 CDRL2 | SASTLQS | 47 |
| Ab-3 CDRL3 | CQQSYSTFTF | 48 |
| Ab-4 CDRL1 | RASQTINTYLN | 49 |
| Ab-4 CDRL2 | SASTLQS | 50 |
| Ab-4 CDRL3 | CQQSYSTFTF | 51 |
| Ab-5 CDRL1 | TGTSSDVGSYYLVS | 52 |
| Ab-5 CDRL2 | EGDKRPS | 53 |
| Ab-5 CDRL3 | CCSHAGRYPYVF | 54 |
| Ab-6 CDRL1 | TGTSSDVGSYSLVS | 55 |
| Ab-6 CDRL2 | EGTKRPS | 56 |
| Ab-6 CDRL3 | CCSYAGSYSYVVF | 57 |
| Ab-7 CDRL1 | RASQSIHTYLN | 58 |
| Ab-7 CDRL2 | AASALAS | 59 |
| Ab-7 CDRL3 | CQQSYSAPPYTF | 60 |

In some instances, an antibody described herein comprises a light chain variable domain complementarity determining region (CDRL) sequence as listed in Table 3. In some instances, an antibody described herein comprises a CDRL1 sequence of any one of SEQ ID NOs: 2417-2586. In some instances, an antibody described herein comprises a sequence that is at least 80% identical to a CDRL1 sequence of any one of SEQ ID NOs: 2417-2586. In some instances, an antibody described herein comprises a sequence that is at least 85% identical to a CDRL1 sequence of any one of SEQ ID NOs: 2417-2586. In some instances, an antibody described herein comprises a sequence that is at least 90% identical to a CDRL1 sequence of any one of SEQ ID NOs: 2417-2586. In some instances, an antibody described herein comprises a sequence that is at least 95% identical to a CDRL1 sequence of any one of SEQ ID NOs: 2417-2586. In some instances, an antibody described herein comprises a CDRL2 sequence of any one of SEQ ID NOs: 2587-2756. In some instances, an antibody described herein comprises a sequence that is at least 80% identical to a CDRL2 sequence of any one of SEQ ID NOs: 2587-2756. In some instances, an antibody described herein comprises a sequence that is at least 85% identical to a CDRL2 sequence of any one of SEQ ID NOs: 2587-2756. In some instances, an antibody described herein comprises a sequence that is at least 90% identical to a CDRL2 sequence of any one of SEQ ID NOs: 2587-2756. In some instances, an antibody described herein comprises a sequence that is at least 95% identical to a CDRL2 sequence of any one of SEQ ID NOs: 2587-2756. In some instances, an antibody described herein comprises a CDRL3 sequence of any one of SEQ ID NOs: 2757-2926. In some instances, an antibody described herein comprises a sequence that is at least 80% identical to a CDRL3 sequence of any one of SEQ ID NOs: 2757-2926. In some instances, an antibody described herein comprises a sequence that is at least 85% identical to a CDRL3 sequence of any one of SEQ ID NOs: 2757-2926. In some instances, an antibody described herein comprises a sequence that is at least 90% identical to a CDRL3 sequence of any one of SEQ ID NOs: 2757-2926. In some instances, an antibody described herein comprises a sequence that is at least 95% identical to a CDRL3 sequence of any one of SEQ ID NOs: 2757-2926.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDRH1, CDRH2, CDRH3) and three CDRs in each light chain variable region (CDRL1, CDRL2, CDRL3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Antibodies used with the devices and systems as described herein may comprise improved binding affinity. In some instances, the SARS-CoV-2 antibody comprises a binding affinity (e.g., K_{D}) to SARS-CoV-2 of less than 1 nM, less than 1.2 nM, less than 2 nM, less than 5 nM, less than 10 nM, less than 11 nm, less than 13.5 nM, less than 15 nM, less than 20 nM, less than 25 nM, or less than 30 nM. In some instances, the SARS-CoV-2 antibody comprises a K_{D} of less than 1 nM. In some instances, the SARS-CoV-2 antibody comprises a K_{D} of less than 1.2 nM. In some instances, the SARS-CoV-2 antibody comprises a K_{D} of less than 2 nM. In some instances, the SARS-CoV-2 antibody comprises a K_{D} of less than 5 nM. In some instances, the SARS-CoV-2 antibody comprises a K_{D} of less than 10 nM. In some instances, the SARS-CoV-2 antibody comprises a K_{D} of less than 13.5 nM. In some instances, the SARS-CoV-2 antibody comprises a K_{D} of less than 15 nM. In some instances, the SARS-CoV-2 antibody comprises a K_{D} of less than 20 nM. In some instances, the SARS-CoV-2 antibody comprises a K_{D} of less than 25 nM. In some instances, the SARS-CoV-2 antibody comprises a K_{D} of less than 30 nM.

In some instances, the ACE2 antibody comprises a binding affinity (e.g., K_{D}) to ACE2 of less than 1 nM, less than 1.2 nM, less than 2 nM, less than 5 nM, less than 10 nM, less than 11 nm, less than 13.5 nM, less than 15 nM, less than 20 nM, less than 25 nM, or less than 30 nM. In some instances, the ACE2 antibody comprises a K_{D} of less than 1 nM. In some instances, the ACE2 antibody comprises a K_{D} of less than 1.2 nM. In some instances, the ACE2 antibody comprises a K_{D} of less than 2 nM. In some instances, the ACE2 antibody comprises a K_{D} of less than 5 nM. In some instances, the ACE2 antibody comprises a K_{D} of less than 10 nM. In some instances, the ACE2 antibody comprises a K_{D} of less than 13.5 nM. In some instances, the ACE2 antibody comprises a K_{D} of less than 15 nM. In some instances, the ACE2 antibody comprises a K_{D} of less than 20 nM. In some instances, the ACE2 antibody comprises a K_{D} of less than 25 nM. In some instances, the ACE2 antibody comprises a K_{D} of less than 30 nM.

In some embodiments, the systems and devices as described herein comprise one or more test lines. In some embodiments, the systems and devices comprise at least 1, 2, 3, 4, 5, 6, or more than 6 test lines. In some embodiments, the one or more test lines comprise the same antibody. In some embodiments, the one or more test lines comprise different antibodies. In some embodiments, the one or more test lines comprise one or more different antibodies. In some embodiments, the one or more test lines comprise at least 2, 3, 4, 5, 6, or more than 6 different antibodies.

The virus may be too small to be seen by the naked eye, or even with assisted vision such as with a light microscope. In some cases, a reagent comprising large particles (e.g. nanobeads, microbeads, colored dyes) is conjugated to the virus to develop a detectible signal. In some cases, the conjugate pad further comprises a conjugate reagent. The conjugate reagent may be used to detect an infectious agent by binding to a region of the target virus. In some cases, the conjugate reagent is coupled to a polypeptide that has affinity to a region of the target. In some cases, the polypeptide is a protein or an antibody as described herein. In some cases, the conjugate reagent provides a signal. The signal may then be detected by a device or in some cases the signal is visible such a color change or a visible band.

In some instances, the conjugate reagent is conjugated to an antibody. In some instances, the conjugate reagent is used for detecting the presence of the virus and generating a detectible signal. In some instances, the signal is a visible band, a fluorescent color, or a colored band. In some instances, the signal is detectible with assisted vision such as with a microscope.

The conjugate reagent can comprise various materials. In some instances, the conjugate reagent is selected from the group consisting of colloidal gold, latex particles, enzymes, colored dyes, paramagnetic particles, gold nanoparticles, gold nanoshells, and fluorescent particles. In some aspects, the conjugate reagent comprises gold nanoparticles, gold nanoshells, or combinations thereof.

Described herein are systems and devices for detecting a virus, wherein the device can be a lateral flow assay (LFA) device.

Devices as described herein can comprise varying dimensions. In some embodiments, the device is at least about 1 mm, 2 mm, 3 mm, 4 mm, 5 mm, 6 mm, 7 mm, 8 mm, 9 mm, 10 mm, 11 mm, 12 mm, 16 mm, 18 mm, 20 mm, or more than 20 mm by about 4 mm, 5 mm, 6 mm, 7 mm, 8 mm, 9 mm, 10 mm, 11 mm, 12 mm, 16 mm, 18 mm, 20 mm, 24 mm, 26 mm, 28 mm, 30 mm, 40 mm, 50 mm, 60 mm, 70 mm, 80 mm, 90 mm, 100 mm, or more than 100 mm. In some instances, the device is at least about 5 mm by about 70 mm. In some instances, the membrane substrate is at least about 4 mm, 5 mm, 6 mm, 7 mm, 8 mm, 9 mm, 10 mm, 11 mm, 12 mm, 16 mm, 18 mm, 20 mm, 24 mm, 26 mm, 28 mm, 30 mm, 40 mm, 50 mm, 60 mm, or more than 60 mm in width. the membrane substrate is at least about 4 mm, 5 mm, 6 mm, 7 mm, 8 mm, 9 mm, 10 mm, 11 mm, 12 mm, 16 mm, 18 mm, 20 mm, 24 mm, 26 mm, 28 mm, 30 mm, 40 mm, 50 mm, 60 mm, 70 mm, 80 mm, 90 mm, 100 mm, or more than 100 mm.

In some embodiments, the device further comprises a housing. In some instances, the housing covers at least a portion of the device. In some instances, the housing comprises a sample application port to allow sample application upstream from or to the test locations and an optic opening around the test locations to allow signal detection at the test locations. The housing can comprise any suitable material. For example, the housing can comprise a plastic material. In some instances, the housing comprises an opaque, translucent, or transparent material.

Systems and devices as described herein can detect the virus in a quick and reliable manner. In some instances, the device is a point of care device. In some instances, the device is a LFA device. In some instances, the device provides a read out in about 9 seconds (s) to about 30 minutes (min). In some instances, the device provides a read out in at least about 9 s, 10 s, 11 s, 12 s, 13 s, 14 s, 15 s, 20 s, 30 s, 40 s, 50 s, 1 min, 1.5 min, 2 min, 3 min, 4 min, 5 min, 10 min, 15 min, 20 min, 25 min, 30 min, or more. In some cases, the device provides a read out in at most about 30 min, 25 min, 20 min, 15 min, 10 min, 5 min, 4 min, 3 min, 2 min, 1.5 min, 1 min, 50 s, 40 s, 30 s, 20 s, 15 s, 14 s, 13 s, 12 s, 11 s, 10 s, 9 s, or less. In some aspects, the device provides a read out in at most about 20 seconds.

In some instances, a fragment of a virus is captured and detected. In some cases, a first portion of the fragment of the virus is detected by a first antibody and a second portion of the fragment of the virus is detected by a second antibody. In some instances, the fragment of the virus comprises a spike protein, a membrane protein, an envelope protein, a nucleocapsid protein, or combinations thereof

Methods of Use

Provided herein are methods of using the systems and devices as described herein for detecting or diagnosing a microbial infection. In some embodiments, the microbial infection is caused by a virus. In some embodiments, the microbial infection is caused by a bacteria. In some embodiments, the microbial infection is caused by a fungus. In some embodiments, the microbial infection is caused by a bacteria.

Described herein are methods for testing a sample to determine the presence of a virus in a sample. In some embodiments, the sample is a biological sample. In some instances, the biological sample is collected from a subject. In some instances, the sample is collected from a human subject or an animal subject. In some instances, the sample is a fluid (e.g., bodily fluid). In some instances, the fluid is saliva, blood, semen, vaginal fluid, or urine.

Provided herein are methods and systems to analyze a biological sample for the presence of a virus comprising improved sensitivity, specificity, reliability, and accuracy. In some instances, the virus is a respiratory virus. In some aspects, the virus is a coronavirus. In some instances, the coronavirus is SARS or MERS. In some aspects, the SARS coronavirus is COVID-19. In some instances, the virus is a human virus, a bovine virus, a swine virus, a feline virus, an avian virus, or an equine virus.

Methods and systems as described herein may have a sensitivity of at least about 70% of detecting the virus. In some instances, the methods and systems as described herein are at least about 75%, 80%, 85%. 90%, 95% or more than 95% sensitive in detecting the virus. In some instances, the methods and systems detect viral titers in a range of about 10³ to about 10⁴ viral particles. In some instances, the methods and systems detect viral titers of about 10¹, 10², 10³, 10⁴, 10⁵, 10⁶, 10⁷, 10⁸, 10⁹, or more than 10⁹ particles. In some instances, the methods and systems detect at least or about 0.25, 0.5, 1, 2.5, 5, 10, 15, 20, 25, 30, 40, 50, or more than 50 ng/mL of virus or viral protein. In some instances, the methods and systems detect at least or about 10 ng/mL of virus or viral protein. In some instances, the viral protein is SARS-CoV-2 spike trimer protein. In some instances, the viral protein is SARS-CoV-2 nucleocapsid protein.

Methods and systems as described herein may have a specificity of at least about 70% for detecting the virus as compared to another virus. In some instances, the methods and systems as described herein are at least about 75%, 80%, 85%. 90%, 95% or more than 95% specific for detecting the virus as compared to another virus. In some instances, the methods and systems as described herein are specific for detecting SARS-CoV-2. In some instances, the methods and systems as described herein distinguish between SARS-CoV, MERS-CoV, CoV-229E, HCoV-NL63, HCoV-OC43, or HCoV-HKU1. In some embodiments, some instances, the methods and systems as described herein distinguish SARS-CoV-2 from SARS-CoV.

Methods and systems as described herein may have an accuracy of at least about 70% of detecting the virus. In some instances, the methods and systems as described herein are at least about 75%, 80%, 85%. 90%, 95% or more than 95% accuracy in detecting the virus.

Methods and systems as described herein may have a reliability of at least about 70% of detecting the virus. In some instances, the methods and systems as described herein are at least about 75%, 80%, 85%. 90%, 95% or more than 95% reliable in detecting the virus.

Sensitivity, specificity, accuracy, and reliability may be improved as compared to a comparable test. In some instances, the test is a PCR-based test. In some instances, the test is RT-PCR, isothermal nucleic acid amplification, a CRISPR-based assay, rolling circle amplification, a nucleic acid hybridization assay (e.g., microarray), a sequencing assay, or immunoassay. In some instances, the immunoassay is an Enzyme-Linked Immunosorbent Assay (ELISA), lateral flow immunoassay, a neutralization assay, a luminescent immunoassay, a biosensor test, or a rapid antigen test.

Methods and systems as described herein may have an improved limit of detection. In some instances, the methods and systems as described herein has a limit of detection of at least about 10³ copies/mL. In some instances, the methods and systems detect viral titers of about 10¹, 10², 10³, 10⁴, 10⁵, 10⁶, 10⁷, 10⁸, 10⁹, or more than 10⁹ copies/mL.

In some instances, the testing methods are performed outside of a laboratory, in a patient's home, in a hospital. In some instances, the testing is performed in the laboratory. The methods can be applied in a handheld device such as a portable microfluidics device. In some aspects, the methods are applied in a portable hand-held device.

In some instances, the device provides a read out in about 1 minute to 30 minutes. In some instances, the device provides a read out in at least about 30 seconds, 40 seconds, 50 seconds, 1 minute, 1.5 minutes, 2 minutes, 3 minutes, 4 minutes, 5 minutes, 10 minutes, 15 minutes, 20 minutes, 25 minutes, 30 minutes, or more. In some cases, the device provides a read out in at most about 30 minutes, 25 minutes, 20 minutes, 15 minutes, 10 minutes, 5 minutes, 4 minutes, 3 minutes, 2 minutes, 1.5 minutes, 1 minutes, or less. In some aspects, the device provides a read out in a range of about 1 minute to about 30 minutes, about 2 minutes to about 25 minutes, about 5 minutes to about 20 minutes, or about 10 minutes to about 15 minutes.

Further described herein are examples of the steps that may be involved in detecting a virus in the systems and devices described herein. In some instances, a sample or fluid is loaded on the sample application pad. In some instances, a running buffer is applied to the sample application pad.

The sample fluid may migrate to the conjugate pad within a predefined period of time, via capillary action. The predefined period of time that it may take for the sample fluid to travel from the sample pad to the conjugate pad may be about 0.5 minutes (min) to 5 min. In some is the sample fluid travel time through the sample pad is at least 0.1 min, 0.2 min, 0.3 min, 0.4 min, 0.5 min, 1 min, 1.5 min, 2 min, 2.5 min, 3 min, 3.5 min, 4 min, 4.5 min, 5 min, or more. In some instances, the travel time is at most 5 min, 4.5 min, 4 min, 3.5 min, 3 min, 2.5 min, 2 min, 1.5 min, 1 min, 0.5 min, 0.4 min, 0.3 min, 0.2 min, 0.1 min, or less.

In some instances, the conjugate reagent on the conjugate pad and the sample fluid comes in contact at the conjugate pad. In some instances, the sample fluid rehydrates the conjugate reagents on the conjugate pad. In some instances, the sample fluid travels through (across) the conjugate pad for a predefined period of time. The predefined period of time for the fluid to travel across the conjugate pad to reach the membrane substrate may be about 0.5 minutes (min) to 5 min. In some instances, the fluid travel time through the conjugate pad is at least 0.1 min, 0.2 min, 0.3 min, 0.4 min, 0.5 min, 1 min, 1.5 min, 2 min, 2.5 min, 3 min, 3.5 min, 4 min, 4.5 min, 5 min, or more. In some instances, the fluid travel time across the conjugate pad is at most 5 min, 4.5 min, 4 min, 3.5 min, 3 min, 2.5 min, 2 min, 1.5 min, 1 min, 0.5 min, 0.4 min, 0.3 min, 0.2 min, 0.1 min, or less. If the sample contains the target for the conjugate reagent or the antibody coupled to the conjugate reagent at the conjugate pad, then the target-conjugate reagent complexes may be formed.

In some instances, the sample or fluid migrates through the membrane substrate towards the first test line. In some instances, the target-conjugate reagent complexes reach the first test line and are captured by immobilized antibodies described herein that are coupled to the first test line. In some aspects, the captured target-conjugate reagent complexes form a visible band at the first test line. In some instances, the fluid migrates across the first test line for a predefined period of time. The predefined period of time that it may take for the fluid to travel across the first test line may be about 0.5 minutes (min) to 5 min. In some instances, the fluid travel time across the first test line is at least 0.1 min, 0.2 min, 0.3 min, 0.4 min, 0.5 min, 1 min, 1.5 min, 2 min, 2.5 min, 3 min, 3.5 min, 4 min, 4.5 min, 5 min, or more. In some instances, the fluid travel time across the first test line is at most 5 min, 4.5 min, 4 min, 3.5 min, 3 min, 2.5 min, 2 min, 1.5 min, 1 min, 0.5 min, 0.4 min, 0.3 min, 0.2 min, 0.1 min, or less. In some instances, the fluid continues to migrate to the second test line.

In some instances, the sample or fluid migrates through the membrane substrate to a second test line. In some embodiments, the second test line is a control line. A visible readout may be included at the first test line, the second test line, or both. In some instances, the visible readout is a visible band, a fluorescent color, or a colored band. In some instances, the signal is a color change. In some aspects, based on the intensity or the color of the signal and/or detectible band indicates the presence, quantity, or potency of the virus. In some instances, the control line is compared to the first test line to determine presence, quantity, or potency of the virus (e.g., SARS-Cov-2).

Results from the device may then be transferred. In some instances, the results are transferred, wirelessly or through a cable, to a computerized device to process and display the information. In some embodiments, the result is transmitted to a software program on a computerized device, where the computerized device has a graphical user interface that displays the assay results.

In some instances, the results are transferred to a database. In some instances, the results from the database are used for bioinformatics applications such as functional genomics and homology searching.

Kits

Devices as described herein may be included in a kit. In some instances, kits are provided to an administering physician, other health care professional, a patient, or a caregiver. In some instances, a kit comprises a container which contains a testing device and instructions for using the device. In some instances, the container contains more than one testing device. The container may contain at least 1, 2, 3, 4, 5, 6, 7, 8, 9, 10, 11, 12, 20, 30, 40, 50, or more testing devices.

The assay kit can also include an amount of a chase buffer, e.g., PBS, sufficient to enable proper flow of the tracer reagent on each of the first and second test lines to the control line. The kit may comprise solutions, agents, and chase buffers that may be required to operate the device.

Additional kit components can include, e.g., an instrument for sample collection, e.g., a sharp instrument for drawing blood, or a swab for collecting saliva, urine, semen, or vaginal fluid, and an instrument for applying the sample to the sample pad, e.g., a dropper.

The kit can optionally also contain one or more other testing devices and diagnostic tools. The kit may also optionally contain therapeutic or other agents. In some cases, the kit comprises an assisted vision tool to help visually observe the readout such as a light source, a light filter, or a magnifier.

The assay kit can further include instructions for use, which can comprise a description of test pattern interpretation, and recommendations for patient action based on the result obtained. In embodiments, the patient is encouraged to seek a confirmatory test should the rapid test of the invention indicate early or intermediate virus infection. In embodiments, contact information for a suitable test facility is provided.

In some embodiments, the instructions for use include a cautionary warning based on the result interpretation. In embodiments, a mobile phone application is made available to the user, so that test results is provided to a practitioner and/or epidemiologist.

Highly Parallel Nucleic Acid Synthesis

Provided herein is a platform approach utilizing miniaturization, parallelization, and vertical integration of the end-to-end process from polynucleotide synthesis to gene assembly within nanowells on silicon to create a revolutionary synthesis platform. Devices described herein provide, with the same footprint as a 96-well plate, a silicon synthesis platform is capable of increasing throughput by a factor of up to 1,000 or more compared to traditional synthesis methods, with production of up to approximately 1,000,000 or more polynucleotides, or 10,000 or more genes in a single highly-parallelized run.

In some embodiments, a drug itself can be optimized using methods described herein. For example, to improve a specified function of an antibody, a variant polynucleotide library encoding for a portion of the antibody is designed and synthesized. A variant nucleic acid library for the antibody can then be generated by processes described herein (e.g., PCR mutagenesis followed by insertion into a vector). The antibody is then expressed in a production cell line and screened for enhanced activity. Example screens include examining modulation in binding affinity to an antigen, stability, or effector function (e.g., ADCC, complement, or apoptosis). Exemplary regions to optimize the antibody include, without limitation, the Fc region, Fab region, variable region of the Fab region, constant region of the Fab region, variable domain of the heavy chain or light chain (V_{H} or V_{L}), and specific complementarity-determining regions (CDRs) of V_{H} or V_{L}.

Substrates

Devices used as a surface for polynucleotide synthesis may be in the form of substrates which include, without limitation, homogenous array surfaces, patterned array surfaces, channels, beads, gels, and the like. Provided herein are substrates comprising a plurality of clusters, wherein each cluster comprises a plurality of loci that support the attachment and synthesis of polynucleotides. In some instances, substrates comprise a homogenous array surface. For example, the homogenous array surface is a homogenous plate. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus is on a three-dimensional surface, e.g., a well, microwell, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some cases, a surface of a substrate is inclusive of one or a plurality of surfaces of a substrate. The average error rates for polynucleotides synthesized within a library described here using the systems and methods provided are often less than 1 in 1000, less than about 1 in 2000, less than about 1 in 3000 or less often without error correction.

Provided herein are surfaces that support the parallel synthesis of a plurality of polynucleotides having different predetermined sequences at addressable locations on a common support. In some instances, a substrate provides support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the surfaces provide support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the substrate provides a surface environment for the growth of polynucleotides having at least 80, 90, 100, 120, 150, 175, 200, 225, 250, 275, 300, 325, 350, 375, 400, 425, 450, 475, 500 bases or more.

Provided herein are methods for polynucleotide synthesis on distinct loci of a substrate, wherein each locus supports the synthesis of a population of polynucleotides. In some cases, each locus supports the synthesis of a population of polynucleotides having a different sequence than a population of polynucleotides grown on another locus. In some instances, each polynucleotide sequence is synthesized with 1, 2, 3, 4, 5, 6, 7, 8, 9 or more redundancy across different loci within the same cluster of loci on a surface for polynucleotide synthesis. In some instances, the loci of a substrate are located within a plurality of clusters. In some instances, a substrate comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a substrate comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a substrate comprises about 10,000 distinct loci. The amount of loci within a single cluster is varied in different instances. In some cases, each cluster includes 1, 2, 3, 4, 5, 6, 7, 8, 9, 10, 20, 30, 40, 50, 60, 70, 80, 90, 100, 120, 130, 150, 200, 300, 400, 500 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci. Alternatively or in combination, polynucleotide synthesis occurs on a homogenous array surface.

In some instances, the number of distinct polynucleotides synthesized on a substrate is dependent on the number of distinct loci available in the substrate. In some instances, the density of loci within a cluster or surface of a substrate is at least or about 1, 10, 25, 50, 65, 75, 100, 130, 150, 175, 200, 300, 400, 500, 1,000 or more loci per mm². In some cases, a substrate comprises 10-500, 25-400, 50-500, 100-500, 150-500, 10-250, 50-250, 10-200, or 50-200 mm². In some instances, the distance between the centers of two adjacent loci within a cluster or surface is from about 10-500, from about 10-200, or from about 10-100 um. In some instances, the distance between two centers of adjacent loci is greater than about 10, 20, 30, 40, 50, 60, 70, 80, 90 or 100 um. In some instances, the distance between the centers of two adjacent loci is less than about 200, 150, 100, 80, 70, 60, 50, 40, 30, 20 or 10 um. In some instances, each locus has a width of about 0.5, 1, 2, 3, 4, 5, 6, 7, 8, 9, 10, 20, 30, 40, 50, 60, 70, 80, 90 or 100 um. In some cases, each locus has a width of about 0.5-100, 0.5-50, 10-75, or 0.5-50 um.

In some instances, the density of clusters within a substrate is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a substrate comprises from about 1 cluster per 10 mm² to about 10 clusters per 1 mm². In some instances, the distance between the centers of two adjacent clusters is at least or about 50, 100, 200, 500, 1000, 2000, or 5000 um. In some cases, the distance between the centers of two adjacent clusters is between about 50-100, 50-200, 50-300, 50-500, and 100-2000 um. In some cases, the distance between the centers of two adjacent clusters is between about 0.05-50, 0.05-10, 0.05-5, 0.05-4, 0.05-3, 0.05-2, 0.1-10, 0.2-10, 0.3-10, 0.4-10, 0.5-10, 0.5-5, or 0.5-2 mm. In some cases, each cluster has a cross section of about 0.5 to about 2, about 0.5 to about 1, or about 1 to about 2 mm. In some cases, each cluster has a cross section of about 0.5, 0.6, 0.7, 0.8, 0.9, 1, 1.1, 1.2, 1.3, 1.4, 1.5, 1.6, 1.7, 1.8, 1.9 or 2 mm. In some cases, each cluster has an interior cross section of about 0.5, 0.6, 0.7, 0.8, 0.9, 1, 1.1, 1.15, 1.2, 1.3, 1.4, 1.5, 1.6, 1.7, 1.8, 1.9 or 2 mm.

In some instances, a substrate is about the size of a standard 96 well plate, for example between about 100 and about 200 mm by between about 50 and about 150 mm. In some instances, a substrate has a diameter less than or equal to about 1000, 500, 450, 400, 300, 250, 200, 150, 100 or 50 mm. In some instances, the diameter of a substrate is between about 25-1000, 25-800, 25-600, 25-500, 25-400, 25-300, or 25-200 mm. In some instances, a substrate has a planar surface area of at least about 100; 200; 500; 1,000; 2,000; 5,000; 10,000; 12,000; 15,000; 20,000; 30,000; 40,000; 50,000 mm² or more. In some instances, the thickness of a substrate is between about 50-2000, 50-1000, 100-1000, 200-1000, or 250-1000 mm.

Surface Materials

Substrates, devices, and reactors provided herein are fabricated from any variety of materials suitable for the methods, compositions, and systems described herein. In certain instances, substrate materials are fabricated to exhibit a low level of nucleotide binding. In some instances, substrate materials are modified to generate distinct surfaces that exhibit a high level of nucleotide binding. In some instances, substrate materials are transparent to visible and/or UV light. In some instances, substrate materials are sufficiently conductive, e.g., are able to form uniform electric fields across all or a portion of a substrate. In some instances, conductive materials are connected to an electric ground. In some instances, the substrate is heat conductive or insulated. In some instances, the materials are chemical resistant and heat resistant to support chemical or biochemical reactions, for example polynucleotide synthesis reaction processes. In some instances, a substrate comprises flexible materials. For flexible materials, materials can include, without limitation: nylon, both modified and unmodified, nitrocellulose, polypropylene, and the like. In some instances, a substrate comprises rigid materials. For rigid materials, materials can include, without limitation: glass; fuse silica; silicon, plastics (for example polytetrafluoroethylene, polypropylene, polystyrene, polycarbonate, and blends thereof, and the like); metals (for example, gold, platinum, and the like). The substrate, solid support or reactors can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

Surface Architecture

Provided herein are substrates for the methods, compositions, and systems described herein, wherein the substrates have a surface architecture suitable for the methods, compositions, and systems described herein. In some instances, a substrate comprises raised and/or lowered features. One benefit of having such features is an increase in surface area to support polynucleotide synthesis. In some instances, a substrate having raised and/or lowered features is referred to as a three-dimensional substrate. In some cases, a three-dimensional substrate comprises one or more channels. In some cases, one or more loci comprise a channel. In some cases, the channels are accessible to reagent deposition via a deposition device such as a material deposition device. In some cases, reagents and/or fluids collect in a larger well in fluid communication one or more channels. For example, a substrate comprises a plurality of channels corresponding to a plurality of loci with a cluster, and the plurality of channels are in fluid communication with one well of the cluster. In some methods, a library of polynucleotides is synthesized in a plurality of loci of a cluster.

Provided herein are substrates for the methods, compositions, and systems described herein, wherein the substrates are configured for polynucleotide synthesis. In some instances, the structure is configured to allow for controlled flow and mass transfer paths for polynucleotide synthesis on a surface. In some instances, the configuration of a substrate allows for the controlled and even distribution of mass transfer paths, chemical exposure times, and/or wash efficacy during polynucleotide synthesis. In some instances, the configuration of a substrate allows for increased sweep efficiency, for example by providing sufficient volume for a growing polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are substrates for the methods, compositions, and systems described herein, wherein the substrates comprise structures suitable for the methods, compositions, and systems described herein. In some instances, segregation is achieved by physical structure. In some instances, segregation is achieved by differential functionalization of the surface generating active and passive regions for polynucleotide synthesis. In some instances, differential functionalization is achieved by alternating the hydrophobicity across the substrate surface, thereby creating water contact angle effects that cause beading or wetting of the deposited reagents.

Employing larger structures can decrease splashing and cross-contamination of distinct polynucleotide synthesis locations with reagents of the neighboring spots. In some cases, a device, such as a material deposition device, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000, 1:3,000, 1:5,000, or 1:10,000). In some cases, a substrate comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a substrate may have the same or different width, height, and/or volume as another well of the substrate. A channel of a substrate may have the same or different width, height, and/or volume as another channel of the substrate. In some instances, the diameter of a cluster or the diameter of a well comprising a cluster, or both, is between about 0.05-50, 0.05-10, 0.05-5, 0.05-4, 0.05-3, 0.05-2, 0.05-1, 0.05-0.5, 0.05-0.1, 0.1-10, 0.2-10, 0.3-10, 0.4-10, 0.5-10, 0.5-5, or 0.5-2 mm. In some instances, the diameter of a cluster or well or both is less than or about 5, 4, 3, 2, 1, 0.5, 0.1, 0.09, 0.08, 0.07, 0.06, or 0.05 mm. In some instances, the diameter of a cluster or well or both is between about 1.0 and 1.3 mm. In some instances, the diameter of a cluster or well, or both is about 1.150 mm. In some instances, the diameter of a cluster or well, or both is about 0.08 mm. The diameter of a cluster refers to clusters within a two-dimensional or three-dimensional substrate.

In some instances, the height of a well is from about 20-1000, 50-1000, 100-1000, 200-1000, 300-1000, 400-1000, or 500-1000 um. In some cases, the height of a well is less than about 1000, 900, 800, 700, or 600 um.

In some instances, a substrate comprises a plurality of channels corresponding to a plurality of loci within a cluster, wherein the height or depth of a channel is 5-500, 5-400, 5-300, 5-200, 5-100, 5-50, or 10-50 um. In some cases, the height of a channel is less than 100, 80, 60, 40, or 20 um.

In some instances, the diameter of a channel, locus (e.g., in a substantially planar substrate) or both channel and locus (e.g., in a three-dimensional substrate wherein a locus corresponds to a channel) is from about 1-1000, 1-500, 1-200, 1-100, 5-100, or 10-100 um, for example, about 90, 80, 70, 60, 50, 40, 30, 20 or 10 um. In some instances, the diameter of a channel, locus, or both channel and locus is less than about 100, 90, 80, 70, 60, 50, 40, 30, 20 or 10 um. In some instances, the distance between the center of two adjacent channels, loci, or channels and loci is from about 1-500, 1-200, 1-100, 5-200, 5-100, 5-50, or 5-30, for example, about 20 um.

Surface Modifications

Provided herein are methods for polynucleotide synthesis on a surface, wherein the surface comprises various surface modifications. In some instances, the surface modifications are employed for the chemical and/or physical alteration of a surface by an additive or subtractive process to change one or more chemical and/or physical properties of a substrate surface or a selected site or region of a substrate surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some cases, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some cases, the adhesion promoter is a chemical with a high surface energy. In some instances, a second chemical layer is deposited on a surface of a substrate. In some cases, the second chemical layer has a low surface energy. In some cases, surface energy of a chemical layer coated on a surface supports localization of droplets on the surface. Depending on the patterning arrangement selected, the proximity of loci and/or area of fluid contact at the loci are alterable.

In some instances, a substrate surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a substrate surface is modified with one or more different layers of compounds. Such modification layers of interest include, without limitation, inorganic and organic layers such as metals, metal oxides, polymers, small organic molecules and the like.

In some instances, resolved loci of a substrate are functionalized with one or more moieties that increase and/or decrease surface energy. In some cases, a moiety is chemically inert. In some cases, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for substrate functionalization comprises: (a) providing a substrate having a surface that comprises silicon dioxide; and (b) silanizing the surface using, a suitable silanizing agent described herein or otherwise known in the art, for example, an organofunctional alkoxysilane molecule. Methods and functionalizing agents are described in U.S. Pat. No. 5,474,796, which is herein incorporated by reference in its entirety.

In some instances, a substrate surface is functionalized by contact with a derivatizing composition that contains a mixture of silanes, under reaction conditions effective to couple the silanes to the substrate surface, typically via reactive hydrophilic moieties present on the substrate surface. Silanization generally covers a surface through self-assembly with organofunctional alkoxysilane molecules. A variety of siloxane functionalizing reagents can further be used as currently known in the art, e.g., for lowering or increasing surface energy. The organofunctional alkoxysilanes are classified according to their organic functions.

Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 min, 1 min, 50 sec, 40 sec, 30 sec, 20 sec and 10 sec.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′-OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example, at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150, 22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides is synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of nucleic acids comprising variant nucleic acids differing at a plurality of codon sites. In some instances, a nucleic acid may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may not be adjacent and separated by 1, 2, 3, 4, 5, 6, 7, 8, 9, 10, or more codons.

In some instances, a nucleic acid may comprise multiple sites of variant codon sites, wherein all the variant codon sites are adjacent to one another, forming a stretch of variant codon sites. In some instances, a nucleic acid may comprise multiple sites of variant codon sites, wherein none the variant codon sites are adjacent to one another. In some instances, a nucleic acid may comprise multiple sites of variant codon sites, wherein some the variant codon sites are adjacent to one another, forming a stretch of variant codon sites, and some of the variant codon sites are not adjacent to one another.

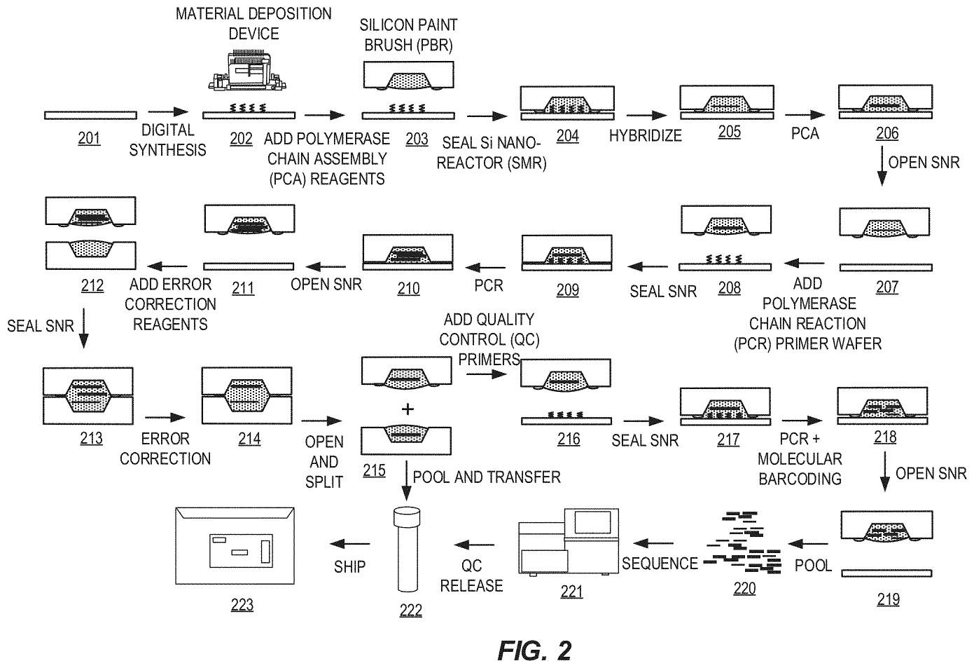

Referring to the Figures, FIG. 2 illustrates an exemplary process workflow for synthesis of nucleic acids (e.g., genes) from shorter nucleic acids. The workflow is divided generally into phases: (1) de novo synthesis of a single stranded nucleic acid library, (2) joining nucleic acids to form larger fragments, (3) error correction, (4) quality control, and (5) shipment. Prior to de novo synthesis, an intended nucleic acid sequence or group of nucleic acid sequences is preselected. For example, a group of genes is preselected for generation.

Once large nucleic acids for generation are selected, a predetermined library of nucleic acids is designed for de novo synthesis. Various suitable methods are known for generating high density polynucleotide arrays. In the workflow example, a device surface layer is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes single nucleotide extension process to extend multiple oligomers in parallel. A deposition device, such as a material deposition device 201, is designed to release reagents in a step wise fashion such that multiple polynucleotides extend, in parallel, one residue at a time to generate oligomers with a predetermined nucleic acid sequence 202. In some instances, polynucleotides are cleaved from the surface at this stage. Cleavage includes gas cleavage, e.g., with ammonia or methylamine.

The generated polynucleotide libraries are placed in a reaction chamber. In this exemplary workflow, the reaction chamber (also referred to as “nanoreactor”) is a silicon coated well, containing PCR reagents and lowered onto the polynucleotide library 203. Prior to or after the sealing 204 of the polynucleotides, a reagent is added to release the polynucleotides from the substrate. In the exemplary workflow, the polynucleotides are released subsequent to sealing of the nanoreactor 205. Once released, fragments of single stranded polynucleotides hybridize in order to span an entire long range sequence of DNA. Partial hybridization 205 is possible because each synthesized polynucleotide is designed to have a small portion overlapping with at least one other polynucleotide in the pool.

After hybridization, a PCA reaction is commenced. During the polymerase cycles, the polynucleotides anneal to complementary fragments and gaps are filled in by a polymerase. Each cycle increases the length of various fragments randomly depending on which polynucleotides find each other. Complementarity amongst the fragments allows for forming a complete large span of double stranded DNA 206.

After PCA is complete, the nanoreactor is separated from the device 207 and positioned for interaction with a device having primers for PCR 208. After sealing, the nanoreactor is subject to PCR 209 and the larger nucleic acids are amplified. After PCR 210, the nanochamber is opened 211, error correction reagents are added 212, the chamber is sealed 213 and an error correction reaction occurs to remove mismatched base pairs and/or strands with poor complementarity from the double stranded PCR amplification products 214. The nanoreactor is opened and separated 215. Error corrected product is next subject to additional processing steps, such as PCR and molecular bar coding, and then packaged 222 for shipment 223.

In some instances, quality control measures are taken. After error correction, quality control steps include for example interaction with a wafer having sequencing primers for amplification of the error corrected product 216, sealing the wafer to a chamber containing error corrected amplification product 217, and performing an additional round of amplification 218. The nanoreactor is opened 219 and the products are pooled 220 and sequenced 221. After an acceptable quality control determination is made, the packaged product 222 is approved for shipment 223.

In some instances, a nucleic acid generate by a workflow such as that in FIG. 2 is subject to mutagenesis using overlapping primers disclosed herein. In some instances, a library of primers are generated by in situ preparation on a solid support and utilize single nucleotide extension process to extend multiple oligomers in parallel. A deposition device, such as a material deposition device, is designed to release reagents in a step wise fashion such that multiple polynucleotides extend, in parallel, one residue at a time to generate oligomers with a predetermined nucleic acid sequence 202.

Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

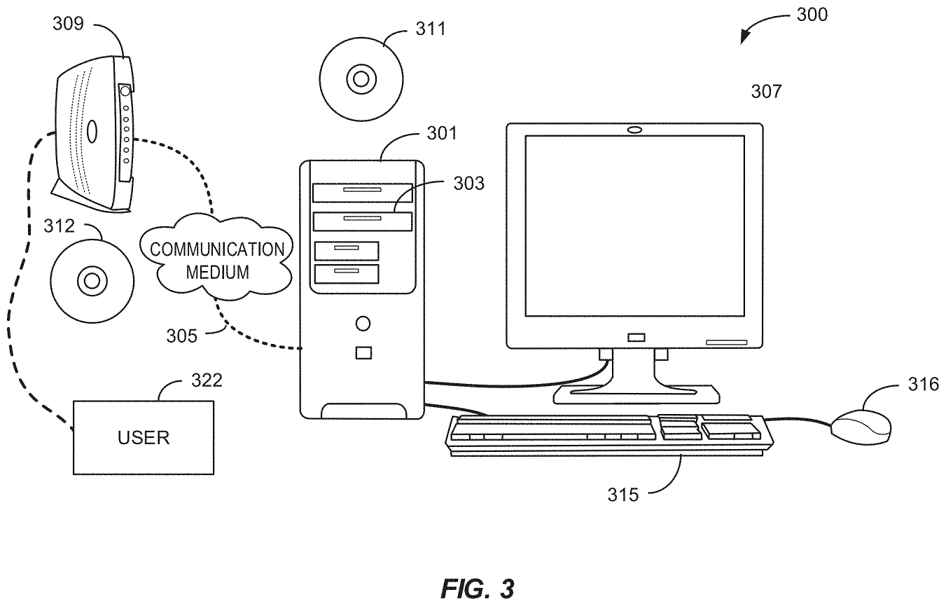

The computer system 300 illustrated in FIG. 3 may be understood as a logical apparatus that can read instructions from media 311 and/or a network port 305, which can optionally be connected to server 309 having fixed media 312. The system, such as shown in FIG. 3 can include a CPU 301, disk drives 303, optional input devices such as keyboard 315 and/or mouse 316 and optional monitor 307. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 322 as illustrated in FIG. 3.

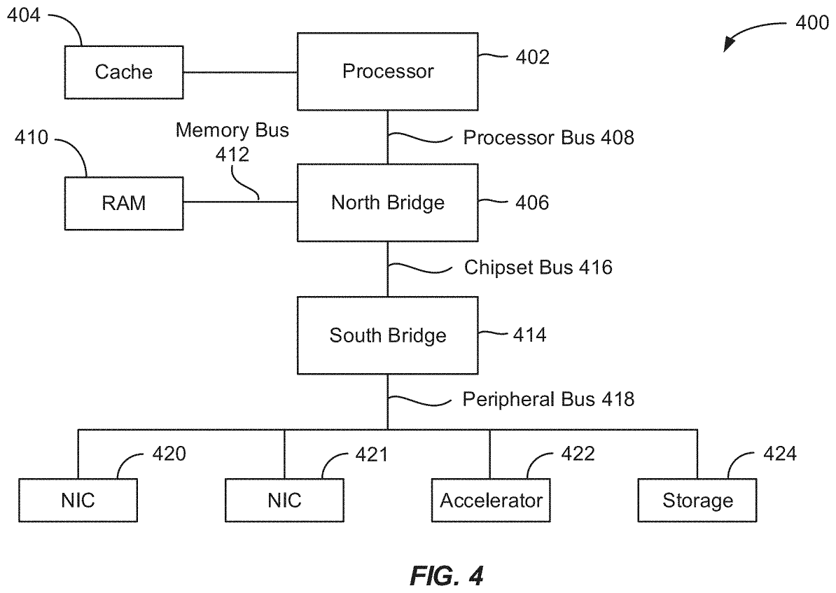

FIG. 4 is a block diagram illustrating a first example architecture of a computer system 400 that can be used in connection with example instances of the present disclosure. As depicted in FIG. 4, the example computer system can include a processor 402 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 4, a high speed cache 404 can be connected to, or incorporated in, the processor 402 to provide a high speed memory for instructions or data that have been recently, or are frequently, used by processor 402. The processor 402 is connected to a north bridge 406 by a processor bus 408. The north bridge 406 is connected to random access memory (RAM) 410 by a memory bus 412 and manages access to the RAM 410 by the processor 402. The north bridge 406 is also connected to a south bridge 414 by a chipset bus 416. The south bridge 414 is, in turn, connected to a peripheral bus 418. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 418. In some alternative architectures, the functionality of the north bridge can be incorporated into the processor instead of using a separate north bridge chip. In some instances, system 400 can include an accelerator card 422 attached to the peripheral bus 418. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 424 and can be loaded into RAM 410 and/or cache 404 for use by the processor. The system 400 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 400 also includes network interface cards (NICs) 420 and 421 connected to the peripheral bus for providing network interfaces to external storage, such as Network Attached Storage (NAS) and other computer systems that can be used for distributed parallel processing.

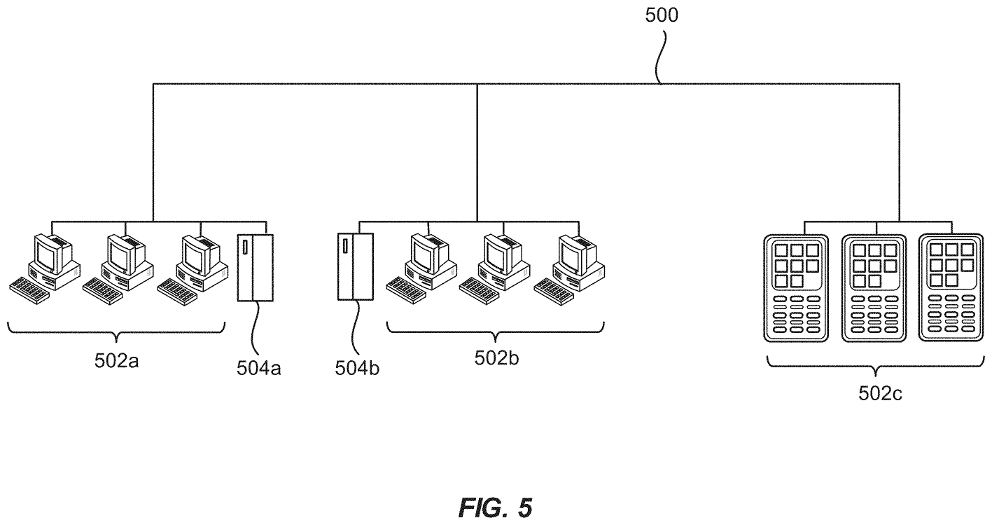

FIG. 5 is a diagram showing a network 500 with a plurality of computer systems 502a, and 502b, a plurality of cell phones and personal data assistants 502c, and Network Attached Storage (NAS) 504a, and 504b. In example instances, systems 502a, 502b, and 502c can manage data storage and optimize data access for data stored in Network Attached Storage (NAS) 504a and 504b. A mathematical model can be used for the data and be evaluated using distributed parallel processing across computer systems 502a, and 502b, and cell phone and personal data assistant systems 502c. Computer systems 502a, and 502b, and cell phone and personal data assistant systems 502c can also provide parallel processing for adaptive data restructuring of the data stored in Network Attached Storage (NAS) 504a and 504b. FIG. 5 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

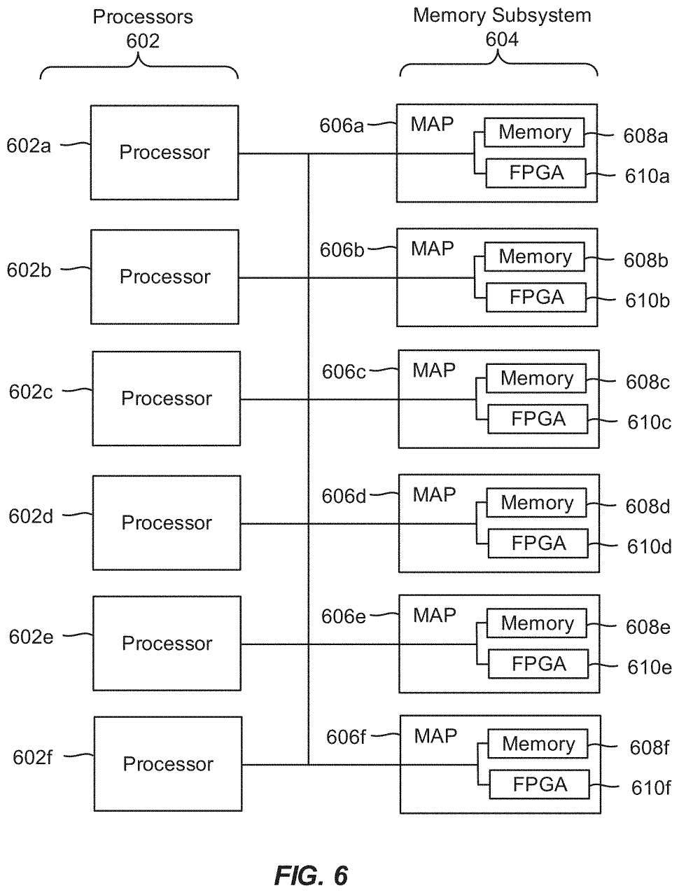

FIG. 6 is a block diagram of a multiprocessor computer system using a shared virtual address memory space in accordance with an example instance. The system includes a plurality of processors 602a-f that can access a shared memory subsystem 604. The system incorporates a plurality of programmable hardware memory algorithm processors (MAPs) 606a-f in the memory subsystem 604. Each MAP 606a-f can comprise a memory 608a-f and one or more field programmable gate arrays (FPGAs) 610a-f The MAP provides a configurable functional unit and particular algorithms or portions of algorithms can be provided to the FPGAs 610a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 608a-f, allowing it to execute tasks independently of, and asynchronously from the respective microprocessor 602a-f In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 4, system on chips (SOCs), application specific integrated circuits (ASICs), or other processing and logic elements. For example, the Set Processor and Optimizer can be implemented with hardware acceleration through the use of a hardware accelerator card, such as accelerator card 422 illustrated in FIG. 4.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Functionalization of a Device Surface

A device was functionalized to support the attachment and synthesis of a library of polynucleotides. The device surface was first wet cleaned using a piranha solution comprising 90% H₂SO₄ and 10% H₂O₂ for 20 minutes. The device was rinsed in several beakers with DI water, held under a DI water gooseneck faucet for 5 min, and dried with N₂. The device was subsequently soaked in NH₄OH (1:100; 3 mL:300 mL) for 5 min, rinsed with DI water using a handgun, soaked in three successive beakers with DI water for 1 min each, and then rinsed again with DI water using the handgun. The device was then plasma cleaned by exposing the device surface to O₂. A SAMCO PC-300 instrument was used to plasma etch O₂ at 250 watts for 1 min in downstream mode.

The cleaned device surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The device surface was resist coated using a Brewer Science 200X spin coater. SPR™ 3612 photoresist was spin coated on the device at 2500 rpm for 40 sec. The device was pre-baked for 30 min at 90° C. on a Brewer hot plate. The device was subjected to photolithography using a Karl Suss MA6 mask aligner instrument. The device was exposed for 2.2 sec and developed for 1 min in MSF 26A. Remaining developer was rinsed with the handgun and the device soaked in water for 5 min. The device was baked for 30 min at 100° C. in the oven, followed by visual inspection for lithography defects using a Nikon L200. A descum process was used to remove residual resist using the SAMCO PC-300 instrument to O₂ plasma etch at 250 watts for 1 min.

The device surface was passively functionalized with a 100 μL solution of perfluorooctyltrichlorosilane mixed with 10 μl light mineral oil. The device was placed in a chamber, pumped for 10 min, and then the valve was closed to the pump and left to stand for 10 min. The chamber was vented to air. The device was resist stripped by performing two soaks for 5 min in 500 mL NMP at 70° C. with ultrasonication at maximum power (9 on Crest system). The device was then soaked for 5 min in 500 mL isopropanol at room temperature with ultrasonication at maximum power. The device was dipped in 300 mL of 200 proof ethanol and blown dry with N₂. The functionalized surface was activated to serve as a support for polynucleotide synthesis.

### Example 2: Synthesis of a 50-Mer Sequence on an Oligonucleotide Synthesis Device

A two dimensional oligonucleotide synthesis device was assembled into a flowcell, which was connected to a flowcell (Applied Biosystems (ABI394 DNA Synthesizer”). The two-dimensional oligonucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described. 5′AGACAATCAACCATTTGGGGTGGACAGCCTTGACCTCTAGACTTCGGCATOTTTTTT TTTT3′ (SEQ ID NO: 4213), where # denotes Thymidine-succinyl hexamide CED phosphoramidite (CLP-2244 from ChemGenes), which is a cleavable linker enabling the release of oligos from the surface during deprotection.

The synthesis was done using standard DNA synthesis chemistry (coupling, capping, oxidation, and deblocking) according to the protocol in Table 4 and an ABI synthesizer.

TABLE 4
Synthesis protocols
Table 4

| General DNA Synthesis | | |
|---|---|---|
| Process Name | Process Step | Time (sec) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| CAPPING (CapA + B, 1:1, | CapA + B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M I2 in 20% pyridine, 10% water, and 70% THF) were roughly ˜100 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/sec, and for Deblock (3% dichloroacetic acid in toluene), roughly ˜300 uL/sec (compared to −50 uL/sec for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip.

### Example 3: Synthesis of a 100-Mer Sequence on an Oligonucleotide Synthesis Device

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGTCATG CTAGCCATACCATGATGATGATGATGATGAGAACCCCGCAT ##TTTTTTTTTT3′ (SEQ ID NO: 4214), where # denotes Thymidine-succinyl hexamide CED phosphoramidite (CLP-2244 from ChemGenes) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument.

All ten samples from the two chips were further PCR amplified using a forward (5′ATGCGGGGTTCTCATCATC3′ (SEQ ID NO: 4215)) and a reverse (5′CGGGATCCTTATCGTCATCG3′ (SEQ ID NO: 4216)) primer in a 50 uL PCR mix (25 uL NEB Q5 mastermix, 2.5 uL 10 uM Forward primer, 2.5 uL 10 uM Reverse primer, 1 uL polynucleotide extracted from the surface, and water up to 50 uL) using the following thermalcycling program:

98° C., 30 sec

98° C., 10 sec; 63° C., 10 sec; 72° C., 10 sec; repeat 12 cycles

72° C., 2 min

The PCR products were also run on a BioAnalyzer, demonstrating sharp peaks at the 100-mer position. Next, the PCR amplified samples were cloned, and Sanger sequenced. Table 5 summarizes the results from the Sanger sequencing for samples taken from spots 1-5 from chip 1 and for samples taken from spots 6-10 from chip 2.

TABLE 5
Sequencing results

| Spot | Error rate | Cycle efficiency |
|---|---|---|
| 1 | 1/763 bp | 99.87% |
| 2 | 1/824 bp | 99.88% |
| 3 | 1/780 bp | 99.87% |
| 4 | 1/429 bp | 99.77% |
| 5 | 1/1525 bp | 99.93% |
| 6 | 1/1615 bp | 99.94% |
| 7 | 1/531 bp | 99.81% |
| 8 | 1/1769 bp | 99.94% |
| 9 | 1/854 bp | 99.88% |
| 10 | 1/1451 bp | 99.93% |

Thus, the high quality and uniformity of the synthesized polynucleotides were repeated on two chips with different surface chemistries. Overall, 89% of the 100-mers that were sequenced were perfect sequences with no errors, corresponding to 233 out of 262.

Table 6 summarizes error characteristics for the sequences obtained from the polynucleotides samples from spots 1-10.

TABLE 6
Error characteristics

| Sample ID/Spot no. | OSA_0046/1 | OSA_0047/2 | OSA_0048/3 | OSA_0049/4 | OSA_0050/5 |
|---|---|---|---|---|---|
| Total Sequences | 32 | 32 | 32 | 32 | 32 |
| Sequencing Quality | 25 of 28 | 27 of 27 | 26 of 30 | 21 of 23 | 25 of 26 |
| Oligo Quality | 23 of 25 | 25 of 27 | 22 of 26 | 18 of 21 | 24 of 25 |
| ROI Match Count | 2500 | 2698 | 2561 | 2122 | 2499 |
| ROI Mutation | 2 | 2 | 1 | 3 | 1 |
| ROI Multi | 0 | 0 | 0 | 0 | 0 |
| Base Deletion | | | | | |
| ROI Small | 1 | 0 | 0 | 0 | 0 |
| Insertion | | | | | |
| ROI Single | 0 | 0 | 0 | 0 | 0 |
| Base Deletion | | | | | |
| Large Deletion | 0 | 0 | 1 | 0 | 0 |
| Count | | | | | |
| Mutation: G > A | 2 | 2 | 1 | 2 | 1 |
| Mutation: T > C | 0 | 0 | 0 | 1 | 0 |
| ROI Error Count | 3 | 2 | 2 | 3 | 1 |
| ROI Error Rate | Err: ~1 in 834 | Err: ~1 in 1350 | Err: ~1 in 1282 | Err: ~1 in 708 | Err: ~1 in 2500 |
| ROI Minus Primer | MP Err: ~1 in 763 | MP Err: ~1 in 824 | MP Err: ~1 in 780 | MP Err: ~1 in 429 | MP Err: ~1 in 1525 |
| Error Rate | | | | | |
| Sample ID/Spot no. | OSA_0051/6 | OSA_0052/7 | OSA_0053/8 | OSA_0054/9 | OSA_0055/10 |
| Total Sequences | 32 | 32 | 32 | 32 | 32 |
| Sequencing Quality | 29 of 30 | 27 of 31 | 29 of 31 | 28 of 29 | 25 of 28 |
| Oligo Quality | 25 of 29 | 22 of 27 | 28 of 29 | 26 of 28 | 20 of 25 |
| ROI Match Count | 2666 | 2625 | 2899 | 2798 | 2348 |
| ROI Mutation | 0 | 2 | 1 | 2 | 1 |
| ROI Multi | 0 | 0 | 0 | 0 | 0 |
| Base Deletion | | | | | |
| ROI Small | 0 | 0 | 0 | 0 | 0 |
| Insertion | | | | | |
| ROI Single | 0 | 0 | 0 | 0 | 0 |
| Base Deletion | | | | | |
| Large Deletion | 1 | 1 | 0 | 0 | 0 |
| Count | | | | | |
| Mutation: G > A | 0 | 2 | 1 | 2 | 1 |
| Mutation: T > C | 0 | 0 | 0 | 0 | 0 |
| ROI Error Count | 1 | 3 | 1 | 2 | 1 |
| ROI Error Rate | Err: ~1 in 2667 | Err: ~1 in 876 | Err: ~1 in 2900 | Err: ~1 in 1400 | Err: ~1 in 2349 |
| ROI Minus Primer | MP Err: ~1 in 1615 | MP Err: ~1 in 531 | MP Err: ~1 in 1769 | MP Err: ~1 in 854 | MP Err: ~1 in 1451 |
| Error Rate | | | | | |

### Example 4: Identification of Antibodies

Antibodies to be used with the lateral flow device were identified.

Briefly, antibodies were identified using phage display. Antibody expressing bacteriophage libraries were panned against the SARS-CoV-2 spike protein for binding, screened for binding after 3-4 panning rounds, and then underwent DNA sequencing to determine the sequence of the antibody being expressed. This process yielded 1,152 sequences (3×384 samples) analyzed via next-generation DNA sequencing (NGS).

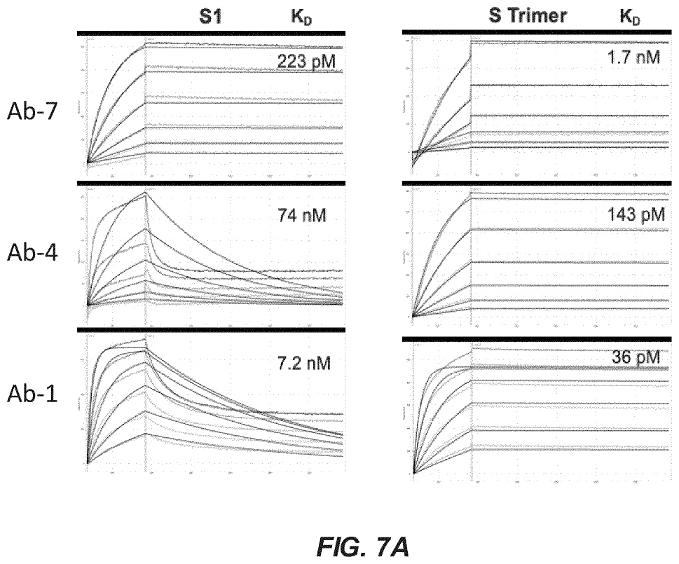

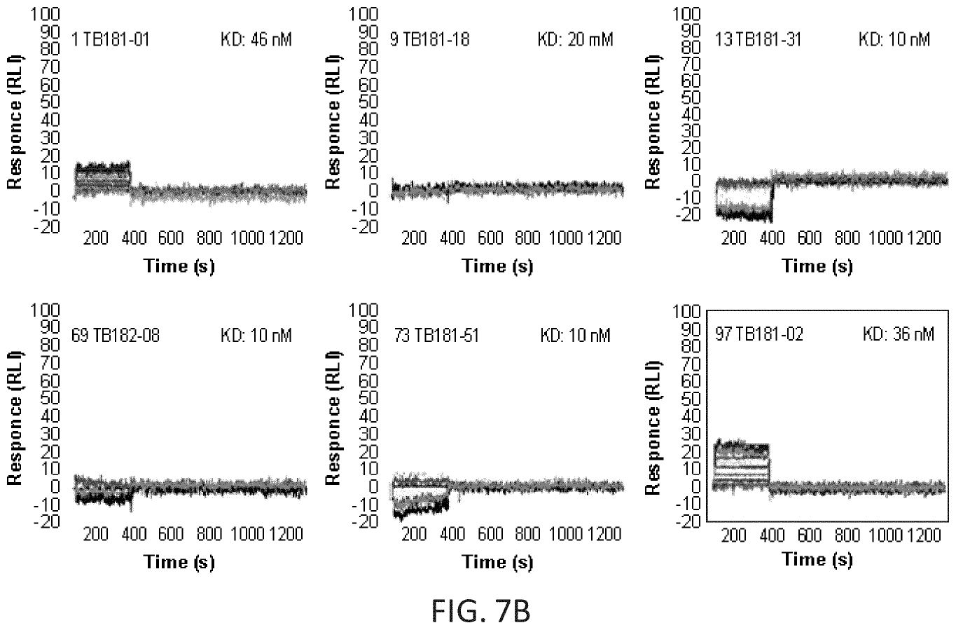

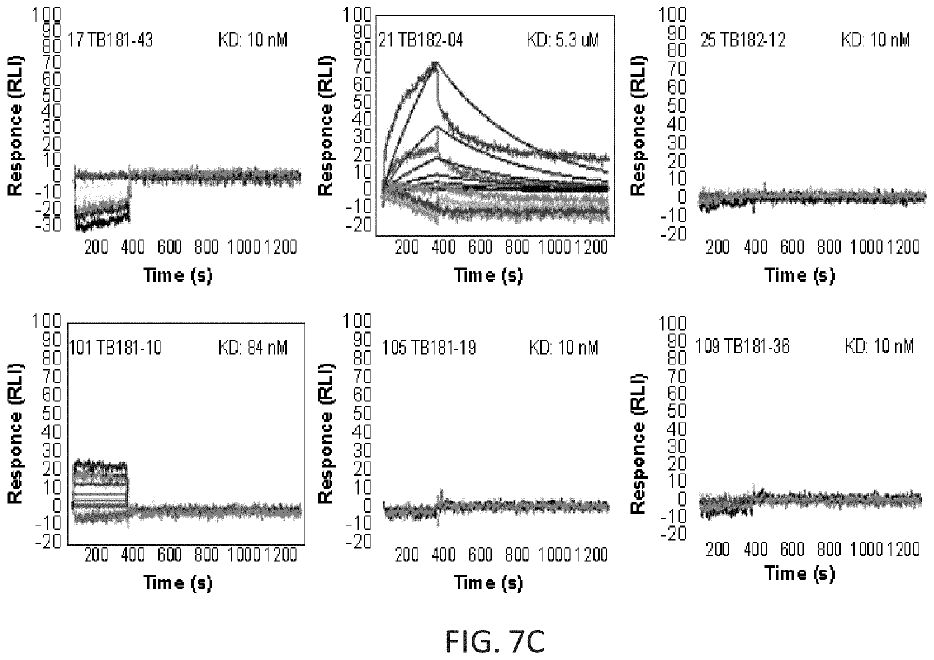

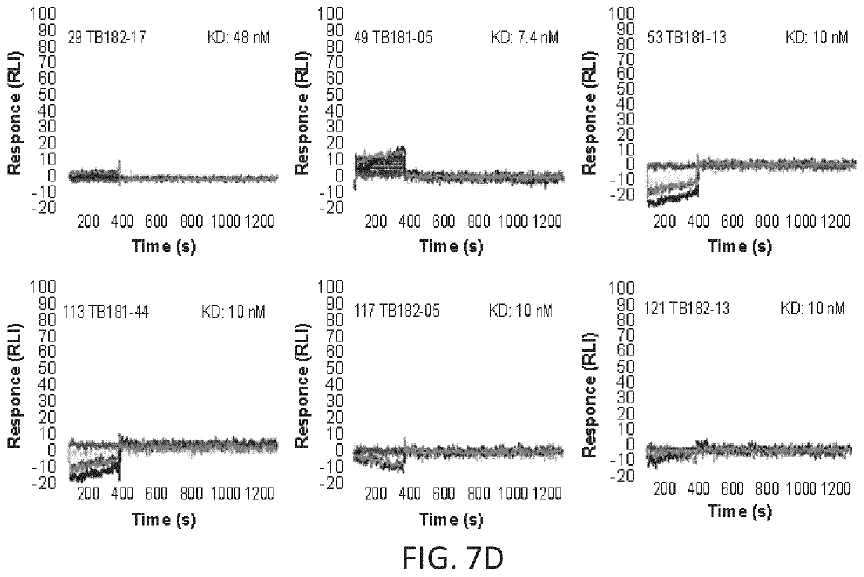

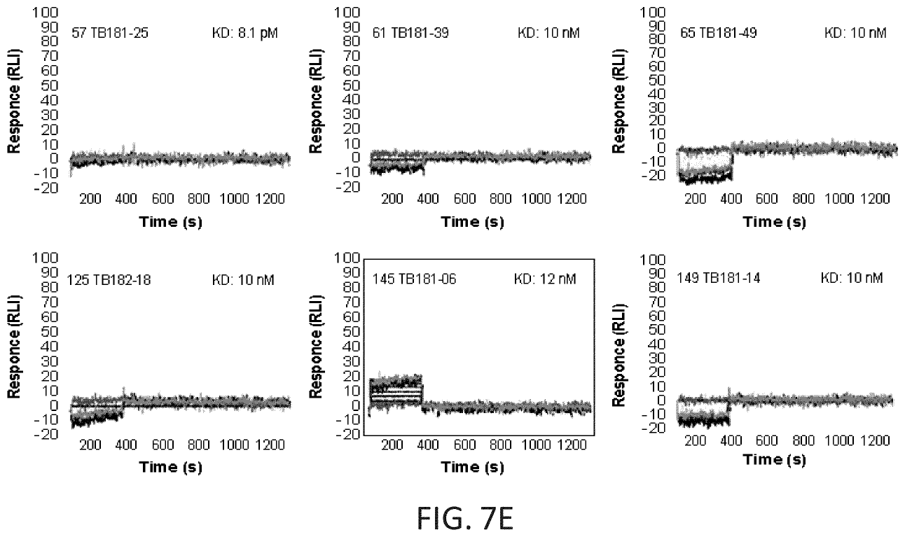

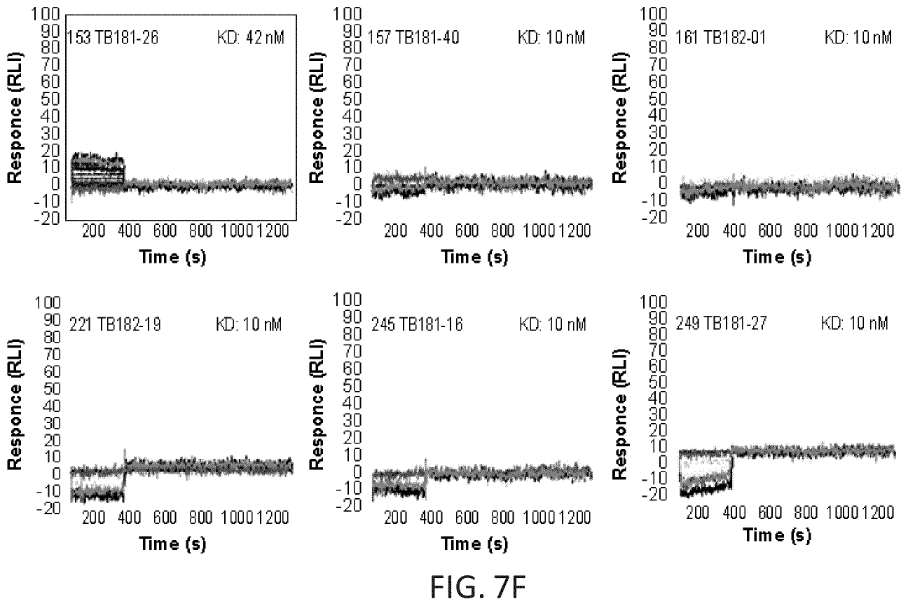

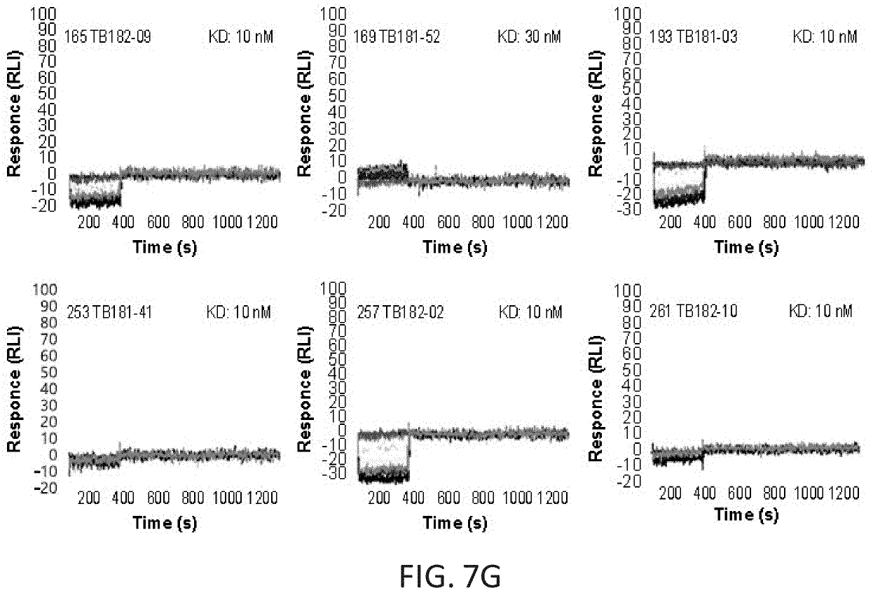

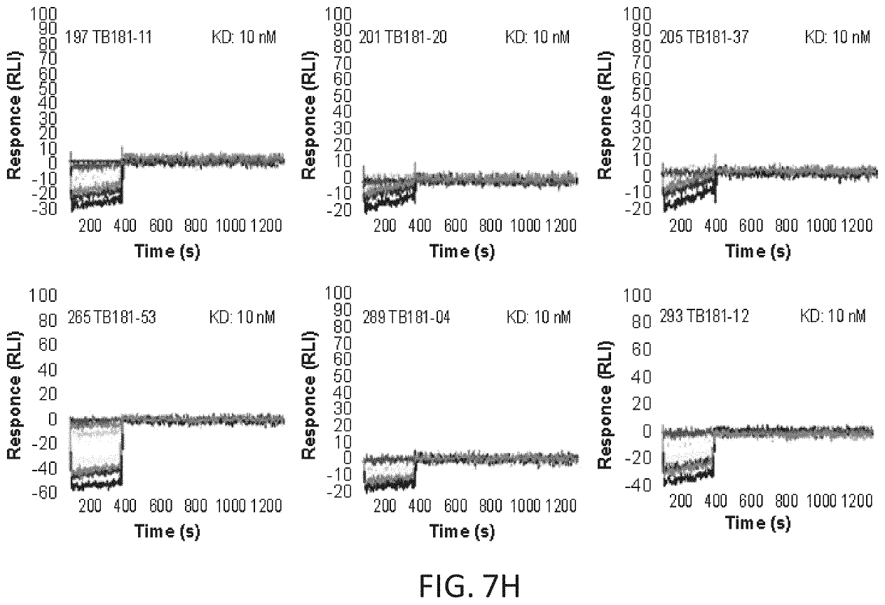

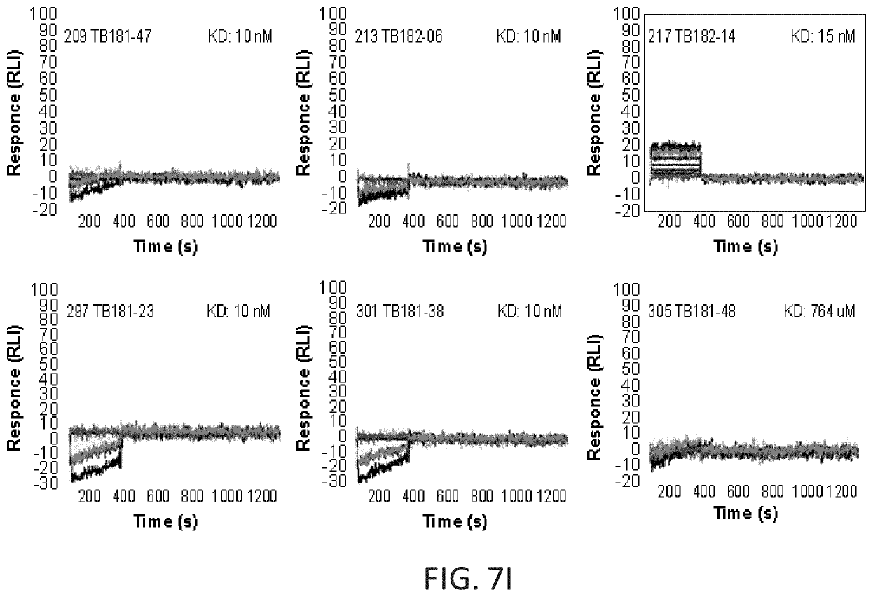

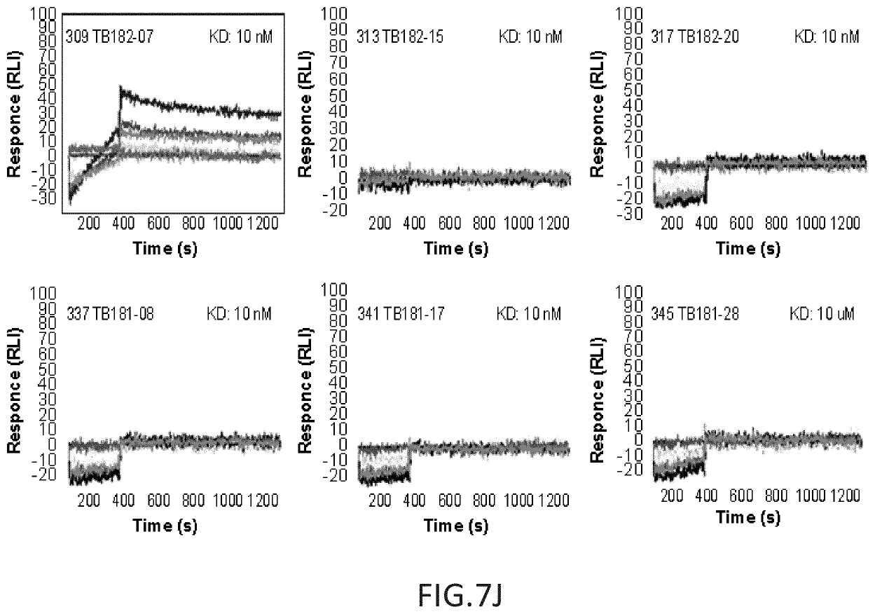

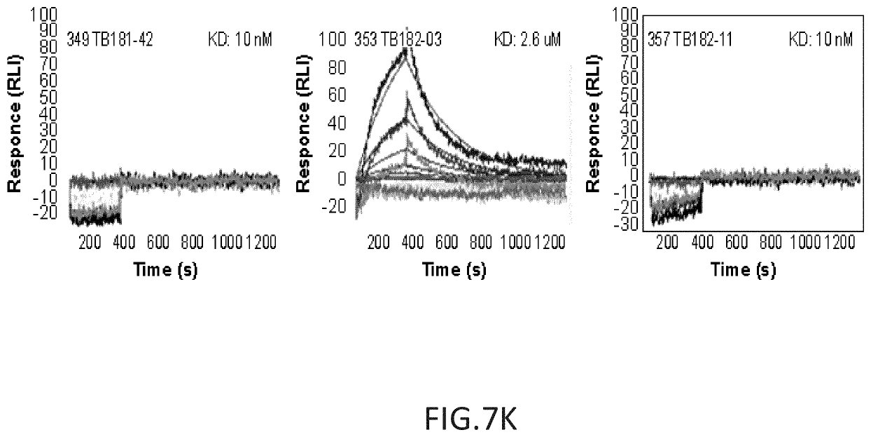

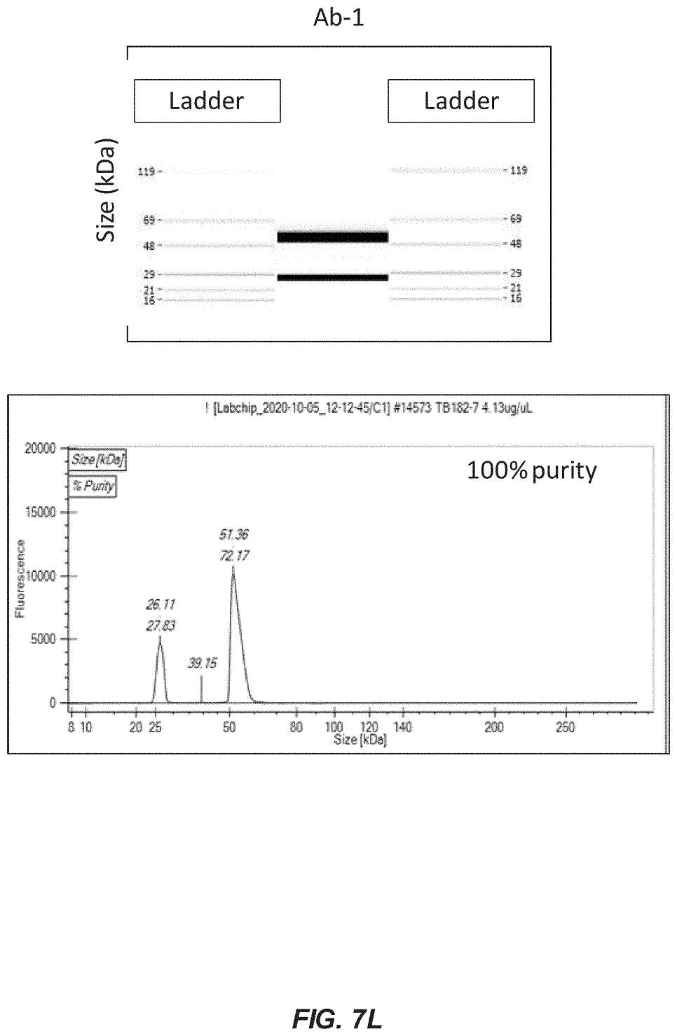

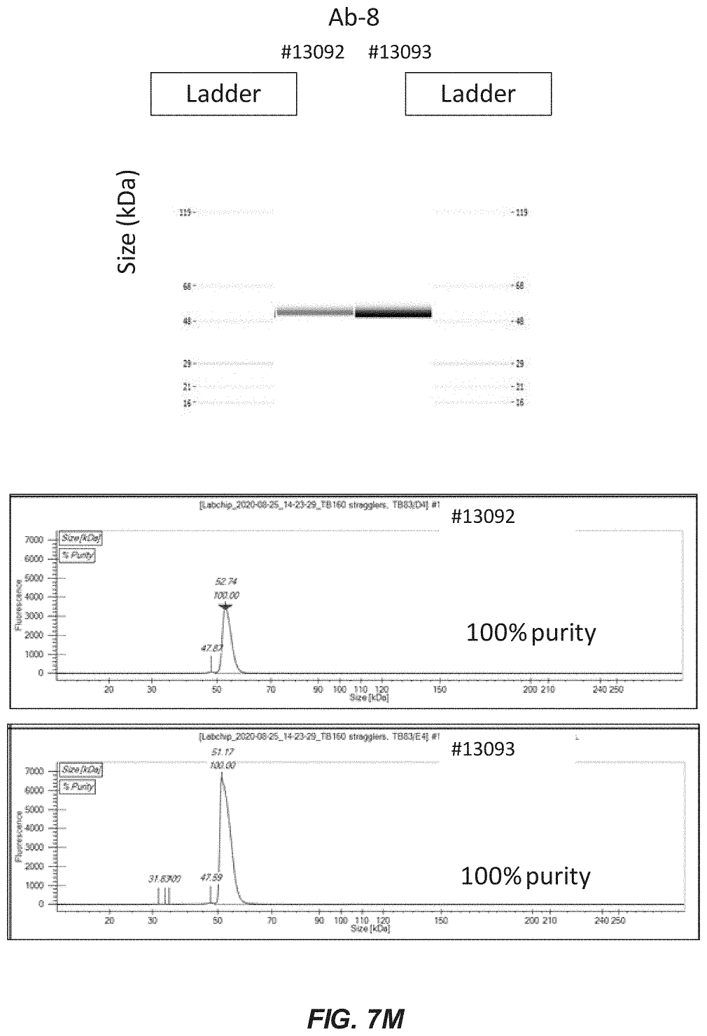

A panel of antibodies were identified that comprise high affinity binding to S1 monomer and S trimer in ELISA (data not shown). Using surface plasmon resonance, many of the antibodies were determined to bind with subnanomolar binding to SARS-CoV-2 S1 monomer and/or S trimer (FIG. 7A). Ab-7 and Ab-4 showed no binding to the related SARS-CoV virus S1 protein (FIG. 7B). For Ab-7 and Ab-4, the SARS-CoV S1 binding affinities were in the micromolar range.

Ab-1 and Ab-8 were further analysis used CE-SDS gel and electropherograms. 2 uL of sample was used and reduced using 34 mM DTT. Data for Ab-1 and Ab-8 are seen in FIGS. 7C-7D and Table 7.

TABLE 7

| | Concentration | Available Yield |
|---|---|---|
| Name | (mg/mL) | (mg) |
| Ab-1 | 4.13 | 103.5 |
| Ab-8 (Lot 13092) | 5.44 | 76.1 |
| Ab-8 (Lot 13093) | 9.35 | 177.6 |

### Example 5: Lateral Flow Device

A lateral flow device using the antibodies identified was designed.

Antibodies described in Example 4 were used in a lateral flow device. Ab-1 captured SARS-CoV-2 spike protein in a concentration dependent manner and bound antigen with either Ab-7, Ab-4, or a control antibody CR3022 (Abcam) was detected (FIG. 8). The data shows that the SARS-CoV-2 spike protein was captured and presented for detection by a complementary sandwich pair with Ab-7 or Ab-4 that was improved as compared to the control antibody.

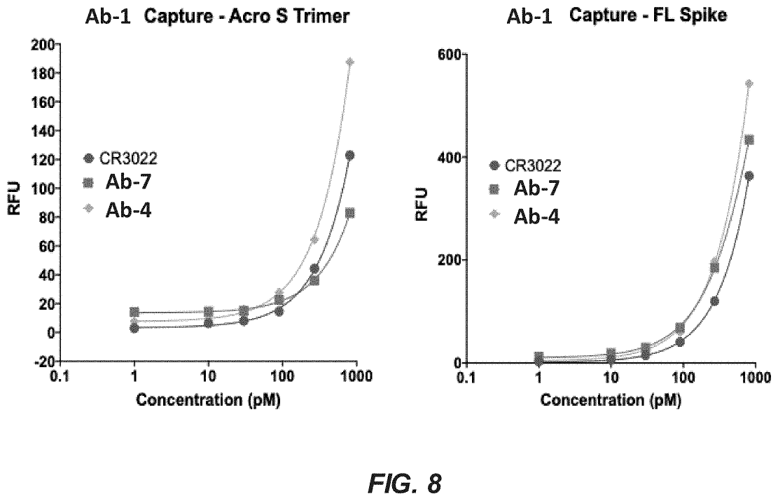

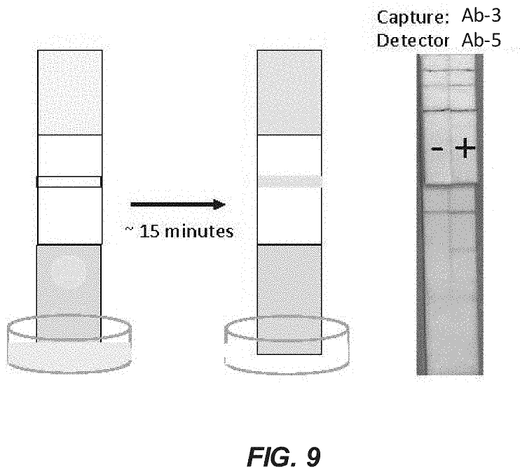

The antibodies were further screened for improved pairs of antibodies. Detector antibodies were conjugated with latex via Amide Beads. The following capture and detector antibody pairs were identified: Ab-1 capture with either Ab-2 or Ab-3 detector; Ab-4 capture with either Ab-2, Ab-5, or Ab-6 detector; Ab-7 capture with either Ab-5 or Ab-1 detector; and Ab-3 capture with Ab-2 detector. Exemplary results using the lateral flow device is seen in FIG. 9.

Initial limit of detection (LOD) assessments were also performed. Ab-3/Ab-2 and Ab-1/Ab-3 was observed to have a linear curve with an unoptimized LOD of about 125 and 63 ng/mL, respectively.

### Example 6: Lateral Flow Assay

A lateral flow assay was performed using the antibodies described herein.

Details of the lateral flow assay are seen in Table 8.

TABLE 8

| Target Analyte | SRAS-CoV-2 Spike Protein |
|---|---|
| Particle System | Colloidal Gold (Lumos) |
| Data Output | Colorimetric (Absorbance) |
| Detector Antibody | Ab-8 |
| Test Line (Capture | Ab-1 |
| Antibody) | |
| Membrane | Unbacked CN140 |
| Waste Pad | A440 (Ahlstrom) |
| Sample Pad | Ahlstrom 6614 treated with PVP-10, PVA, |
| | IGEPAL CA-60 in Citrate buffer |
| Conjugate Pad | Ahlstrom 8950 treated with BSA and |
| | Tween-20 in Borate buffer |
| Antibody Loading | 6 μg antibody per mL per OD gold |
| Conjugate | OD10 |
| Concentration | |
| Conjugate Spray Rate | 10 μL/cm |
| Conjugate Diluent | Colloidal gold resuspension buffer + |
| Buffer | 10% sucrose + 5% trehalose |
| Assay Performance | Performance at Feasibility |
| Sensitivity | 10 ng/ml |
| Time to result | 15 minutes |
| Matrix | Pure Sal Saliva |

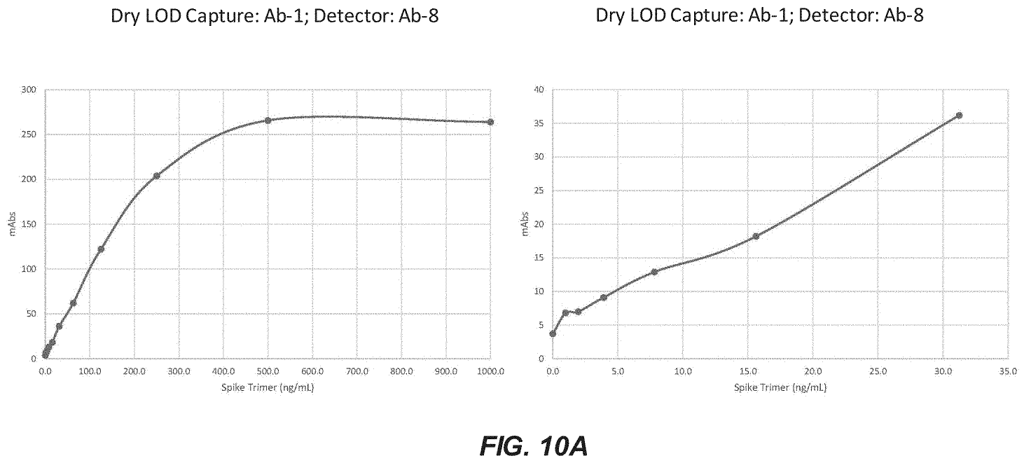

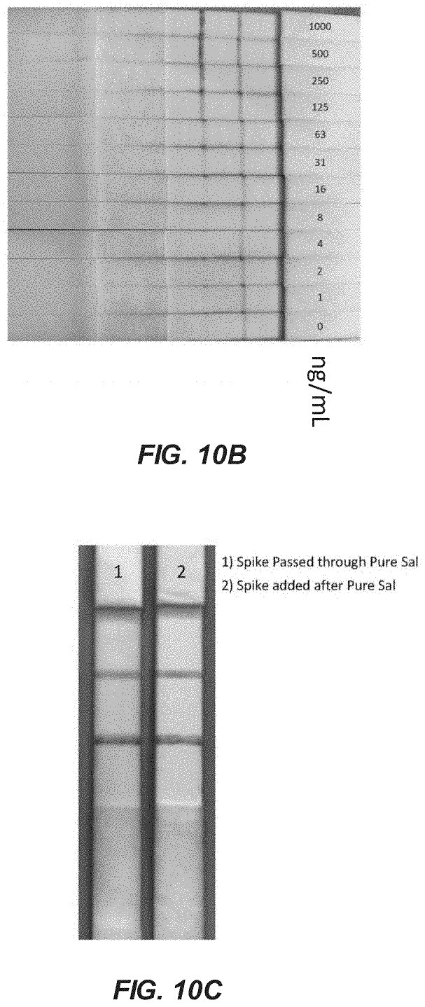

Using the lateral flow assay, a dry test strip limit of detection was tested using saliva. The data is seen in FIGS. 10A-10B. FIG. 10A shows the dry system successfully detected 7.8 ng/mL of SARS-CoV-2 spike trimer protein that was spiked into salvia and showed a linear curve as the concentration of spike protein increases. FIG. 10B shows the limit of detection is close to 10 ng/mL SARS-CoV-2 spike trimer protein. The lateral flow device was also assayed for whether the spike trimer can pass through the device. As seen in FIG. 10C, the spike trimer passed through the device and a signal was detected.

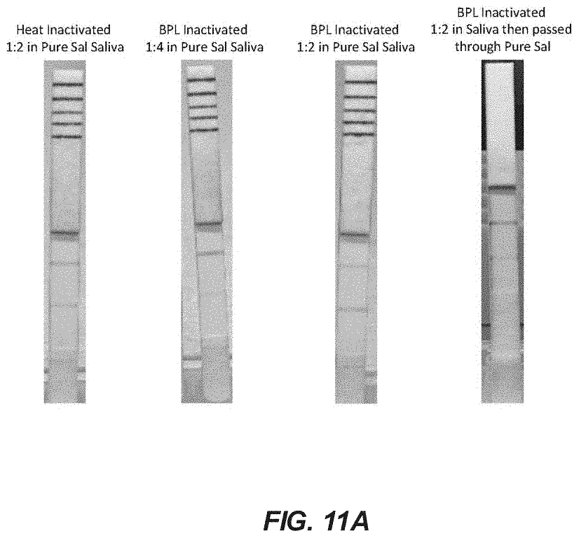

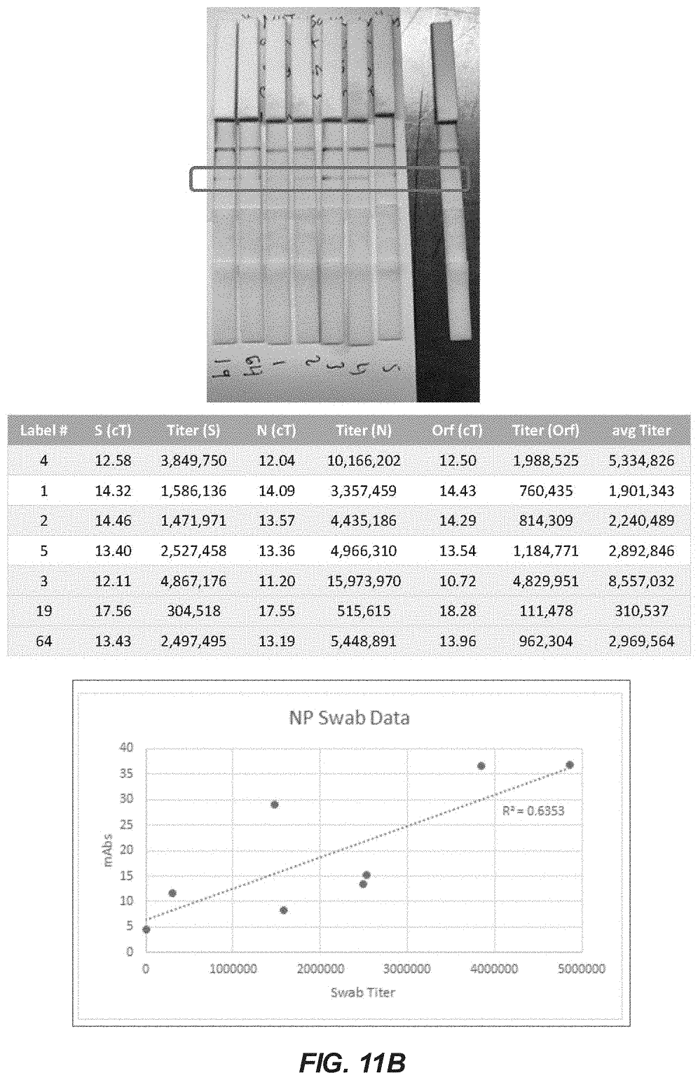

The lateral flow assay was then tested using inactivated virus and live virus on swabs. Spike inactivated virus in saliva was passed through the lateral flow device as was spike inactivated virus in raw saliva. FIG. 11A shows data on test strips testing inactivated virus. As seen in FIG. 11A, positive signal was observed for both the heat and BPL inactivated virus. Signal was also observed when the virus mixed with raw saliva was passed through the lateral flow device. FIG. 11B shows data from live virus on swabs. Swab samples in saline were tested using strips. 60 uL of sample was added to the strip and a photo was taken at 15 minutes. All positive swab showed a test line at varying intensities and very little background was observed from the negative swab (FIG. 11B).

This Example shows that the lateral flow assay is able to detect SARS-CoV-2 protein in saliva samples.

### Example 7. Rapid Antigen Detection Test Kit

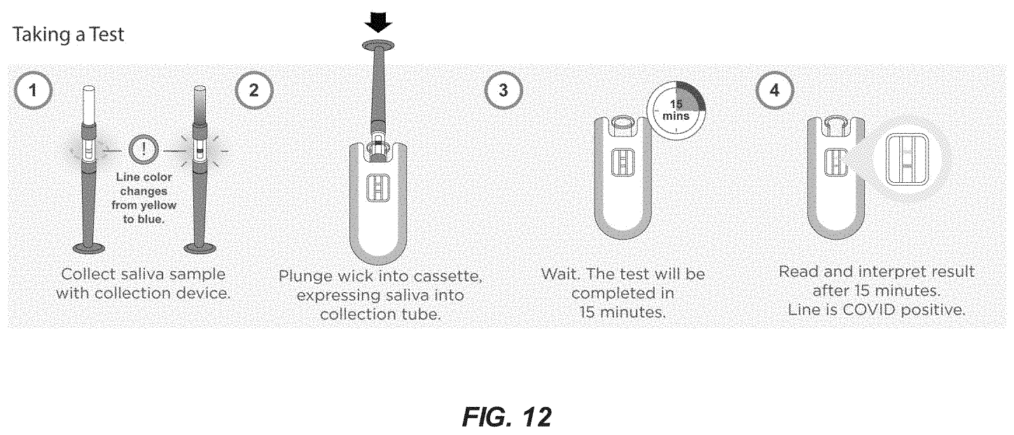

An exemplary schema of the rapid antigen detection (RAD) test kit comprising a lateral flow device is seen in FIG. 12.

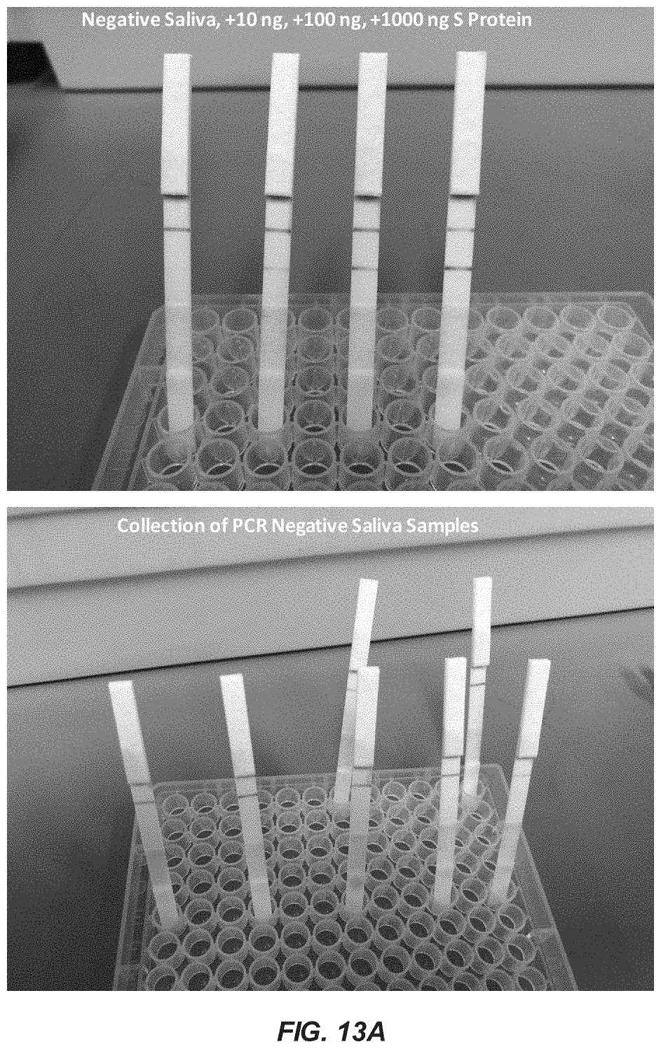

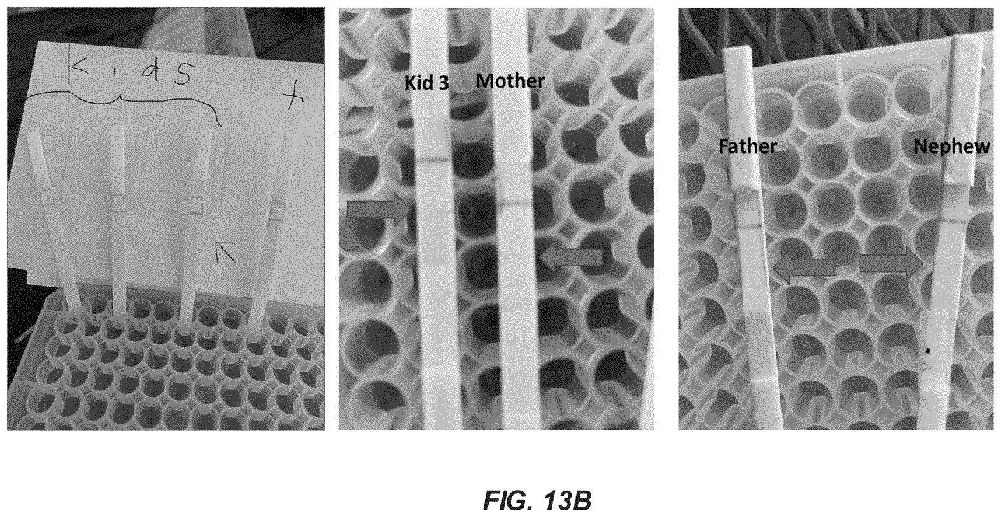

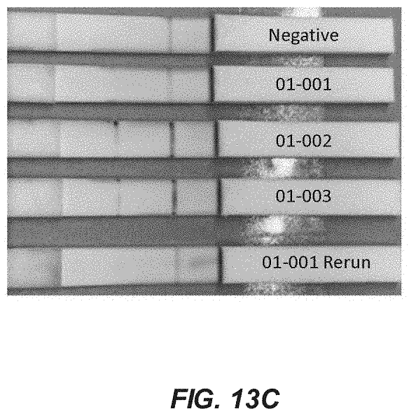

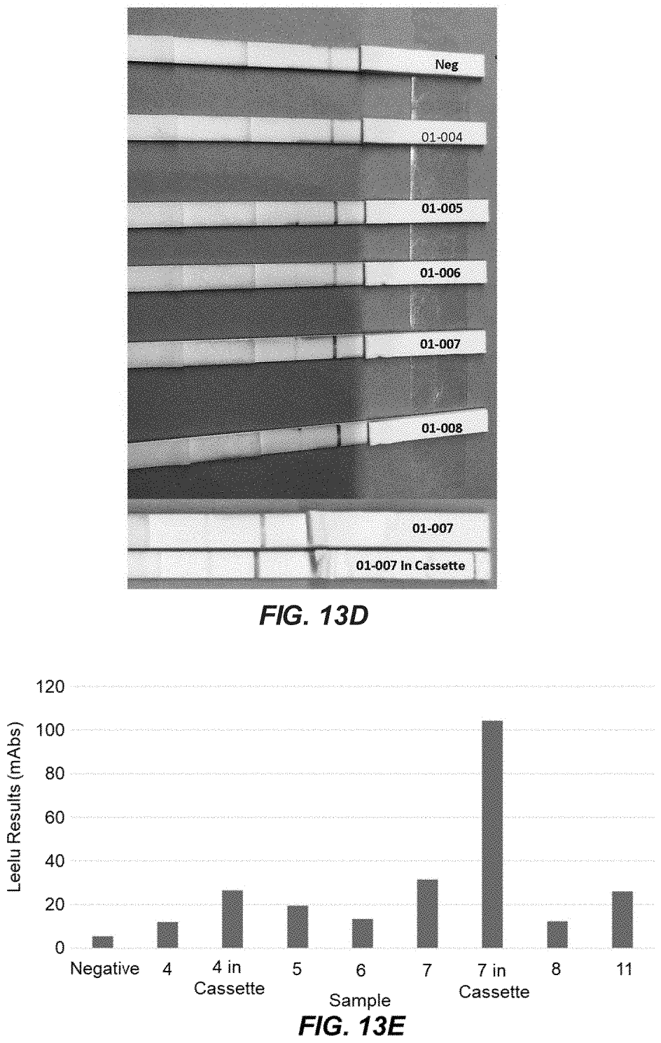

Spike protein was added to saliva and was measured using the lateral flow device. Data from the lateral flow device was compared to results from PCR. As seen in FIGS. 13A-13B, the lateral flow device gave similar results as using PCR. The lateral flow device was also used to test SARS-CoV-2 positive saliva samples. As seen in FIG. 13C, the lateral flow device positively identified two of the three samples that were SARS-CoV-2 positive. FIG. 13D shows data using the lateral flow device to detect SARS-CoV-2 in saliva samples from five additional SARS-CoV-2 positive samples. Using the cassette improved the signal of the test line as seen in FIG. 13E.

### Example 8. Multiplexed SARS-CoV-2 Antigen Test

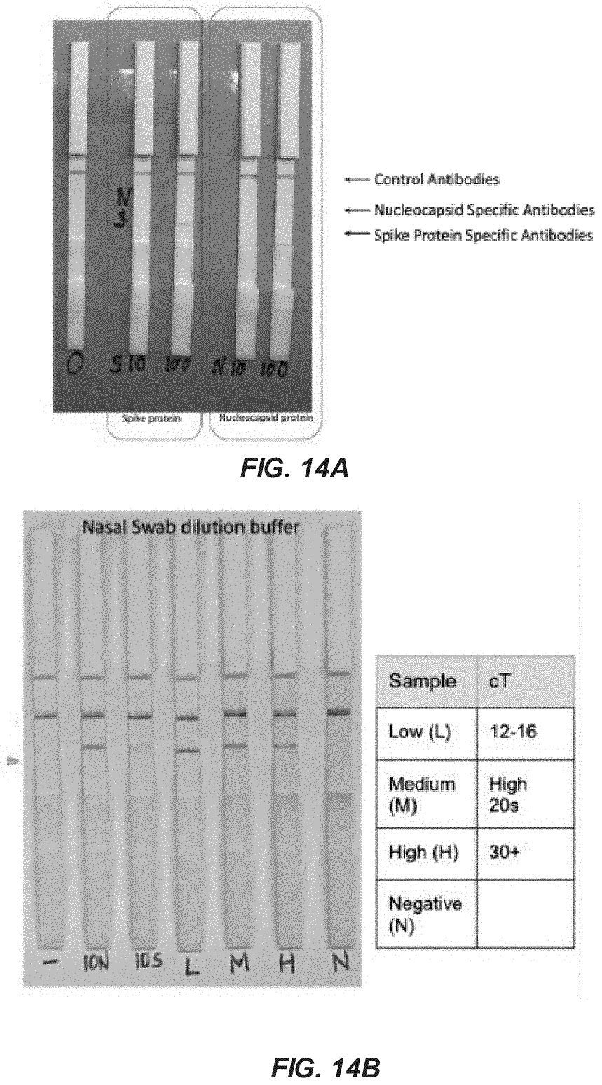

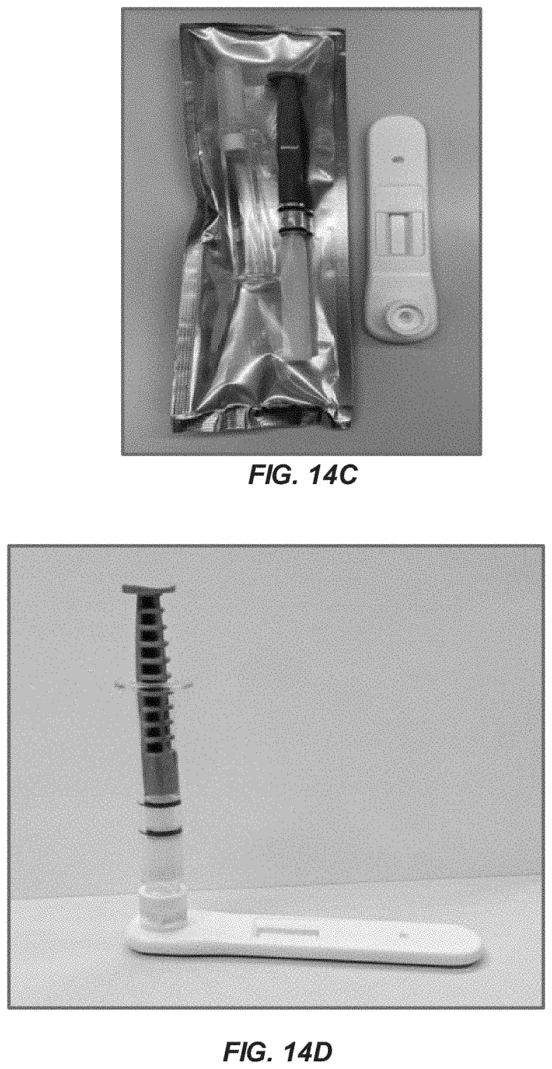

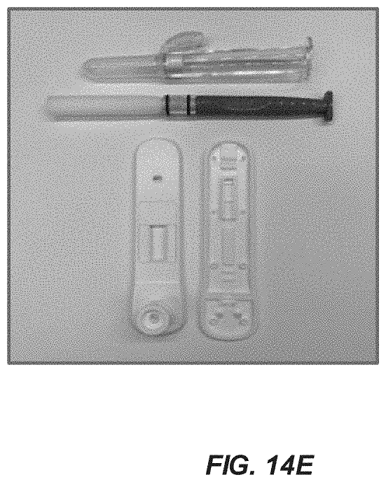

A SARS-CoV-2 lateral flow assay containing a cocktail of antibodies that can detect multiple SARS-CoV-2 specific antigens, specifically the Spike and Nucleocapsid proteins, was developed. The lateral flow assay contains a capture and detector antibody for each antigen. The nucleocapsid specific antibodies are commercially available and the spike protein antibodies were created at Twist Bioscience with Ab-9 detector and Ab-10 capture antibodies. As seen in FIG. 14A, a 3-line system with recombinant protein spiked into negative saliva demonstrates the specificity of the test. As seen in FIG. 14B, a 2-line system with recombinant protein spiked into transfer buffer and nasal swabs from covid-19 positive patients demonstrated the specificity of the test.

### Example 9: Integrated Cassette for Saliva Detection

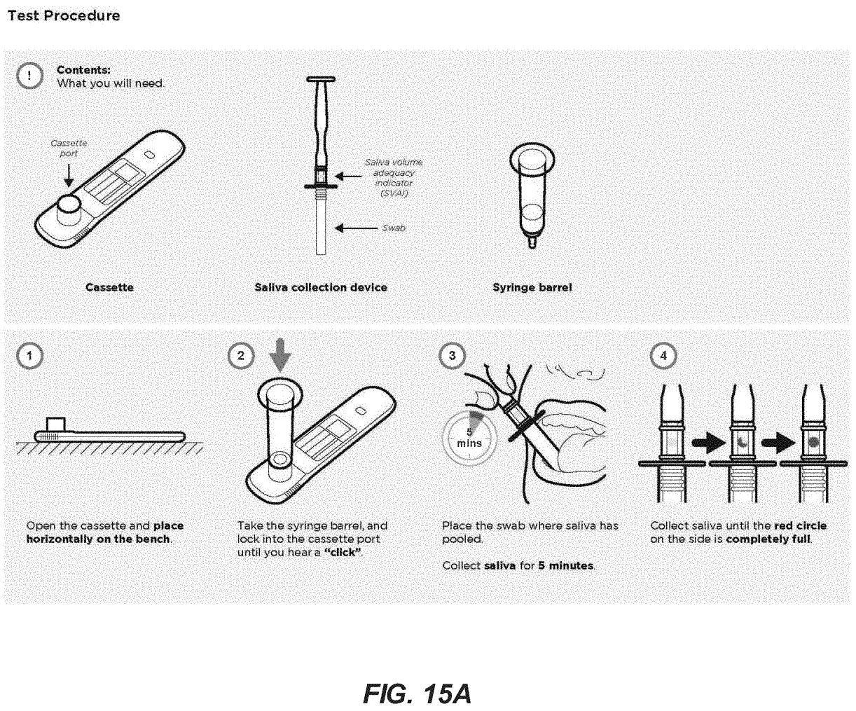

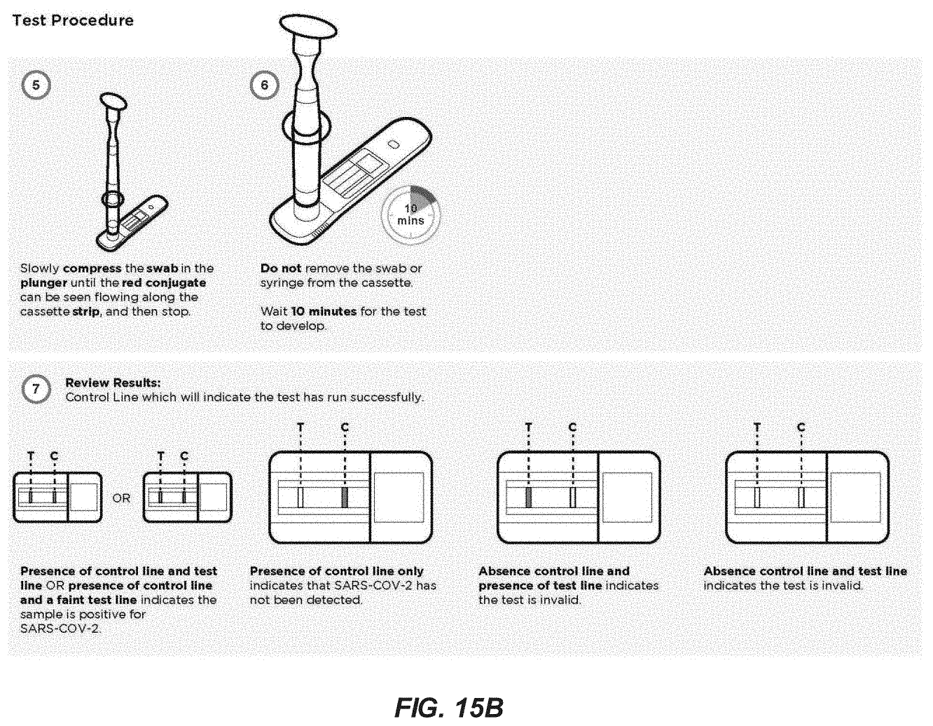

A kit for a rapid saliva detection assay is shown in FIGS. 14C-14E. FIG. 14C depicts the kit as assembled. FIG. 14D depicts the individual components of the kit, which includes a saliva collection device and a lateral flow cassette. The use is depicted in FIGS. 15A-15B. The cassette is opened and placed horizontally on the bench. The syringe barrel is locked into the cassette port. The swab is placed in the mouth of the subject where saliva has pooled until the red circle on the saliva collection device is completely full. The swab is compressed until the red conjugate can be seen flowing along the cassette string. After 10 minutes, the assay is complete. Presence of a control line and presence of the test line indicates that SARS-COV-2 has been detected. Presence of the control line only indicates that SARS-COV-2 has not been detected. Presence of the test line only indicates the test is invalid.

### Example 10: Open Well Cassette for Saliva Detection

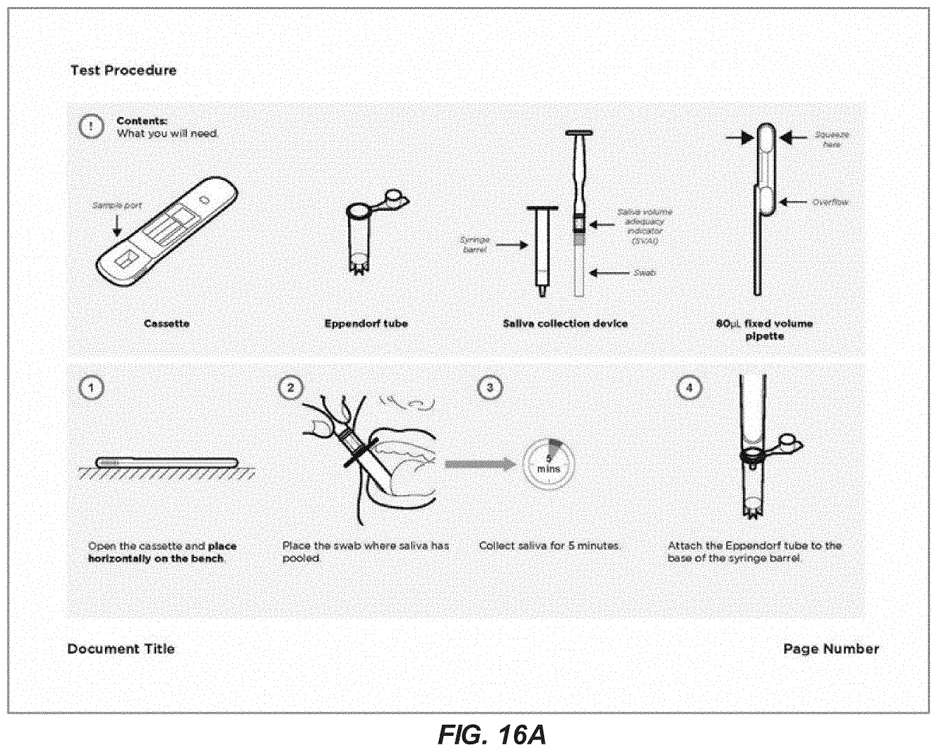

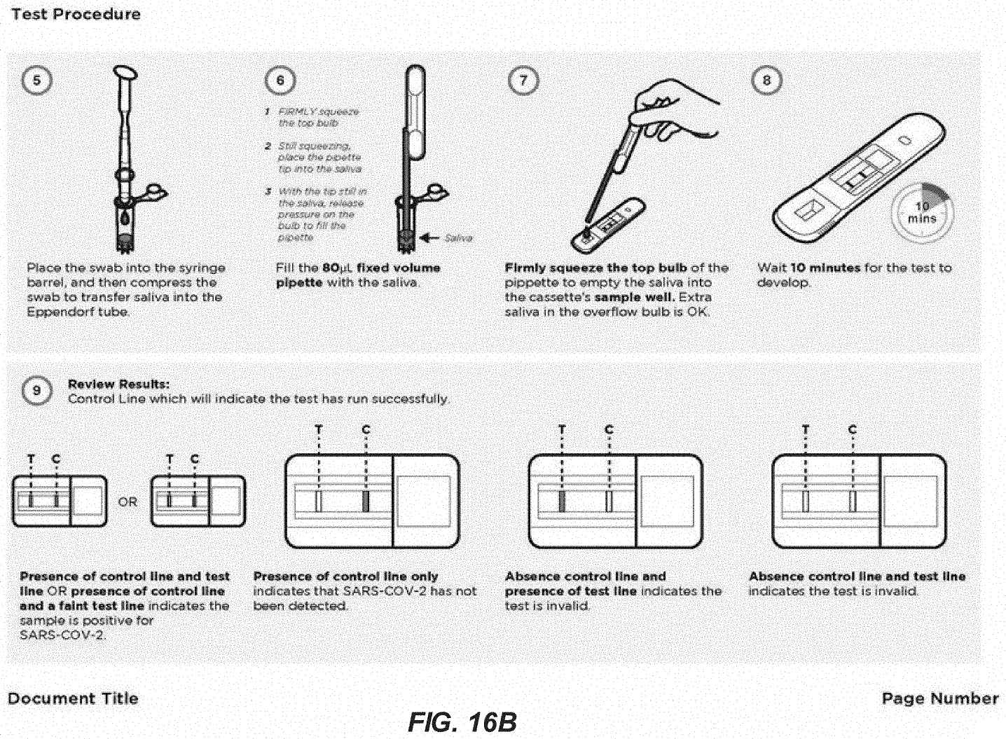

An Open Well Cassette is used to detect SARS-COV-2 as depicted in FIGS. 16A-16B. The swab is placed in the mouth to collect saliva for 5 minutes. The Eppendorf tube is attached to the base of the syringe barrel. The swab is placed in the syringe barrel and compressed to transfer saliva into the Eppendorf tube. The 80 μL fixed volume pipette is filled with saliva. The saliva is emptied from the pipette into the sample well. After 10 minutes, the assay is complete. Presence of a control line and presence of the test line indicates that SARS-COV-2 has been detected. Presence of the control line only indicates that SARS-COV-2 has not been detected. Presence of the test line only indicates the test is invalid.

### Example 11: Detection of Viral Load Using Open Well Cassette

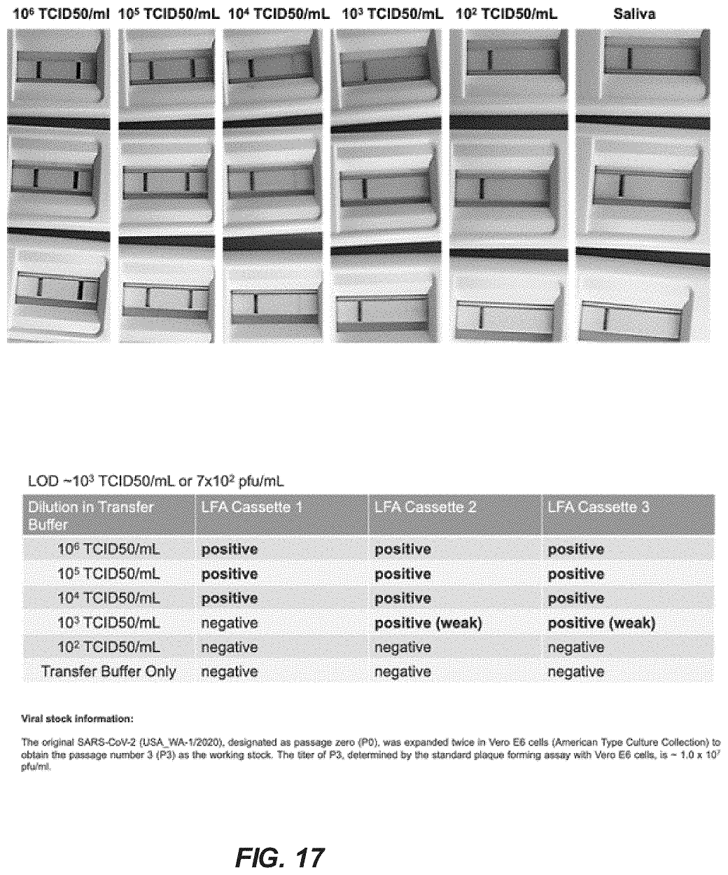

Coronavirus was spiked into saliva to a final concentration of 10⁶ TCID50/mL, 10⁵ TCID50/mL, 10⁴ TCID50/mL, 10³ TCID50/mL, 10² TCID50/mL, or transfer buffer only. The saliva was analyzed using the open well cassette device described in Example 10. As depicted in FIG. 17, the open well cassette device was able to detect virus levels at levels as low as 10³ TCID50/mL. This results in detection of virus at viral levels consistently found in infectious individuals.

### Example 12: Detection of Viral Load

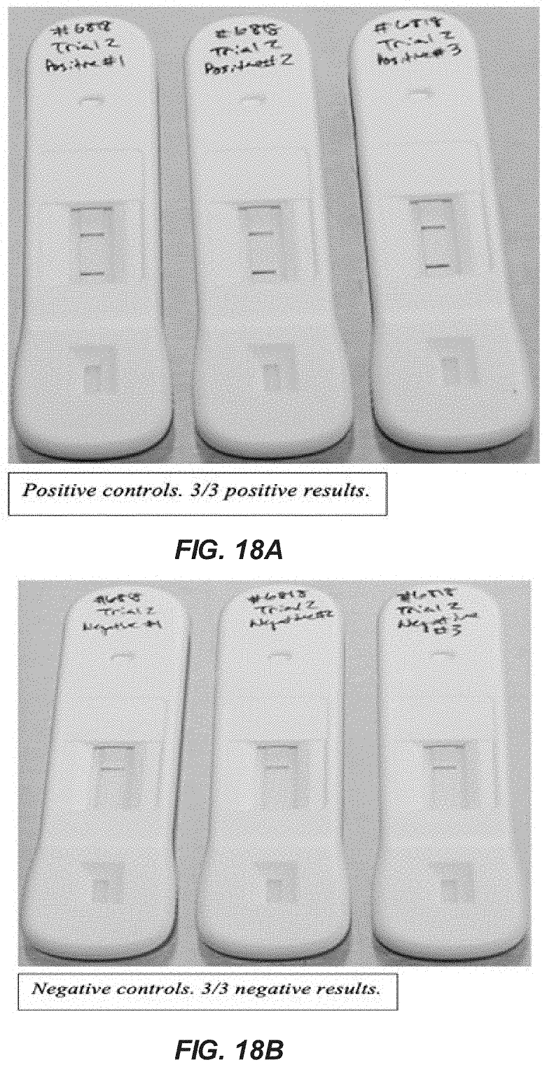

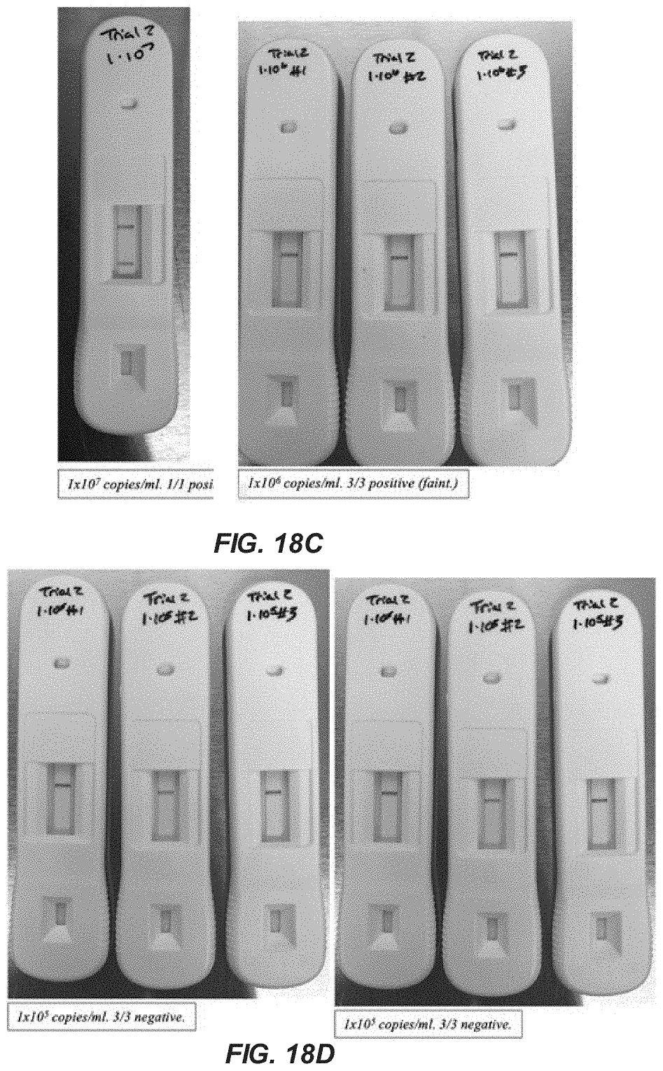

Live virus was spiked into viral samples. The concentrations were 10⁷ copies/mL, 10⁶ copies/mL, and 10⁵ copies/mL. 10⁶ copies per mL is approximately 2.16×10⁴ TCID50/mL. The saliva samples, as well as positive and negative controls were analyzed using an open well device with VHH trimer and commercial gold conjugate. As depicted in FIG. 18A and FIG. 18B, all cassettes testing positive controls displayed both the test and the control line, while all cassettes testing negative controls displayed only the control line. As depicted in FIG. 18C, the cassettes were able to detect viral loads of 10⁷ copies/mL and 10⁶ copies/mL, as indicated by both the control and test line. As depicted in FIG. 18D, cassettes tested with concentrations of 10³ copies per mL displayed only the control line.

### Example 13: Clinical Trial

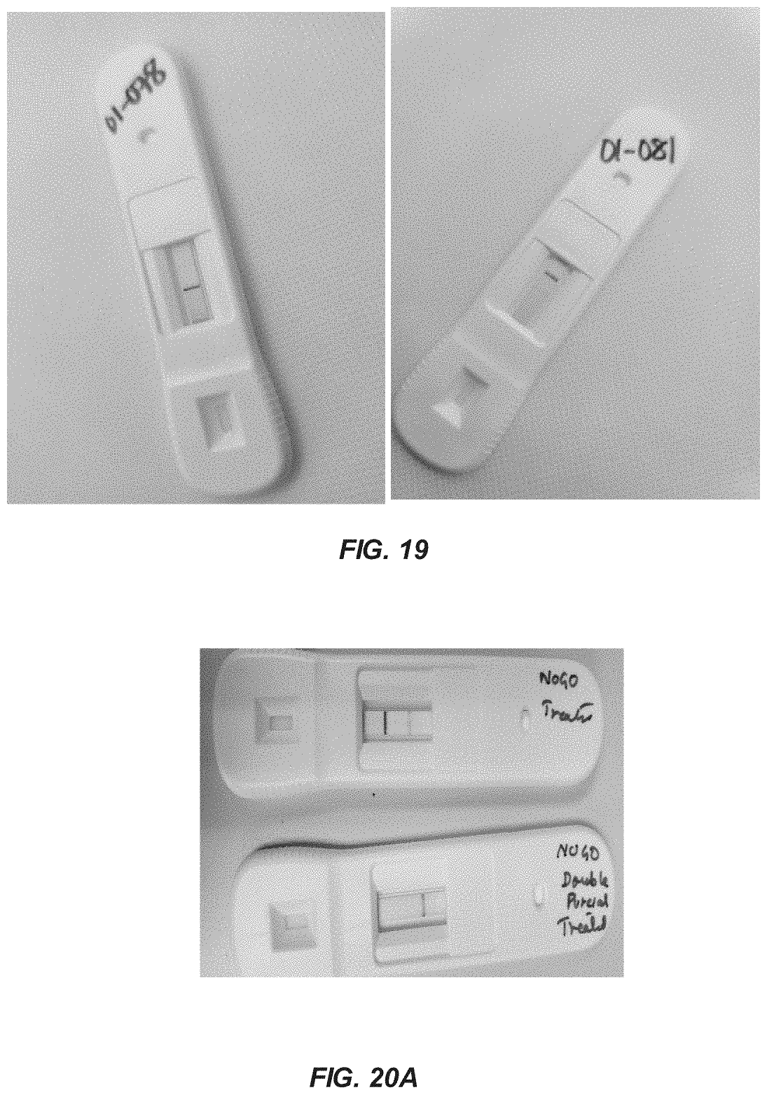

Patients with symptoms of COVID-19 were given both a PCR test and the saliva assay described in Example 10. The saliva was tested immediately and the samples were identified as positive or negative by eye. The results were recorded by pictures taken in a light box. FIG. 19 depicts representative samples of the results. 10 of the 10 subjects tested were identified as positive for SARS-CoV-2 by both the saliva assay and the PCR analysis.

### Example 14: Double Purification of Saliva

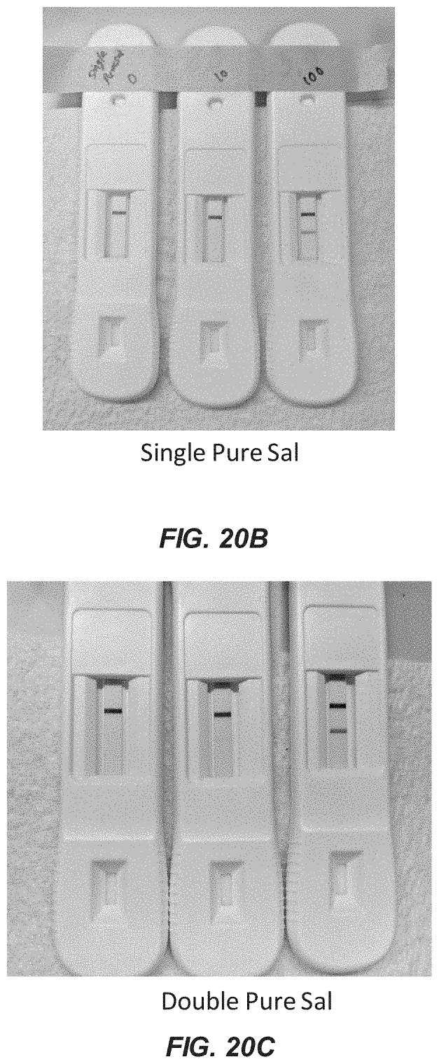

Saliva was spiked with spike protein. Single purification and double purification of the saliva was performed. The saliva was then analyzed using the lateral flow cassette. Results are depicted in FIGS. 20A-20C. When compared to single purification of saliva (FIG. 20B), double purification of saliva (FIG. 20C) resulted in loss of nonspecific binding while resulting in no loss of signal.

### Example 15: Optimization of Conjugation of the Ab-10 Spike Trimer

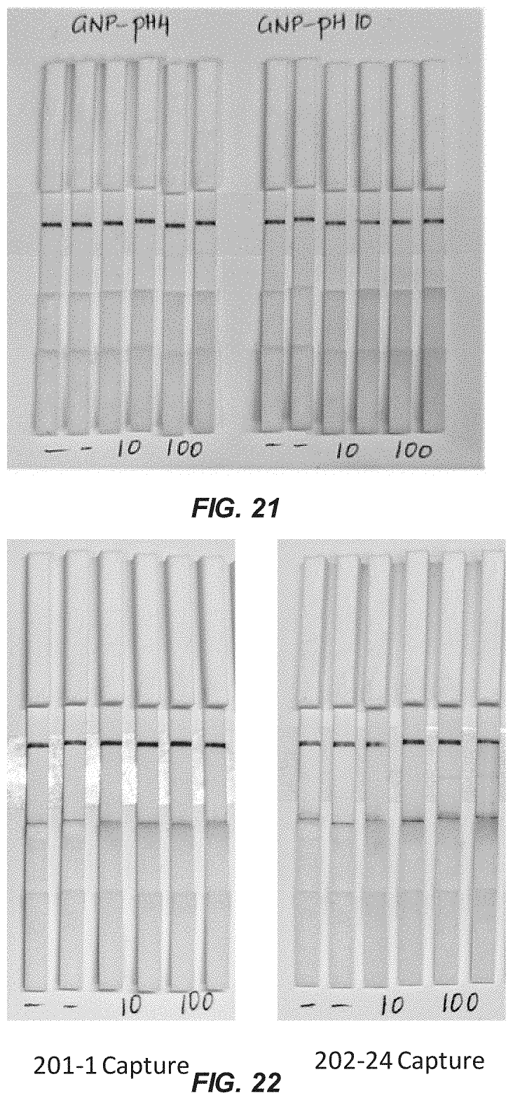

The 201-1 spike trimer was conjugated to gold nanoparticles. Lateral flow strips using the conjugated trimer were tested at a pH of 4 and a pH of 10. The results are depicted in FIG. 21. Conjugation was successful at high pH values, however the signal was poor.

Biotinylated Ab-10 was conjugated to gold nanospheres and used as a detector antibody. Ab-10 and Ab-9 were tested as capture antibodies. The results are depicted in FIG. 22. Conjugation was successful, but binding to both captures was poor.

### Example 16: Nucleocapsid Antibody Screening

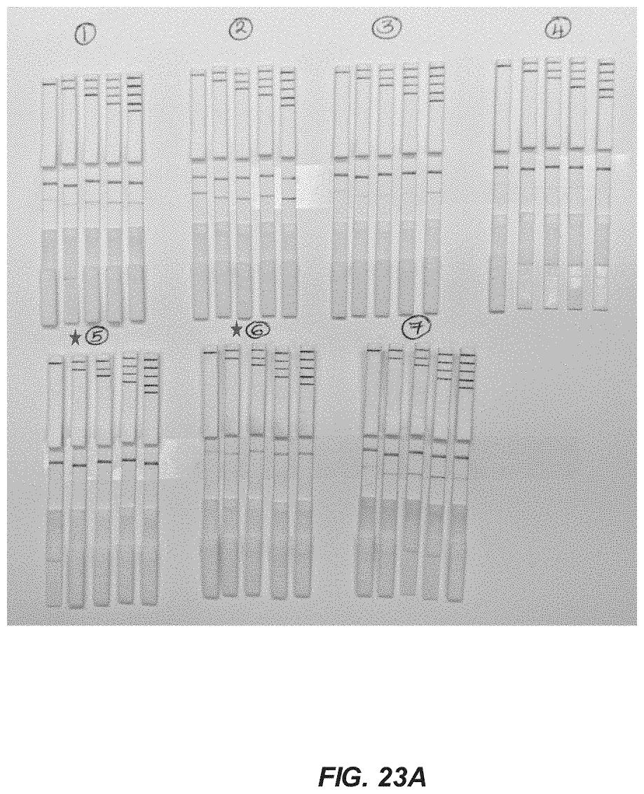

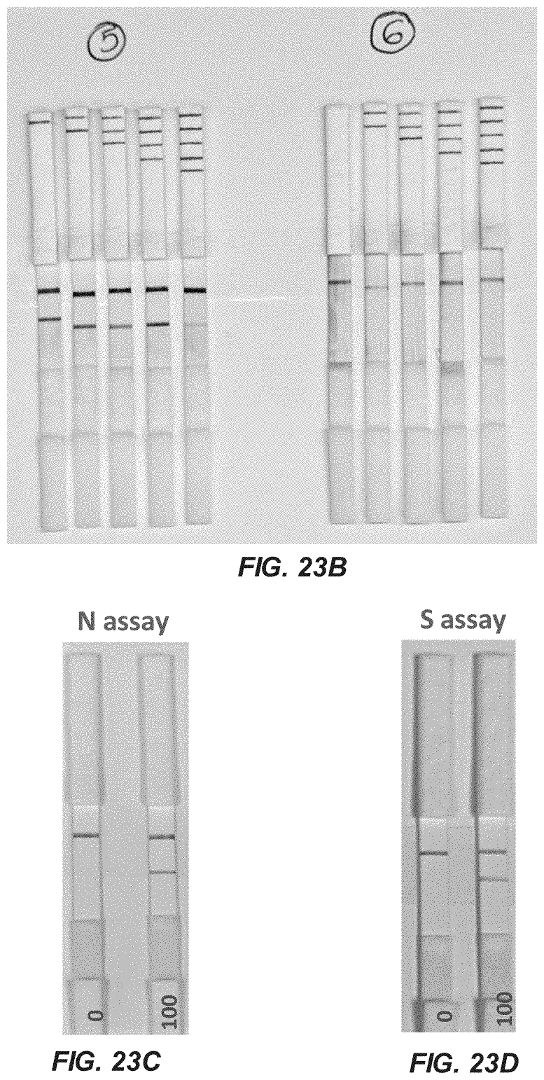

7 conjugates were screened against 5 nucleocapsid antibodies in a lateral flow assay using buffer only. The results are depicted in FIG. 23A. Two potential candidate detector antibodies were identified. The two candidate antibodies were tested against capture antibodies. with nucleocapsid protein. Results are depicted in FIG. 23B. Conjugate 5 was the better detector antibody. Ab-89 (4^{th} test strip) showed the strongest capture.

The assay was optimized to result nonspecific binding on the N assay with the 5B-1-Ab-89 pair. A new buffer of 150 mM Tris, pH 8.8, 2% IGEPAL, 0.1% PVP, 0.05% PVA and 0.5% Tween20 was found to reduce nonspecific binding, as depicted in FIG. 23C. Further, this buffer did not affect the binding of the S assay, as depicted in FIG. 23D.

### Example 17: Combined Nucleocapsid and Spike Assay

A lateral flow strip assay to detect both nucleocapsid and spike protein was developed. The detector-capture pairs for nucleocapsid were 5B1 and Ab-89, and the detector-capture pair for spike protein was Ab-9 and Ab-10.

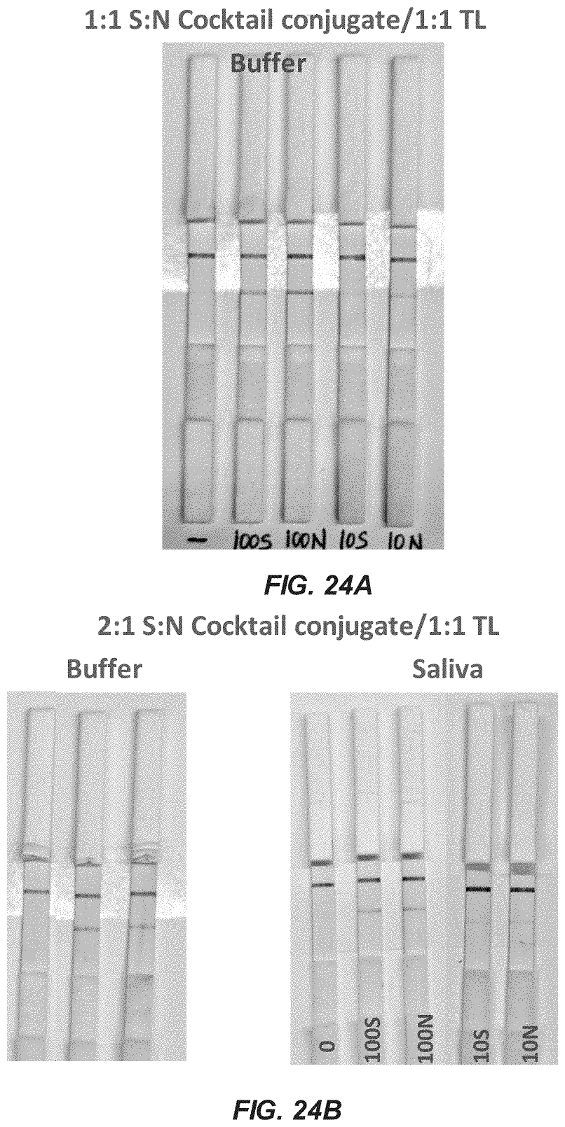

Different ratios of antibody pairs were tested. A 1:1 mix of both capture and detector antibodies resulted in nonspecific binding, as depicted in FIG. 24A. A 2:1 spike: nucleocapsid for the detector antibodies and a 1:1 spike: nucleocapsid ratio for the capture antibodies resulted in no nonspecific binding, as depicted in FIG. 24B.

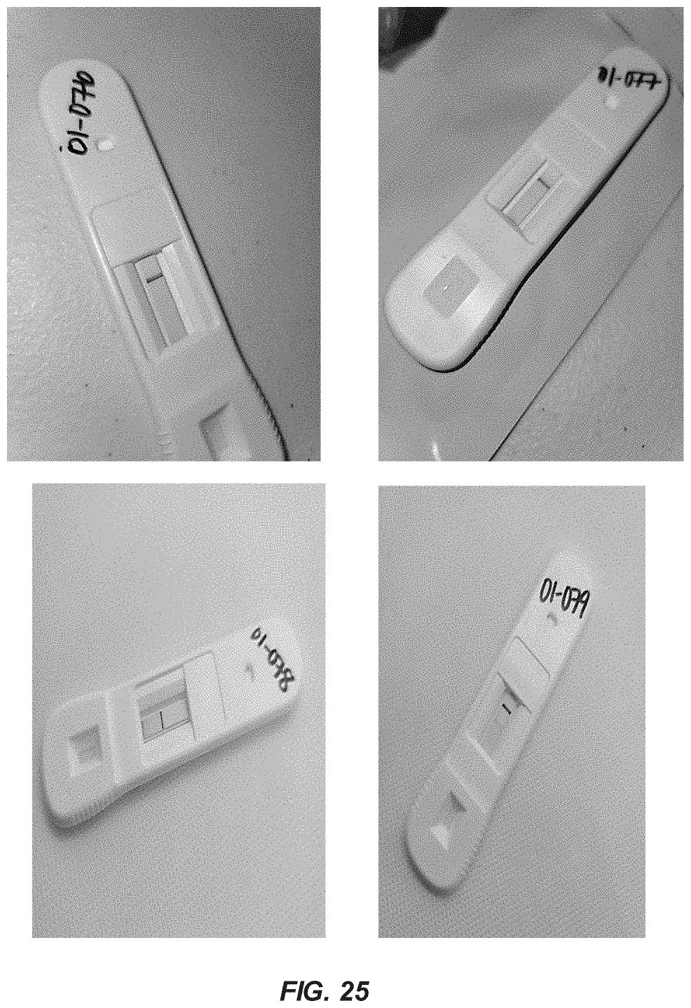

Testing clinical samples resulted in all positive hits, as depicted in FIG. 25.

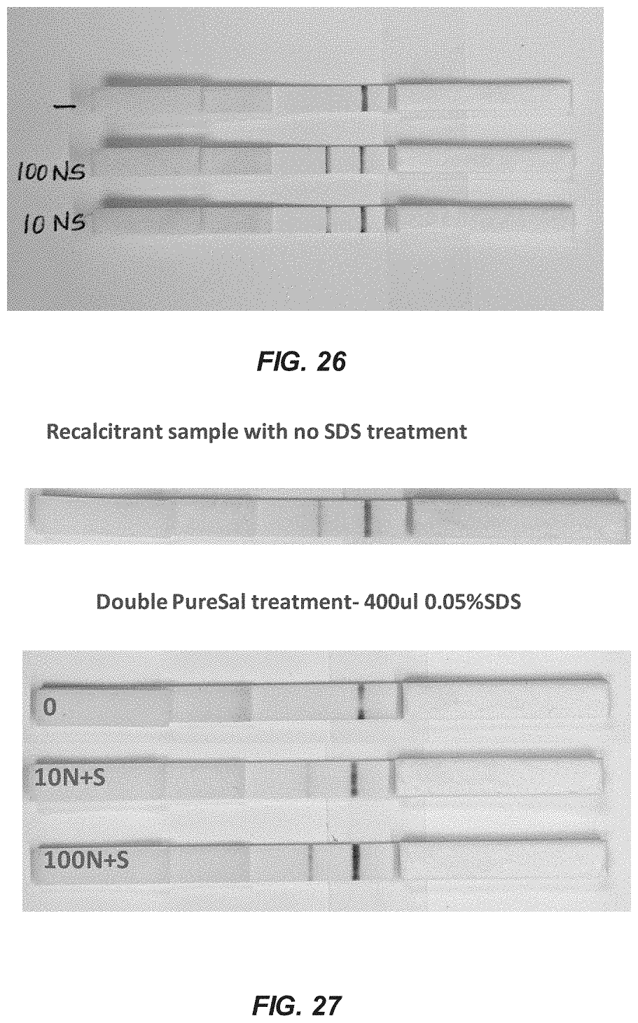

The buffer was optimized to reduce nonspecific binding for a 1:1 ratio of both detector and capture antibodies. A buffer of 150 mM Tris, pH 9; 2% IGEPAL, 0.1% PVP, 0.05% PVA, and 0.5% Tween20 resulted in no nonspecific binding, as depicted in FIG. 26.

Surfactants were added to reduce nonspecific binding to the nucleocapsid and spike assay. The sample pad was switched from CO48 to DVA. As depicted in FIG. 27, nonspecific binding was eliminated in the Double PurSal samples treated with 400 μL of 0.05% SDS/

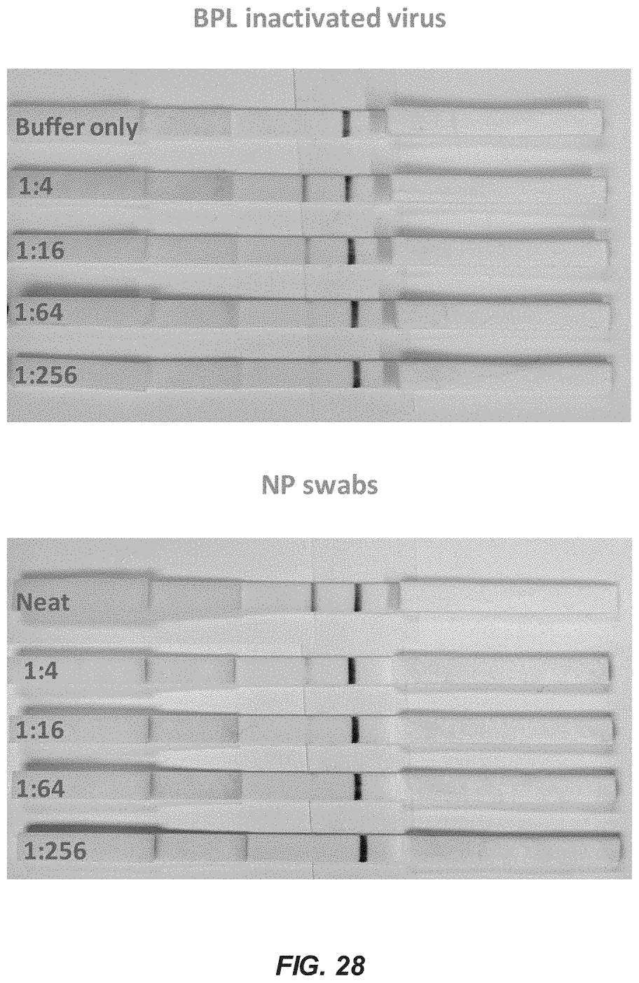

A dose titration of inactivated virus samples and a dose titration of frozen nasopharyngeal samples was run on the nucleocapsid and spike lateral assay strips. As depicted in FIG. 28, in both the inactivated viral samples and the nasal pharyngeal samples, the virus could be detected up to a dilution of 1:256.

### Example 18: Comparison of Nucleocapsid and Spike Lateral Flow Assay to Spike Lateral Flow Assay

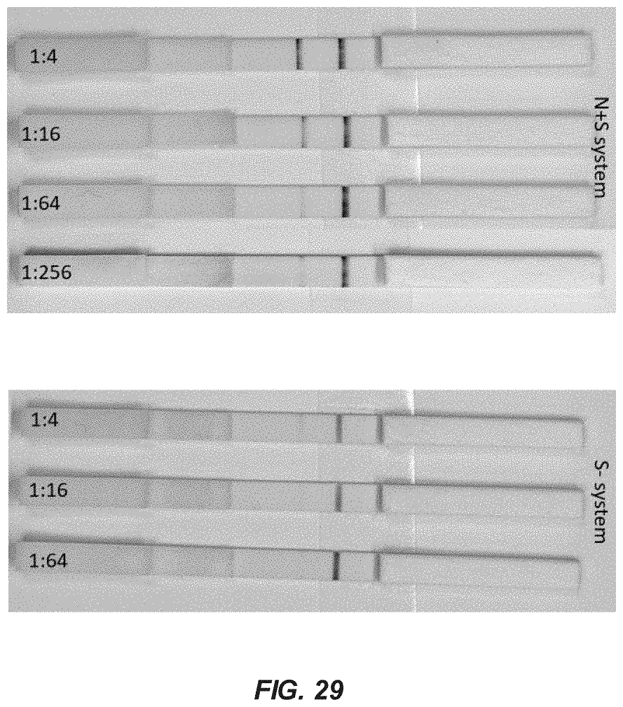

The nucleocapsid and spike combined lateral flow assay strips were compared to strips detecting spike alone. The strips were tested using dilutions of inactivated virus in saliva. As seen in FIG. 29, the nucleocapsid and spike (N+S) strips were able to detect virus at dilutions as low as 1:256. In contrast, the spike only strips (S) were able to detect virus at dilutions of 1:16 (faint)

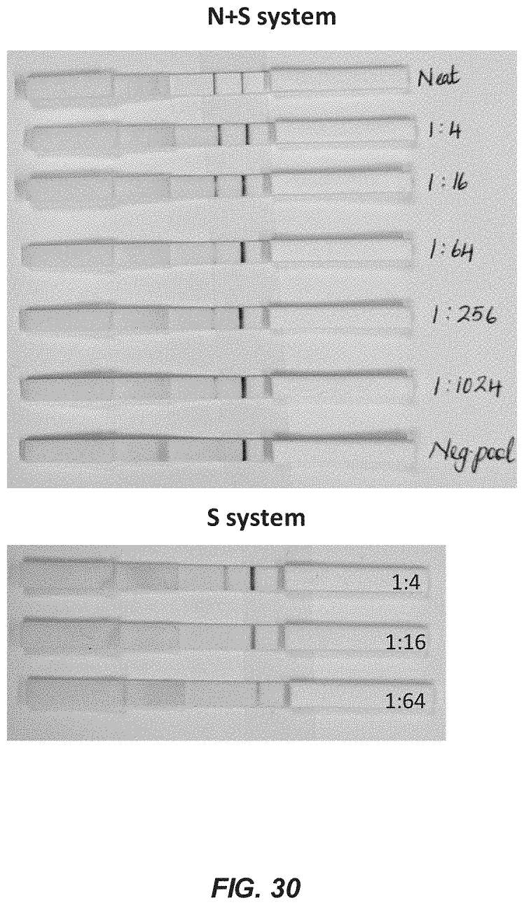

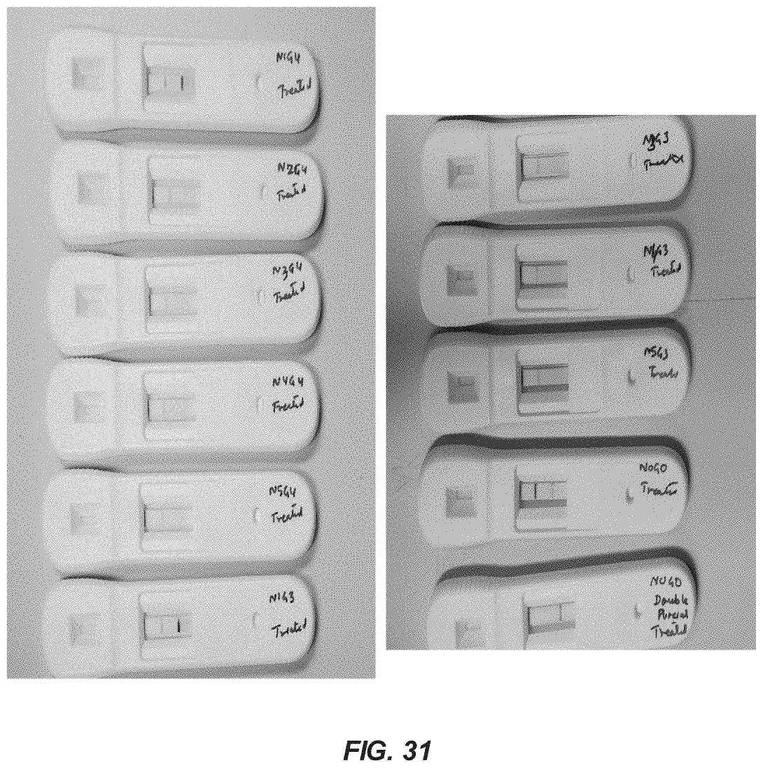

The N+S strips were compared to the S strips using nasopharyngeal swab samples. The results are depicted in FIG. 30. The N+S assay was able to detect virus at a dilution as low as 1:1024. The S assay was able to detect virus at a dilution of 1:16 (faint).

### Example 19: Use of Mucolytic Agents

Saliva spiked with spike protein was combined with mucolytic agents. The saliva was then analyzed on the lateral flow cassette. As depicted in FIG. 31, there was a weakening or loss of the control line. Nonspecific binding was persistent.

### Example 20: Exemplary Sequences

Tables 9-14 demonstrate exemplary sequences for use in the assays described herein.

TABLE 9
Variable Domain Heavy Chain CDR Sequences

| | SEQ ID | | SEQ ID | | SEQ ID | |
|---|---|---|---|---|---|---|
| Variant | NO | CDRH1 | NO | CDRH2 | NO | CDRH3 |
| 1-1 | 148 | FTFSSYAMN | 883 | SAISGSGVSTYYA | 1618 | CAKGDSGSYYGSSYFDYW |
| 1-2 | 149 | FTFSSYGMS | 884 | SAISGSGGNTYYA | 1619 | CTRVRRGSGVAPYSSSWGRYY |
| | | | | | | FDYW |
| 1-3 | 150 | FRFSSYSMS | 885 | SAISGSGGSSYYA | 1620 | CAKDGSGTIFGVVIAKYYFDYW |
| 1-4 | 151 | FTFSAYAMS | 886 | SAISGSGGSTHYA | 1621 | CASWGPLWSGSPNDAFDIW |
| 1-5 | 152 | FFSSYAMG | 887 | SAISGSGYSTYYA | 1622 | CARVRSYDSTAYDEPLDALDIW |
| 1-6 | 153 | FTFSSFAMS | 888 | SAISGSGVSTYYA | 1623 | CGRDARSSGYNGYDLFDIW |
| 1-7 | 154 | FTFSAYAMS | 889 | SAISGSGGSYYA | 1624 | CAKGPLVGWYFDLW |
| 1-8 | 155 | FTFGSYAMS | 890 | SLISGSGGSTYYA | 1625 | CASWGPLWSGSPNDAFDIW |
| 1-9 | 156 | FTFSAYAMS | 891 | SAISGSGGSTFYA | 1626 | CTRQGDSSGWYDGWFDPW |
| 1-10 | 157 | FIFSSYAMS | 892 | SIISGSGGSTYYA | 1627 | CIATVVSPLDYW |
| 1-11 | 158 | FTFSDYAMS | 893 | STISGSGGSTYYA | 1628 | CARDESSSSLNWFDPW |
| 1-12 | 159 | FTFSSYAMI | 894 | SAISGSAGSTYYA | 1629 | CASPDPLGSVADLDYW |
| 1-13 | 160 | FTFGSYAMS | 895 | SAISGSGGTTYYA | 1630 | CARVWSSSSVFDYW |
| 1-14 | 161 | FTFSRYAMS | 896 | SAISGSGASTYYA | 1631 | CAKDRGGGSYYGTFDYW |
| 1-15 | 162 | STFSSYAMS | 897 | SAISGSGATYYA | 1632 | CTRVRVAGYSSSWYDAFDIW |
| 1-16 | 163 | FTFSSYAMT | 898 | SAISGSGGNTYYA | 1633 | CVKGTIPIFGVIRSAFDYW |
| 1-17 | 164 | FTFSSYVMS | 899 | SSISGSGGSTYYA | 1634 | CARGSGSYSFFDYW |
| 1-18 | 165 | FTFSSYAN | 900 | SAISGSGVSTYYA | 1635 | CATTPGPWIQLWFGGGFDYW |
| 1-19 | 166 | FTFSSYDMS | 901 | SAISGSAGSTTMR | 1636 | CAKDGLVVAGTFDYW |
| 1-20 | 167 | FTFSGYAMS | 902 | SALSGSGGSTYYA | 1637 | CARGALLEWLSRFDNW |
| 1-21 | 168 | FTLSSYAMS | 903 | SAISGSGGTTYYA | 1638 | CARDLGAADLIDYW |
| 1-22 | 169 | FIFSSYAMS | 904 | SAISGSGGTYYA | 1639 | CVRVPAAAGKGVPGIFDIW |
| 1-23 | 170 | FTFSSYAMG | 905 | SAIRGSGGSTYYA | 1640 | CARVRQGLRRTWYYFDYW |
| 1-24 | 171 | STFSSYAMS | 906 | SAIGGSGGSTYYA | 1641 | CAKEYSSSWFDPW |
| 1-25 | 172 | FTFSSYTMS | 907 | SAISVSGGSTYYA | 1642 | CAKREDYDFWSGRGAFDIW |
| 1-26 | 173 | FTFSSYAMY | 908 | SAISGSGGTYYA | 1643 | CAKDIGYSSSWSFDYW |
| 1-27 | 174 | FTFRSYAMS | 909 | SAISGSGRSTYY | 1644 | CARDDYSDYRPFDYW |
| 1-28 | 175 | FTFSSYTMS | 910 | SAISGSGGSIYYA | 1645 | CAHRPSLQWLDWWFDPW |
| 1-29 | 176 | FTFSSQAMS | 911 | SIISGSGGSTYYA | 1646 | CAKDGASGWPNWHFDLW |
| 1-30 | 177 | FTFSSYPMS | 912 | SAISGSGGRTYYA | 1647 | CAKGAAAGPFDYW |
| 1-31 | 178 | FTFSSYAMT | 913 | SAISGGTTYYA | 1648 | CAKEEYYYDSSGPNWFDPW |
| 1-32 | 179 | FTFSSYAMS | 914 | TAISVSGGSTYYA | 1649 | WAPQGGTTVPTGRFDPW |
| 1-33 | 180 | FTFSSYAMS | 915 | SAISGSSGSTYYA | 1650 | CSRGGGPAAGFHGLDVW |
| 1-34 | 181 | FTFSSYAVS | 916 | SAISASGGSTYYA | 1651 | CARAAKRQQLFPRNYFDYW |
| 1-35 | 182 | FTFSSYPMS | 917 | SAIRGSGGSTYYA | 1652 | CALHYGSGRSFDYW |
| 1-36 | 183 | FTFSSYGMS | 918 | SAISGSGGATYYA | 1653 | CARPGGRIVGALWGAFDYW |
| 3-1 | 184 | RTFCRYSMG | 919 | ATWRPANTNYA | 1654 | CAKNWGDAGTTWFEKSGW |
| 3-2 | 185 | NIFSRYIMG | 920 | AAISRTGGSTYYA | 1655 | CAIDPDGEW |
| 3-3 | 186 | RTLAGYTMG | 921 | AEIYPSGNGVYYA | 1656 | CAADVRDSIWRSW |
| 3-4 | 187 | STLSRYSMG | 922 | AAIARRERVYA | 1657 | CARLSCHDYSCYSAFDFW |
| 3-5 | 188 | SIFSSAAMG | 923 | AISWRTGTTYYA | 1658 | CAAAGSMGWNHLRDYDW |
| 3-6 | 189 | TFSGYLMG | 924 | AGIWRSGVSLYYA | 1659 | CAARSGWGAAMRSADFRW |
| 3-7 | 190 | RTFSSYDMG | 925 | AIIKSDGSTYYA | 1660 | CARSPRFSGVVVRPGLDLW |
| 3-8 | 191 | SISSYFMG | 926 | SSIGIAGTPTLYA | 1661 | CAACSDYYCSGVGAVW |
| 3-9 | 192 | PTFSTYAMG | 927 | AAVINGGTTNYA | 1662 | CAKDSWDSSGYSYHYYYYGM |
| | | | | | | DVW |
| 3-10 | 193 | IIGSFRTMG | 928 | GFTGSGRSQYYA | 1663 | CARGDIAVIQVLDYW |
| 3-11 | 194 | GTFASYGMG | 929 | AGIWEDSSAAHYA | 1664 | CAYSGIGTDW |
| 3-12 | 195 | LTFRNYAMG | 930 | AGITSGGTRNYA | 1665 | CAAGWGDSAW |
| 3-13 | 196 | SISTINVMG | 931 | AAISWGGGLTVYA | 1666 | CAAFDGYTGSDW |
| 3-14 | 197 | GTLSSYIG | 932 | ATVRSGSITNYA | 1667 | CAADLTDIWEGIREYDEYAW |
| 3-15 | 198 | RTFRRYPMG | 933 | VAVTWSGGSTYYA | 1668 | CAAGLRGRQYSW |
| 3-16 | 199 | STFSIDVMG | 934 | AAISWSGESTLYA | 1669 | CAAFDGYSGSDW |
| 3-17 | 200 | RTSSSAVMG | 935 | AAINRGGSTIYV | 1670 | CATGPYRSYFARSYLW |
| 3-18 | 201 | GTFSSYRMG | 936 | SAISWNDGGADYA | 1671 | CAATQWGSSGWKQARWYDW |
| 3-19 | 202 | TIFASAMG | 937 | AFSSSGGSTYYA | 1672 | CAKDPIAAADPGDSVSFDYW |
| 3-20 | 203 | FGIDAMG | 938 | ATITEGGATNVGSTS | 1673 | CALNVWRTSSDW |
| 3-21 | 204 | NIIGGNHMG | 939 | GAITSSRSTVYA | 1674 | CAAVTTQTYGYDW |
| 3-22 | 205 | RTFSRYDMG | 940 | GGTRSGSTNYA | 1675 | CARHSDYSGLSNFDYW |
| 3-23 | 206 | QPAPELRGYGMG | 941 | AAVIGSSGTTYYA | 1676 | CAKAKATVGLRAPFDYW |
| 3-24 | 207 | INFSRYGMG | 942 | ASITYLGRTNYA | 1677 | CALRVRPYGQYDW |
| 3-25 | 208 | RTFRRYAMG | 943 | AAINWSGARTYYA | 1678 | CAVSKPLNYYTYYDARRYDW |
| 3-26 | 209 | GTFGHYAMG | 944 | AAVSWSGSSTYYA | 1679 | CAVSQPLNYYTYYDARRYDW |
| 3-27 | 210 | FTLDDYAMG | 945 | AAISWSTGSTYYA | 1680 | CAASQAPITIATMMKPFYDW |
| 3-28 | 211 | FTFRRYDMG | 946 | SAISGGLAYYA | 1681 | CAVDLSGDAVYDW |
| 3-29 | 212 | INFSRNAMG | 947 | ASITHQDRPIYA | 1682 | CALPVGPYGQYDW |
| 3-30 | 213 | RTFTTYGMG | 948 | ASITYLGRTYYA | 1683 | CALRVRPYGQYDW |
| 3-31 | 214 | STFSINAMG | 949 | AGITSSGGYTNYA | 1684 | CAADGVPEYSDYASGPVW |
| 7-1 | 215 | FTFSNYAMR | 950 | SAISGSGGSTYYA | 1685 | CARHTGRYSSGSTGWFHYW |
| 7-2 | 216 | FAFSRHAMS | 951 | SDIGGSGSTTYYA | 1686 | CARTTFDNWFDPW |
| 7-3 | 217 | RTFSINAMG | 952 | AGITRSAVSTITSE | 1687 | CAADGVPEYSDYASGPVW |
| | | | | GTANYA | | |
| 7-4 | 218 | FTFSSYGMN | 953 | SASSGSGGSTYYA | 1688 | ARREYIESGFDSW |
| 7-5 | 219 | RTFSTDAMG | 954 | AAISSGGSTNYA | 1689 | CAATRGRSTRLVLPSLVEW |
| 7-6 | 220 | RIFYPMG | 955 | AAVRWSSTGIYYT | 1690 | CAAALSEVWRGSENLREGYD |
| | | | | QYA | | W |
| 7-7 | 221 | FTFGSYDMG | 956 | TAINWSGARTAYA | 1691 | CAARSVYSYEYNW |
| 7-8 | 222 | STFTINAMG | 957 | SGISHNGGTTNYA | 1692 | CAADGVPEYSDYASGPVW |
| 7-9 | 223 | GTFSSIGMG | 958 | AAISWDGGATAYA | 1693 | CAKEDVGKPFDW |
| 7-10 | 224 | RTYAMG | 959 | AEINWSGSSTYYA | 1694 | CAVDGPFGW |
| 7-11 | 225 | LPFSTKSMG | 960 | AAIHWSGLTSYA | 1695 | CAADRAADFFAQRDEYDW |
| 7-12 | 226 | RTIVPYTMG | 961 | AAISPSAFTEYA | 1696 | CAARRWGYDW |
| 7-13 | 227 | LRLNMHRMG | 962 | AAISGWSGGTNYA | 1697 | CAKIGTLWW |
| 7-14 | 228 | STFSINAMG | 963 | AGISRGGTTNYA | 1698 | CAADGVPEYSDYASGPVW |
| 7-15 | 229 | STLPYHAMG | 964 | ASISRFFGTAYYA | 1699 | CAPTFAAGASEYHW |
| 7-16 | 230 | FTFTSYAIS | 965 | SAISGSGGSTDYA | 1700 | CARGAYGSGTYDYW |
| 7-17 | 231 | FSLDYYGMG | 966 | AAITSGGTPHYA | 1701 | CASAYNPGIGYDW |
| 7-18 | 232 | LTDRRYTMG | 967 | ASITLGGSTAYA | 1702 | CAKEDVGKPFDW |
| 7-19 | 233 | RTFRRYTMG | 968 | ASITSSGVNAYA | 1703 | CAKEDVGKPFDW |
| 7-20 | 234 | PTFSIYAMG | 969 | AGISWNGGSTNYA | 1704 | CALRRRFGGQEW |
| 7-21 | 235 | RTISRYTMG | 970 | ASITSGGSTAYA | 1705 | CAKEDVGKPFDW |
| 7-22 | 236 | RTITRYTMG | 971 | ASITSGGSTAYA | 1706 | CAKQDVGKPFDW |
| 7-23 | 237 | FTFENHAMG | 972 | AEIYPSGSTIYA | 1707 | CAARILSRNW |
| 7-24 | 238 | FTFSRHAMN | 973 | STITGSGGSTNYA | 1708 | CAREVGLYYYGSGSSSRRLLG |
| | | | | | | RIDYYFDYW |
| 7-25 | 239 | FTFDDYSMG | 974 | ASIEWDGSTYYA | 1709 | CAAFDGYTGSDW |
| 7-26 | 240 | STFSINAMG | 975 | AGITSSGGYTNYA | 1710 | CAADGVPEYSDYASGPVW |
| 7-27 | 241 | QTFNMG | 976 | AEINWSGSSTYYA | 1711 | CAVDGPFGW |
| 7-28 | 242 | NTFSDNPMG | 977 | AILAWDSGSTYYA | 1712 | CTTDYSKLAITKLSYW |
| 7-29 | 243 | RTHSIYPMG | 978 | ASITSYGDTNYA | 1713 | CAARRWIPPGPIW |
| 7-30 | 244 | RTFSMHAMG | 979 | ASISSQGRTNYA | 1714 | CAAEVRNGSDYLPIDW |
| 7-31 | 245 | FTFSNYSMG | 980 | AAIHWNGDSTAYA | 1715 | CAAQTEDSAQYIW |
| 7-32 | 246 | STFSVNAMG | 981 | AGVTRGGYTNYA | 1716 | CAADGVPEYSDYASGPVW |
| 7-33 | 247 | SIGSINAMG | 982 | AGISNGGTTNYA | 1717 | CAADGVPEYSDYASGPVW |
| 7-34 | 248 | RTFGSYDMG | 983 | AFIHRSGGSTYYA | 1718 | CATFPAIVTDSDYDLGNDW |
| 7-35 | 249 | GTFGHYAMG | 984 | AAVSWSGSSTYYA | 1719 | CAVSQPLNYYTYYDARRYDW |
| 7-36 | 250 | FGFGSYDMG | 985 | TAINWSGARAYYA | 1720 | CAARSVYSYDYNW |
| 7-37 | 251 | STLSINAMG | 986 | AGITRSGSVTNYA | 1721 | CAADGVPEYSDYASGPVW |
| 7-38 | 252 | RPFSEYTMG | 987 | SSIHWGGRGTNYA | 1722 | CAAELHSSDYTSPGAYAW |
| 7-39 | 253 | RTFSNYPMG | 988 | AAITWSGDSTNYA | 1723 | CALPSNIITTDYLRVYW |
| 7-40 | 254 | RTFRRYTMG | 989 | ASITKFGSTNYA | 1724 | CAKEDVGKPFDW |
| 7-41 | 255 | RTFSTYVMG | 990 | ASISSRGITHYA | 1725 | CAKEDVGKPFDW |
| 7-42 | 256 | FTLDYYGMG | 991 | AAITSGGTPHYG | 1726 | CASAYNPGIGYDW |
| 7-43 | 257 | FTFGHYAMG | 992 | AAVSWSGSTTYYA | 1727 | CAVSHPLNYYTYYDARRYDW |
| 7-44 | 258 | FTFEDYAMG | 993 | AAITRGSNTTDYA | 1728 | CAARRWMGGSYFDPGNYDW |
| 7-45 | 259 | RTLSRYTMG | 994 | ASITSGGSTNYA | 1729 | CAKEDVGKPFDW |
| 8-1 | 260 | RTFASYAMG | 995 | GAISRSGDSTYYA | 1730 | CARAPFYCTTTKCQDNYYYM |
| | | | | | | DVW |
| 8-2 | 261 | GTYHAMG | 996 | AGITSDDRTNYA | 1731 | CARERRYYDSSGYPYYFDYW |
| 8-3 | 262 | TTLDYYAMG | 997 | AAISWSGGSTAYA | 1732 | CAREDYYDSSGYSW |
| 8-4 | 263 | GTLSRSRMG | 998 | AFIGSDTLYA | 1733 | CANLAYYDSSGYYDYW |
| 8-5 | 264 | GTFSFYNMG | 999 | AFISGNGGTSYA | 1734 | CAVVAMRMVTTEGPDVLDVW |
| 8-6 | 265 | FTFDYYAMG | 1000 | SAIDSEGRTSYA | 1735 | CARWGPFDIW |
| 8-7 | 266 | FPFSIWPMG | 1001 | AAVRWSSTGIYYT | 1736 | CTRSEYSSGWYDYW |
| | | | | QYA | | |
| 8-8 | 267 | FAESSSMG | 1002 | AAISWSGDITIYA | 1737 | CARGAPYFDHGSKSYRLFYFDYW |
| 8-9 | 268 | FTFGTTTMG | 1003 | AAISWSTGIAHYA | 1738 | CARGGPNYYASGRYPWFDPW |
| 8-10 | 269 | FIGNYHAMG | 1004 | AAVTWSGGTTNYA | 1739 | CAREGYYYDSSGYPYYFDYW |
| 4A-1 | 270 | RTFSDDTMG | 1005 | GGISWSGGNTYYA | 1740 | CATDPPLFW |
| 4A-2 | 271 | RTFGDYIMG | 1006 | AAINWSAGYTAYA | 1741 | CARASPNTGWHFDRW |
| 4A-3 | 272 | RTFSDDAMG | 1007 | AAINWSGGTTRYA | 1742 | CATDPPLFW |
| 4A-4 | 273 | RTFGDYIMG | 1008 | AAINWIAGYTADA | 1743 | CAEPSPNTGWHFDHW |
| 4A-5 | 274 | RTFGDDTMG | 1009 | AAINWSGGNTYYA | 1744 | CATDPPLFW |
| 4A-6 | 275 | RTFGDDTMG | 1010 | AAINWTGGYTPYA | 1745 | CATDPPLFW |
| 4A-7 | 276 | RTFGDYIMG | 1011 | AAINWSGGYTAYA | 1746 | CATASPNTGWHFDHW |
| 4A-8 | 277 | RTFGDYIMG | 1012 | GGINWSGGYTYYA | 1747 | CATDPPLFW |
| 4A-9 | 278 | RTFGDYIMG | 1013 | AAINWSGGYTHYA | 1748 | CATDPPLFW |
| 4A-10 | 279 | RTFSDDTMG | 1014 | AAIHWSGSSTRYA | 1749 | CATDPPLFW |
| 4A-11 | 280 | RTFGDYAMG | 1015 | APINWSGGSTYYA | 1750 | CATDPPLFW |
| 4A-12 | 281 | RTFGDDTMG | 1016 | AAINWSGGNTPYA | 1751 | CATDPPLFW |
| 4A-13 | 282 | RTFGDDTMG | 1017 | AAINWSGDNTHYA | 1752 | CATDPPLFW |
| 4A-14 | 283 | RTFSDDTMG | 1018 | AAINWSGGTTRYA | 1753 | CATDPPLFW |
| 4A-15 | 284 | RTFSDDTMG | 1019 | AAINWSGDSTYYA | 1754 | CATDPPLFW |
| 4A-16 | 285 | RTFSDYTMG | 1020 | AAINWSGGYTYYA | 1755 | CATDPPLFW |
| 4A-17 | 286 | RTFGDDTMG | 1021 | AAINWSGGNTDYA | 1756 | CATDPPLFW |
| 4A-18 | 287 | RTFGDYIMG | 1022 | AAINWSGGYTPYA | 1757 | CATDPPLFW |
| 4A-19 | 288 | RTFSDDTMG | 1023 | AAINWSGGSTYYA | 1758 | CATDPPLFW |
| 4A-20 | 289 | RTFGDDIMG | 1024 | AAIHWSAGYTRYA | 1759 | CATDPPLFWGHVDLW |
| 4A-21 | 290 | RTFSDDTMG | 1025 | AGMTWSGSSTFYA | 1760 | CATDPPLFW |
| 4A-22 | 291 | RTFGDYIMG | 1026 | AAINWSGDNTHYA | 1761 | CATDPPLFW |
| 4A-23 | 292 | RTFSDDAMG | 1027 | AGISWNGGSIYYA | 1762 | CATDPPLFW |
| 4A-24 | 293 | RTFSDYTMG | 1028 | AAINWSGGTTYYA | 1763 | CATDPPLFW |
| 4A-25 | 294 | GTFSRYAMG | 1029 | SAVDSGGSTYYA | 1764 | CAASPSLRSAWQW |
| 4A-26 | 295 | RTFSDDTMG | 1030 | AAVNWSGGSTYYA | 1765 | CATDPPLFW |
| 4A-27 | 296 | RTFGDYIMG | 1031 | AAINWSAGYTAYA | 1766 | CARATPNTGWHFDHW |
| 4A-28 | 297 | RTFGDDTMG | 1032 | AAINWNGGNTHYA | 1767 | CATDPPLFW |
| 4A-29 | 298 | RTFGDDTMG | 1033 | AAINWSGGYTYYA | 1768 | CATDPPLFW |
| 4A-30 | 299 | RTFGDYTMG | 1034 | AAINWTGGYTYYA | 1769 | CATDPPLFW |
| 4A-31 | 300 | RTFGDYIMG | 1035 | AAINWSAGYTAYA | 1770 | CATASPNTGWHFDHW |
| 4A-32 | 301 | FTFDDYEMG | 1036 | AAISWRGGTTYYA | 1771 | CAADRRGLASTRAGDYDW |
| 4A-33 | 302 | FTFSRHDMG | 1037 | AGINWESGSTNYA | 1772 | CAADRGVYGGRWYRTSQYTW |
| 4A-34 | 303 | RTFGDYIMG | 1038 | AAINWSADYTAYA | 1773 | CATDPPLFCWHFDHW |
| 4A-35 | 304 | QLANFASYAMG | 1039 | AAITRSGSSTVYA | 1774 | CATTMNPNPRW |
| 4A-36 | 305 | RTFGDYIMG | 1040 | AAINWSAGYTAYA | 1775 | CATAPPLFCWHFDHW |
| 4A-37 | 306 | RTFGDYGMG | 1041 | ATINWSGALTHYA | 1776 | CATLPFYDFWSGYYTGYYYMDVW |
| 4A-38 | 307 | RTFSDDTMG | 1042 | AAITWSGGRTRYA | 1777 | CATDRPLFW |
| 4A-39 | 308 | RTFSNAAMG | 1043 | ARILWTGASRNYA | 1778 | CATTENPNPRW |
| 4A-40 | 309 | RTFSDDTMG | 1044 | AGINWSGNGVYYA | 1779 | CATDPPLFW |
| 4A-41 | 310 | RTFGDYIMG | 1045 | AAINWSGGTTPYA | 1780 | CATDPPLFCCHVDLW |
| 4A-42 | 311 | RTFGDDTMG | 1046 | AAINWSGGYTPYA | 1781 | CATDPPLFWGHVDLW |
| 4A-43 | 312 | RTFSDDTMG | 1047 | AAINWSGGSTDYA | 1782 | CATDPPLFW |
| 4A-44 | 313 | RTFGDYIMG | 1048 | AAINWSAGYTAYA | 1783 | CATARPNTGWHFDHW |
| 4A-45 | 314 | RTFSDDAMG | 1049 | AAINWSGGSTRYA | 1784 | CATDPPLFW |
| 4A-46 | 315 | RTFGDYIMG | 1050 | AAINWSAGYTPYA | 1785 | CATDPPLFWGHVDLW |
| 4A-47 | 316 | FTFGDYVMG | 1051 | AAINWNAGYTAYA | 1786 | CAKASPNTGWHFDHW |
| 4A-48 | 317 | RTFSDDAMG | 1052 | GRINWSGGNTYYA | 1787 | CATDPPLFW |
| 4A-49 | 318 | RTFGDYIMG | 1053 | AAINWSAGYTAYA | 1788 | CARASPNTGWHFDHW |
| 4A-50 | 319 | GTFSNSGMG | 1054 | AVVNWSGRRTYYA | 1789 | CAVPWMDYNRRDW |
| 2A-1 | 320 | FTFSNYATD | 1055 | SIISGSGGATYYA | 1790 | CAKGGYCSSDTCWWEYWLDPW |
| 2A-2 | 321 | FTFSRHAMN | 1056 | SGISGSGDETYYA | 1791 | CARDLPASYYDSSGYYWHNG |
| | | | | | | MDVW |
| 2A-3 | 322 | FTFSDFAMA | 1057 | SAISGSGDITYYA | 1792 | CAREADCLPSPWYLDLW |
| 2A-4 | 323 | FTFSDFAMA | 1058 | SAITGTGDITYYA | 1793 | CAREADGLHSPW |
| 2A-5 | 324 | FTFSDFAMA | 1059 | SAISGSGDITYYA | 1794 | CAREADGLHSPWHFDLW |
| 2A-6 | 325 | FTFSDFAMA | 1060 | SAISGSGDITYYA | 1795 | CAREADGLHSPWHFDLW |
| 2A-7 | 326 | FTFSDFAMA | 1061 | SAITGSGDITYYA | 1796 | CAREADGLHSPWHFDLW |
| 2A-8 | 327 | FTFSDFAMA | 1062 | SAISGSGDITYYA | 1797 | CAREADGLHSPWHFDLW |
| 2A-9 | 328 | FTFPRYAMS | 1063 | STISGSGSTTYYA | 1798 | CARLIDAFDIW |
| 2A-10 | 329 | FTFSAFAMG | 1064 | SAITASGDITYYA | 1799 | CARQSDGLPSPWHFDLG |
| 2A-11 | 330 | FTFSNYPMN | 1065 | STISGSGGNTFYA | 1800 | CVRHDEYSFDYW |
| 2A-12 | 331 | FTFSDYPMN | 1066 | STISGSGGITFYA | 1801 | CVRHDEYSFDYW |
| 2A-13 | 332 | FTFSDYPMN | 1067 | SAISGSGDNTYYA | 1802 | CVRHDEYSFDYW |
| 2A-14 | 333 | FTFSDYPMN | 1068 | SAITGSGDITYYA | 1803 | CVRHDEYSFDYW |
| 2A-15 | 334 | FTFSDYPMN | 1069 | STISGSGGITFYA | 1804 | CVRHDEYSFDYW |
| 3A-1 | 335 | FMFGNYAMS | 1070 | AAISGSGGSTYYA | 1805 | CAKDRGYSSSWYGGFDYW |
| 3A-2 | 336 | FTFRSHAMN | 1071 | SAISGSGGSTNYA | 1806 | CARGLKFLEWLPSAFDIW |
| 3A-3 | 337 | FTFRNYAMA | 1072 | SGISGSGGTTYYG | 1807 | CARGTRFLEWSLPLDVW |
| 3A-4 | 338 | FTFRNHAMA | 1073 | SGISGSGGTTYYG | 1808 | CARGTRFLQWSLPLDVW |
| 3A-5 | 339 | FTITNYAMS | 1074 | SGISGSGAGTYYA | 1809 | CARHAWWKGAGFFDHW |
| 3A-6 | 340 | FTIPNYAMS | 1075 | SGISGAGASTYYA | 1810 | CARHTWWKGAGFFDHW |
| 3A-7 | 341 | FTIPNYAMS | 1076 | SGISGSGASTYYA | 1811 | CARHTWWKGAGFFDHW |
| 3A-8 | 342 | FTITNYAMS | 1077 | SGISGSGASTYYA | 1812 | CARHTWWKGAGFFDHW |
| 3A-9 | 343 | FTITNYAMS | 1078 | SGISGSGAGTYYA | 1813 | CARHTWWKGAGFFDHW |
| 3A-10 | 344 | FTFRSHAMS | 1079 | SSISGGGASTYYA | 1814 | CARVKYLTTSSGWPRPYFDNW |
| 3A-11 | 345 | FTIRNYAMS | 1080 | SSISGGGASTYYA | 1815 | CARVKYLTTSSGWPRPYFDNW |
| 3A-12 | 346 | FTFRSHAMS | 1081 | SSISGGGASTYYA | 1816 | CARVKYLTTSSGWPRPYFDNW |
| 3A-13 | 347 | FTFRSHAMS | 1082 | SSISGGGASTYYA | 1817 | CARVKYLTTSSGWPRPYFDNW |
| 3A-14 | 348 | FTFRSYAMS | 1083 | SSISGGGASTYYA | 1818 | CARVKYLTTSSGWPRPYFDNW |
| 3A-15 | 349 | FTFSAYSMS | 1084 | SAISGSGGSRYYA | 1819 | CGRSKWPQANGAFDIW |
| 2A-1 | 350 | FTFSNYATD | 1085 | SIISGSGGATYYA | 1820 | CAKGGYCSSDTCWWEYWLDPW |
| 2A-10 | 351 | FTFSAFAMG | 1086 | SAITASGDITYYA | 1821 | CARQSDGLPSPWHFDLG |
| 2A-5 | 352 | FTFSDFAMA | 1087 | SAISGSGDITYYA | 1822 | CAREADGLHSPWHFDLW |
| 2A-2 | 353 | FTFSRHAMN | 1088 | SGISGSGDETYYA | 1823 | CARDLPASYYDSSGYYWHNG |
| | | | | | | MDVW |
| 2A-4 | 354 | FTFSDFAMA | 1089 | SAISGSGDITYYA | 1824 | CAREADGLHSPWHFDLW |
| 2A-6 | 355 | FTFSNYPMN | 1090 | STISGSGGNTFYA | 1825 | CVRHDEYSFDYW |
| 2A-11 | 356 | FTFSDFAMA | 1091 | SAITGSGDITYYA | 1826 | CAREADGLHSPWHFDLW |
| 2A-12 | 357 | FTFSDYPMN | 1092 | STISGSGGITFYA | 1827 | CVRHDEYSFDYW |
| 2A-13 | 358 | FTFSDYPMN | 1093 | SAISGSGDNTYYA | 1828 | CVRHDEYSFDYW |
| 2A-14 | 359 | FTFSDFAMA | 1094 | SAITGTGDITYYA | 1829 | CAREADGLHSPW |
| 2A-7 | 360 | FTFSDYPMN | 1095 | SAITGSGDITYYA | 1830 | CVRHDEYSFDYW |
| 2A-8 | 361 | FTFSDFAMA | 1096 | SAISGSGDITYYA | 1831 | CAREADGLHSPWHFDLW |
| 2A-15 | 362 | FTFSDFAMA | 1097 | SAISGSGDITYYA | 1832 | CAREADGLHSPWHFDLW |
| 2A-9 | 363 | FTFPRYAMS | 1098 | STISGSGSTTYYA | 1833 | CARLIDAFDIW |
| 2A-21 | 364 | FTFPRYAMS | 1099 | STISGSGSTTYYA | 1834 | CARLIDAFDIW |
| 2A-22 | 365 | FTFTTYALS | 1100 | SGISGSGDETYYA | 1835 | CTTGDDFWSGGNWFDPW |
| 2A-23 | 366 | FTFSRHAMN | 1101 | SGITGSGDETYYA | 1836 | CARDLPASYYDSSGYYWHNG |
| | | | | | | MDVW |
| 2A-24 | 367 | FVFSSYAMS | 1102 | SAISGSGGSSYYA | 1837 | CARVGGGYWYGIDVW |
| 2A-25 | 368 | FTLSSYVMS | 1103 | SGISGGGASTYYA | 1838 | CARGYSRNWYPSWFDPW |
| 2A-26 | 369 | FTFSTYAMS | 1104 | SSIGGSGSTTYYA | 1839 | CAGGWYLDYW |
| 2A-27 | 370 | FTYSNYAMT | 1105 | SAISGSSGSTYYA | 1840 | CASLCIVDPFDIW |
| 2A-28 | 371 | FTFSNYPMN | 1106 | STISGSGGNTFYA | 1841 | CVRHDEYSFDYW |
| 3A-10 | 372 | FTFRSHAMS | 1107 | SSISGGGASTYYA | 1842 | CARVKYLTTSSGWPRPYFDNW |
| 3A-4 | 373 | FTFSAYSMS | 1108 | SAISGSGGSRYYA | 1843 | CGRSKWPQANGAFDIW |
| 3A-7 | 374 | FMFGNYAMS | 1109 | AAISGSGGSTYYA | 1844 | CAKDRGYSSSWYGGFDYW |
| 3A-1 | 375 | FTFRNHAMA | 1110 | SGISGSGGTTYYG | 1845 | CARGTRFLQWSLPLDVW |
| 3A-5 | 376 | FTIPNYAMS | 1111 | SGISGAGASTYYA | 1846 | CARHTWWKGAGFFDHW |
| 3A-6 | 377 | FTFRNYAMA | 1112 | SGISGSGGTTYYG | 1847 | CARGTRFLEWSLPLDVW |
| 3A-15 | 378 | FTIRNYAMS | 1113 | SSISGGGASTYYA | 1848 | CARVKYLTTSSGWPRPYFDNW |
| 3A-3 | 379 | FTIPNYAMS | 1114 | SGISGSGASTYYA | 1849 | CARHTWWKGAGFFDHW |
| 3A-11 | 380 | FTITNYAMS | 1115 | SGISGSGAGTYYA | 1850 | CARHAWWKGAGFFDHW |
| 3A-8 | 381 | FTFRSHAMS | 1116 | SSISGGGASTYYA | 1851 | CARVKYLTTSSGWPRPYFDNW |
| 3A-2 | 382 | FTITNYAMS | 1117 | SGISGSGASTYYA | 1852 | CARHTWWKGAGFFDHW |
| 3A-12 | 383 | FTFRSHAMN | 1118 | SAISGSGGSTNYA | 1853 | CARGLKFLEWLPSAFDIW |
| 3A-14 | 384 | FTFRSHAMS | 1119 | SSISGGGASTYYA | 1854 | CARVKYLTTSSGWPRPYFDNW |
| 3A-9 | 385 | FTFRSYAMS | 1120 | SSISGGGASTYYA | 1855 | CARVKYLTTSSGWPRPYFDNW |
| 3A-13 | 386 | FTITNYAMS | 1121 | SGISGSGAGTYYA | 1856 | CARHTWWKGAGFFDHW |
| 3A-16 | 387 | FTFTNFAMS | 1122 | SAISGRGGGTYYA | 1857 | CARDAHGYYYDSSGYDDW |
| 3A-17 | 388 | FTFRSYPMS | 1123 | STISGSGGITYYA | 1858 | CAKGVYGSTVTTCHW |
| 3A-18 | 389 | FTLTSYAMS | 1124 | SAISGSGVDTYYA | 1859 | CARPTNWGFDYW |
| 3A-19 | 390 | FTFINYAMS | 1125 | STISTSGGNTYYA | 1860 | CARADSNWASSAYW |
| 3A-2 | 391 | FPFSTYAMS | 1126 | SGISVSGGFTYYA | 1861 | CARDPYSYGYYYYYGMDVW |
| 3A-21 | 392 | FTFSTYAMG | 1127 | SGISGGGVSTYYA | 1862 | CARARNWGPSDYW |
| 3A-22 | 393 | FIFSDYAMT | 1128 | SAISGSAFYA | 1863 | CARDATYSSSWYNWFDPW |
| 3A-23 | 394 | FTFSDYAMT | 1129 | SDISGSGGSTYYA | 1864 | CARGTVTSFDFW |
| 3A-24 | 395 | FTFSIYAMG | 1130 | SFISGSGGSTYYA | 1865 | CAKDYHSASWFSAAADYW |
| 3A-25 | 396 | FTFASYAMT | 1131 | SAISESGGSTYYA | 1866 | CAREGQEYSSGSSYFDYW |
| 3A-26 | 397 | FTFSEYAMS | 1132 | SAITGSGGSTYYG | 1867 | CARGSQTPYCGGDCPETFDYW |
| 3A-27 | 398 | FTFDDYAMS | 1133 | SGISGGGTSTYYA | 1868 | CARDLYSSGWYGFDYW |
| 3A-28 | 399 | FTFNNYAMN | 1134 | SAISGSVGSTYYA | 1869 | CARDNYDFWSGYYTNWFDPW |
| 3A-29 | 400 | FTFTNHAMS | 1135 | SAISGSGSNIYYA | 1870 | CARDSLSVTMGRGVVTYYYY |
| | | | | | | GMDFW |
| 4A-51 | 401 | PGTAIMG | 1136 | ARISTSGGSTKYA | 1871 | CARTTVIIPPLIW |
| 4A-52 | 402 | RSFSNSVMG | 1137 | ARITWNGGSTYYA | 1872 | CATTENPNPRW |
| 4A-53 | 403 | RTFGDDTMG | 1138 | AAVSWSGSGVYYA | 1873 | CATDPPLFW |
| 4A-54 | 404 | RTFSDARMG | 1139 | GAVSWSGGTTVYA | 1874 | CATTEDPYPRW |
| 4A-49 | 405 | RTFGDYIMG | 1140 | AAINWSAGYTAYA | 1875 | CARASPNTGWHFDHW |
| 4A-55 | 406 | SGLSINAMG | 1141 | AAISWSGGSTYTAYA | 1876 | CAAYQAGWGDW |
| 4A-39 | 407 | RTFSNAAMG | 1142 | ARILWTGASRNYA | 1877 | CATTENPNPRW |
| 4A-56 | 408 | FSLDYYGMG | 1143 | AAISWNGDFTAYA | 1878 | CAKRANPTGAYFDYW |
| 4A-33 | 409 | FTFSRHDMG | 1144 | AGINWESGSTNYA | 1879 | CAADRGVYGGRWYRTSQYTW |
| 4A-57 | 410 | LTFRNYAMG | 1145 | AAIGSGGYTDYA | 1880 | CAVKPGWVARDPSQYNW |
| 4A-25 | 411 | GTFSRYAMG | 1146 | SAVDSGGSTYYA | 1881 | CAASPSLRSAWQW |
| 4A-58 | 412 | FTLDYYDMG | 1147 | AAVTWSGGSTYYA | 1882 | CAADRRGLASTRAADYDW |
| 4A-59 | 413 | RTFGDYIMG | 1148 | AAINWSAGYTPYA | 1883 | CATAPPLFCWHFDLW |
| 4A-6 | 414 | RTFGDDIMG | 1149 | AAIHWSAGYTRYA | 1884 | CATDPPLFWGHVDLW |
| 4A-61 | 415 | RTFGDYIMG | 1150 | AAINWSADYTPYA | 1885 | CATAPPNTGWHFDHW |
| 4A-3 | 416 | RTFGDYIMG | 1151 | AAINWSAGYTAYA | 1886 | CATATPNTGWHFDHW |
| 4A-62 | 417 | RTFSDDTMG | 1152 | AAINWSGGSTDYA | 1887 | CATDPPLFW |
| 4A-43 | 418 | RTFGDDTMG | 1153 | AGINWSGGNTYYA | 1888 | CATDPPLFW |
| 4A-5 | 419 | RTFGDYIMG | 1154 | AAINWTGGYTSYA | 1889 | CATDPPLFW |
| 4A-42 | 420 | RTFGDDTMG | 1155 | AAINWSGGNTYYA | 1890 | CATDPPLFW |
| 4A-63 | 421 | RTFSDYTMG | 1156 | AAINWSGGYTYYA | 1891 | CATDPPLFW |
| 4A-6 | 422 | RTFGDYGMG | 1157 | ATINWSGALTHYA | 1892 | CATLPFYDFWSGYYTGYYYM |
| | | | | | | DVW |
| 4A-40 | 423 | RTFSDDTMG | 1158 | AGVTWSGSSTFYA | 1893 | CATDPPLFW |
| 4A-21 | 424 | RTFSDDIMG | 1159 | AAISWSGGNTHYA | 1894 | CATDPPLFW |
| 4A-64 | 425 | RTFGDYIMG | 1160 | AAINWSAGYTAYA | 1895 | CATASPNTGWHFDHW |
| 4A-47 | 426 | FTFDDDYVMG | 1161 | AAVSGSGDDTYYA | 1896 | CAADRRGLASTRAADYDW |
| 4A-65 | 427 | RTFGDYIMG | 1162 | AAINWSAGYTAYA | 1897 | CATEPPLSCWHFDLW |
| 4A-18 | 428 | RTFGDYIMG | 1163 | AAINWSGGYTPYA | 1898 | CATAPPNTGWHFDHW |
| 4A-66 | 429 | RTFGDDTMG | 1164 | AAINWSAGYTPYA | 1899 | CATDPPLFCCHFDLW |
| 4A-36 | 430 | RTFSDDTMG | 1165 | AAISWSGGFIRYA | 1900 | CATDPPLFW |
| 4A-67 | 431 | RTFSDDTMG | 1166 | AAINWSGDSTYYA | 1901 | CATDPPLFW |
| 4A-16 | 432 | RTFSDDTMG | 1167 | AAINWSGGTTRYA | 1902 | CATDPPLFW |
| 4A-11 | 433 | RTFSDDAMG | 1168 | AAIHWSGSSTRYA | 1903 | CATDPPLFW |
| 4A-68 | 434 | RTFSDDTMG | 1169 | GTINWSGGSTYYA | 1904 | CATDPPLFW |
| 4A-34 | 435 | RTFGDYIMG | 1170 | AAINWSGGYTPYA | 1905 | CATDPPLFW |
| 4A-28 | 436 | RTFGDDTMG | 1171 | AAINWNGGNTHYA | 1906 | CATDPPLFW |
| 4A-69 | 437 | RTFSDDAMG | 1172 | AAINWSGGTTRYA | 1907 | CATDPPLFW |
| 4A-7 | 438 | RTFGDYIMG | 1173 | AAINWSAGYTPYA | 1908 | CATDPPLFWGHVDLW |
| 4A-71 | 439 | RTFSDDTMG | 1174 | ASINWSGGSTYYA | 1909 | CATDPPLFW |
| 4A-23 | 440 | RTFSDDAMG | 1175 | AGISWNGGSIYYA | 1910 | CATDPPLFW |
| 4A-9 | 441 | FTFDDYEMG | 1176 | AAISWRGGTTYYA | 1911 | CAADRRGLASTRAGDYDW |
| 4A-72 | 442 | RTFGDDTMG | 1177 | AAINWSGGYTPYA | 1912 | CATDPPLFWGHVDLW |
| 4A-73 | 443 | RTFSDDAMG | 1178 | AAINWSGGSTRYA | 1913 | CATDPPLFW |
| 4A-29 | 444 | VTLDDYAMG | 1179 | AVINWSGGSTDYA | 1914 | CARGGGWVPSSTSESLNWYFD |
| | | | | | | RW |
| 4A-41 | 445 | RTFGDYIMG | 1180 | AAINWSGGTTPYA | 1915 | CATDPPLFCCHVDLW |
| 4A-74 | 446 | LTFSDDTMG | 1181 | AAVSWSGGNTYYA | 1916 | CATDPPLFW |
| 4A-75 | 447 | RTFGDDTMG | 1182 | AAINWTGGYTPYA | 1917 | CATDPPLFW |
| 4A-31 | 448 | RTFGDYIMG | 1183 | ATINWTAGYTYYA | 1918 | CATDPPLFCWHFDHW |
| 4A-32 | 449 | RTFGDDTMG | 1184 | AAINWSGGNTDYA | 1919 | CATDPPLFW |
| 4A-15 | 450 | RTFGDYTMG | 1185 | AAINWSGGNTYYA | 1920 | CATDPPLFW |
| 4A-14 | 451 | RTFSDDTMG | 1186 | AGINWSGNGVYYA | 1921 | CATDPPLFW |
| 4A-76 | 452 | RTFGDYAMG | 1187 | APINWSGGSTYYA | 1922 | CATDPPLFW |
| 4A-50 | 453 | GTFSNSGMG | 1188 | AVVNWSGRRTYYA | 1923 | CAVPWMDYNRRDW |
| 4A-17 | 454 | QLANFASYAMG | 1189 | AAITRSGSSTVYA | 1924 | CATTMNPNPRW |
| 4A-37 | 455 | RTFSDDIMG | 1190 | AAINWTGGSTYYA | 1925 | CATDPPLFW |
| 4A-44 | 456 | RTFGDYIMG | 1191 | AAINWSAGYTAYA | 1926 | CATARPNTGWHFDHW |
| 4A-77 | 457 | RTFSDDTMG | 1192 | GSINWSGGSTYYA | 1927 | CATDPPLFW |
| 4A-78 | 458 | RTFSDDTMG | 1193 | AGMTWSGSSTFYA | 1928 | CATDPPLFW |
| 4A-79 | 459 | RTFGDYIMG | 1194 | AAINWSGDYTDYA | 1929 | CATDPPLFW |
| 4A-8 | 460 | RTFGDYIMG | 1195 | GGINWSGGYTYYA | 1930 | CATDPPLFW |
| 4A-81 | 461 | RTFSDDTMG | 1196 | AAVNWSGGSTYYA | 1931 | CATDPPLFW |
| 4A-82 | 462 | RTFGDYAMG | 1197 | AAINWSGGYTRYA | 1932 | CATDPPLFW |
| 4A-83 | 463 | RTFGDDTMG | 1198 | AAINWSGGYTPYA | 1933 | CATDPPLFW |
| 4A-35 | 464 | RTFGDYIMG | 1199 | AAINWSAGYTAYA | 1934 | CARASPNTGWHFDRW |
| 4A-45 | 465 | RTFGDYIMG | 1200 | AAINWSGGYTHYA | 1935 | CATDPPLFW |
| 4A-84 | 466 | RTFSDDTMG | 1201 | AAITWSGGRTRYA | 1936 | CATDRPLFW |
| 4A-85 | 467 | RTFGDYIMG | 1202 | AAINWSGGYTAYA | 1937 | CATASPNTGWHFDHW |
| 4A-86 | 468 | RTFSDDTMG | 1203 | AAIHWSGSSTRYA | 1938 | CATDPPLFW |
| 4A-87 | 469 | RTFSDYTMG | 1204 | AAINWSGGTTYY | 1939 | CATDPPLFW |
| 4A-88 | 470 | RTFGDDTMG | 1205 | AAINWSGDNTHY | 1940 | CATDPPLFW |
| 4A-89 | 471 | FAFGDNWIG | 1206 | ASISSGGTTAYA | 1941 | CAHRGGWLRPWGYW |
| 4A-9 | 472 | RTFSDDAMG | 1207 | GRINWSGGNTYYA | 1942 | CATDPPLFW |
| 4A-91 | 473 | RTFSDDTMG | 1208 | GGISWSGGNTYYA | 1943 | CATDPPLFW |
| 4A-92 | 474 | RTFSDDTMG | 1209 | AAINWSGGSTYYA | 1944 | CATDPPLFW |
| 4A-46 | 475 | RTFGDDTMG | 1210 | AAINWSGGYTYYA | 1945 | CATDPPLFW |
| 4A-20 | 476 | RTFGDYIMG | 1211 | AAINWSADYTAYA | 1946 | CATDPPLFCWHFDHW |
| 4A-93 | 477 | RTFSDDAMG | 1212 | AAINWSGSSTYYA | 1947 | CATDPPLFW |
| 4A-4 | 478 | RTFGDYIMG | 1213 | AAINWIAGYTADA | 1948 | CAEPSPNTGWHFDHW |
| 4A-2 | 479 | RTFGDDTMG | 1214 | AAINWSGGNTPYA | 1949 | CATDPPLFW |
| 4A-94 | 480 | RTFSDDTMG | 1215 | AAINWSGDNTHYA | 1950 | CATDPPLFW |
| 4A-95 | 481 | RTFGDYIMG | 1216 | AAINWSAGYTAYA | 1951 | CATAPPLFCWHFDHW |
| 4A-12 | 482 | FTFGDYVMG | 1217 | AAINWNAGYTAYA | 1952 | CAKASPNTGWHFDHW |
| 4A-30 | 483 | RTFGDYTMG | 1218 | AAINWTGGYTYYA | 1953 | CATDPPLFW |
| 4A-27 | 484 | RTFGDYIMG | 1219 | AAINWSAGYTAYA | 1954 | CARATPNTGWHFDHW |
| 4A-22 | 485 | RTFGDYIMG | 1220 | AAINWSGDNTHYA | 1955 | CATDPPLFW |
| 4A-96 | 486 | RTFGDYIMG | 1221 | AAINWSAGYTPYA | 1956 | CATDPPLFCCHFDHW |
| 4A-97 | 487 | RTFGDYIMG | 1222 | AAINWSAGYTAYA | 1957 | CATAPPNTGWHFDHW |
| 4A-98 | 488 | FTWGDYTMG | 1223 | AAINWSGGNTYYAA | 1958 | CAADRRGLASTRAADYDW |
| 4A-99 | 489 | IPSTLRAMG | 1224 | AAVSSLGPFTRYA | 1959 | CAAKPGWVARDPSQYNW |
| 4A-100 | 490 | FSFDDDYVMG | 1225 | AAINWSGGSTYYA | 1960 | CAADRRGLASTRAADYDW |
| 4A-101 | 491 | RTFSNAAMG | 1226 | ARILWTGASRSYA | 1961 | CATTENPNPRW |
| 4A-102 | 492 | GTFGVYHMG | 1227 | AAINMSGDDSAYA | 1962 | CAILVGPGQVEFDHW |
| 4A-103 | 493 | FTFSSYYMG | 1228 | ARISGSTFYA | 1963 | CAALPFVCPSGSYSDYGDEYDW |
| 4A-104 | 494 | RTFSGDFMG | 1229 | GRINWSGGNTYYA | 1964 | CPTDPPLFW |
| 4A-105 | 495 | STLRDYAMG | 1230 | AAITWSGGSTAYA | 1965 | CASLLAGDRYFDYW |
| 4A-106 | 496 | FTFDDYTMG | 1231 | AAITDNGGSKYYA | 1966 | CAADRRGLASTRAADYDW |
| 4A-107 | 497 | GTFSSYGMG | 1232 | AAINWSGASTYYA | 1967 | CARDWRDRTWGNSLDYW |
| 4A-108 | 498 | FSFDDDYVMG | 1233 | AAISWSEDNTYYA | 1968 | CAADRRGLASTRAADYDW |
| 4A-109 | 499 | FSFDDDYVMG | 1234 | AAVSGSGDDTYYA | 1969 | CAADRRGLASTRAADYDW |
| 4A-110 | 500 | NIAAINVMG | 1235 | AAISASGRRTDYA | 1970 | CARRVYYYDSSGPPGVTFDIW |
| 4A-111 | 501 | IITSRYVMG | 1236 | AAISTGGSTIYA | 1971 | CARQDSSSPYFDYW |
| 4A-112 | 502 | FSFDDDYVMG | 1237 | AAISNSGLSTYYA | 1972 | CAADRRGLASTRAADYDW |
| 4A-113 | 503 | SISSINVMG | 1238 | ATMRWSTGSTYYA | 1973 | CAQRVRGFFGPLRTTPSWYEW |
| 4A-114 | 504 | LTFILYRMG | 1239 | AAINNFGTTKYA | 1974 | CARTHYDFWSGYTSRTPNYFD |
| | | | | | | YW |
| 4A-115 | 505 | GTFSVYHMG | 1240 | AAISWSGGSTAYA | 1975 | CAAVNTWTSPSFDSW |
| 4A-116 | 506 | RAFSTYGMG | 1241 | AGINWSGDTPYYA | 1976 | CAREVGPPPGYFDLW |
| 4A-117 | 507 | RTFSDIAMG | 1242 | ASINWGGGNTYYA | 1977 | CAAKGIWDYLGRRDFGDW |
| 4A-118 | 508 | RTFSSARMG | 1243 | AAISWSGDNTHYA | 1978 | CATTENPNPRW |
| 4A-119 | 509 | FAFSSYAMG | 1244 | ATINGDDYTYYA | 1979 | CVATPGGYGLW |
| 4A-120 | 510 | ITFRRHDMG | 1245 | AAIRWSSSSTVYA | 1980 | CAADRGVYGGRWYRTSQYTW |
| 4A-121 | 511 | TAASFNPMG | 1246 | AAITSGGSTNYA | 1981 | CAAIAYEEGVYRWDW |
| 4A-122 | 512 | NINIINYMG | 1247 | AAIHWNGDSTAYA | 1982 | CASGPPYSNYFAYW |
| 4A-123 | 513 | FTFDDYAMG | 1248 | AAISGSGGSTAYA | 1983 | CAKIMGSGRPYFDHW |
| 4A-124 | 514 | NIFTRNVMG | 1249 | AAITSSGSTNYA | 1984 | CARPSSDLQGGVDYW |
| 4A-125 | 515 | RTFSSIAMG | 1250 | ASINWGGGNTIYA | 1985 | CAAKGIWDYLGRRDFGDW |
| 4A-126 | 516 | IPSTLRAMG | 1251 | AAVSSLGPFTRYA | 1986 | CAAKPGWVARDPSEYNW |
| 4A-127 | 517 | FTLDDSAMG | 1252 | AAITNGGSTYYA | 1987 | CARFARGSPYFDFW |
| 4A-128 | 518 | SISSFNAMG | 1253 | AAIDWDGSTAYA | 1988 | CARGGGYYGSGSFEYW |
| 4A-129 | 519 | NIFSDNIIG | 1254 | AYYTSGGSIDYA | 1989 | CARGTAVGRPPPGGMDVW |
| 4A-130 | 520 | SISSIGAMG | 1255 | AAISSSGSSTVYA | 1990 | CARVPPGQAYFDSW |
| 4A-131 | 521 | FTFDDYGMG | 1256 | ATITWSGDSTYYA | 1991 | CAKGGSWYYDSSGYYGRW |
| 4A-132 | 522 | RTFSNYTMG | 1257 | SAISWSTGSTYYA | 1992 | CAADRYGPPWYDW |
| 4A-133 | 523 | STNYMG | 1258 | AAISMSGDDTIYA | 1993 | CARIGLRGRYFDLW |
| 4A-134 | 524 | GTFSSVGMG | 1259 | AVINWSGARTYYA | 1994 | CAVPWMDYNRRDW |
| 4A-135 | 525 | RIFTNTAMG | 1260 | AAINWSGGSTAYA | 1995 | CARTSGSYSFDYW |
| 4A-136 | 526 | EEFSDHWMG | 1261 | GAIHWSGGRTYYA | 1996 | CAADRRGLASTRAADYDW |
| 4A-137 | 527 | RTFSSIAMG | 1262 | AAINWSGARTAYA | 1997 | CAAKGIWDYLGRRDFGDW |
| 4A-138 | 528 | STSSLRTMG | 1263 | AAISSRDGSTIYA | 1998 | CARDDSSSPYFDYW |
| 4A-139 | 529 | GGTFGSYAMG | 1264 | AAISIASGASGGTT | 1999 | CATTMNPNPRW |
| | | | | NYA | | |
| 4A-140 | 530 | RTFSNAAMG | 1265 | ARITWNGGSTFYA | 2000 | CATTENPNPRW |
| 4A-141 | 531 | IILSDNAMG | 1266 | AAISWLGESTYYA | 2001 | CAADRRGLASTRAADYDW |
| 4A-142 | 532 | RTFGDYIMG | 1267 | AAINWNGGYTAYA | 2002 | CATTSPNTGWHYYRW |
| 4A-143 | 533 | FNFNWYPMG | 1268 | AAISWTGVSTYTAYA | 2003 | CARWGPGPAGGSPGLVGFDYA |
| 4A-144 | 534 | SIRSVSVMG | 1269 | AAISWSGVGTAYA | 2004 | CAAYQRGWGDW |
| 4A-145 | 535 | MTFRLYAMG | 1270 | GAINWLSESTYYA | 2005 | CAAKPGWVARDPSEYNW |
| 4A-146 | 536 | RTFSDDAMG | 1271 | AAINWSGGSTYYA | 2006 | CATDPPLFW |
| 4A-147 | 537 | GTFSVYAMG | 1272 | AAISMSGDDAAYA | 2007 | CAKISKDDGGKPRGAFFDSW |
| 4A-148 | 538 | FALGYYAMG | 1273 | AAISSRDGSTAYA | 2008 | CARLATGPQAYFHHW |
| 4A-149 | 539 | FNLDDYAMG | 1274 | AAISWDGGATAYA | 2009 | CARVGRGTTAFDSW |
| 4A-150 | 540 | NTFSGGFMG | 1275 | ASIRSGARTYYA | 2010 | CAQRVRGFFGPLRTTPSWYEW |
| 4A-151 | 541 | SIRSINIMG | 1276 | AAISWSGGSTVYA | 2011 | CASLLAGDRYFDYW |
| 5A-1 | 542 | GTFSSIGMG | 1277 | AAISWDGGATAYA | 2012 | CAKEDVGKPFDW |
| 5A-2 | 543 | LRFDDYAMG | 1278 | AIKFSGGTTDYA | 2013 | CASWDGLIGLDAYEYDW |
| 5A-3 | 544 | SIFSIDVMG | 1279 | AGISWSGDSTLYA | 2014 | CAAFDGYTGSDW |
| 5A-4 | 545 | FTLADYAMG | 1280 | AVITCSGGSTDYA | 2015 | CAADDCYIGCGW |
| 5A-5 | 546 | RTFSSIAMG | 1281 | AEITEGGISPSGDN | 2016 | CAAELHSSDYTSPGAESDYGW |
| | | | | IYYA | | |
| 5A-6 | 547 | PTFSSYAMMG | 1282 | AAINNFGTTKYA | 2017 | CAASASDYGLGLELFHDEYNW |
| 5A-7 | 548 | STGYMG | 1283 | AAIHSGGSTNYA | 2018 | CATVATALIW |
| 5A-8 | 549 | RPFSEYTMG | 1284 | SSIHWGGRGTNYA | 2019 | CAAELHSSDYTSPGAYAW |
| 5A-9 | 550 | LTLSTYGMG | 1285 | AHIPRSTYSPYYA | 2020 | CAAIGDGAVW |
| 5A-10 | 551 | FTFNNHNMG | 1286 | AAISSYSHTAYA | 2021 | CALQPFGASNYRW |
| 5A-11 | 552 | GIYRVMG | 1287 | ASISSGGGINYA | 2022 | CAAESWGRQW |
| 5A-12 | 553 | YTDSNLWMG | 1288 | AINRSTGSTSYA | 2023 | CATSGSGSPNW |
| 5A-13 | 554 | FTFDYYTMG | 1289 | AAIRSSGGLFYA | 2024 | CAAYLDGYSGSW |
| 5A-14 | 555 | GIFSINVMG | 1290 | SAIRWNGGNTAYA | 2025 | CAGFDGYTGSDW |
| 5A-15 | 556 | FTFDGAAMG | 1291 | ATIRWTNSTDYA | 2026 | CARGRYGIVERW |
| 5A-16 | 557 | RTHSIYPMG | 1292 | AAIHSGGATVYA | 2027 | CAARRWIPPGPIW |
| 5A-17 | 558 | PTFSIYAMG | 1293 | AGIRWSDVYTQYA | 2028 | CALDIDYRDW |
| 5A-18 | 559 | LTFDDNIHVMG | 1294 | AAIHWSGGSTIYA | 2029 | CAADVYPQDYGLGYVEGKMY |
| | | | | | | YGMDW |
| 5A-19 | 560 | LTLDYYAMG | 1295 | ASINWSGGSTYYA | 2030 | CAAYGSGEFDW |
| 5A-20 | 561 | RTIVPYTMG | 1296 | AAISPSAFTEYA | 2031 | CAARRWGYDW |
| 5A-21 | 562 | GTFTTYHMG | 1297 | AHISTGGATNYA | 2032 | CATFPAIVTDSDYDLGNDW |
| 5A-22 | 563 | FTFNVFAMG | 1298 | AAINWSDSRTDYA | 2033 | CASGSDNRARELSRYEYVW |
| 5A-23 | 564 | SIFSIDVMG | 1299 | AAISWSGESTLYA | 2034 | CAAFDGYSGSDW |
| 5A-24 | 565 | FTFSSYSMG | 1300 | AAISSYSHTAYA | 2035 | CALQPFGASSYRW |
| 5A-25 | 566 | NTFSINVMG | 1301 | AAIHWSGDSTLYA | 2036 | CAAFDGYSGNHW |
| 5A-26 | 567 | RTISSYIMG | 1302 | ARIYTGGDTIYA | 2037 | CAARTSYNGRYDY1DDYSW |
| 5A-27 | 568 | RANSINWMG | 1303 | ATITPGGNTNYA | 2038 | CAAAAGSTWYGTLYEYDW |
| 5A-28 | 569 | GTFSVFAMG | 1304 | AEITAGGSTYYA | 2039 | CAVDGPFGW |
| 5A-29 | 570 | FTFDDYPMG | 1305 | ASVLRGGYTWYA | 2040 | CAKDWATGLAW |
| 5A-30 | 571 | FALGYYAMG | 1306 | AGIRWTDAYTEYA | 2041 | CAADVSPSYGSRWYW |
| 5A-31 | 572 | RTLDIHVMG | 1307 | AVINWTGESTLYA | 2042 | CAAFDGYTGNYW |
| 5A-32 | 573 | FTPDNYAMG | 1308 | AALGWSGVTTYH | 2043 | CASDESDAANW |
| | | | | YYA | | |
| 5A-33 | 574 | FTFDDYAMG | 1309 | ATIMWSGNTTYYA | 2044 | CATNDDDV |
| 5A-34 | 575 | RTFSRYIMG | 1310 | AAISWSGGDNTYYA | 2045 | CAAYRIVVGGTSPGDWRW |
| 5A-35 | 576 | PTFSIYAMG | 1311 | AGISWNGGSTNYA | 2046 | CALRRRFGGQEW |
| 5A-36 | 577 | RTFSLNAMG | 1312 | AAISCGGGSTYA | 2047 | CAADNDMGYCSW |
| 5A-37 | 578 | STFSINAMG | 1313 | GGISRSGATTNYA | 2048 | CAADGVPEYSDYASGPVW |
| 5A-38 | 579 | RTFSMHAMG | 1314 | ASISSQGRTNYA | 2049 | CAAEVRNGSDYLPIDW |
| 5A-39 | 580 | VTLDLYAMG | 1315 | AGIRWTDAYTEYA | 2050 | CAVDIDYRDW |
| 5A-40 | 581 | LPFTINVMG | 1316 | AAIHWSGLTTFYA | 2051 | CAELDGYFFAHW |
| 5A-41 | 582 | RAFSNYAMG | 1317 | AWINNRGTTDYA | 2052 | CASTDDYGVDW |
| | | | | DSGSTYYA | | |
| 5A-42 | 583 | FTPDDYAMG | 1318 | ASIGYSGRSNSYN | 2053 | CAIAHGSSTYNW |
| | | | | YYA | | |
| 5A-43 | 584 | FTLNYYGMG | 1319 | AAITSGGAPHYA | 2054 | CASAYDRGIGYDW |
| 5A-44 | 585 | LPFSTKSMG | 1320 | AAIHWSGLTSYA | 2055 | CAADRAADFFAQRDEYDW |
| 5A-45 | 586 | RTFSINAMG | 1321 | AAISWSGESTQYA | 2056 | CAAFDGGSGTQW |
| 5A-46 | 587 | EEFSDHWMG | 1322 | AAIHWSGDSTHRN | 2057 | CATVGITLNW |
| | | | | YA | | |
| 5A-47 | 588 | FTFGSYDMG | 1323 | TAINWSGARTAYA | 2058 | CAARSVYSYEYNW |
| 5A-48 | 589 | LPLDLYAMG | 1324 | AGIRWSDAYTEYA | 2059 | CALDIDYRHW |
| 5A-49 | 590 | RTSTVNGMG | 1325 | ASISQSGAATAYA | 2060 | CAADRTYSYSSTGYYW |
| 5A-50 | 591 | FSLDYYGMG | 1326 | AAITSGGTPHYA | 2061 | CASAYNPGIGYDW |
| 5A-51 | 592 | RPNSINWMG | 1327 | ATITPGGNTNYA | 2062 | CAAAAGTTWYGTLYEYDW |
| 5A-52 | 593 | EKFSDHWMG | 1328 | ATITFSGARTAYA | 2063 | CAALIKPSSTDRIFEEW |
| 5A-53 | 594 | LTVVPYAMG | 1329 | AAIRRSAVTNYA | 2064 | CAARRWGYHYW |
| 5A-54 | 595 | TTFNFNVMG | 1330 | AVISWTGESTLYA | 2065 | CAAFDGYTGRDW |
| 5A-55 | 596 | IDVNRNAMG | 1331 | AAITWSGGWRYYA | 2066 | CATTFGDAGIPDQYDFGW |
| 5A-56 | 597 | RTFSSNMG | 1332 | ARIFGGDRTLYA | 2067 | CADINGDW |
| 5A-57 | 598 | GTFSMGWIR | 1333 | GCIGWITYYA | 2068 | CAPFGW |
| 5A-58 | 599 | CTLDYYAMG | 1334 | AGIRWTDAYTEYA | 2069 | CAADVSPSYGGRWYW |
| 5A-59 | 600 | LTFSLYRMC | 1335 | SCISNIDGSTYYA | 2070 | CAADLLGDSDYEPSSGFGW |
| 5A-60 | 601 | RSFSSHRMG | 1336 | AAIMWSGSHRNYA | 2071 | CAAIAYEEGVYRWDW |
| 5A-61 | 602 | RIIVPNTMG | 1337 | TGISPSAFTEYA | 2072 | CAAHGWGCHW |
| 5A-62 | 603 | SIFIISMG | 1338 | TGINWSGGSTTYA | 2073 | CAASAIGSGALRRFEYDW |
| 5A-63 | 604 | FSLDYYDMG | 1339 | AALGWSGGSTDYA | 2074 | CAAGNGGRYGIVERW |
| 5A-64 | 605 | TSISNRVMG | 1340 | ARIYTGGDTLYA | 2075 | CAARKIYRSLSYYGDYDW |
| 5A-65 | 606 | NIDRLYAMG | 1341 | AAIDSDGSTDYA | 2076 | CAALIDYGLGFPIEW |
| 5A-66 | 607 | NTFTINVMG | 1342 | AAINWNGGTTLYA | 2077 | CAAFDGYSGIDW |
| 5A-67 | 608 | FNVNDYAMG | 1343 | AGITSSVGVTNYA | 2078 | CAADIFFVNW |
| 5A-68 | 609 | FTFDHYTMG | 1344 | AAISGSENVTSYA | 2079 | CAAEPYIPVRTMRHMTFLTW |
| 6A-1 | 610 | RTFGNYNMG | 1345 | ATINSLGGTSYA | 2080 | CARVDYYMDVW |
| 6A-2 | 611 | FTMSSSWMG | 1346 | TVISGVGTSYA | 2081 | CARGPDSSGYGFDYW |
| 6A-3 | 612 | FTFSPSWMG | 1347 | ATINEYGGRNYA | 2082 | CARVDRDFDYW |
| 6A-4 | 613 | FTRDYYTMG | 1348 | AAISRSGSLTSYA | 2083 | CANLAYYDSSGYYDYW |
| 6A-5 | 614 | RTFTMG | 1349 | ASTNSAGSTNYA | 2084 | CTTVDQYFDYW |
| 6A-6 | 615 | TTLDYYAMG | 1350 | AAISWSGGSTAYA | 2085 | CAREDYYDSSGYSW |
| 6A-7 | 616 | FTFSSYWMG | 1351 | ATINWSGVTAYA | 2086 | CARADDYFDYW |
| 6A-8 | 617 | FTLSGIWMG | 1352 | AIITTGGRTTYA | 2087 | CAGYSTFGSSSAYYYYSMDVG |
| 6A-9 | 618 | FTFDYYAMG | 1353 | SAIDSEGRTSYA | 2088 | CARWGPFDIW |
| 6A-10 | 619 | SIASIHAMG | 1354 | AAISRSGGFGSYA | 2089 | CARDDKYYDSSGYPAYFQHW |
| 6A-11 | 620 | LAFNAYAMG | 1355 | ATIGWSGANTYYA | 2090 | CASDPPGW |
| 6A-12 | 621 | STYTTYSMG | 1356 | AAISGSENVTSYA | 2091 | CARVDDYMDVW |
| 6A-13 | 622 | LTFNDYAMG | 1357 | AHIPRSTYSPYYA | 2092 | CAFLVGPQGVDHGAFDVW |
| 6A-14 | 623 | ITFRFKAMG | 1358 | AAVSWDGRNTYYA | 2093 | CASDYYYMDVW |
| 6A-15 | 624 | STVLINAMG | 1359 | AAVRWSDDYTYYA | 2094 | CAKEGRAGSLDYW |
| 6A-16 | 625 | FTFDDAAMG | 1360 | AHISWSGGSTYYA | 2095 | CATFGATVTATNDAFDIW |
| 6A-17 | 626 | NTGSTGYMG | 1361 | AGVINDGSTVYA | 2096 | CARLATSHQDGTGYLFDYW |
| 6A-18 | 627 | LTFRNYAMG | 1362 | AGMMWSGGTTTYA | 2097 | CAREGYYYDSSGYLNYFDYW |
| 6A-19 | 628 | SILSIAVMG | 1363 | AAISPSAVTTYYA | 2098 | CAIGYYDSSGYFDYW |
| 6A-20 | 629 | STLPYHAMG | 1364 | AAITWNGASTSYA | 2099 | CARDRYYDTSASYFESETW |
| 6A-21 | 630 | TLFKINAMG | 1365 | AAITSSGSNIDYTYYA | 2100 | CARSNTGWYSFDYW |
| 6A-22 | 631 | RTFSEVVMG | 1366 | ATIHSSGSTSYA | 2101 | CVRVTSDYSMDSW |
| 6A-23 | 632 | SIFSMNTMG | 1367 | ALINRSGGGINYA | 2102 | CVRLSSGYYDFDYW |
| 6A-24 | 633 | FTLDYYAMG | 1368 | AAINWSGDNTHYA | 2103 | CARAPFYCTTTKCQDNYYYM |
| | | | | | | DVW |
| 6A-25 | 634 | LTFGTYTMG | 1369 | AAISRFGSTYYA | 2104 | CARGGDYDFWSVDYMDVW |
| 6A-26 | 635 | DTFSTSWMG | 1370 | ATINTGGGTNYA | 2105 | CARVTTSFDYW |
| 6A-27 | 636 | ITFRFKAMG | 1371 | ASISRSGTTYYA | 2106 | CATDYSAFDMW |
| 6A-28 | 637 | DTYGSYWMG | 1372 | ATITSDDRTNYA | 2107 | CARVTSSLSGMDVW |
| 6A-29 | 638 | YTLKNYYAMG | 1373 | AAIIWTGESTLDA | 2108 | CAREGYYDSSGYYW |
| 6A-30 | 639 | FAFGDSWMG | 1374 | ATINWSGVTAYA | 2109 | CARADGYFDYW |
| 6A-31 | 640 | DTFSANRMG | 1375 | ASITWSSANTYYA | 2110 | CATFNWNDEGFDFW |
| 6A-32 | 641 | FTLDYYDMG | 1376 | ALISWSGGSTYYA | 2111 | CATDFYGWGTRERDAFDIW |
| 6A-33 | 642 | TFQRINHMG | 1377 | ATINTGGQPNYA | 2112 | CASLIAAQDYYFDYW |
| 6A-34 | 643 | SAFRSNAMG | 1378 | AHISWSSKSTYYA | 2113 | CATYCSSTSCFDYW |
| 6A-35 | 644 | FTLAYYAMG | 1379 | AAISMSGDDTIYA | 2114 | CARELGYSSTVWPW |
| 6A-36 | 645 | FDFSVSWMG | 1380 | TAITWSGDSTNYA | 2115 | CASLLHTGPSGGNYFDYW |
| 6A-37 | 646 | HTFSTSWMG | 1381 | ATINSLGGTNYA | 2116 | CARVSSGDYGMDVW |
| 6A-38 | 647 | NTFSGGFMG | 1382 | AVISSLSSKSYA | 2117 | CAKVDSGYDYW |
| 6A-39 | 648 | FTFSPSWMG | 1383 | AAISWSGGSTAYA | 2118 | CHGLGEGDPYGDYEGYFDLW |
| 6A-40 | 649 | FTFSDYWMG | 1384 | ARVWWNGGSAYYA | 2119 | CAREVLRQQVVLDYW |
| 6A-41 | 650 | FTFSTSWMG | 1385 | ASINEYGGRNYA | 2120 | CAGLHYYYDSSGYNPTEYYG |
| | | | | | | MDVW |
| 6A-42 | 651 | DTYGSYWMG | 1386 | AVITSGGSTNYA | 2121 | CTHVQNSYYYAMDVW |
| 6A-43 | 652 | RTFSSYAMMG | 1387 | ASVNWDASQINYA | 2122 | CTTLGAVYFDSSGYHDYFDYW |
| 6A-44 | 653 | GTFGVYHMG | 1388 | GRITWTDGSTYYA | 2123 | CFGLLEVYDMTFDYW |
| 6A-45 | 654 | NMFSINAMG | 1389 | TLISWSSGRTSYA | 2124 | CASLGYCSGGSCFDYW |
| 6A-46 | 655 | LTFSAMG | 1390 | ALIRRDGSTIYA | 2125 | CAALGILFGYDAFDIW |
| 6A-47 | 656 | RTFSMHAMG | 1391 | ASITYGGNINYA | 2126 | CAKEGYYDSTGYRTYFQQW |
| 6A-48 | 657 | FTVSNYAMG | 1392 | ASVNWSGGITSYA | 2127 | CATTGTVTLGYW |
| 6A-49 | 658 | STVLINAMG | 1393 | AAISWSPGRTDYA | 2128 | CARDCSGGSCYSGDYW |
| 6A-50 | 659 | FSFDRWAMG | 1394 | ASLATGGNTNYA | 2129 | CARVTNYDAFDIW |
| 6A-51 | 660 | YTYSSYVMG | 1395 | AAISRFGSTYYA | 2130 | CARDSGEHFWDSGYIDHW |
| 6A-52 | 661 | DTYGSYWMG | 1396 | AAITSGGSTVYA | 2131 | CARVDSRFDYW |
| 6A-53 | 662 | ISINTNVMG | 1397 | AAISTGSVTIYA | 2132 | CARVDDFGYFDLW |
| 6A-54 | 663 | FEFENHWMG | 1398 | AHITAGGLSNYA | 2133 | CGRHWGIYDSSGFSSFDYW |
| 6A-55 | 664 | FTMSSSWMG | 1399 | ARITSGGSTGYA | 2134 | CASVDGYFDYW |
| 6A-56 | 665 | NIFRSNMG | 1400 | AGITWNGDTTYYA | 2135 | CARALGVTYQFDYW |
| 6A-57 | 666 | LTFDDHSMG | 1401 | AAVPLSGNTYYA | 2136 | CASFSGGPADFDYW |
| 6A-58 | 667 | RAVSTYAMG | 1402 | AAISGSENVTSYA | 2137 | CLSVTGDTEDYGVFDTW |
| 6A-59 | 668 | ISGSVFSRTPMG | 1403 | SSIYSDGSNTYYA | 2138 | CAHWSWELGDWFDPW |
| 6A-60 | 669 | DTYGSYWMG | 1404 | ATISQSGAATAYA | 2139 | CAGLLRYSGTYYDAFDVW |
| 6A-61 | 670 | DTYGSYWMG | 1405 | AAINWSGGSTNYA | 2140 | CAGLGWNYMDYW |
| 6A-62 | 671 | STFSGNWMG | 1406 | AVISWTGGSTYYA | 2141 | CATHNSLSGFDYW |
| 6A-63 | 672 | QTFNMG | 1407 | AAIGSGGSTSYA | 2142 | CWRLGNDYFDYW |
| 6A-64 | 673 | IPSIHAMG | 1408 | AAINWSHGVTYYA | 2143 | CGGGYGYHFDYW |
| 6A-65 | 674 | LPFSTLHMG | 1409 | ASLSIFGATGYA | 2144 | CWMYYYDSSGYYGNYYYGMDVW |
| 6A-66 | 675 | LTFSLFAMG | 1410 | AAISSGGSTDYA | 2145 | CARGNTKYYYDSSGYSSAFDYW |
| 6A-67 | 676 | SFSNYAMG | 1411 | AAISSSGALTSYA | 2146 | CWIVGPGPLDGMDVW |
| 6A-68 | 677 | FTLSDRAMG | 1412 | AHITAGGLSNYA | 2147 | CVHLASQTGAGYFDLW |
| 6A-69 | 678 | GTFSSVGMG | 1413 | AGISRSGGTYYA | 2148 | CARYDFWSGYPYW |
| 6A-70 | 679 | FNLDDYADMG | 1414 | AAIGWGGGSTRYA | 2149 | CAREILWFGEFGEPNVW |
| 6A-71 | 680 | ITFSNDAMG | 1415 | AIITSSDTNDTTNYA | 2150 | CARLHYYDSSGYFDYW |
| 6A-72 | 681 | STLSINAMG | 1416 | AAIDWSGGSTAYA | 2151 | CARDSSATRTGPDYW |
| 6A-73 | 682 | HTFSGYAMG | 1417 | AVITREGSTYYA | 2152 | CARLGGEGFDYW |
| 6A-74 | 683 | FAFGDSWMG | 1418 | AAITSGGSTDYA | 2153 | CARGLLWFGELFGYW |
| 6A-75 | 684 | GTFSTYWMG | 1419 | AAISRSGGNTYYA | 2154 | CVRHSGTDGDSSFDYW |
| 6A-76 | 685 | LAFDFDGMG | 1420 | AAINSGGSTYYA | 2155 | CARFFRAHDYW |
| 6A-77 | 686 | FTFDRSWMG | 1421 | AAVTEGGTTSYA | 2156 | CARADYDFDYW |
| 6A-78 | 687 | RTYDAMG | 1422 | ASVTSGGYTHYA | 2157 | CAKFGRKIVGATELDYW |
| 6A-79 | 688 | SISSIDYMG | 1423 | SWISSSDGSTYYA | 2158 | CARSPSFSQIYYYYYMDVW |
| 6A-80 | 689 | GTFSFYNMG | 1424 | AFISGNGGTSYA | 2159 | CAVVAMRMVTTEGPDVLDVW |
| 6A-81 | 690 | FIGNYHAMG | 1425 | AAVTWSGGTTNYA | 2160 | CAREGYYYDSSGYPYYFDYW |
| 6A-82 | 691 | SSLDAYGMG | 1426 | AAISWGGGSIYYA | 2161 | CARLSQGMVALDYW |
| 6A-83 | 692 | SIASIHAMG | 1427 | AAITWSGAITSYA | 2162 | CAKDGGYGELHYGMEVW |
| 6A-84 | 693 | FTPDDYAMG | 1428 | AAINSGGSYTYYA | 2163 | CARDRGPW |
| 6A-85 | 694 | GTFSVFAMG | 1429 | SAINWSGGSLLYA | 2164 | CALFGDFDYW |
| 6A-86 | 695 | PISGINRMG | 1430 | AVITSNGRPSYA | 2165 | CVRLSSGYFDFDYW |
| 6A-87 | 696 | TSIMVGAMG | 1431 | AIIRGDGRTSYA | 2166 | CARFAGWDAFDIW |
| 6A-88 | 697 | RTFSTHWMG | 1432 | AVINWSGGSIYYA | 2167 | CARLSSDGYNYFDFW |
| 6A-89 | 698 | TIFASAMG | 1433 | AVVNWNGSSTVYA | 2168 | CTTVDQYFNYW |
| 6A-90 | 699 | FPFSIWPMG | 1434 | AAVRWSSTYYA | 2169 | CATGECDGGSCSLAYW |
| 6A-91 | 700 | RTFGNYAMG | 1435 | ASISSSGVSKHYA | 2170 | CVRFGSSWARDLDQW |
| 6A-92 | 701 | FLFDSYASMG | 1436 | ATIWRRGNTYYA | 2171 | CTETGTAAW |
| | | | | NYA | | |
| 6A-93 | 702 | LPFSTKSMG | 1437 | AAISMSGLTSYA | 2172 | CLKVLGGDYEADNWFDYW |
| 6A-94 | 703 | NIFRIETMG | 1438 | AGIIRSGGETLYA | 2173 | CARSLYYDRSGSYYFDYW |
| 6A-95 | 704 | IPSSIRAMG | 1439 | AV1RWTGGSTYYA | 2174 | CARDIGYYDSSGYYNDGGFDYW |
| 6A-96 | 705 | FTLSGNWMG | 1440 | AIITSGGRTNYA | 2175 | CAGHATFGGSSSSYYYGMDVW |
| 6A-97 | 706 | FTFSSLAMG | 1441 | AAITWSGDITNYA | 2176 | CLRLSSSGFDHW |
| 6A-98 | 707 | TFGHYAMG | 1442 | AAINWSSRSTVYA | 2177 | CAKSDGLMGELRSASAFDIW |
| 6A-99 | 708 | IPFRSRTMG | 1443 | AGISRSGASTAYA | 2178 | CTHANDYGDYW |
| 6A-100 | 709 | GTFSTSWMG | 1444 | AHITAGGLSNYA | 2179 | CARLLVREDWYFDLW |
| 6A-101 | 710 | GTFSLFAMG | 1445 | AAISWTGDSTYYK | 2180 | CAYNNSSGEYW |
| | | | | YYA | | |
| 6A-102 | 711 | SSFSAYAMG | 1446 | SAIDSEGTTTYA | 2181 | CAGDYNFWSGFDHW |
| 6A-103 | 712 | RTSSPIAMG | 1447 | AVRWSDDYTYYA | 2182 | CAKKLGGYYAFDIW |
| 6A-104 | 713 | LTFNQYTMG | 1448 | ASITDGGSTYYA | 2183 | CARDSRYMDVW |
| 6A-105 | 714 | PTFSSMG | 1449 | AAISWDGGATAYA | 2184 | CAIEIVVGGIYW |
| 6A-106 | 715 | IPSTLRAMG | 1450 | AATSWSGGSKYYA | 2185 | CATDLYYMDVW |
| 6A-107 | 716 | GVGFSVTNMG | 1451 | AVISSSSSTNYA | 2186 | CTTFNWNDEGFDYW |
| 6A-108 | 717 | GTFGSYGMG | 1452 | AAIRWSGGITYYA | 2187 | CARERYWNPLPYYYYGMDVW |
| 6A-109 | 718 | GTFSTYAMG | 1453 | ASIDWSGLTSYA | 2188 | CARGPFYMYCSGTKCYSTNW |
| | | | | | | FDPW |
| 6A-110 | 719 | PIYAVNRMG | 1454 | AGIWRSGGHRDYA | 2189 | CARGEIDILTGYWYDYW |
| 6A-111 | 720 | FTFSNYWMG | 1455 | GGISRSGVSTSYA | 2190 | CTTLLYYYDSSGYSFDAFDIW |
| 6A-112 | 721 | GTFSAYHMG | 1456 | TIIDNGGPTSYA | 2191 | CTALLYYFDNSGYNFDPFDIW |
| 9A-1 | 722 | RTFSRLAMG | 1457 | AAISRSGRSTSYA | 2192 | CAARRSQILFTSRTDYEW |
| 9A-2 | 723 | SFSIAAMG | 1458 | ATINYSGGGTYYA | 2193 | CAAVN1FDESAYAAFACYDVVW |
| 9A-3 | 724 | RTFSRYAMG | 1459 | AAISRSGKSTYYA | 2194 | CAASSVFSDLRYRKNPKW |
| 9A-4 | 725 | RTFSKYAMG | 1460 | ALITPSSRTTYYA | 2195 | CAIAGRGRW |
| 9A-5 | 726 | RTFRRYAMG | 1461 | ASINWGGGNTYYA | 2196 | CAKTKRTGIFTTARMVDW |
| 9A-6 | 727 | RTFSRFAMG | 1462 | AAIRWSGGRTVYA | 2197 | CAIEPGTIRNWRNRVPFARGNF |
| | | | | | | GW |
| 9A-7 | 728 | LGIAFSRRTAMG | 1463 | AAISWRGGNTYYA | 2198 | CAARRWIPPGPIW |
| 9A-8 | 729 | RTFRRYPMG | 1464 | AAISRSGGSTYYA | 2199 | CAAKRLRSFASGGSYDW |
| 9A-9 | 730 | GTLRGYGMG | 1465 | ASISRSGGSTYYA | 2200 | CAARRRVTLFTSRADYDW |
| 9A-10 | 731 | RMFSSRSMG | 1466 | ALINRSGGSQFYA | 2201 | CAARRWIPPGPIW |
| 9A-11 | 732 | RTFGRRAMG | 1467 | AGISRGGGTNYA | 2202 | CAAKGIWDYLGRRDFGDW |
| 10A-1 | 733 | LSSPPFDDFPMG | 1468 | SSIYSDDGDSMYA | 2203 | CARQTFDFWSASLGGNFWYFDLW |
| 10A-2 | 734 | GTFSSYSMG | 1469 | SAISWIIGSGGTTNYA | 2204 | CTAGAGDSW |
| 10A-3 | 735 | SIFSTRTMG | 1470 | ASITKFGSTNYA | 2205 | CTRGGGRFFDWLYLRW |
| 10A-4 | 736 | RTLWRSNMG | 1471 | ASISSFGSTKYA | 2206 | CARGHGRYFDWLLFARPPDYW |
| 10A-5 | 737 | RSLGIYRMG | 1472 | AAITSGGRKNYA | 2207 | CAKRTIFGVGRWLDPW |
| 10A-6 | 738 | TTLTFRIMG | 1473 | PAISSTGLASYT | 2208 | CSKDRAPNCFACCPNGFDVW |
| 10A-7 | 739 | SRFSGRFNILNMG | 1474 | ARIGYSGQSISYA | 2209 | CARGRFLGGTEW |
| 10A-8 | 740 | TLFKINAMG | 1475 | AQINRHGVTYYA | 2210 | CARGRTIFFGGGRYFDYW |
| 10A-9 | 741 | IPFRSRTMG | 1476 | AGITGSGRSQYYA | 2211 | CARGARIFGSVAPWRGGNYY |
| | | | | | | GMDVW |
| 10A-10 | 742 | FTFSSFRMG | 1477 | AGISRGGSTNYA | 2212 | CARASGLWFRRPHVW |
| 10A-11 | 743 | RNFRRNSMG | 1478 | AGISWSGARTHYA | 2213 | CARVSRRPRSPPGYYYGMDVW |
| 10A-12 | 744 | RNLRMYRMG | 1479 | ATIRWSDGSTYYA | 2214 | CTRARLRYFDWLFPTNFDYW |
| 10A-13 | 745 | GLTFSSNTMG | 1480 | ASISSSGRTSYA | 2215 | CARRVRRLWFRSYFDLW |
| 10A-14 | 746 | FTLAYYAMG | 1481 | AAISWSGRNINYA | 2216 | CARERARWFGKFSVSW |
| 10A-15 | 747 | RTFSSFPMG | 1482 | AAISWSGSTSYA | 2217 | SACGRLGFGAW |
| 10A-16 | 748 | ISSSKRNMG | 1483 | ATWTSRGITTYA | 2218 | CARGGPPRLWGSYRRKYFDYW |
| 10A-17 | 749 | RTFSIYAMG | 1484 | ARITRGGITKYA | 2219 | CARGLGWLLGYYW |
| 10A-18 | 750 | RMYNSYSMG | 1485 | ARISPGGTFYA | 2220 | CTTSARSGWFWRYFDSW |
| 10A-19 | 751 | RTFRSYGMG | 1486 | ASISRSGTTMYA | 2221 | CARRGLLQWFGAPNSWFDPW |
| 10A-20 | 752 | RTIRTMG | 1487 | ATINSRGITNYA | 2222 | CTTERDGLLWFRELFRPSW |
| 10A-21 | 753 | RSFSFNAMG | 1488 | ARISRFGRTNYA | 2223 | CAKVHSYVWGGHSDYW |
| 10A-22 | 754 | RTYYAMG | 1489 | GAIDWSGRRITYA | 2224 | CARVRFSRLGGVIGRPIDSW |
| 10A-23 | 755 | RAFRRYTMG | 1490 | ASITKFGSTNYA | 2225 | CAKDRGVLWFGELWYW |
| 10A-24 | 756 | RTFSNYRMG | 1491 | ASINRGGSTKYA | 2226 | CASGKGGSATIFHLSRRPLYFD |
| | | | | | | YW |
| 10A-25 | 757 | ITFSPYAMG | 1492 | ATINWSGGYTVYA | 2227 | CAKRKNRGPLWFGGGGWGYW |
| 10A-26 | 758 | RTFSGFTMSST | 1493 | AGIITNGSTNYA | 2228 | CARRVAYSSFWSGLRKHMDV |
| | | WMG | | | | W |
| 10A-27 | 759 | RTFRRYSMG | 1494 | ASITPGGNTNYA | 2229 | CASRRRWLTPYIFW |
| 10A-28 | 760 | SIFSIGMG | 1495 | ARIWWRSGATYYA | 2230 | CAAISIFGRLKW |
| 10A-29 | 761 | RTFTSYRMG | 1496 | AEISSSGGYTYYA | 2231 | CARVGPLRFLAQRPRLRPDYW |
| 10A-30 | 762 | RTFSSFRFRAMG | 1497 | ALIFSGGSTYYA | 2232 | CAREWGRWLQRGSYW |
| 10A-31 | 763 | RTFGSYGMG | 1498 | ATISIGGRTYYA | 2233 | CARGSGSGFMWYHGNNNYDR |
| | | | | | | WRYW |
| 10A-32 | 764 | RTFRSYPMG | 1499 | ASINRGGSTNYA | 2234 | CARGRYDFWSGYYRWFDPW |
| 10A-33 | 765 | RTFSRSDMG | 1500 | AAISWSGGSTSYA | 2235 | CATVPPPRRFLEWLPRRLTYIW |
| 10A-34 | 766 | RTFRRYTMG | 1501 | ASMRGSRSYYA | 2236 | CARMSGFPFLDYW |
| 10A-35 | 767 | SIFRLSTMG | 1502 | ASISSFGSTYYA | 2237 | CARTRGIFLWFGESFDYW |
| 10A-36 | 768 | IAFRIRTMG | 1503 | ASITSGGSTNYA | 2238 | CARGGPRFGGFRGYFDPW |
| 10A-37 | 769 | FTFTSYRMG | 1504 | AGISRFFGTAYYA | 2239 | CARVTRWFGGLDVW |
| 10A-38 | 770 | RTFSRYVMG | 1505 | ASISRFGRTNYA | 2240 | CARHHGLGILWWGTMDVW |
| 10A-39 | 771 | RTFSMG | 1506 | ASISRFGRTNYA | 2241 | CAKRSTWLPQHFDSW |
| 10A-40 | 772 | RTFSTYTMG | 1507 | ARIWRSGGNTYYA | 2242 | CARGVRGVFRAYFDHW |
| 10A-41 | 773 | RNLRMYRMG | 1508 | ALISRVGVTSYA | 2243 | CARGTSFFNFWSGSLGRVGFD |
| | | | | | | SW |
| 10A-42 | 774 | ITIRTHAMG | 1509 | ATISRSGGNTYYA | 2244 | CTTAGVLRYFDWFRRPYW |
| 10A-43 | 775 | RTFRRYHMG | 1510 | AAITSGGRTNYA | 2245 | CTTDGLRYFDWFPWASAFDIW |
| 10A-44 | 776 | RTFRRYTMG | 1511 | AVISWSGGSTKYA | 2246 | CARKGRWSGMNVW |
| 10A-45 | 777 | RTFSWYPMG | 1512 | ASISWGGARTYYA | 2247 | CARSTGPRGSGRYRAHWFDSW |
| 10A-46 | 778 | RTFTSYRMG | 1513 | AAITWNSGRTRYA | 2248 | CSPSSWPFYFGAW |
| 10A-47 | 779 | RPLRRYVMG | 1514 | AAITNGGSTKYA | 2249 | CARGTPWRLLWFGTLGPPPAF |
| | | | | | | DYW |
| 10A-48 | 780 | RTFRRYAMG | 1515 | AAINRSGSTEYA | 2250 | CARQHQDFWTGYYTVW |
| 10A-49 | 781 | RTFRRYTMG | 1516 | ASISRSGTTYYA | 2251 | CAKEGWRWLQLRGGFDYW |
| 10A-50 | 782 | RTLSTYNMG | 1517 | ASISRFGRTNYA | 2252 | CARRGKLSAAMHWFDPW |
| 10A-51 | 783 | RFFSTRVMG | 1518 | ARIWPGGSTYYA | 2253 | CARDRGIFGVSRW |
| 10A-52 | 784 | RFFSICSMG | 1519 | AGINWRSGGSTYYA | 2254 | CARGSGWWEYW |
| 10A-53 | 785 | RMFSSRSNMG | 1520 | ASISSGGTTAYA | 2255 | CARGFGRRFLEWLPRFDYW |
| 10A-54 | 786 | RTFSSARMG | 1521 | AGINMISSTKYA | 2256 | CAHFRRFLPRGYVDYW |
| 10A-55 | 787 | RTFRRYTMG | 1522 | ARIAGGSTYYA | 2257 | CARQQYYDFWSGYFRSGYFDLW |
| 10A-56 | 788 | HTFRNYGMG | 1523 | AAITSSGSTNYA | 2258 | CATVPPPRRFLEWLPRRLTYTW |
| 10A-57 | 789 | RTFSRYAMG | 1524 | ASITKFGSTNYA | 2259 | CAKERESRFLKWRKTDW |
| 10A-58 | 790 | RNLRMYRMG | 1525 | ASISRFGRTNYA | 2260 | CARHDSIGLFRHGMDVW |
| 10A-59 | 791 | RTFRRYAMG | 1526 | ARISSGGSTSYA | 2261 | CARDRGFGFWSGLRGYFDLW |
| 10A-60 | 792 | IPASMYLG | 1527 | AAITSGGRTSYA | 2262 | CAKRKKRGPLWFGGGGWGYW |
| 10A-61 | 793 | IPFRSRTFSAY | 1528 | AQITRGGSTNYA | 2263 | CARRHWFGFDYW |
| | | AMG | | | | |
| 9-1 | 794 | FTFSSYAMH | 1529 | AVISYDGNHEYYA | 2264 | CARGYKGYYYMDVW |
| 9-2 | 795 | FSFNNYGMH | 1530 | AVISFDGSNEYYA | 2265 | CAKENWLGYFDPW |
| 9-3 | 796 | FTFGTYAMH | 1531 | AVVSTEGGTTYYA | 2266 | CAGSYGAYFDYW |
| 9-4 | 797 | FDFSDYYMH | 1532 | AVISYDGSNKYYA | 2267 | CAREEPVYGMDVW |
| 9-5 | 798 | FTFGTYAMH | 1533 | AVVSTEGGTTYYA | 2268 | CAGSYGAYFDYW |
| 9-6 | 799 | FTFSGYAMH | 1534 | AVISYDGSNEYYA | 2269 | CARTNSGSYYGPFDYW |
| 9-7 | 800 | FTFSSYAMH | 1535 | AVISYDGNHEYYA | 2270 | CARGYKGYYYMDVW |
| 9-8 | 801 | FTFSSYAMH | 1536 | AVISYDGNHEYYA | 2271 | CARGYKGYYYMDVW |
| 9-9 | 802 | FIFRSYAMH | 1537 | AVISYDGSSKYYA | 2272 | CARPSSGSYFPPFDYW |
| 9-10 | 803 | FTFSDYGMH | 1538 | AVVSYDGTTKYYA | 2273 | CAKENWLGYFDPW |
| 9-11 | 804 | FTFSNFPMH | 1539 | AVISYDGSLKYYA | 2274 | CARYQGGYMDVW |
| 9-12 | 805 | FTFSRFAMH | 1540 | AVISYDGSNKYYA | 2275 | CARDTGLGFDPW |
| 9-13 | 806 | FTFNNYAMH | 1541 | AVISYDGNNKYYA | 2276 | CAKTMGGSYFDAFDIW |
| 9-14 | 807 | FTFSDYTMH | 1542 | AVISYEGSIKYYA | 2277 | CARSSSGSYPSLVDYW |
| 9-15 | 808 | #N/A | 1543 | #N/A | 2278 | CARDYWVDYFKPG |
| 10-1 | 809 | FTFSRYAMH | 1544 | AVISYDGTNEYYA | 2279 | CARDTGLGFDPW |
| 10-2 | 810 | FTFSRYAMH | 1545 | AVISYDGTNEYYA | 2280 | CARDTGLGFDPW |
| 10-3 | 811 | FTFSRYAMH | 1546 | AVISYDGTNEYYA | 2281 | CARDTGLGFDPW |
| 10-4 | 812 | FTFSRYAMH | 1547 | AVISYDGTNEYYA | 2282 | CARDTGLGFDPW |
| 10-5 | 813 | FTFSRYAMH | 1548 | AVISYDGTNEYYA | 2283 | CARDTGLGFDPW |
| 10-6 | 814 | FTFSRYAMH | 1549 | AVISYDGTNEYYA | 2284 | CARDTGLGFDPW |
| 11-1 | 815 | FTFGSYGMH | 1550 | AVISYDGGDEYYA | 2285 | CARDISRYGYYGMDVW |
| 11-2 | 816 | FTFGTYAMH | 1551 | AVVSTEGGTTYYA | 2286 | CAGSYGAYFDYW |
| 11-3 | 817 | FTFSNFAMH | 1552 | AVISYDGNHEYYA | 2287 | CAKTNSGSYGGMFDYW |
| 11-4 | 818 | FTFDNYAMH | 1553 | AVISDDGRNKYYA | 2288 | CAKDNYYDSSGYYGGGMDVW |
| 11-5 | 819 | FTFSSFAMH | 1554 | AVISYDGSNKYYA | 2289 | CARSRSGSYSSYFDYW |
| 11-6 | 820 | FTFGTYAMH | 1555 | AVVSTEGGTTYYA | 2290 | CAGEYYDSSGSSIDYW |
| 11-7 | 821 | FTFSSYAMH | 1556 | AVISYDGSNQYYA | 2291 | CARAKGGGYRGAFDIW |
| 11-8 | 822 | FTFSSYAMH | 1557 | AVISYDGSNTYYA | 2292 | CARPRGGSYWTYFDYW |
| 11-9 | 823 | FTFGTYAMH | 1558 | AVVSTEGGTTYYA | 2293 | CAGSYGAYFDYW |
| 11-10 | 824 | FIFNNYGMH | 1559 | AVISYDGSNIYYA | 2294 | CARDYNDGIGSYTGAFDSW |
| 11-11 | 825 | FTFDNYAMH | 1560 | AVISYDGSNKYYA | 2295 | CLREGILWDVW |
| 11-12 | 826 | FTFSSQAMH | 1561 | AVISYDGSNKYYA | 2296 | CAKTEGGTYGGAFDIW |
| 11-13 | 827 | FSFSSYGMH | 1562 | AVISYDGSDKYYA | 2297 | CAKDNYYDSSGYYGGGMDVW |
| 11-14 | 828 | FTFSSYSMH | 1563 | AVISYDGSHKYYA | 2298 | CARDGWGYFDYW |
| 11-15 | 829 | FIFSNYGMH | 1564 | AVISYDGSDKYYA | 2299 | CARDDYMYGFEHW |
| 11-16 | 830 | FTFSDHYMH | 1565 | AVISYDGSNEYYA | 2300 | CAKDLGPAGVDYW |
| 11-17 | 831 | FIFSSYAMH | 1566 | AVISYDGSNKYYA | 2301 | CARSRSGSYSSWFDYW |
| 11-18 | 832 | FTFGTYAMH | 1567 | AVISYDGNNKYYA | 2302 | CAKTGSGSYYSWFDYW |
| 11-19 | 833 | FTFSSYAMH | 1568 | AVISYDGTNDYYA | 2303 | CARTRGGSYFTPFDYW |
| 11-20 | 834 | FTFDDYAMH | 1569 | AVISYDGSNKYYA | 2304 | CASPHSGSYWAAFDIW |
| 12-12-1 | 835 | FTFSYYGMH | 1570 | AVTSYDGSNKYYA | 2305 | CARPQGGSYFAAFDIW |
| 12-2 | 836 | FIFRSYAMH | 1571 | AVISYDGSSKYYA | 2306 | CARPSSGSYFPPFDYW |
| 12-3 | 837 | FTFSSYAMH | 1572 | AVISYDGSNQYYA | 2307 | CAKTRTGSYFSAFDIW |
| 12-4 | 838 | FTFSYYGMH | 1573 | AVISYDGTNDYYA | 2308 | CAKPHSGSYRGYFDYW |
| 12-5 | 839 | FTFSYYGMH | 1574 | AVTSYDGSNKYYS | 2309 | CARPKSGSYATYFDYW |
| 12-6 | 840 | FIFRNYAMH | 1575 | AVISYDGSNKYYA | 2310 | CARPRGGSYHGAFDIW |
| 12-7 | 841 | FTFSIYAMH | 1576 | AVISYDGTNEYYA | 2311 | CAKSRGGSYYGAFDYW |
| 12-8 | 842 | FTFNNYVMH | 1577 | AVISYDGTNDYYA | 2312 | CARGESGSYWGAFDYW |
| 12-9 | 843 | FTFSSYGMH | 1578 | AVISYDGTTEYYA | 2313 | CARPSSGSYLGFFDYW |
| 12-10 | 844 | FIFRSYAMH | 1579 | AVISYDGSIKYYA | 2314 | CARTRGGSYYGAFDYW |
| 12-11 | 845 | FSFGGYGMH | 1580 | AVISYDGSNEYYA | 2315 | CAKSYSGSYSSYFDYW |
| 12-12 | 846 | FAFSSHAME | 1581 | AVISYDGSNKYYA | 2316 | CAKAYSGSYMGYFDYW |
| 12-13 | 847 | FSFSTYGMH | 1582 | AVISYDGSNKYYA | 2317 | CARPLSGSYWSWFDPW |
| 12-14 | 848 | FTFSSYSMH | 1583 | AVISYDGSNKYYA | 2318 | CARGKGGGYYSSFDFW |
| 12-15 | 849 | FSFGGYGMH | 1584 | AVISYDGSNKYYA | 2319 | CARPYSGSYISWFDYW |
| 12-16 | 850 | FIFRSYAMH | 1585 | AVISYDGSSKYYA | 2320 | CARTLGGSYFAAFDIW |
| 12-17 | 851 | FTFGSYGMH | 1586 | AVISYDGNHEYYA | 2321 | CARPHSGSYTAYFDYW |
| 12-18 | 852 | FTFSSYAMH | 1587 | AVISYDGSNQYYA | 2322 | CARGYGGSYSYFDYW |
| 12-19 | 853 | FAFSSYAMH | 1588 | AVISYDGTYEYYA | 2323 | CARSLGGSYFSGMDVW |
| 12-20 | 854 | FSFGGYGMH | 1589 | AVISYDGSNKYYA | 2324 | CARSKGGSYYGPFDYW |
| 12-21 | 855 | FSFGGYGMH | 1590 | AVISYDGSNKYYA | 2325 | CARPKGGNYWNAFDIW |
| 12-22 | 856 | FTFSSYGMH | 1591 | AVISYDGNHEYYA | 2326 | CARPKSGSYVSYFDYW |
| 12-23 | 857 | FIFSSYAMH | 1592 | AVISYDGSNKYYA | 2327 | CARPRGGNYLNYFDYW |
| 12-24 | 858 | FTFSNFPMH | 1593 | AVISYDGNNKYYA | 2328 | CAKDHGDHYFDYW |
| 12-25 | 859 | FTFSSYAMH | 1594 | AVISYDGSNQYYA | 2329 | CARDKGGSYYGPFDYW |
| 12-26 | 860 | FTFSNYAMH | 1595 | AVISYDGSNEYYA | 2330 | CAKSGSGSYFSPFDYW |
| 12-27 | 861 | FSFGGYGMH | 1596 | AVISYDGSTKYYA | 2331 | CARPRGGSYKDAFDIW |
| 12-28 | 862 | FTFSSYAMH | 1597 | AVISYDGTNEYYA | 2332 | CARAHGGSYFSGMDVW |
| 12-29 | 863 | FSFSNYGMH | 1598 | AVISYDGNNKYYA | 2333 | CARSKGGSYYGPFDDW |
| 12-30 | 864 | FTFSGYAMH | 1599 | AVISYDGSNKYYA | 2334 | CARSRGGSYYAPFDYW |
| 12-31 | 865 | #N/A | 1600 | #N/A | 2335 | CARPLGGSYFAAFDIW |
| 12-32 | 866 | FTFGTYAMH | 1601 | AVISYDGNNKYYA | 2336 | CAKTMSGSYFSAFDIW |
| 12-33 | 867 | FTFSSYAMH | 1602 | AVISYDGSNQYYA | 2337 | CARPHGGNYFDWFDPW |
| 12-34 | 868 | FIFRSYAMH | 1603 | AVISYDGSSKYYA | 2338 | CARPSGGSYFDPFDYW |
| 12-35 | 869 | FTFSSSSMH | 1604 | AVISYDGSNKYYA | 2339 | CAKVDSGSYVGYFDYW |
| 12-36 | 870 | FSFNNYGMH | 1605 | AVISYDGSNDYYA | 2340 | CARPNSGSYSNYFDYW |
| 12-37 | 871 | FTFSSYAMH | 1606 | AVISYDGSNQYYA | 2341 | CARSRSGSYLAYFDYW |
| 12-38 | 872 | FTFSSYAMH | 1607 | AVISYDGSNQYYA | 2342 | CARAAGGSYSSWFDPW |
| 12-39 | 873 | FTFSSYAMH | 1608 | AVISYDGNHEYYA | 2343 | CARAHSGSYFSHFDYW |
| 12-40 | 874 | FTFSSYAMH | 1609 | AVISYDGSNTYYA | 2344 | CARPTSGSYFSWFDPW |
| 12-41 | 875 | FIFSSYAMH | 1610 | AVISYDGSNKYYA | 2345 | CARPNSGSYWGPFDYW |
| 12-42 | 876 | FTFGSYGMH | 1611 | AVISYDGSHKYYA | 2346 | CARALGGNYYYFDYW |
| 12-43 | 877 | FIFSSYGMH | 1612 | AVISYDGSNEYYA | 2347 | CARPRSGSYLSAFDYW |
| 13-1 | 878 | FTFSSYSMH | 1613 | AVISYDGRNQYYA | 2348 | CAKGYGGNYYYMDGW |
| 13-2 | 879 | FTFSSYAMH | 1614 | AVISYDGNNKYYA | 2349 | CARTYGGSYYSAFDYW |
| 13-3 | 880 | FSFNNHAMH | 1615 | AVISYDGSDKYYA | 2350 | CARNLLRGYGMDVW |
| 13-4 | 881 | FAFDDYAMH | 1616 | AVISYDGSNKYYA | 2351 | CATLGYGDYPDYW |
| 13-5 | 882 | FIFRSYAMH | 1617 | AVISYDGSSKYYA | 2352 | CARPLGGGYQDAFDIW |

TABLE 10
Variable Region, Heavy Chain Complementary
Determining Region 3 (CDRH3)

| Variant | SEQ ID NO | CDRH3 Sequence |
|---|---|---|
| 1N-1 | 2353 | CAKEDVGKPFYW |
| 1N-2 | 2354 | CAKEDVGKPFFW |
| 1N-3 | 2355 | CAKEDVGKPFDW |
| 1N-4 | 2356 | CAKEDVGKPFCW |
| 1N-5 | 2357 | CAKEDVGKPFDW |
| 1N-6 | 2358 | CAKEDVGKPFDW |
| 1N-7 | 2359 | CAKEDVGKPFDW |
| 1N-8 | 2360 | CAKEDVGKPLDW |
| 1N-9 | 2361 | CAKEDVGPPFDW |
| 1N-10 | 2362 | CAKEDVGKPFHW |
| 1N-11 | 2363 | CAKEDVGPPFYW |
| 1N-12 | 2364 | CWKEDVGKPGDW |
| 1N-13 | 2365 | CAKEDVGKPFCW |
| 1N-14 | 2366 | CRKEDVGKPFFW |
| 1N-15 | 2367 | CYKEDVGKPFYW |
| 1N-16 | 2368 | CHKEDVGKPFYW |
| 1N-17 | 2369 | CFKEDVGKPFFW |
| 1N-18 | 2370 | CYKEDVGKPFFW |
| 1N-19 | 2371 | CFKEDVGKPFWW |
| 1N-20 | 2372 | CWKEDVGKPFDE |
| 1N-21 | 2373 | CARVDRDFDYW |
| 1N-22 | 2374 | CARVDRDFDYW |
| 1N-23 | 2375 | CARVTRDFDYW |
| 1N-24 | 2376 | CARVWRDFDYW |
| 1N-25 | 2377 | CARVDRDFDYW |
| 1N-26 | 2378 | CARVDRDFDYW |
| 1N-27 | 2379 | CARVDRDFDYW |
| 1N-28 | 2380 | CARVDRDFDYW |
| 1N-29 | 2381 | CARVDRDFDYW |
| 1N-30 | 2382 | CARVYRDFDYW |
| 1N-31 | 2383 | CARVDRDFDYW |
| 1N-32 | 2384 | CARVTRDFDYW |
| 1N-33 | 2385 | CARVDRDFDYW |
| 1N-34 | 2386 | CARVDRDFDYW |
| 1N-35 | 2387 | CARVSRDFDYW |
| 1N-36 | 2388 | CARVDGDFDYW |
| 1N-37 | 2389 | CARVDRDFDYW |
| 1N-38 | 4177 | CARVTRDFDYW |
| 1N-39 | 4178 | CARVDRDFDYW |
| 1N-40 | 4179 | CARVYRDFDYW |
| 1N-41 | 4180 | CARVTRDFDYW |
| 1N-42 | 4181 | CARVDRDFDYW |
| 1N-43 | 4182 | CARVDYDFDYW |
| 1N-44 | 4183 | CARVDRDFDYW |
| 1N-45 | 4184 | CARVDRDFDYW |
| 1N-46 | 4185 | CWRLGNDYFDYW |
| 1N-47 | 4186 | CWRLGNDYFDYW |
| 1N-48 | 4187 | CWRLGNDYFDYW |
| 1N-49 | 4188 | CWRLGNDYFDYW |
| 1N-50 | 4189 | CWRYGNDYFDYW |
| 1N-51 | 4190 | CWRFGNDYFDYW |
| 1N-52 | 4191 | CWRLGNDYFDYW |
| 1N-53 | 4192 | CWRLGNDYFDYW |
| 1N-54 | 4193 | CWRLGNDYFDYW |
| 1N-55 | 4194 | CWRLGNDYFDYW |
| 1N-56 | 4195 | CWRLGNDYFDYW |
| 1N-57 | 4196 | CWRLGNDYFDYW |
| 1N-58 | 4197 | CWREGNDYFDYW |
| 1N-59 | 4198 | CWREGNDYFDYW |
| 1N-60 | 4199 | CWRLGNDYFDYW |
| 1N-61 | 4200 | CWRGGNDYFDYW |
| 1N-62 | 4201 | CWRLGNDYFDYW |
| 1N-63 | 4202 | CWRLGNDYFDYW |
| 1N-64 | 4203 | CWRLGNDYFDYW |
| 1N-65 | 4204 | CWRYGNDYFDYW |
| 1N-66 | 4205 | CWRYGNDYFDYW |
| 1N-67 | 4206 | CWRLGNDYFDYW |
| 1N-68 | 4207 | CWRLTNDYFDYW |
| 1N-69 | 4208 | CWRLGNDYFDYW |
| 1N-70 | 4209 | CWRLGNDYFDYW |
| 1N-71 | 4210 | CWRYGNDYFDYW |
| 1N-72 | 4211 | CWRYGNDYFDYW |
| 1N-73 | 4212 | CWRLGNDYFDYW |
| 1N-74 | 2390 | CWRLGNDYFDYW |
| 1N-75 | 2391 | CWRYGNDYFDYW |
| 1N-76 | 2392 | CWRLGNDYFDYW |
| 1N-77 | 2393 | CWRFGNDYFDYW |
| 1N-78 | 2394 | CPRLGNDYFDYW |
| 1N-79 | 2395 | CWRTGNDYFDYW |
| 1N-80 | 2396 | CWRHGNDYFDYW |
| 1N-81 | 2397 | CAGDYDFWSGFDHW |
| 1N-82 | 2398 | CARHYYDSSDYYPHYYYYGMDVW |
| 1N-83 | 2399 | CARHMGYYDSGTYFDYFDYW |
| 1N-84 | 2400 | CTTVDQYFDYW |
| 1N-85 | 2401 | CARYDFWSGYPYW |
| 1N-86 | 2402 | CAKFAVYDYWSGTSFDYW |
| 1N-87 | 2403 | CTSLVGLTAGFADYW |
| 1N-88 | 2404 | CAAFDGYTGSDW |
| 1N-89 | 2405 | CAGDYDFWSGFDHW |
| 1N-90 | 2406 | CARADNYFDYW |
| 1N-91 | 2407 | CAAFDGYTGSDW |
| 1N-92 | 2408 | CARDYGDYYYFDYW |
| 1N-93 | 2409 | CAKGPSSGYAFDIW |
| 1N-94 | 2410 | CTTVDQYFDYW |
| 1N-95 | 2411 | CTTVDQYFDYW |
| 1N-96 | 2412 | CAAFDGYSGSDW |
| 1N-97 | 2413 | CTTFNWNDEGFDYW |
| 1N-98 | 2414 | CAAFDGYTGSDW |
| 1N-99 | 2415 | CVRSNMAGFDHW |
| 1N-100 | 2416 | CAAFDGYSGSDW |

TABLE 11
Variable Domain Light Chain Sequences

| | SEQ ID | | SEQ ID | | SEQ ID | |
|---|---|---|---|---|---|---|
| Variant | NO | CDRL1 | NO | CDRL2 | NO | CDRL3 |
| 2A-1 | 2417 | RASQSIHRFLN | 2587 | AASNLHS | 2757 | CQQSYGLPPTF |
| 2A-2 | 2418 | RASQTINTYLN | 2588 | SASTLQS | 2758 | CQQSYSTFTF |
| 2A-3 | 2419 | RASQNIHTYLN | 2589 | AASTFAK | 2759 | CQQSYSAPPYTF |
| 2A-4 | 2420 | RASQSIDTYLN | 2590 | AASALAS | 2760 | CQQSYSAPPYTF |
| 2A-5 | 2421 | RASQSIHTYLN | 2591 | AASALAS | 2761 | CQQSYSAPPYTF |
| 2A-6 | 2422 | RASQSIDTYLN | 2592 | AASALAS | 2762 | CQQSYSAPPYTF |
| 2A-7 | 2423 | RASQSIDTYLN | 2593 | AASALAS | 2763 | CQQSYSAPPYTF |
| 2A-8 | 2424 | RASQSIDTYLN | 2594 | AASALAS | 2764 | CQQSYSAPPYTF |
| 2A-9 | 2425 | RASQRIGTYLN | 2595 | AASNLEG | 2765 | CQQNYSTTWTF |
| 2A-10 | 2426 | RASQSIHISLN | 2596 | LASPLAS | 2766 | CQQSYSAPPYTF |
| 2A-11 | 2427 | RASQSIGNYLN | 2597 | GVSSLQS | 2767 | CQQSHSAPLTF |
| 2A-12 | 2428 | RASQSIDNYLN | 2598 | GVSALQS | 2768 | CQQSHSAPPYFF |
| 2A-13 | 2429 | RASQSIDTYLN | 2599 | GASALES | 2769 | CQQSHSAPPYFF |
| 2A-14 | 2430 | RASQSIDTYLN | 2600 | GVSALQS | 2770 | CQQSYSAPPYFF |
| 2A-15 | 2431 | RASQSIDNYLN | 2601 | GVSALQS | 2771 | CQQSHSAPLTF |
| 3A-1 | 2432 | RASQTIYSYLN | 2602 | ATSTLQG | 2772 | CQHRGTF |
| 3A-2 | 2433 | RTSQSINTYLN | 2603 | GASNVQS | 2773 | CQQSYRIPRTF |
| 3A-3 | 2434 | RASRSISRYLN | 2604 | AASSLQA | 2774 | CQQSYSSLLTF |
| 3A-4 | 2435 | RASRSIRRYLN | 2605 | ASSSLQA | 2775 | CQQSYSTLLTF |
| 3A-5 | 2436 | RASQSIGRYLN | 2606 | AASSLKS | 2776 | CQQSYSLPRTF |
| 3A-6 | 2437 | RASQSIGKYLN | 2607 | ASSSLQS | 2777 | CQQSYSPPFTF |
| 3A-7 | 2438 | RASQSIGRYLN | 2608 | ASSSLQS | 2778 | CQQSYSLPRTF |
| 3A-8 | 2439 | RASQSIGRYLN | 2609 | AASSLKS | 2779 | CQQSYSLPLTF |
| 3A-9 | 2440 | RASQSIGRYLN | 2610 | AASSLKS | 2780 | CQQSYSLPRTF |
| 3A-10 | 2441 | RASQSIRKYLN | 2611 | ASSTLQR | 2781 | CQQSLSTPFTF |
| 3A-11 | 2442 | RASQSIGKYLN | 2612 | ASSTLQR | 2782 | CQQSLSPPFTF |
| 3A-12 | 2443 | RASQSIGKYLN | 2613 | ASSTLQR | 2783 | CQQSLSTPFTF |
| 3A-13 | 2444 | RASQSIGKYLN | 2614 | ASSTLQR | 2784 | CQQSFSPPFTF |
| 3A-14 | 2445 | RASQSIGKYLN | 2615 | ASSTLQR | 2785 | CQQSFSTPFTF |
| 3A-15 | 2446 | RASQNIKTYLN | 2616 | AASKLQS | 2786 | CQQSYSTSPTF |
| 2A-1 | 2447 | RASQSIHRFLN | 2617 | AASNLHS | 2787 | CQQSYGLPPTF |
| 2A-10 | 2448 | RASQSIHISLN | 2618 | LASPLAS | 2788 | CQQSYSAPPYTF |
| 2A-5 | 2449 | RASQSIHTYLN | 2619 | AASALAS | 2789 | CQQSYSAPPYTF |
| 2A-2 | 2450 | RASQTINTYLN | 2620 | SASTLQS | 2790 | CQQSYSTFTF |
| 2A-4 | 2451 | RASQSIDTYLN | 2621 | AASALAS | 2791 | CQQSYSAPPYTF |
| 2A-6 | 2452 | RASQSIGNYLN | 2622 | GVSSLQS | 2792 | CQQSHSAPLTF |
| 2A-11 | 2453 | RASQSIDTYLN | 2623 | AASALAS | 2793 | CQQSYSAPPYTF |
| 2A-12 | 2454 | RASQSIDNYLN | 2624 | GVSALQS | 2794 | CQQSHSAPPYFF |
| 2A-13 | 2455 | RASQSIDTYLN | 2625 | GASALES | 2795 | CQQSHSAPPYFF |
| 2A-14 | 2456 | RASQSIDTYLN | 2626 | AASALAS | 2796 | CQQSYSAPPYTF |
| 2A-7 | 2457 | RASQSIDTYLN | 2627 | GVSALQS | 2797 | CQQSYSAPPYFF |
| 2A-8 | 2458 | RASQSIDTYLN | 2628 | AASALAS | 2798 | CQQSYSAPPYTF |
| 2A-15 | 2459 | RASQSIDNYLN | 2629 | GVSALQS | 2799 | CQQSHSAPLTF |
| 2A-9 | 2460 | RASQRIGTYLN | 2630 | AASNLEG | 2800 | CQQNYSTTWTF |
| 2A-21 | 2461 | RASQSIHTYLN | 2631 | AASALAS | 2801 | CQQSYSAPPYTF |
| 2A-22 | 2462 | RASQSIHTYLN | 2632 | AASALAS | 2802 | CQQSYSAPPYTF |
| 2A-23 | 2463 | RASQTINTFLN | 2633 | SASTLQS | 2803 | CQQSYSTFTF |
| 2A-24 | 2464 | RASQTIRTYLN | 2634 | DASTLQR | 2804 | CQQSYRTPPWTF |
| 2A-25 | 2465 | RSSQSISSYLN | 2635 | GASRLRS | 2805 | CQQGYSAPWTF |
| 2A-26 | 2466 | RASQSISGSLN | 2636 | AESRLHS | 2806 | CQQSYSPPQTF |
| 2A-27 | 2467 | RASRSISTYLN | 2637 | AASNLQG | 2807 | CQQSHSIPRTF |
| 2A-28 | 2468 | RASQSIHTYLN | 2638 | AASALAS | 2808 | CQQSYSAPPYTF |
| 3A-10 | 2469 | RASQSIRKYLN | 2639 | ASSTLQR | 2809 | CQQSLSTPFTF |
| 3A-4 | 2470 | RASQNIKTYLN | 2640 | AASKLQS | 2810 | CQQSYSTSPTF |
| 3A-7 | 2471 | RASQTIYSYLN | 2641 | ATSTLQG | 2811 | CQHRGTF |
| 3A-1 | 2472 | RASRSIRRYLN | 2642 | ASSSLQA | 2812 | CQQSYSTLLTF |
| 3A-5 | 2473 | RASQSIGKYLN | 2643 | ASSSLQS | 2813 | CQQSYSPPFTF |
| 3A-6 | 2474 | RASRSISRYLN | 2644 | AASSLQA | 2814 | CQQSYSSLLTF |
| 3A-15 | 2475 | RASQSIGKYLN | 2645 | ASSTLQR | 2815 | CQQSLSPPFTF |
| 3A-3 | 2476 | RASQSIGRYLN | 2646 | ASSSLQS | 2816 | CQQSYSLPRTF |
| 3A-11 | 2477 | RASQSIGRYLN | 2647 | AASSLKS | 2817 | CQQSYSLPRTF |
| 3A-8 | 2478 | RASQSIGKYLN | 2648 | ASSTLQR | 2818 | CQQSLSTPFTF |
| 3A-2 | 2479 | RASQSIGRYLN | 2649 | AASSLKS | 2819 | CQQSYSLPLTF |
| 3A-12 | 2480 | RTSQSINTYLN | 2650 | GASNVQS | 2820 | CQQSYRIPRTF |
| 3A-14 | 2481 | RASQSIGKYLN | 2651 | ASSTLQR | 2821 | CQQSFSPPFTF |
| 3A-9 | 2482 | RASQSIGKYLN | 2652 | ASSTLQR | 2822 | CQQSFSTPFTF |
| 3A-13 | 2483 | RASQSIGRYLN | 2653 | AASSLKS | 2823 | CQQSYSLPRTF |
| 3A-16 | 2484 | RASQIIGSYLN | 2654 | TTSNLQS | 2824 | CQQSYITPWTF |
| 3A-17 | 2485 | RASQSISRYIN | 2655 | EASSLES | 2825 | CQQSHITPLTF |
| 3A-18 | 2486 | RASQSIYTYLN | 2656 | SASNLHS | 2826 | CQQSDTTPWTF |
| 3A-19 | 2487 | RASQSIATYLN | 2657 | GASSLEG | 2827 | CQQTFSSPFTF |
| 3A-2 | 2488 | RASQNINTYLN | 2658 | SASSLQS | 2828 | CQQSSLTPWTF |
| 3A-21 | 2489 | RASQGIATYLN | 2659 | YASNLQS | 2829 | CQQSYSTRFTF |
| 3A-22 | 2490 | RASERISNYLN | 2660 | TASNLES | 2830 | CQQSYTPPRTF |
| 3A-23 | 2491 | RASQSISSSLN | 2661 | AASRLQD | 2831 | CQQSYSTPRSF |
| 3A-24 | 2492 | RASQSISSHLN | 2662 | RASTLQS | 2832 | CQQTYNTPQTF |
| 3A-25 | 2493 | RASQSISSYLI | 2663 | AASRLHS | 2833 | CQQGYNTPRTF |
| 3A-26 | 2494 | RASPSISTYLN | 2664 | TASRLQT | 2834 | CQQTYSTPSSF |
| 3A-27 | 2495 | RASQNIAKYLN | 2665 | GASGLQS | 2835 | CQQSHSPPITF |
| 3A-28 | 2496 | RASQSIGTYLN | 2666 | AASNLHS | 2836 | CQESYSAPYTF |
| 3A-29 | 2497 | RASQSISPYLN | 2667 | KASSLQS | 2837 | CQQSSSTPYTF |
| 9-1 | 2498 | RASQGVSNYLA | 2668 | DASNRAT | 2838 | CQQRYSWVTF |
| 9-2 | 2499 | RASQSVSSSLA | 2669 | DASNRAT | 2839 | CQQRINWPRSF |
| 9-3 | 2500 | RASQSVNSYLA | 2670 | DVSNRAT | 2840 | CQQFSNWPTF |
| 9-4 | 2501 | RASQSVGTSLA | 2671 | GASNRAT | 2841 | CQQRSNWQPF |
| 9-5 | 2502 | RATQYVNSYLA | 2672 | DVSNRAT | 2842 | CQQFSNWPTF |
| 9-6 | 2503 | RASQSVGTSLA | 2673 | GASNRAT | 2843 | CQLRSNWYTF |
| 9-7 | 2504 | RASQGVSNYLA | 2674 | DASNRAT | 2844 | CQQRYSWVTF |
| 9-8 | 2505 | RASQGVSNYLA | 2675 | DASNRAT | 2845 | CQQRYSWVTF |
| 9-9 | 2506 | RASQSVDSRLA | 2676 | DTSNRAT | 2846 | CQQRSTWPPVF |
| 9-10 | 2507 | RASQSVRHHLA | 2677 | DASNRAT | 2847 | CQQRTDWPRAF |
| 9-11 | 2508 | RASQSVGNFLA | 2678 | DASNRAT | 2848 | CQQSSTWPLTF |
| 9-12 | 2509 | RASESISTYLA | 2679 | DASNRAT | 2849 | CQQRSGLITF |
| 9-13 | 2510 | RASQSVGDFLA | 2680 | DTSNRAT | 2850 | CQQRSNLTF |
| 9-14 | 2511 | RASQTIRNSLN | 2681 | ASSSLQS | 2851 | CQQTHSIPKTF |
| 9-15 | 2512 | RASQSVSSSLA | 2682 | DASNRAT | 2852 | CQQRINWPRSF |
| 10-1 | 2513 | RASQDVSTYLA | 2683 | DASNRAT | 2853 | CQQRRDWPQTF |
| 10-2 | 2514 | RASQDVSTYLA | 2684 | DASNRAT | 2854 | CQQRRDWPQTF |
| 10-3 | 2515 | RASQDVSTYLA | 2685 | DASNRAT | 2855 | CQQRRDWPQTF |
| 10-4 | 2516 | RASQDVSTYLA | 2686 | DASNRAT | 2856 | CQQRRDWPQTF |
| 10-5 | 2517 | RASQDVSTYLA | 2687 | DASNRAT | 2857 | CQQRRDWPQTF |
| 10-6 | 2518 | RASQDVSTYLA | 2688 | DASNRAT | 2858 | CQQRRDWPQTF |
| 11-1 | 2519 | RASQSLGSFLA | 2689 | DASNRAT | 2859 | CQQRALWPRLTF |
| 11-2 | 2520 | RASQSVNSYLA | 2690 | DVSNRAT | 2860 | CQQFSNWPTF |
| 11-3 | 2521 | RASQNIGNHLA | 2691 | DASNRAT | 2861 | CQQRDNGPPEGTF |
| 11-4 | 2522 | RASQSVGSYLA | 2692 | DAVNRAT | 2862 | CQQRFTWPTTF |
| 11-5 | 2523 | RASQSITDYLA | 2693 | DASNRAT | 2863 | CHQRNNWPPTF |
| 11-6 | 2524 | RASQSVDSSLA | 2694 | DASNRAT | 2864 | CQQQSNWPGTF |
| 11-7 | 2525 | RASQSIGSYLA | 2695 | DGSNRAT | 2865 | CQQRTNWPLFSF |
| 11-8 | 2526 | RASQTVTNYLA | 2696 | DTSNRAT | 2866 | CQHRDDWPPTF |
| 11-9 | 2527 | RASQSVSYYLA | 2697 | DSSNRAT | 2867 | CQQRSNWQGNF |
| 11-10 | 2528 | RASQSVSTSLA | 2698 | DATNRAT | 2868 | CQQHYSWPLTF |
| 11-11 | 2529 | RASHNINNFLA | 2699 | DTSNRAT | 2869 | CQQGRNWPPSSF |
| 11-12 | 2530 | RASQSVGTSLA | 2700 | GASNRAT | 2870 | CQERSNWPDTF |
| 11-13 | 2531 | RASQSVSSQLA | 2701 | DTSNRAT | 2871 | CQQRYNWPSTF |
| 11-14 | 2532 | RASQSVDSRLA | 2702 | DASNRAT | 2872 | CQQRTNLPPSITF |
| 11-15 | 2533 | RASQSVGSYLA | 2703 | DAVNRAT | 2873 | CQQRSDSITF |
| 11-16 | 2534 | #N/A | 2704 | #N/A | 2874 | #N/A |
| 11-17 | 2535 | RASQNIGSHLA | 2705 | DVSNRAT | 2875 | CQQRDYWPPYTF |
| 11-18 | 2536 | RASQSLTSYLA | 2706 | DASNRAT | 2876 | CQQRHYWPPITF |
| 11-19 | 2537 | RASQSIGSYLA | 2707 | DASNRAT | 2877 | CQQRDSWPHTF |
| 11-20 | 2538 | RASQSVGSYLA | 2708 | DAVNRAT | 2878 | CQQRSLWPF |
| 12-1 | 2539 | RASQSVSSHLA | 2709 | DVSNRAT | 2879 | CQQRDTFTF |
| 12-2 | 2540 | RASQSVDSRLA | 2710 | DTSNRAT | 2880 | CQQRSTWPPVF |
| 12-3 | 2541 | RASQSVGDFLA | 2711 | DTSNRAT | 2881 | CQYRSNFTF |
| 12-4 | 2542 | RASQSVGSHLA | 2712 | DASNRAT | 2882 | CQQISNWPLTF |
| 12-5 | 2543 | RASQNVGQSLA | 2713 | DASNRAT | 2883 | CQQRENWPPTF |
| 12-6 | 2544 | RASQSLGNYLA | 2714 | DSSNRAT | 2884 | CQQRNWPYTF |
| 12-7 | 2545 | RASQSLGNYLA | 2715 | DSSNRAT | 2885 | CQQRTDWPPSF |
| 12-8 | 2546 | RASQNIGNHLA | 2716 | DVSNRAT | 2886 | CQQRKSWPPFTF |
| 12-9 | 2547 | RASQSVSTSLA | 2717 | DATNRAT | 2887 | CQRRTDWPPTF |
| 12-10 | 2548 | RASQSVNSDLA | 2718 | DASNRAT | 2888 | CQQRTDWPPATF |
| 12-11 | 2549 | RASQSVGSYLA | 2719 | DAVNRAT | 2889 | CQQRFTWPTTF |
| 12-12 | 2550 | RASQSVSSSLA | 2720 | DASNRAT | 2890 | CQHRDDWPPTF |
| 12-13 | 2551 | RASQSVGSYLA | 2721 | DAVNRAT | 2891 | CQQRNSWPPATF |
| 12-14 | 2552 | RASQSVGSYLA | 2722 | DAVNRAT | 2892 | CQQVSNWPLTF |
| 12-15 | 2553 | RASQSVSSHLA | 2723 | DVSNRAT | 2893 | CQVRSDWPPLTF |
| 12-16 | 2554 | RASQSLDSYLA | 2724 | DVSNRAT | 2894 | CQQRRGWPPVTF |
| 12-17 | 2555 | RASQSVSKFLA | 2725 | DASNRAT | 2895 | CHQHSDWPLTF |
| 12-18 | 2556 | RASQSIGGSLA | 2726 | DASNRAT | 2896 | CQQRYSYFTF |
| 12-19 | 2557 | RASQSISRYLA | 2727 | DVSNRAT | 2897 | CQQSSNWPLFTF |
| 12-20 | 2558 | RASQSLGNYLA | 2728 | DSSNRAT | 2898 | CQQRNTWPGVTF |
| 12-21 | 2559 | RASQSVNSDLA | 2729 | DASNRAT | 2899 | CQERSLF |
| 12-22 | 2560 | RASQSVRHHLA | 2730 | DASNRAT | 2900 | CQERSDWPITF |
| 12-23 | 2561 | RASQSVDSRLA | 2731 | DASNRAT | 2901 | CQQRSTWPPVF |
| 12-24 | 2562 | RASQSFGDSLA | 2732 | DASNRAT | 2902 | CQQRSIPITF |
| 12-25 | 2563 | RASQSVNSYLA | 2733 | DVSNRAT | 2903 | CQERGNWPPFTF |
| 12-26 | 2564 | RASQSVSTSLA | 2734 | DISNRAT | 2904 | CQQRRSGLTF |
| 12-27 | 2565 | RASDTVSSYLA | 2735 | DTSNRAT | 2905 | CQQRASWPLSF |
| 12-28 | 2566 | RASQSVRHHLA | 2736 | DASNRAT | 2906 | CQQSGSWPLTF |
| 12-29 | 2567 | RASQIISSYLA | 2737 | DTSNRAT | 2907 | CQVRSNWPPLTF |
| 12-30 | 2568 | RASHNIGTYLA | 2738 | DVSNRAT | 2908 | CQQRADWPQTF |
| 12-31 | 2569 | #N/A | 2739 | #N/A | 2909 | #N/A |
| 12-32 | 2570 | RASQSIGSYLA | 2740 | DVSNRAT | 2910 | CQQRDSFTF |
| 12-33 | 2571 | RASQDVSTYLA | 2741 | DASNRAT | 2911 | CQQRAYWPGTF |
| 12-34 | 2572 | RASQSVGNFLA | 2742 | DASNRAT | 2912 | CQHRRLLTF |
| 12-35 | 2573 | RASQRVSSYLA | 2743 | DAFNRAT | 2913 | CQQRQDWPLTF |
| 12-36 | 2574 | RASQGISTYLA | 2744 | DASNRAT | 2914 | CQQRRRWPPTF |
| 12-37 | 2575 | RASESVSESLA | 2745 | DASNRAT | 2915 | CQQRTHGVTF |
| 12-38 | 2576 | RASQSVSTSLA | 2746 | DATNRAT | 2916 | CQQRQKWPLTF |
| 12-39 | 2577 | RASESISTYLA | 2747 | DASNRAT | 2917 | CQQRRNSLTF |
| 12-40 | 2578 | RASQSVNSDLA | 2748 | DASNRAT | 2918 | CQQRSTWSPLTF |
| 12-41 | 2579 | RASQNVGQSLA | 2749 | DASNRAT | 2919 | CQLRTNWPPVTF |
| 12-42 | 2580 | RASQSVDSRLA | 2750 | DTSNRAT | 2920 | CQQRSSNWTF |
| 12-43 | 2581 | RASQSVGKSLA | 2751 | DTSNRAT | 2921 | CQQRGSFPLTF |
| 13-1 | 2582 | RASQSVGDFLA | 2752 | DTSNRAT | 2922 | CQQRSIRGTF |
| 13-2 | 2583 | RASDTVSSYLA | 2753 | DTSNRAT | 2923 | CQQRGGWPPAF |
| 13-3 | 2584 | RASQSIGDYLN | 2754 | EASSLQS | 2924 | CLHTYLPPYSF |
| 13-4 | 2585 | RASQSITRYLN | 2755 | AASSLQS | 2925 | CQQTYNFPHTF |
| 13-5 | 2586 | RASQSIGSYLA | 2756 | DVSNRAT | 2926 | CQQRHHWPPVTF |

TABLE 12
Variable Domain Heavy Chain Sequences

| | SEQ | |
|---|---|---|
| Variant | ID NO | Sequence |
| 1-1 | 2927 | EVQLLESGGGLVQPGGSLRLSCAASGFTFSSYAMNWVRQAPGKGLEWVSAISGSGVST |
| | | YYADSVKGRFTISRDNSKNTLYLQMNSLRAEDTAVYYCAKGDSGSYYGSSYFDYWGQ |
| | | GTLVTVSS |
| 1-2 | 2928 | EVQLLESGGGLVQPGGSLRLSCAASGFTFSSYGMSWVRQAPGKGLEWVSAISGSGGNT |
| | | YYADSVKGRFTISRDNSKNTLYLQMNSLRAEDTAVYYCTRVRRGSGVAPYSSSWGRY |
| | | YFDYWGQGTLVTVSS |
| 1-3 | 2929 | EVQLLESGGGLVQPGGSLRLSCAASGFRFSSYSMSWVRQAPGKGLEWVSAISGSGGSS |
| | | YYADSVKGRFTISRDNSKNTLYLQMNSLRAEDTAVYYCAKDGSGTIFGVVIAKYYFDY |
| | | WGQGTLVTVSS |
| 1-4 | 2930 | EVQLLESGGGLVQPGGSLRLSCAASGFTFSAYAMSWVRQAPGKGLEWVSAISGSGGST |
| | | HYADSVKGRFTISRDNSKNTLYLQNSLRAEDTAVYYCASWGPLWSGSPNDAFDIWGQ |
| | | GTLVTVSS |
| 1-5 | 2931 | EVQLLESGGGLVQPGGSLRLSCAASGFFSSYAMGWVRQAPGKGLEWVSAISGSGYSTY |
| | | YADSVKGRFTISRDNSKNTLYLQMNSLRAEDTAVYYCARVRSYDSTAYDEPLDALDI |
| | | WGQGTLVTVSS |
| 1-6 | 2932 | EVQLLESGGGLVQPGGSLRLSCAASGFTFSSFAMSWVRQAPGKGLEWVSAISGSGVST |
| | | YYADSVKGRFTISRDNSKNTLYLQMNSLRAEDTAVYYCGRDARSSGYNGYDLFDIWG |
| | | QGTLVTVSS |
| 1-7 | 2933 | EVQLLESGGGLVQPGGSLRLSCAASGFTFSAYAMSWRQAPGKGLEWVSAISGSGGSYY |
| | | ADSVKGRFTISRDNSKNTLYLQMNSLRAEDTAVYYCAKGPLVGWYFDLWGQGTLVT |
| | | VSS |
| 1-8 | 2934 | EVQLLESGGGLVQPGGSLRLSCAASGFTFGSYAMSWVRQAPGKGLEWVSLISGSGGST |
| | | YYADSVKGRFTISRDNSKNTLYLQMNSLRAEDTAVYYCASWGPLWSGSPNDAFDIWG |
| | | QGTLVTVSS |
| 1-9 | 2935 | EVQLLESGGGLVQPGGSLRLSCAASGFTFSAYAMSWVRQAPGKGLEWVSAISGSGGST |
| | | FYADSVKGRFTISRDNSKNTLYLQMNSLRAEDTAVYYCTRQGDSSGWYDGWFDPWG |
| | | QGTLVTVSS |
| 1-10 | 2936 | EVQLLESGGGLVQPGGSLRLSCAASGFIFSSYAMSWVRQAPGKGLEWVSIISGSGGSTY |
| | | YADSVKGRFTISRDNSKNTLYLQMNSLRAEDTAVYYCIATVVSPLDYWGQGTLVTVSS |
| 1-11 | 2937 | EVQLLESGGGLVQPGGSLRLSCAASGFTFSDYAMSWVRQAPGKGLEWVSTISGSGGST |
| | | YYADSVKGRFTISRDNSKNTLYLQMNSLRAEDTAVYYCARDESSSSLNWFDPWGQGT |
| | | LVTVSS |
| 1-12 | 2938 | EVQLLESGGGLVQPGGSLRLSCAASGFTFSSYAMIWVRQAPGKGLEWVSAISGSAGST |
| | | YYADSVKGRFTISRDNSKNTLYLQMNSLRAEDTAVYYCASPDPLGSVADLDYWGQGT |
| | | LVTVSS |
| 1-13 | 2939 | EVQLLESGGGLVQPGGSLRLSCAASGFTFGSYAMSWVRQAPGKGLEWVSAISGSGGTT |
| | | YYADSVKGRFTISRDNSKNTLYLQMNSLRAEDTAVYYCARVWSSSSVFDYWGQGTLV |
| | | TVSS |
| 1-14 | 2940 | EVQLLESGGGLVQPGGSLRLSCAASGFTFSRYAMSWRQAPGKGLEWVSAISGSGASTY |
| | | YADSVKGRFTISRDNKNTLYLQMNSLRAEDTAVYYCAKDRGGGSYYGTFDYWGQGT |
| | | LVTVSS |
| 1-15 | 2941 | EVQLLESGGGLVQPGGSLRLSCAASGSTFSSYAMSWVRQAPGKGLEWVSAISGSGATY |
| | | YADSVKGRFTISRDNSKNTLYLQMNSLRAEDTAVYYCTRVRVAGYSSSWYDAFDIWG |
| | | QGTLVTVSS |
| 1-16 | 2942 | EVQLLESGGGLVQPGGSLRLSCAASGFTFSSYAMTWVRQAPGKGLEWVSAISGSGGNT |
| | | YYADSVKGRFTISRDNSKNTLYLQMNSLRAEDTAVYYCVKGTIPIFGVIRSAFDYWGQ |
| | | GTLVTVSS |
| 1-17 | 2943 | EVQLLESGGGLVQPGGSLRLSCAASGFTFSSYVMSWVRQAPGKGLEWVSSISGSGGST |
| | | YYADSVKGRFTISRDNSKNTLYLQMNSLRAEDTAVYYCARGSGSYSFFDYWGQGTLV |
| | | TVSS |
| 1-18 | 2944 | EVQLLESGGGLVQPGGSLRLSCAASGFTFSSYANWVRQAPGKGLEWVSAISGSGVSTY |
| | | YADSVKGRFTISRDNSKNTLYLQMNSLRAEDTAVYYCATTPGPWIQLWFGGGFDYWG |
| | | QGTLVTVSS |
| 1-19 | 2945 | EVQLLESGGGLVQPGGSLRLSCAASGFTFSSYDMSWVRQAPGKGLEWVSAISGSAGST |
| | | TMRDSVKGRFTISRDNSKNTLYLQMNSLRAEDTAVYYCAKDGLVVAGTFDYWGQGT |
| | | LVTVSS |
| 1-20 | 2946 | EVQLLESGGGLVQPGGSLRLSCAASGFTFSGYAMSWVRQAPGKGLEWVSALSGSGGS |
| | | TYYADSVKGRFTISRDNSKNTLYLQMNSLRAEDTAVYYCARGALLEWLSRFDNWGQG |
| | | TLVTVSS |
| 1-21 | 2947 | EVQLLESGGGLVQPGGSLRLSCAASGFTLSSYAMSWVRQAPGKGLEWVSAISGSGGTT |
| | | YYADSVKGFTISRDNSKNTLYLQMNSLRAEDTAVYYCARDLGAADLIDYWGQGTLVT |
| | | VSS |
| 1-22 | 2948 | EVQLLESGGGLVQPGGSLRLSCAASGFIFSSYAMSWVRQAPGKGLEWISAISGSGGTYY |
| | | ADSVKGRFTISRDNSKNTLYLQMNSPRAEDTAVYYCVRVPAAAGKGVPGIFDIWGQGT |
| | | LVTVSS |
| 1-23 | 2949 | EVQLLESGGGLVQPGGSLRLSCAASGFTFSSYAMGWVRQAPGKGLEWVSAIRGSGGST |
| | | YYADSVKGRFTISRDNSKNTLYLQMNSLRAEDTAVYYCARVRQGLRRTWYYFDYWG |
| | | QGTLVTVSS |
| 1-24 | 2950 | EVQLLESGGGLVQPGGSLRLSCAASGSTFSSYAMSWVRQAPGKGLEWVSAIGGSGGST |
| | | YYADSVKGRFTISRDNSKNTLYLQMNSLRAEDTAVYYCAKEYSSSWFDPWGQGTLVT |
| | | VSS |
| 1-25 | 2951 | EVQLLESGGGLVQPGGSLSCAASGFTFSSYTMSWVRQAPGKGLEWVSAISVSGGSTYY |
| | | ADSVKGRFTISRDNSKNTLYLQMNSLRAEDTAVYYCAKREDYDFWSGRGAFDIWGQG |
| | | TLVTIS |
| 1-26 | 2952 | EVQLLESGGGLVQPGGSLRLSCAASGFTFSSYAMYWVRQAPGKGLEWVSAISGSGGTY |
| | | YADSVKGRFTISRDNSKNTLYLQMNSLRAEDTAVYYCAKDIGYSSSWSFDYWGQGTL |
| | | VTVSS |
| 1-27 | 2953 | EVQLLESGGGLVQPGGSLRLSCAASGFTFRSYAMSWVRQAPGKGLEWVSAISGSGRST |
| | | YYASVKGRFTISRDNSKNTLYLQMNSLRAEDTAVYYCARDDYSDYRPFDYWGQGTLV |
| | | TVSS |
| 1-28 | 2954 | EVQLLESGGGLVQPGGSLRLSCAASGFTFSSYTMSWVRQAPGKGLEWVSAISGSGGSIY |
| | | YADSVKGRFTISRDNSKNTLYLQMNSLRAEDTAVYYCAHRPSLQWLDWWFDPWGQG |
| | | TLVTVSS |
| 1-29 | 2955 | EVQLLESGGGLVQPGGSLRLSCAASGFTFSSQAMSWVRQAPGKGLEWVSIISGSGGSTY |
| | | YADSVKGRFTISRDNSKNTLYLQMNSLRAEDTAVYYCAKDGASGWPNWHFDLWGQG |
| | | TLVTVSS |
| 1-30 | 2956 | EVQLLESGGGLVQPGGSLRLSCAASGFTFSSYPMSWVRQAPGKGLEWVSAISGSGGRT |
| | | YYADSVKGRFTISRDNSKNTLYLQMNSLRAEDTAVYYCAKGAAAGPFDYWGQGTLV |
| | | TVSS |
| 1-31 | 2957 | EVQLLESGGGLVQPGGSLRLSCAASGFTFSSYAMTWVRQAPGKGLEWVSAISGGTTYY |
| | | ADSVKGRFTISRDNSKNTLYLQMNSLRAEDTAVYYCAKEEYYYDSSGPNWFDPWGQG |
| | | TLVTVSS |
| 1-32 | 2958 | EVQLLESGGGLVQPGGSLRLSCAASGFTFSSYAMSWVRQAPGKGLEWVTAISVSGGST |
| | | YYADSVKGRFTISRDNSKNTLYLQNSLKTQETAGYYWAPQGGTTVPTGRFDPWGQRT |
| | | LVTVSS |
| 1-33 | 2959 | EVQLLESGGGLVQPGGSLRLSCAASGFTFSSYAMSWVRQAPGKGLEWVSAISGSSGST |
| | | YYADSVKGRFTISRDNSKNTLYLQMNSLRAEDTAVYYCSRGGGPAAGFHGLDVWGQ |
| | | GTLVTVSS |
| 1-34 | 2960 | EVQLLESGGGLVQPGGSLRLSCAASGFTFSSYAVSWVRQAPGKGLEWVSAISASGGST |
| | | YYADSVKGRFTISRDNSKNTLYLQMNSLRAEDTAVYYCARAAKRQQLFPRNYFDYWG |
| | | QGTLVTVSS |
| 1-35 | 2961 | EVQLLESGGGLVQPGGSLRLSCAASGFTFSSYPMSWVRQAPGKGLEWVSAIRGSGGST |
| | | YYADSVKGRFTISRDNSKNTLYLQMNSLRAEDTAVYYCALHYGSGRSFDYWGQGTLV |
| | | TVSS |
| 1-36 | 2962 | EVQLLESGGGLVQPGGSLRLSCAASGFTFSSYGMSWVRQAPGKGLEWVSAISGSGGAT |
| | | YYADSVKGRFTISRDNSKNTLYLQMNSLRAEDTAVYYCARPGGRIVGALWGAFDYW |
| | | GQGTLVTVSS |
| 3-1 | 2963 | EVQLVESGGGLVQPGGSLRLSCAASGRTFCRYSMGWFRQAPGKERELVATWRPANTN |
| | | YADSVKGRFTISADNSKNTAYLQMNSLKPEDTAVYYCAKNWGDAGTTWFEKSGWGQ |
| | | GTLVTSS |
| 3-2 | 2964 | EVQLVESGGGLVQPGGSLRLSCAASGNIFSRYIMGWFRQAPGKERELVAAISRTGGSTY |
| | | YADSVKGRFTISADNSKNTAYLQMNSLKPEDTAVYYCAIDPDGEWGQGTLVTVSS |
| 3-3 | 2965 | EVQLVESGGGLVQPGGSLRLSCAASGRTLAGYTMGWFRQAPGKERELLAEIYPSGNGV |
| | | YYADSVKGRFTISADNSKNTAYLQMNSLKPEDTAVYYCAADVRDSIWRSWGQGTLVT |
| | | VSS |
| 3-4 | 2966 | EVQLVESGGGLVQPGGSLRLSCAASGSTLSRYSMGWFRQAPGKEREFVAAIARRERVY |
| | | ADSVKGRFTISADNSKNTAYLQMNSLKPEDTAVYYCARLSCHDYSCYSAFDFWGQGT |
| | | LVTVSS |
| 3-5 | 2967 | EVQLVESGGGLVQPGGSLRLSCAASGSIFSSAAMGWFRQAPGKEREFEAISWRTGTTY |
| | | YADSVKGRFTISADNSKNTAYLQMNSLKPEDTAVYYCAAAGSMGWNHLRDYDWGQ |
| | | GTLVT |
| 3-6 | 2968 | EVQLVESGGGLVQPGGSLRLSCAATFSGYLMGWFRQAPGKEREFVAGIWRSGVSLYY |
| | | ADSVKGRFTISADNSKNTAYLQMNSLKPEDTAVYYCAARSGWGAAMRSADFRWGQG |
| | | TLVTVSS |
| 3-7 | 2969 | EVQLVESGGGLVQPGGSLRLSCAASGRTFSSYDMGWFRQAPGKERERVAIIKSDGSTY |
| | | YADSVKGRFTISADNSKNTAYLQMNSLKPEDTAVYYCARSPRFSGVVVRPGLDLWGQ |
| | | GTLVTVSS |
| 3-8 | 2970 | EVQLVESGGGLVQPGGSLRLSCAASGSISSYFMGWFRQAPGKEREWVSSIGIAGTPTLY |
| | | ADSVKGRFTISADNSKNTAYLQMNSLKPEDTAVYYCAACSDYYCSGVGAVWGQGTL |
| | | VTVSS |
| 3-9 | 2971 | EVQLVESGGGLVQPGGSLRLSCAASGPTFSTYAMGWFRQAPGKEREFVAAVINGGTTN |
| | | YADSVKGRFTISADNSKNTAYLQMNSLKPEDTAVYYCAKDSWDSSGYSYHYYYYGM |
| | | DVWGQGTLVTVSS |
| 3-10 | 2972 | EVQLVESGGGLVQPGGSLRLSCAASGIIGSFRTMGWFRQAPGKERELAGFTGSGRSQY |
| | | YADSVKGRFTISADNSKNTAYLQMNSLKPEDTAVYYCARGDIAVIQVLDYWGQGTLV |
| | | TVSS |
| 3-11 | 2973 | EVQLVESGGGLVQPGGSLRLSCAASGGTFASYGMGWFRQAPGKEREWVAGIWEDSSA |
| | | AHYAESVKGRFTISADNSKNTAYLQMNSLKPEDTAVYYCAYSGIGTDWGQGTLVTVS |
| | | S |
| 3-12 | 2974 | EVQLVESGGGLVQPGGSLRLSCAASGLTFRNYAMGWFRQAPGKERELVAGITSGGTR |
| | | NYADSVKGRFTISADNSKNTAYLQMNSLKPEDTAVYYCAAGWGDSAWGQGTLVTVS |
| | | S |
| 3-13 | 2975 | EVQLVESGGGLVQPGGSLRLSCAASGSISTINVMGWFRQAPGKEREFVAAISWGGGLT |
| | | VYADSVKGRFTISADNSKNTAYLQMNSLKPEDTAVYYCAAFDGYTGSDWGQGTLVT |
| | | VSS |
| 3-14 | 2976 | EVQLVESGGGLVQPGGSLRLSCAASGGTLSSYIGWFRQAPGKERELVATVRSGSITNYA |
| | | DSVKGRFTISADNSKNTAYLQMNSLKPEDTAVYYCAADLTDIWEGIREYDEYAWGQG |
| | | TLVTVSS |
| 3-15 | 2977 | EVQLVESGGGLVQPGGSLRLSCAASGRTFRRYPMGWFRQAPGKEREFVVAVTWSGGS |
| | | TYYADSVKGRFTISADNSKNTAYLQMNSLKPEDTAVYYCAAGLRGRQYSWGQGTLVT |
| | | VSS |
| 3-16 | 2978 | EVQLVESGGGLVQPGGSLRLSCAASGSTFSIDVMGWFRQAPGKEREFVAAISWSGESTL |
| | | YADSVKGRFTISADNSKNTAYLQMNSLKPEDTAVYYCAAFDGYSGSDWGQGTLVTVS |
| | | S |
| 3-17 | 2979 | EVQLVESGGGLVQPGGSLRLSCAASGRTSSSAVMGWFRQAPGKEREFVAAINRGGSTI |
| | | YVDSVKGRFTISADNSKNTAYLQMNSLKPEDTAVYYCATGPYRSYFARSYLWGQGTL |
| | | VTVSS |
| 3-18 | 2980 | EVQLVESGGGLVQPGGSLRLSCAASGGTFSSYRMGWFRQAPGKEREWVSAISWNDGG |
| | | ADYADSVKGRFTISADNSKNTAYLQMNSLKPEDTAVYYCAATQWGSSGWKQARWY |
| | | DWGQGTLVTVSS |
| 3-19 | 2981 | EVQLVESGGGLVQPGGSLRLSCAASGTIFASAMGWFRQAPGKERELVAFSSSGGSTYY |
| | | ADSVKGRFTISADNSKNTAYLQMNSLKPEDTAVYYCAKDPIAAADPGDSVSFDYWGQ |
| | | GTLVTVSS |
| 3-20 | 2982 | EVQLVESGGGLVQPGGSLRLSCAASGFGIDAMGWFRQAPGKEREFVATITEGGATNVG |
| | | STSYSVKGRFTISADNSKNTAYLQMNSLKPEDTAVYYCALNVWRTSSDWGQGTLVTV |
| | | SS |
| 3-21 | 2983 | EVQLVESGGGLVQPGGSLRLSCAASGNIIGGNHMGWFRQAPGKEREFVGAITSSRSTV |
| | | YADSVKGRFTISADNSKNTAYLQMNSLKPEDTAVYYCAAVTTQTYGYDWGQGTLVT |
| | | VSS |
| 3-22 | 2984 | EVQLVESGGGLVQPGGSLRLSCAASGRTFSRYDMGWFRQAPGKEREFVGGTRSGSTN |
| | | YADSVKGRFTISADNSKNTAYLQMNSLKPEDTAVYYCARHSDYSGLSNFDYWGQGTL |
| | | VTVSS |
| 3-23 | 2985 | EVQLVESGGGLVQPGGSLRLSCAAGRQPAPELRGYGMGWFRQAPGKEREFVAAVIGS |
| | | SGTTYYADSVKGRFTISADNSKNTAYLQMNSLKPEDTAVYYCAKAKATVGLRAPFDY |
| | | WGQGTLVTVSS |
| 3-24 | 2986 | EVQLVESGGGLVQPGGSLRLSCAASGINFSRYGMGWFRQAPGKEREFVASITYLGRTN |
| | | YADSVKGRFTISADNSKNTAYLQMNSLKPEDTAVYYCALRVRPYGQYDWGQGTLVT |
| | | VSS |
| 3-25 | 2987 | EVQLVESGGGLVQPGGSLRLSCAASGRTFRRYAMGWFRQAPGKEREFVAAINWSGAR |
| | | TYYADSVKGRFTISADNSKNTAYLQMNSLKPEDTAVYYCAVSKPLNYYTYYDARRYD |
| | | WGQGTLVTVSS |
| 3-26 | 2988 | EVQLVESGGGLVQPGGSLRLSCAASGGTFGHYAMGWFRQAPGKEREFVAAVSWSGSS |
| | | TYYADSVKGRFTISADNSKNTAYLQMNSLKPEDTAVYYCAVSQPLNYYTYYDARRYD |
| | | WGQGTLVTVSS |
| 3-27 | 2989 | EVQLVESGGGLVQPGGSLRLSCAASGFTLDDYAMGWFRQAPGKEREFVAAISWSTGST |
| | | YYADSVKGRFTISADNSKNTAYLQMNSLKPEDTAVYYCAASQAPITIATMMKPFYDW |
| | | GQGTLVTVS |
| 3-28 | 2990 | EVQLVESGGGLVQPGGSLRLSCAASGFTFRRYDMGWFRQAPGKEREFVSAISGGLAYY |
| | | ADSVKGRFTISADNSKNTAYLQMNSLKPEDTAVYYCAVDLSGDAVYDWGQGTLVTV |
| | | SS |
| 3-29 | 2991 | EVQLVESGGGLVQPGGSLRLSCAASGINFSRNAMGWFRQAPGKERELVASITHQDRPIY |
| | | ADSEKGLFTITEDNKKNTDHLMMNLLKPEDTAVYYCALPVGPYGQYDWGQGTLVTW |
| | | S |
| 3-30 | 2992 | EVQLVESGGGLVQPGGSLRLSCAASGRTFTTYGMGWFRQAPGKEREFVASITYLGRTY |
| | | YADSVKGRFTISADNSKNTAYLQMNSLKPEDTAVYYCALRVRPYGQYDWGQGTLVT |
| | | VSS |
| 3-31 | 2993 | EVQLVESGGGLVQPGGSLRLSCAASGSTFSINAMGWFRQAPGKEREFVAGITSSGGYT |
| | | NYADSVKGRFTISADNSKNTAYLQMNSLKPEDTAVYYCAADGVPEYSDYASGPVWG |
| | | QGTLVTVSS |
| 7-1 | 2994 | EVQLLESGGGLVQPGGSLRLSCAASGFTFSNYAMRWVRQAPGKGLEWVSAISGSGGST |
| | | YYADSVRGRFTISRDNSKNTLYLQMNSLRAEDTAVYYCARHTGRYSSGSTGWFHYWG |
| | | QGTLVTVSS |
| 7-2 | 2995 | EVQLVESGGGLVQPGGSLRLSCAASGFAFSRHAMSWFRQAPGKEREFVSDIGGSGSTT |
| | | YYADSVKGRFTISADNSKNTAYLQMNSLKPEDTAVYYCARHTDNWFDPWGQGTLVT |
| | | VSS |
| 7-3 | 2996 | EVQLVESGGGLVQPGGSLRLSCAASGRTFSINAMGWFRQAPGKEREFVAGITRSAVSTI |
| | | TSEGTANYADSVKGRFTISADNSKNTAYLQMNSLKPEDTAVYYCAADGVPEYSDYAS |
| | | GPVWGQGTLVTVSS |
| 7-4 | 2997 | EVQLLESGGGLVQPGESLRLSCAASGFTFSSYGMNWVRQAPGKGLEWVSASSGSGGST |
| | | YYADSVKGRFTISRDNSKNTLYLQMNSLRAEDTAYYCARREYIESGFDSWGQGTLVTV |
| | | SS |
| 7-5 | 2998 | EVQLVESGGGLVQPGGSLRLSCAASGRTFSTDAMGWFRQAPGKEREFVAAISSGGSTN |
| | | YADSVKGRFTISADNSKNTAYLQMNSLKPEDTAVYYCAATRGRSTRLVLPSLVEWGQ |
| | | GTLVTVSS |
| 7-6 | 2999 | EVQLVESGGGLVQPGGSLRLSCAASGRIFYPMGWFRQAPGKEREFVAAVRWSSTGIYY |
| | | TQYADSVKGRFTISADNSKNTAYLQMNSLKPEDTAVYYCAAALSEVWRGSENLREGY |
| | | DWGQGTLVTVSS |
| 7-7 | 3000 | EVQLVESGGGLVQPGGSLRLSCAASGFTFGSYDMGWFRQAPGKEREFVTAINWSGAR |
| | | TAYADSVKGRFTISADNSKNTAYLQMNSLKPEDTAVYYCAARSVYSYEYNWGQGTLV |
| | | TVSS |
| 7-8 | 3001 | EVQLVESGGGLVQPGGSLRLSCAASGSTFTINAMGWFRQAPGKEREFVSGISHNGGTT |
| | | NYADSVKGRFTISADNSKNTAYLQMNSLKPEDTAVYYCAADGVPEYSDYASGPVWG |
| | | QGTLVTVSS |
| 7-9 | 3002 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCAKEDVGKPFDWGQGTLVT |
| | | VSS |
| 7-10 | 3003 | EVQLVESGGGLVQPGGSLRLSCAASGRTYAMGWFRQAPGKEREFVAEINWSGSSTYY |
| | | ADSVKGRFTISADNSKNTAYLQMNSLKPEDTAVYYCAVDGPFGWGQGTLVTVSS |
| 7-11 | 3004 | EVQLVESGGGLVQPGGSLRLSCAASGLPFSTKSMGWFRQAPGKEREFVAAIHWSGLTS |
| | | YADSVKGRFTISADNSKNTAYLQMNSLKPEDTAVYYCAADRAADFFAQRDEYDWGQ |
| | | GTLVTVSS |
| 7-12 | 3005 | EVQLVESGGGLVQPGGSLRLSCAASGRTIVPYTMGWFRQAPGKEREFVAAISPSAFTEY |
| | | ADSVKGRFTISADNSKNTAYLQMNSLKPEDTAVYYCAARRWGYDWGQGTLVTVSS |
| 7-13 | 3006 | EVQLVESGGGLVQPGGSLRLSCAASGLRLNMHRMGWFRQAPGKEREFVAAISGWSGG |
| | | TNYADSVKGRFTISADNSKNTAYLQMNSLKPEDTAVYYCAKIGTLWWGQGTLVTVSS |
| 7-14 | 3007 | EVQLVESGGGLVQPGGSLRLSCAASGSTFSINAMGWFRQAPGKEREFVAGISRGGTTN |
| | | YADSVKGRFTISADNSKNTAYLQMNSLKPEDTAVYYCAADGVPEYSDYASGPVWGQ |
| | | GTLVTVSS |
| 7-15 | 3008 | EVQLVESGGGLVQPGGSLRLSCAASGSTLPYHAMGWFRQAPGKEREFVASISRFFGTA |
| | | YYADSVKGRFTISADNSKNTAYLQMNSLKPEDTAVYYCAPTFAAGASEYHWGQGTLV |
| | | TVSS |
| 7-16 | 3009 | EVQLVESGGGLVQPGGSLRLSCAASGFTFTSYAISWFRQAPGKEREFVSAISGSGGSTD |
| | | YADSVKGRFTISADNSKNTAYLQMNSLKPEDTAVYYCARGAYGSGTYDYWGQGTLV |
| | | TVSS |
| 7-17 | 3010 | EVQLVESGGGLVQPGGSLRLSCAASGFSLDYYGMGWFRQAPGKEREFVAAITSGGTPH |
| | | YADSVKGRFTISADNSKNTAYLQMNSLKPEDTAVYYCASAYNPGIGYDWGQGTLVTV |
| | | SS |
| 7-18 | 3011 | EVQLVESGGGLVQPGGSLRLSCAASGLTDRRYTMGWFRQAPGKEREFVASITLGGSTA |
| | | YADSVKGRFTISADNSKNTAYLQMNSLKPEDTAVYYCAKEDVGKPFDWGQGTLVTVS |
| | | S |
| 7-19 | 3012 | EVQLVESGGGLVQPGGSLRLSCAASGRTFRRYTMGWFRQAPGKEREFVASITSSGVNA |
| | | YADSVKGRFTISADNSKNTAYLQMNSLKPEDTAVYYCAKEDVGKPFDWGQGTLVTVS |
| | | S |
| 7-20 | 3013 | EVQLVESGGGLVQPGGSLRLSCAASGPTFSIYAMGWFRQAPGKEREFVAGISWNGGST |
| | | NYADSVKGRFTISADNSKNTAYLQMNSLKPEDTAVYYCALRRRFGGQEWGQGTLVTV |
| | | SS |
| 7-21 | 3014 | EVQLVESGGGLVQPGGSLRLSCAASGRTISRYTMGWFRQAPGKEREFVASITSGGSTAY |
| | | ADSVKGRFTISADNSKNTAYLQMNSLKPEDTAVYYCAKEDVGKPFDWGQGTLVTVSS |
| 7-22 | 3015 | EVQLVESGGGLVQPGGSLRLSCAASGRTITRYTMGWFRQAPGKEREFVASITSGGSTA |
| | | YADSVKGRFTISADNSKNTAYLQMNSLKPEDTAVYYCAKQDVGKPFDWGQGTLVTVS |
| | | S |
| 7-23 | 3016 | EVQLVESGGGLVQPGGSLRLSCAASGFTFENHAMGWFRQAPGKEREFVAEIYPSGSTIY |
| | | ADSVKGRFTISADNSKNTAYLQMNSLKPEDTAVYYCAARILSRNWGQGTLVTVSS |
| 7-24 | 3017 | EVQLVESGGGLVQPGGSLRLSCAASGFTFSRHAMNWFRQAPGKEREFVSTITGSGGST |
| | | NYADSVKGRFTISADNSKNTAYLQMNSLKPEDTAVYYCAREVGLYYYGSGSSSRRLL |
| | | GRIDYYFDYWGQGTLVTVSS |
| 7-25 | 3018 | EVQLVESGGGLVQPGGSLRLSCAASGFTFDDYSMGWFRQAPGKEREFVASIEWDGSTY |
| | | YADSVKGRFTISADNSKNTAYLQMNSLKPEDTAVYYCAAFDGYTGSDWGQGTLVTVS |
| | | S |
| 7-26 | 3019 | EVQLVESGGGLVQPGGSLRLSCAASGSTFSINAMGWFRQAPGKEREFVAGITSSGGYT |
| | | NYADSVKGRFTISADNSKNTAYLQMNSLKPEDTAVYYCAADGVPEYSDYASGPVWG |
| | | QGTLVTVSS |
| 7-27 | 3020 | EVQLVESGGGLVQPGGSLRLSCAASGQTFNMGWFRQAPGKEREFVAEINWSGSSTYY |
| | | ADSVKGRFTISADNSKNTAYLQMNSLKPEDTAVYYCAVDGPFGWGQGTLVTVSS |
| 7-28 | 3021 | EVQLVESGGGLVQPGGSLRLSCAASGNTFSDNPMGWFRQAPGKEREFVAILAWDSGST |
| | | YYADSVKGRFTISADNSKNTAYLQMNSLKPEDTAVYYCTTDYSKLAITKLSYWGQGT |
| | | LVT |
| 7-29 | 3022 | EVQLVESGGGLVQPGGSLRLSCAASGRTHSIYPMGWFRQAPGKEREFVASITSYGDTN |
| | | YADSVKGRFTISADNSKNTAYLQMNSLKPEDTAVYYCAARRWIPPGPIWGQGTLVTVS |
| | | S |
| 7-30 | 3023 | EVQLVESGGGLVQPGGSLRLSCAASGRTFSMHAMGWFRQAPGKEREFVASISSQGRTN |
| | | YADSVKGRFTISADNSKNTAYLQMNSLKPEDTAVYYCAAEVRNGSDYLPIDWGQGTL |
| | | VTVSS |
| 7-31 | 3024 | EVQLVESGGGLVQPGGSLRLSCAASGFTFSNYSMGWFRQAPGKEREFVAAIHWNGDS |
| | | TAYADSVKGRFTISADNSKNTAYLQMNSLKPEDTAVYYCAAQTEDSAQYIWGQGTLV |
| | | TVSS |
| 7-32 | 3025 | EVQLVESGGGLVQPGGSLRLSCAASGSTFSVNAMGWFRQAPGKEREFVAGVTRGGYT |
| | | NYADSVKGRFTISADNSKNTAYLQMNSLKPEDTAVYYCAADGVPEYSDYASGPVWG |
| | | QGTLVTVSS |
| 7-33 | 3026 | EVQLVESGGGLVQPGGSLRLSCAASGSIGSINAMGWFRQAPGKEREFVAGISNGGTTN |
| | | YADSVKGRFTISADNSKNTAYLQMNSLKPEDTAVYYCAADGVPEYSDYASGPVWGQ |
| | | GTLVTVSS |
| 7-34 | 3027 | EVQLVESGGGLVQPGGSLRLSCAASGRTFGSYDMGWFRQAPGKEREFVAFIHRSGGST |
| | | YYADSVKGRFTISADNSKNTAYLQMNSLKPEDTAVYYCATFPAIVTDSDYDLGNDWG |
| | | QGTLVTVSS |
| 7-35 | 3028 | EVQLVESGGGLVQPGGSLRLSCAASGGTFGHYAMGWFRQAPGKEREFVAAVSWSGSS |
| | | TYYADSVKGRFTISADNSKNTAYLQMNSLKPEDTAVYYCAVSQPLNYYTYYDARRYD |
| | | WGQGTLVTVSS |
| 7-36 | 3029 | EVQLVESGGGLVQPGGSLRLSCAASGFGFGSYDMGWFRQAPGKEREFVTAINWSGAR |
| | | AYYADSVKGRFTISADNSKNTAYLQMNSLKPEDTAVYYCAARSVYSYDYNWGQGTL |
| | | VTVSS |
| 7-37 | 3030 | EVQLVESGGGLVQPGGSLRLSCAASGSTLSINAMGWFRQAPGKEREFVAGITRSGSVT |
| | | NYADSVKGRFTISADNSKNTAYLQMNSLKPEDTAVYYCAADGVPEYSDYASGPVWG |
| | | QGTLVTVSS |
| 7-38 | 3031 | EVQLVESGGGLVQPGGSLRLSCAASGRPFSEYTMGWFRQAPGKEREFVSSIHWGGRGT |
| | | NYADSVKGRFTISADNSKNTAYLQMNSLKPEDTAVYYCAAELHSSDYTSPGAYAWGQ |
| | | GTLVTVSS |
| 7-39 | 3032 | EVQLVESGGGLVQPGGSLRLSCAASGRTFSNYPMGWFRQAPGKEREFVAAITWSGDST |
| | | NYADSVKGRFTISADNSKNTAYLQMNSLKPEDTAVYYCALPSNIITTDYLRVYWGQGT |
| | | LVTVSS |
| 7-40 | 3033 | EVQLVESGGGLVQPGGSLRLSCAASGRTFRRYTMGWFRQAPGKEREFVASITKFGSTN |
| | | YADSVKGRFTISADNSKNTAYLQMNSLKPEDTAVYYCAKEDVGKPFDWGQGTLVTVS |
| | | S |
| 7-41 | 3034 | EVQLVESGGGLVQPGGSLRLSCAASGRTFSTYVMGWFRQAPGKEREFVASISSRGITHY |
| | | ADSVKGRFTISADNSKNTAYLQMNSLKPEDTAVYYCAKEDVGKPFDWGQGTLVTVSS |
| 7-42 | 3035 | EVQLVESGGGLVQPGGSLRLSCAASGFTLDYYGMGWFRQAPGKEREFVAAITSGGTPH |
| | | YGDSVKGRFTISADNSKNTAYLQMNSLKPEDTAVYYCASAYNPGIGYDWGQGTLVTV |
| | | SS |
| 7-43 | 3036 | EVQLVESGGGLVQPGGSLRLSCAASGFTFGHYAMGWFRQAPGKEREFVAAVSWSGST |
| | | TYYADSVKGRFTISADNSKNTAYLQMNSLKPEDTAVYYCAVSHPLNYYTYYDARRYD |
| | | WGQGTLVTVSS |
| 7-44 | 3037 | EVQLVESGGGLVQPGGSLRLSCAASGFTFEDYAMGWFRQAPGKEREGVAAITRGSNTT |
| | | DYADSVKGRFTISADNSKNTAYLQMNSLKPKDTAVYYCAARRWMGGSYFDPGNYDW |
| | | GQGTLVTVSS |
| 7-45 | 3038 | EVQLVESGGGLVQPGGSLRLSCAASGRTLSRYTMGWFRQAPGKEREFVASITSGGSTN |
| | | YADSVKGRFTISADNSKNTAYLQMNSLKPEDTAVYYCAKEDVGKPFDWGQGTLVTVS |
| | | S |
| 8-1 | 3039 | EVQLVESGGGLVQPGGSLRLSCAASGRTFASYAMGWFRQAPGKEREFVGAISRSGDST |
| | | YYADSVKGRFTISADNSKNTAYLQMNSLKPEDTAVYYCARAPFYCTTTKCQDNYYYM |
| | | DVWGQGTLVTVSS |
| 8-2 | 3040 | EVQLVESGGGLVQPGGSLRLSCAASGGTYHAMGWFRQAPGKEREFVAGITSDDRTNY |
| | | ADSVKGRFTISADNSKNTAYLQMNSLKPEDTAVYYCARERRYYDSSGYPYYFDYWGQ |
| | | GTLVTVSS |
| 8-3 | 3041 | EVQLVESGGGLVQPGGSLRLSCAASGTTLDYYAMGWFRQAPGKEREFVAAISWSGGS |
| | | TAYADSVKGRFTISADNSKNTAYLQMNSLKPEDTAVYYCAREDYYDSSGYSWGQGTL |
| | | VTVSS |
| 8-4 | 3042 | EVQLVESGGGLVQPGGSLRLSCAASGGTLSRSRMGWFRQAPGKEREFVAFIGSDTLYA |
| | | DSVKGRFTISADNSKNTAYLQMNSLKPEDTAVYYCANLAYYDSSGYYDYWGQGTLV |
| | | TVSS |
| 8-5 | 3043 | EVQLVESGGGLVQPGGSLRLSCAASGGTFSFYNMGWFRQAPGKEREFVAFISGNGGTS |
| | | YADSVKGRFTISADNSKNTAYLQMNSLKPEDTAVYYCAVVAMRMVTTEGPDVLDVW |
| | | GQGTLVTVSS |
| 8-6 | 3044 | EVQLVESGGGLVQPGGSLRLSCAASGFTFDYYAMGWFRQAPGKEREFVSAIDSEGRTS |
| | | YADSVKGRFTISADNSKNTAYLQMNSLKPEDTAVYYCARWGPFDIWGQGTLVTVSS |
| 8-7 | 3045 | EVQLVESGGGLVQPGGSLRLSCAASGFPFSIWPMGWFRQAPGKEREFVAAVRWSSTGI |
| | | YYTQYADSVKGRFTISADNSKNTAYLQMNSLKPEDTAVYYCTRSEYSSGWYDYWGQ |
| | | GTLVTVSS |
| 8-8 | 3046 | EVQLVESGGGLVQPGGSLRLSCAASGFAESSSMGWFRQAPGKEREFVAAISWSGDITIY |
| | | ADSVKGRFTISADNSKNTAYLQMNSLKPEDTAVYYCARGAPYFDHGSKSYRLFYFDY |
| | | WGQGTLVTVSS |
| 8-9 | 3047 | EVQLVESGGGLVQPGGSLRLSCAASGFTFGTTTMGWFRQAPGKEREFVAAISWSTGIA |
| | | HYADSVKGRFTISADNSKNTAYLQMNSLKPEDTAVYYCARGGPNYYASGRYPWFDP |
| | | WGQGTLVTVSS |
| 8-10 | 3048 | EVQLVESGGGLVQPGGSLRLSCAASGFIGNYHAMGWFRQAPGKEREFVAAVTWSGGT |
| | | TNYADSVKGRFTISADNSKNTAYLQMNSLKPEDTAVYYCAREGYYYDSSGYPYYFDY |
| | | WGQGTLVTVSS |
| 2A-1 | 3049 | EVQLLESGGGLVQPGGSLRLSCAASGFTFSNYATDWVRQAPGKGLEWVSIISGSGGAT |
| | | YYADSVKGRFTISRDNSKNTLYLQMNSLRAEDTAVYYCAKGGYCSSDTCWWEYWLD |
| | | PWGQGTLVTVSS |
| 2A-10 | 3050 | EVQLLESGGGLVQPGGSLRLSCAASGFTFSAFAMGWVRQAPGKGLEWVSAITASGDIT |
| | | YYADSVKGRFTISRDNSKNTLYLQMNSLRAEDTAVYYCARQSDGLPSPWHFDLGGQG |
| | | TLVTVSS |
| 2A-5 | 3051 | EVQLLESGGGLVQPGGSLRLSCAASGFTFSDFAMAWVRQAPGKGLEWVSAISGSGDIT |
| | | YYADSVKGRFTISRDNSKNTLYLQMNSLRAEDTAVYYCAREADGLHSPWHFDLWGQ |
| | | GTLVTVSS |
| 2A-2 | 3052 | EVQLLESGGGLVQPGGSLRLSCAASGFTFSRHAMNWVRQAPGKGLEWVSGISGSGDET |
| | | YYADSVKGRFTISRDNSKNTLYLQMNSLRAEDTAVYYCARDLPASYYDSSGYYWHNG |
| | | MDVWGQGTLVTVSS |
| 2A-4 | 3053 | EVQLLESGGGLVQPGGSLRLSCAASGFTFSDFAMAWVRQAPGKGLEWVSAISGSGDIT |
| | | YYADSVKGRFTISRDNSKNTLYLQMNSLRAEDTAVYYCAREADGLHSPWHFDLWGQ |
| | | GTLVTVSS |
| 2A-6 | 3054 | EVQLLESGGGLVQPGGSLRLSCAASGFTFSNYPMNWVRQAPGKGLEWVSTISGSGGNT |
| | | FYADSVKGRFTISRDNSKNTLYLQMNSLRAEDTAVYYCVRHDEYSFDYWGQGTLVTV |
| | | SS |
| 2A-11 | 3055 | EVQLLESGGGLVQPGGSLRLSCAASGFTFSDFAMAWVRQAPGKGLEWVSAITGSGDIT |
| | | YYADSVKGRFTISRDNSKNTLYLQMNSLRAEDTAVYYCAREADGLHSPWHFDLWGQ |
| | | GTLVTVSS |
| 2A-12 | 3056 | EVQLLESGGGLVQPGGSLRLSCAASGFTFSDYPMNWVRQAPGKGLEWVSTISGSGGIT |
| | | FYADSVKGRFTISRDNSKNTLYLQMNSLRAEDTAVYYCVRHDEYSFDYWGQGTLVTV |
| | | SS |
| 2A-13 | 3057 | EVQLLESGGGLVQPGGSLRLSCAASGFTFSDYPMNWVRQAPGKGLEWVSAISGSGDNT |
| | | YYADSVKGRFTISRDNSKNTLYLQMNSLRAEDTAVYYCVRHDEYSFDYWGQGTLVTV |
| | | SS |
| 2A-14 | 3058 | EVQLLESGGGLVQPGGSLRLSCAASGFTFSDFAMAWVRQAPGKGLEWVSAITGTGDIT |
| | | YYADSVKGRFTISRDNSKNTLYLQMNSLRAEDTAVYYCAREADGLHSPWGQGTLVTV |
| | | SS |
| 2A-7 | 3059 | EVQLLESGGGLVQPGGSLRLSCAASGFTFSDYPMNWVRQAPGKGLEWVSAITGSGDIT |
| | | YYADSVKGRFTISRDNSKNTLYLQMNSLRAEDTAVYYCVRHDEYSFDYWGQGTLVTV |
| | | SS |
| 2A-8 | 3060 | EVQLLESGGGLVQPGGSLRLSCAASGFTFSDFAMAWVRQAPGKGLEWVSAISGSGDIT |
| | | YYADSVKGRFTISRDNSKNTLYLQMNSLRAEDTAVYYCAREADGLHSPWHFDLWGQ |
| | | GTLVTVSS |
| 2A-15 | 3061 | EVQLLESGGGLVQPGGSLRLSCAASGFTFSDFAMAWVRQAPGKGLEWVSAISGSGDIT |
| | | YYADSVKGRFTISRDNSKNTLYLQMNSLRAEDTAVYYCAREADGLHSPWHFDLWGQ |
| | | GTLVTVSS |
| 2A-9 | 3062 | EVQLLESGGGLVQPGGSLRLSCAASGFTFPRYAMSWVRQAPGKGLEWVSTISGSGSTT |
| | | YYADSVKGRFTISRDNSKNTLYLQMNSLRAEDTAVYYCARLIDAFDIWGQGTLVTVSS |
| 2A-21 | 3063 | EVQLLESGGGLVQPGGSLRLSCAASGFTFPRYAMSWVRQAPGKGLEWVSTISGSGSTT |
| | | YYADSVKGRFTISRDNSKNTLYLQMNSLRAEDTAVYYCARLIDAFDIWGQGTLVTVSS |
| 2A-22 | 3064 | EVQLLESGGGLVQPGGSLRLSCAASGFTFTTYALSWVRQAPGKGLEWVSGISGSGDET |
| | | YYADSVKGRFTISRDNSKNTLYLQMNSLRAEDTAVYYCTTGDDFWSGGNWFDPWGQ |
| | | GTLVTVSS |
| 2A-23 | 3065 | EVQLLESGGGLVQPGGSLRLSCAASGFTFSRHAMNWVRQAPGKGLEWVSGITGSGDE |
| | | TYYADSVKGRFTISRDNSKNTLYLQMNSLKAEDTAVYYCARDLPASYYDSSGYYWHN |
| | | GMDVWGQGTLVTVSS |
| 2A-24 | 3066 | EVQLLESGGGLVQPGGSLRLSCAASGFVFSSYAMSWVRQAPGKGLEWVSAISGSGGSS |
| | | YYADSVKGRFTISRDNSKNTLYLQMNSLRAEDTAVYYCARVGGGYWYGIDVWGQGT |
| | | LVTVSS |
| 2A-25 | 3067 | EVQLLESGGGLVQPGGSLRLSCAASGFTLSSYVMSWVRQAPGKGLEWVSGISGGGAST |
| | | YYADSVKGRFTISRDNSKNTLYLQMNSLRAEDTAVYYCARGYSRNWYPSWFDPWGQ |
| | | GTLVTVSS |
| 2A-26 | 3068 | EVQLLESGGGLVQPGGSLRLSCAASGFTFSTYAMSWVRQAPGKGLEWVSSIGGSGSTT |
| | | YYADSVKGRFTISRDNSKNTLYLQMNSLRAEDTAVYYCAGGWYLDYWGQGTLVTVS |
| | | S |
| 2A-27 | 3069 | EVQLLGSGGGLVQPGGSLRLSCAASGFTYSNYAMTWVRQAPGKGLEWVSAISGSSGST |
| | | YYADSVKGRFTISRDNSKNTLYLQMNSLRAEDTAVYYCASLCIVDPFDIWGQGTLVTV |
| | | SS |
| 2A-28 | 3070 | EVQLLESGGGLVQPGGSLRLSCAASGFTFSNYPMNWVRQAPGKGLEWVSTISGSGGNT |
| | | FYADSVKGRFTISRDNSKNTLYLQMNSLRAEDTAVYYCVRHDEYSFDYWGQGTLVTV |
| | | SS |
| 3A-10 | 3071 | EVQLLESGGGLVQPGGSLRLSCAASGFTFRSHAMSWVRQAPGKGLEWVSSISGGGAST |
| | | YYADSVKGRFTISRDNSKNTLYLQMNSLRAEDTAVYYCARVKYLTTSSGWPRPYFDN |
| | | WGQGTLVTVSS |
| 3A-4 | 3072 | EVQLLESGGGLVQPGGSLRLSCAASGFTFSAYSMSWVRQAPGKGLEWVSAISGSGGSR |
| | | YYADSVKGRFTISRDNSKNTLYLQMNSLRAEDTAVYYCGRSKWPQANGAFDIWGQGT |
| | | LVTVSS |
| 3A-7 | 3073 | EVQLLESGGGLVQPGGSLRLSCAASGFMFGNYAMSWVRQAPGKGLEWVAAISGSGGS |
| | | TYYADSVKGRFTISRDNSKNTLYLQMNSLRAEDTAVYYCAKDRGYSSSWYGGFDYW |
| | | GQGTLVTVSS |
| 3A-1 | 3074 | EVQLLESGGGLVQPGGSLRLSCAASGFTFRNHAMAWVRQAPGKGLEWVSGISGSGGT |
| | | TYYGDSVKGRFTISRDNSKNTLYLQMNSLRAEDTAVYYCARGTRFLQWSLPLDVWGQ |
| | | GTLVTVSS |
| 3A-5 | 3075 | EVQLLESGGGLVQPGGSLRLSCAASGFTIPNYAMSWVRQAPGKGLEWVSGISGAGAST |
| | | YYADSVKGRFTISRDNSKNTLYLQMNSLRAEDTAVYYCARHTWWKGAGFFDHWGQG |
| | | TLVTVSS |
| 3A-6 | 3076 | EVQLLESGGGLVQPGGSLRLSCAASGFTFRNYAMAWVRQAPGKGLEWVSGISGSGGT |
| | | TYYGDSVKGRFTISRDNSKNTLYLQMNSLRAEDTAVYYCARGTRFLEWSLPLDVWGQ |
| | | GTLVTVSS |
| 3A-15 | 3077 | EVQLLESGGGLVQPGGSLRLSCAASGFTIRNYAMSWVRQAPGKGLEWVSSISGGGAST |
| | | YYADSVKGRFTISRDNSKNTLYLQMNSLRAEDTAVYYCARVKYLTTSSGWPRPYFDN |
| | | WGQGTLVTVSS |
| 3A-3 | 3078 | EVQLLESGGGLVQPGGSLRLSCAASGFTIPNYAMSWVRQAPGKGLEWVSGISGSGAST |
| | | YYADSVKGRFTISRDNSKNTLYLQMNSLRAEDTAVYYCARHTWWKGAGFFDHWGQG |
| | | TLVTVSS |
| 3A-11 | 3079 | EVQLLESGGGLVQPGGSLRLSCAASGFTITNYAMSWVRQAPGKGLEWVSGISGSGAGT |
| | | YYADSVKGRFTISRDNSKNTLYLQMNSLRAEDTAVYYCARHAWWKGAGFFDHWGQ |
| | | GTLVTVSS |
| 3A-8 | 3080 | EVQLLESGGGLVQPGGSLRLSCAASGFTFRSHAMSWVRQAPGKGLEWVSSISGGGAST |
| | | YYADSVKGRFTISRDNSKNTLYLQMNSLRAEDTAVYYCARVKYLTTSSGWPRPYFDN |
| | | WGQGTLVTVSS |
| 3A-2 | 3081 | EVQLLESGGGLVQPGGSLRLSCAASGFTITNYAMSWVRQAPGKGLEWVSGISGSGAST |
| | | YYADSVKGRFTISRDNSKNTLYLQMNSLRAEDTAVYYCARHTWWKGAGFFDHWGQG |
| | | TLVTVSS |
| 3A-12 | 3082 | EVQLLESGGGLVQPGGSLRLSCAASGFTFRSHAMNWVRQAPGKGLEWVSAISGSGGST |
| | | NYADSVKGRFTISRDNSKNTLYLQMNSLRAEDTAVYYCARGLKFLEWLPSAFDIWGQ |
| | | GTLVTVSS |
| 3A-14 | 3083 | EVQLLESGGGLVQPGGSLRLSCAASGFTFRSHAMSWVRQAPGKGLEWVSSISGGGAST |
| | | YYADSVKGRFTISRDNSKNTLYLQMNSLRAEDTAVYYCARVKYLTTSSGWPRPYFDN |
| | | WGQGTLVTVSS |
| 3A-9 | 3084 | EVQLLESGGGLVQPGGSLRLSCAASGFTFRSYAMSWVRQAPGKGLEWVSSISGGGAST |
| | | YYADSVKGRFTISRDNSKNTLYLQMNSLRAEDTAVYYCARVKYLTTSSGWPRPYFDN |
| | | WGQGTLVTVSS |
| 3A-13 | 3085 | EVQLLESGGGLVQPGGSLRLSCAASGFTITNYAMSWVRQAPGKGLEWVSGISGSGAGT |
| | | YYADSVKGRFTISRDNSKNTLYLQMNSLRAEDTAVYYCARHTWWKGAGFFDHWGQG |
| | | TLVTVSS |
| 3A-16 | 3086 | EVQLLESGGGLVQPGGSLRLSCAASGFTFTNFAMSWVRQAPGKGLEWVSAISGRGGGT |
| | | YYADSVKGRFTISRDNSKNTLYLQMNSLRAEDTAVYYCARDAHGYYYDSSGYDDWG |
| | | QGTLVTVSS |
| 3A-17 | 3087 | EVQLLESGGGLVQPGGSLRLSCAASGFTFRSYPMSWVRQAPGKGLEWVSTISGSGGITY |
| | | YADSVKGRFTISRDNSKNTLYLQMNSLRAEDTAVYYCAKGVYGSTVTTCHWGQGTLV |
| | | TVSS |
| 3A-18 | 3088 | EVQLLESGGGLVQPGGSLRLSCAASGFTLTSYAMSWVRQAPGKGLEWVSAISGSGVDT |
| | | YYADSVKGRFTISRDNSKNTLYLQMNSLRAEDTAVYYCARPTNWGFDYWGQGTLVT |
| | | VSS |
| 3A-19 | 3089 | EVQLLESGGGLVQPGGSLRLSCAASGFTFINYAMSWVRQAPGKGLEWVSTISTSGGNT |
| | | YYADSVKGRFTISRDNSKNTLYLQMNSLRAEDTAVYYCARADSNWASSAYWGQGTL |
| | | VTVSS |
| 3A-2 | 3090 | EVQLLESGGGLVQPGGSLRLSCAASGFPFSTYAMSWVRQAPGKGLEWVSGISVSGGFT |
| | | YYADSVKGRFTISRDNSKNTLYLQMNSLRAEDTAVYYCARDPYSYGYYYYYGMDVW |
| | | GQGTLVTVSS |
| 3A-21 | 3091 | EVQLLESGGGLVQPGGSLRLSCAASGFTFSTYAMGWVRQAPGKGLEWVSGISGGGVS |
| | | TYYADSVKGRFTISRDNSKNTLYLQMNSLRAEDTAVYYCARARNWGPSDYWGQGTL |
| | | VTVSS |
| 3A-22 | 3092 | EVQLLESGGGLVQPGGSLRLSCAASGFIFSDYAMTWVRQAPGKGLEWVSAISGSAFYA |
| | | DSVKGRFTISRDNSKNTLYLQMNSLRAEDTAVYYCARDATYSSSWYNWFDPWGQGTL |
| | | VTVSS |
| 3A-23 | 3093 | EVQLLESGGGLVQPGGSLRLSCAASGFTFSDYAMTWVRQAPGKGLEWVSDISGSGGST |
| | | YYADSVKGRFTISRDNSKNTLYLQMNSLRAEDTAVYYCARGTVTSFDFWGQGTLVTV |
| | | SS |
| 3A-24 | 3094 | EVQLLESGGGLVQPGGSLRLSCAASGFTFSIYAMGWVRQAPGKGLEWVSFISGSGGST |
| | | YYADSVKGRFTISRDNSKNTLYLQMNSLRAEDTAVYYCAKDYHSASWFSAAADYWG |
| | | QGTLVTVSS |
| 3A-25 | 3095 | EVQLLESGGGLVQPGGSLRLSCAASGFTFASYAMTWVRQAPGKGLEWVSAISESGGST |
| | | YYADSVKGRFTISRDNSKNTLYLQMNSLRAEDTAVYYCAREGQEYSSGSSYFDYWGQ |
| | | GTLVTVSS |
| 3A-26 | 3096 | EVQLLESGGGLVQPGGSLRLSCAASGFTFSEYAMSWVRQAPGKGLEWVSAITGSGGST |
| | | YYGDSVKGRFTISRDNSKNTLYLQMNSLRAEDTAVYYCARGSQTPYCGGDCPETFDY |
| | | WGQGTLVTVSS |
| 3A-27 | 3097 | EVQLLESGGGLVQPGGSLRLSCAASGFTFDDYAMSWVRQAPGKGLEWVSGISGGGTS |
| | | TYYADSVKGRFTISRDNSKNTLYLQMNSLRAEDTAVYYCARDLYSSGWYGFDYWGQ |
| | | GTLVTVSS |
| 3A-28 | 3098 | EVQLLESGGGLVQPGGSLRLSCAASGFTFNNYAMNWVRQAPGKGLEWVSAISGSVGS |
| | | TYYADSVKGRFTISRDNSKNTLYLQMNSLRAEDTAVYYCARDNYDFWSGYYTNWFD |
| | | PWGQGTLVTVSS |
| 3A-29 | 3099 | EVQLLESGGGLVQPGGSLRLSCAASGFTFTNHAMSWVRQAPGKGLEWVSAISGSGSNI |
| | | YYADSVKGRFTISRDNSKNTLYLQMNSLRAEDTAVYYCARDSLSVTMGRGVVTYYYY |
| | | GMDFWGQGTLVTVSS |
| 4A-51 | 3100 | EVQLVESGGGLVQPGGSLRLSCAASGPGTAIMGWFRQAPGKEREFVARISTSGGSTKY |
| | | ADSVKGRFTISADNSKNTAYLQMNSLKPEDTAVYYCARTTVTTPPLIWGQGTLVTVSS |
| 4A-52 | 3101 | EVQLVESGGGLVQPGGSLRLSCAASGRSFSNSVMGWFRQAPGKEREFVARITWNGGST |
| | | YYADSVKGRFTISADNSKNTAYLQMNSLKPEDTAVYYCATTENPNPRWGQGTLVTVS |
| | | S |
| 4A-53 | 3102 | EVQLVESGGGLVQPGGSLRLSCAASGRTFGDDTMGWFRQAPGKEREFVAAVSWSGSG |
| | | VYYADSVKGRFTITADNSKNTAYLQMNSLKPENTAVYYCATDPPLFWGQGTLVTVSS |
| 4A-54 | 3103 | EVQLVESGGGLVQPGGSLRLSCAASGRTFSDARMGWFRQAPGKEREFVGAVSWSGGT |
| | | TVYADSVKGRFTISADNSKNTAYLQMNSLKPEDTAVYYCATTEDPYPRWGQGTLVTV |
| | | SS |
| 4A-49 | 3104 | EVQLVESGGGLVQPGGSLRLSCAASGRTFGDYIMGWFRQAPGKERESVAAINWSAGY |
| | | TAYADSVKGRFTISADNSKNTAYLQMNSLKPEDTAVYYCARASPNTGWHFDHWGQG |
| | | TLVTVSS |
| 4A-55 | 3105 | EVQLVESGGGLVQPGGSLRLSCAASGSGLSINAMGWFRQAPGKERESVAAISWSGGST |
| | | YTAYADSVKGRFTISADNSKNTAYLQMNSLKPEDTAVYYCAAYQAGWGDWGQGTLV |
| | | TVSS |
| 4A-39 | 3106 | EVQLVESGGGLVQPGGSLRLSCAASGRTFSNAAMGWFRQAPGKEREFVARILWTGAS |
| | | RNYADSVKGRFTISADNSKNTAYLQMNSLKPEDTAVYYCATTENPNPRWGQGTLVTV |
| | | SS |
| 4A-56 | 3107 | EVQLVESGGGLVQPGGSLRLSCAASGFSLDYYGMGWFRQAPGKERESVAAISWNGDF |
| | | TAYADSVKGRFTISADNSKNTAYLQMNSLKPEDTAVYYCAKRANPTGAYFDYWGQG |
| | | TLVTVSS |
| 4A-33 | 3108 | EVQLVESGGGLVQPGGSLRLSCAASGFTFSRHDMGWFRQAPGKEREFVAGINWESGST |
| | | NYADSVKGRFTISADNSKNTAYLQMNSLKPEDTAVYYCAADRGVYGGRWYRTSQYT |
| | | WGQGTLVTVSS |
| 4A-57 | 3109 | EVQLVESGGGLVQPGGSLRLSCAASGLTFRNYAMGWFRQAPGKEREFVAAIGSGGYT |
| | | DYADSVKGRFTISADNSKNTAYLQMNSLKPEDTAVYYCAVKPGWVARDPSQYNWGQ |
| | | GTLVTVSS |
| 4A-25 | 3110 | EVQLVESGGGLVQPGGSLRLSCAASGGTFSRYAMGWFRQAPGKEREWVSAVDSGGST |
| | | YYADSVKGRFTISADNSKNTAYLQMNSLKPEDTAVYYCAASPSLRSAWQWGQGTLVT |
| | | VSS |
| + | | |
| 4A-58 | 3111 | EVQLVESGGGLVQPGGSLRLSCAASGFTLDYYDMGWFRQAPGKEREFVAAVTWSGGS |
| | | TYYADSVKGRFTISADNSKNTAYLQMNSLKPEDTAVYYCAADRRGLASTRAADYDW |
| | | GQGTLVTVSS |
| 4A-59 | 3112 | EVQLVESGGGLVQPGGSLRLSCAASGRTFGDYIMGWFRQAPGKEREFVAAINWSAGY |
| | | TPYADSVKGRFTISADNSKNTAYLQMNSLKPEDTAVYYCATAPPLFCWHFDLWGQGT |
| | | LVTVSS |
| 4A-6 | 3113 | EVQLVESGGGLVQPGGSLRLSCAASGRTFGDDIMGWFRQAPGKEREFVAAIHWSAGY |
| | | TRYADSVKGRFTISADNSKNTAYLQMNSLKPEDTAVYYCATDPPLFWGHVDLWGQGT |
| | | LVTVSS |
| 4A-61 | 3114 | EVQLVESGGGLVQPGGSLRLSCAASGRTFGDYIMGWFRQAPGKEREIVAAINWSADYT |
| | | PYADSVKGRFTISADNSKNTAYLQMNSLKPEDTAVYYCATAPPNTGWHFDHWGQGTL |
| | | VTVSS |
| 4A-3 | 3115 | EVQLVESGGGLVQPGGSLRLSCAASGRTFGDYIMGWFRQAPGKEREIVAAINWSAGYT |
| | | AYADSVKGRFTISADNSKNTAYLQMNSLKPEDTAVYYCATATPNTGWHFDHWGQGT |
| | | LVTVSS |
| 4A-62 | 3116 | EVQLVESGGGLVQPGGSLRLSCAASGRTFSDDTMGWFRQAPGKEREFVAAINWSGGS |
| | | TDYADSVKGRFTISADNSKNTAYLQMNSLKPEDTAVYYCATDPPLFWGQGTLVTVSS |
| 4A-43 | 3117 | EVQLVESGGGLVQPGGSLRLSCAASGRTFGDDTMGWFRQAPGKEREFVAGINWSGGN |
| | | TYYADSVKGRFTISADNSKNTAYLQMNSLKPEDTAVYYCATDPPLFWGQGTLVTVSS |
| 4A-5 | 3118 | EVQLVESGGGLVQPGGSLRLSCAASGRTFGDYIMGWFRQAPGKEREFVAAINWTGGY |
| | | TSYADSVKGRFTISADNSKNTAYLQMNSLKPEDTAVYYCATDPPLFWGQGTLVTVSS |
| 4A-42 | 3119 | EVQLVESGGGLVQPGGSLRLSCAASGRTFGDDTMGWFRQAPGKERECVAAINWSGGN |
| | | TYYADSVKGRFTISADNSKNTAYLQMNSLKPEDTAVYYCATDPPLFWGQGTLVTVSS |
| 4A-63 | 3120 | EVQLVESGGGLVQPGGSLRLSCAASGRTFSDYTMGWFRQAPGKEREFVAAINWSGGY |
| | | TYYADSVKGRFTISADNSKNTAYLQMNSLKPEDTAVYYCATDPPLFWGQGTLVTVSS |
| 4A-6 | 3121 | EVQLVESGGGLVQPGGSLRLSCAASGRTFGDYGMGWFRQAPGKEREFVATINWSGAL |
| | | THYADSVKGRFTISADNSKNTAYLQMNSLKPEDTAVYYCATLPFYDFWSGYYTGYYY |
| | | MDVWGQGTLVTVSS |
| 4A-40 | 3122 | EVQLVESGGGLVQPGGSLRLSCAASGRTFSDDTMGWFRQAPGKEREFLAGVTWSGSS |
| | | TFYADSVKGRFTISADNSKNTAYLQMNSLKPEDTAVYYCATDPPLFWGQGTLVTVSS |
| 4A-21 | 3123 | EVQLVESGGGLVQPGGSLRLSCAASGRTFSDDIMGWFRQAPGKEREFVAAISWSGGNT |
| | | HYADSVKGRFTISADNSKNTAYLQMNSLKPEDTAVYYCATDPPLFWGQGTLVTVSS |
| 4A-64 | 3124 | EVQLVESGGGLVQPGGSLRLSCAASGRTFGDYIMGWFRQAPGKERESVAAINWSAGY |
| | | TAYADSVKGRFTISADNSKNTAYLQMNSLKPEDTAVYYCATASPNTGWHFDHWGQG |
| | | TLVTVSS |
| 4A-47 | 3125 | EVQLVESGGGLVQPGGSLRLSCAASGFTFDDDYVMGWFRQAPGKEREFVAAVSGSGD |
| | | DTYYADSVKGRFTISADNSKNTAYLQMNSLKPEDTAVYYCAADRRGLASTRAADYD |
| | | WGQGTLVTVSS |
| 4A-65 | 3126 | EVQLVESGGGLVQPGGSLRLSCAASGRTFGDYIMGWFRQAPGKEREFVAAINWSAGY |
| | | TAYADSVKGRFTISADNSKNTAYLQMNSLKPEDTAVYYCATEPPLSCWHFDLWGQGT |
| | | LVTVSS |
| 4A-18 | 3127 | EVQLVESGGGLVQPGGSLRLSCAASGRTFGDYIMGWFRQAPGKEREIVAAINWSGGYT |
| | | PYADSVKGRFTISADNSKNTAYLQMNSLKPEDTAVYYCATAPPNTGWHFDHWGQGTL |
| | | VTVSS |
| 4A-66 | 3128 | EVQLVESGGGLVQPGGSLRLSCAASGRTFGDDTMGWFRQAPGKEREIVAAINWSAGY |
| | | TPYADSVKGRFTISADNSKNTAYLQMNSLKPEDTAVYYCATDPPLFCCHFDLWGQGTL |
| | | VTVSS |
| 4A-36 | 3129 | EVQLVESGGGLVQPGGSLRLSCAASGRTFSDDTMGWFRQAPGKEREFVAAISWSGGTT |
| | | RYADSVKGRFTISADNSKNTAYLQMNSLKPEDTAVYYCATDPPLFWGQGTLVTVSS |
| 4A-67 | 3130 | EVQLVESGGGLVQPGGSLRLSCAASGRTFSDDTMGWFRQAPGKEREFVAAINWSGDS |
| | | TYYADSVKGRFTISADNSKNTAYLQMNSLKPEDTAVYYCATDPPLFWGQGTLVTVSS |
| 4A-16 | 3131 | EVQLVESGGGLVQPGGSLRLSCAASGRTFSDDTMGWFRQAPGKEREFVAAINWSGGT |
| | | TRYADSVKGRFTISADNSKNTAYLQMNSLKPEDTAVYYCATDPPLFWGQGTLVTVSS |
| 4A-11 | 3132 | EVQLVESGGGLVQPGGSLRLSCAASGRTFSDDAMGWFRQAPGKEREFVAAIHWSGSST |
| | | RYADSVKGRFTISADNSKNTAYLQMNSLKPEDTAVYYCATDPPLFWGQGTLVTVSS |
| 4A-68 | 3133 | EVQLVESGGGLVQPGGSLRLSCAASGRTFSDDTMGWFRQAPGKERELVGTINWSGGST |
| | | YYADSVKGRFTISADNSKNTAYLQMNSLKPEDTAVYYCATDPPLFWGQGTLVTVSS |
| 4A-34 | 3134 | EVQLVESGGGLVQPGGSLRLSCAASGRTFGDYIMGWFRQAPGKEREFVAAINWSGGY |
| | | TPYADSVKGRFTISADNSKNTAYLQMNSLKPEDTAVYYCATDPPLFWGQGTLVTVSS |
| 4A-28 | 3135 | EVQLVESGGGLVQPGGSLRLSCAASGRTFGDDTMGWFRQAPGKERELVAAINWNGGN |
| | | THYADSVKGRFTISADNSKNTAYLQMNSLKPEDTAVYYCATDPPLFWGQGTLVTVSS |
| 4A-69 | 3136 | EVQLVESGGGLVQPGGSLRLSCAASGRTFSDDAMGWFRQAPGKEREFVAAINWSGGT |
| | | TRYADSVKGRFTISADNSKNTAYLQMNSLKPEDTAVYYCATDPPLFWGQGTLVTVSS |
| 4A-7 | 3137 | EVQLVESGGGLVQPGGSLRLSCAASGRTFGDYIMGWFRQAPGKERESVAAINWSAGY |
| | | TPYADSVKGRFTISADNSKNTAYLQMNSLKPEDTAVYYCATDPPLFWGHVDLWGQGT |
| | | LVTVSS |
| 4A-71 | 3138 | EVQLVESGGGLVQPGGSLRLSCAASGRTFSDDTMGWFRQAPGKEREWVASINWSGGS |
| | | TYYADSVKGRFTISADNSKNTAYLQMNSLKPEDTAVYYCATDPPLFWGQGTLVTVSS |
| 4A-23 | 3139 | EVQLVESGGGLVQPGGSLRLSCAASGRTFSDDAMGWFRQAPGKEREFVAGISWNGGSI |
| | | YYADSVKGRFTISADNSKNTAYLQMNSLKPEDTAVYYCATDPPLFWGQGTLVTVSS |
| 4A-9 | 3140 | EVQLVESGGGLVQPGGSLRLSCAASGFTFDDYEMGWFRQAPGKEREFVAAISWRGGT |
| | | TYYADSVKGRFTISADNSKNTAYLQMNSLKPEDTAVYYCAADRRGLASTRAGDYDW |
| | | GQGTLVTVSS |
| 4A-72 | 3141 | EVQLVESGGGLVQPGGSLRLSCAASGRTFGDDTMGWFRQAPGKEREFVAAINWSGGY |
| | | TPYADSVKGRFTISADNSKNTAYLQMNSLKPEDTAVYYCATDPPLFWGHVDLWGQGT |
| | | LVTVSS |
| 4A-73 | 3142 | EVQLVESGGGLVQPGGSLRLSCAASGRTFSDDAMGWFRQAPGKEREFVAAINWSGGS |
| | | TRYADSVKGRFTISADNSKNTAYLQMNSLKPEDTAVYYCATDPPLFWGQGTLVTVSS |
| 4A-29 | 3143 | EVQLVESGGGLVQPGGSLRLSCAASGVTLDDYAMGWFRQAPGKEREFVAVINWSGGS |
| | | TDYADSVKGRFTISADNSKNTAYLQMNSLKPEDTAVYYCARGGGWVPSSTSESLNWY |
| | | FDRWGQGTLVTVSS |
| 4A-41 | 3144 | EVQLVESGGGLVQPGGSLRLSCAASGRTFGDYIMGWFRQAPGKERESVAAINWSGGTT |
| | | PYADSVKGRFTISADNSKNTAYLQMNSLKPEDTAVYYCATDPPLFCCHVDLWGQGTL |
| | | VTVSS |
| 4A-74 | 3145 | EVQLVESGGGLVQPGGSLRLSCAASGLTFSDDTMGWFRQAPGKEREFVAAVSWSGGN |
| | | TYYADSVKGRFTISADNSKNTAYLQMNSLKPEDTAVYYCATDPPLFWGQGTLVTVSS |
| 4A-75 | 3146 | EVQLVESGGGLVQPGGSLRLSCAASGRTFGDDTMGWFRQAPGKEREFVAAINWTGGY |
| | | TPYADSVKGRFTISADNSKNTAYLQMNSLKPEDTAVYYCATDPPLFWGQGTLVTVSS |
| 4A-31 | 3147 | EVQLVESGGGLVQPGGSLRLSCAASGRTFGDYIMGWFRQAPGKEREFVATINWTAGY |
| | | TYYADSVKGRFTISADNSKNTAYLQMNSLKPEDTAVYYCATDPPLFCWHFDHWGQGT |
| | | LVTVSS |
| 4A-32 | 3148 | EVQLVESGGGLVQPGGSLRLSCAASGRTFGDDTMGWFRQAPGKEREFVAAINWSGGN |
| | | TDYADSVKGRFTISADNSKNTAYLQMNSLKPEDTAVYYCATDPPLFWGQGTLVTVSS |
| 4A-15 | 3149 | EVQLVESGGGLVQPGGSLRLSCAASGRTFGDYTMGWFRQAPGKEREFVAAINWSGGN |
| | | TYYADSVKGRFTISADNSKNTAYLQMNSLKPEDTAVYYCATDPPLFWGQGTLVTVSS |
| 4A-14 | 3150 | EVQLVESGGGLVQPGGSLRLSCAASGRTFSDDTMGWFRQAPGKEREFVAGINWSGNG |
| | | VYYADSVKGRFTISADNSKNTAYLQMNSLKPEDTAVYYCATDPPLFWGQGTLVTVSS |
| 4A-76 | 3151 | EVQLVESGGGLVQPGGSLRLSCAASGRTFGDYAMGWFRQAPGKERELVAPINWSGGS |
| | | TYYADSVKGRFTISADNSKNTAYLQMNSLKPEDTAVYYCATDPPLFWGQGTLVTVSS |
| 4A-50 | 3152 | EVQLVESGGGLVQPGGSLRLSCAASGGTFSNSGMGWFRQAPGKERELVAVVNWSGRR |
| | | TYYADSVKGRFTISADNSKNTAYLQMNSLKPEDTAVYYCAVPWMDYNRRDWGQGTL |
| | | VTVSS |
| 4A-17 | 3153 | EVQLVESGGGLVQPGGSLRLSCAASGQLANFASYAMGWFRQAPGKEREFVAAITRSGS |
| | | STVYADSVKGRFTISADNSKNTAYLQMNSLKPEDTAVYYCATTMNPNPRWGQGTLVT |
| | | VSS |
| 4A-37 | 3154 | EVQLVESGGGLVQPGGSLRLSCAASGRTFSDDIMGWFRQAPGKEREFVAAINWTGGST |
| | | YYADSVKGRFTISADNSKNTAYLQMNSLKPEDTAVYYCATDPPLFWGQGTLVTVSS |
| 4A-44 | 3155 | EVQLVESGGGLVQPGGSLRLSCAASGRTFGDYIMGWFRQAPGKEREIVAAINWSAGYT |
| | | AYADSVKGRFTISADNSKNTAYLQMNSLKPEDTAVYYCATARPNTGWHFDHWGQGT |
| | | LVTVSS |
| 4A-77 | 3156 | EVQLVESGGGLVQPGGSLRLSCAASGRTFSDDTMGWFRQAPGKEREWVGSINWSGGS |
| | | TYYADSVKGRFTISADNSKNTAYLQMNSLKPEDTAVYYCATDPPLFWGQGTLVTVSS |
| 4A-78 | 3157 | EVQLVESGGGLVQPGGSLRLSCAASGRTFSDDTMGWFRQAPGKEREFVAGMTWSGSS |
| | | TFYADSVKGRFTISADNSKNTAYLQMNSLKPEDTAVYYCATDPPLFWGQGTLVTVSS |
| 4A-79 | 3158 | EVQLVESGGGLVQPGGSLRLSCAASGRTFGDYIMGWFRQAPGKERECVAAINWSGDY |
| | | TDYADSVKGRFTISADNSKNTAYLQMNSLKPEDTAVYYCATDPPLFWGQGTLVTVSS |
| 4A-8 | 3159 | EVQLVESGGGLVQPGGSLRLSCAASGRTFGDYIMGWFRQAPGKEREFVGGINWSGGY |
| | | TYYADSVKGRFTISADNSKNTAYLQMNSLKPEDTAVYYCATDPPLFWGQGTLVTVSS |
| 4A-81 | 3160 | EVQLVESGGGLVQPGGSLRLSCAASGRTFSDDTMGWFRQAPGKEREFVAAVNWSGGS |
| | | TYYADSVKGRFTISADNSKNTAYLQMNSLKPEDTAVYYCATDPPLFWGQGTLVTVSS |
| 4A-82 | 3161 | EVQLVESGGGLVQPGGSLRLSCAASGRTFGDYAMGWFRQAPGKEREFVAAINWSGGY |
| | | TRYADSVKGRFTISADNSKNTAYLQMNSLKPEDTAVYYCATDPPLFWGQGTLVTVSS |
| 4A-83 | 3162 | EVQLVESGGGLVQPGGSLRLSCAASGRTFGDDTMGWFRQAPGKEREFVAAINWSGGY |
| | | TPYADSVKGRFTISADNSKNTAYLQMNSLKPEDTAVYYCATDPPLFWGQGTLVTVSS |
| 4A-35 | 3163 | EVQLVESGGGLVQPGGSLRLSCAASGRTFGDYIMGWFRQAPGKERESVAAINWSAGY |
| | | TAYADSVKGRFTISADNSKNTAYLQMNSLKPEDTAVYYCARASPNTGWHFDRWGQG |
| | | TLVTVSS |
| 4A-45 | 3164 | EVQLVESGGGLVQPGGSLRLSCAASGRTFGDYIMGWFRQAPGKEREFVAAINWSGGY |
| | | THYADSVKGRFTISADNSKNTAYLQMNSLKPEDTAVYYCATDPPLFWGQGTLVTVSS |
| 4A-84 | 3165 | EVQLVESGGGLVQPGGSLRLSCAASGRTFSDDTMGWFRQAPGKEREFVAAITWSGGR |
| | | TRYADSVKGRFTISADNSKNTAYLQMNSLKPEDTAVYYCATDRPLFWGQGTLVTVSS |
| 4A-85 | 3166 | EVQLVESGGGLVQPGGSLRLSCAASGRTFGDYIMGWFRQAPGKERESVAAINWSGGY |
| | | TAYADSVKGRFTISADNSKNTAYLQMNSLKPEDTAVYYCATASPNTGWHFDHWGQG |
| | | TLVTVSS |
| 4A-86 | 3167 | EVQLVESGGGLVQPGGSLRLSCAASGRTFSDDTMGWFRQAPGKEREFVAAIHWSGSST |
| | | RYADSVKGRFTISADNSKNTAYLQMNSLKPEDTAVYYCATDPPLFWGQGTLVTVSS |
| 4A-87 | 3168 | EVQLVESGGGLVQPGGSLRLSCAASGRTFSDYTMGWFRQAPGKEREWVAAINWSGGT |
| | | TYYADSVKGRFTISADNSKNTAYLQMNSLKPEDTAVYYCATDPPLFWGQGTLVTVSS |
| 4A-88 | 3169 | EVQLVESGGGLVQPGGSLRLSCAASGRTFGDDTMGWFRQAPGKEREFVAAINWSGDN |
| | | THYADSVKGRFTISADNSKNTAYLQMNSLKPEDTAVYYCATDPPLFWGQGTLVTVSS |
| 4A-89 | 3170 | EVQLVESGGGLVQPGGSLRLSCAASGFAFGDNWIGWFRQAPGKEREWVASISSGGTTA |
| | | YADNVKGRFTIIADNSKNTAYLQMNSLKPEDTAVYYCAHRGGWLRPWGYWGQGTLV |
| | | TVSS |
| 4A-9 | 3171 | EVQLVESGGGLVQPGGSLRLSCAASGRTFSDDAMGWFRQAPGKEREFVGRINWSGGN |
| | | TYYADSVKGRFTISADNSKNTAYLQMNSLKPEDTAVYYCATDPPLFWGQGTLVTVSS |
| 4A-91 | 3172 | EVQLVESGGGLVQPGGSLRLSCAASGRTFSDDTMGWFRQAPGKEREFVGGISWSGGN |
| | | TYYADSVKGRFTISADNSKNTAYLQMNSLKPEDTAVYYCATDPPLFWGQGTLVTVSS |
| 4A-92 | 3173 | EVQLVESGGGLVQPGGSLRLSCAASGRTFSDDTMGWFRQAPGKEREFVAAINWSGGS |
| | | TYYADSVKGRFTISADNSKNTAYLQMNSLKPEDTAVYYCATDPPLFWGQGTLVTVSS |
| 4A-46 | 3174 | EVQLVESGGGLVQPGGSLRLSCAASGRTFGDDTMGWFRQAPGKEREFVAAINWSGGY |
| | | TYYADSVKGRFTISADNSKNTAYLQMNSLKPEDTAVYYCATDPPLFWGQGTLVTVSS |
| 4A-20 | 3175 | EVQLVESGGGLVQPGGSLRLSCAASGRTFGDYIMGWFRQAPGKEREFVAAINWSADY |
| | | TAYADSVKGRFTISADNSKNTAYLQMNSLKPEDTAVYYCATDPPLFCWHFDHWGQGT |
| | | LVTVSS |
| 4A-93 | 3176 | EVQLVESGGGLVQPGGSLRLSCAASGRTFSDDAMGWFRQAPGKEREFVAAINWSGSST |
| | | YYADSVKGRFTISADNSKNTAYLQMNSLKPEDTAVYYCATDPPLFWGQGTLVTVSS |
| 4A-4 | 3177 | EVQLVESGGGLVQPGGSLRLSCAASGRTFGDYIMGWFRQAPGKEREMVAAINWIAGY |
| | | TADADSVRRLFTITADNNKNTAHLMMNLLKPENTAVYYCAEPSPNTGWHFDHWGQG |
| | | TLVTVSS |
| 4A-2 | 3178 | EVQLVESGGGLVQPGGSLRLSCAASGRTFGDDTMGWFRQAPGKEREFVAAINWSGGN |
| | | TPYADSVKGRFTISADNSKNTAYLQMNSLKPEDTAVYYCATDPPLFWGQGTLVTVSS |
| 4A-94 | 3179 | EVQLVESGGGLVQPGGSLRLSCAASGRTFSDDTMGWFRQAPGKEREFVAAINWSGDN |
| | | THYADSVKGRFTISADNSKNTAYLQMNSLKPEDTAVYYCATDPPLFWGQGTLVTVSS |
| 4A-95 | 3180 | EVQLVESGGGLVQPGGSLRLSCAASGRTFGDYIMGWFRQAPGKEREIVAAINWSAGYT |
| | | AYADSVKGRFTISADNSKNTAYLQMNSLKPEDTAVYYCATAPPLFCWHFDHWGQGTL |
| | | VTVSS |
| 4A-12 | 3181 | EVQLVESGGGLVQPGGSLRLSCAASGFTFGDYVMGWFRQAPGKEREIVAAINWNAGY |
| | | TAYADSVRGLFTITADNSKNTAYLQMNSLKPEDTAVYYCAKASPNTGWHFDHWGQG |
| | | TLVTVSS |
| 4A-30 | 3182 | EVQLVESGGGLVQPGGSLRLSCAASGRTFGDYTMGWFRQAPGKEREFVAAINWTGGY |
| | | TYYADSVKGRFTISADNSKNTAYLQMNSLKPEDTAVYYCATDPPLFWGQGTLVTVSS |
| 4A-27 | 3183 | EVQLVESGGGLVQPGGSLRLSCAASGRTFGDYIMGWFRQAPGKEREIVAAINWSAGYT |
| | | AYADSVKGLFTITADNSKNTAYLQMNILKPEDTAVYYCARATPNTGWHFDHWGQGTL |
| | | VTVSS |
| 4A-22 | 3184 | EVQLVESGGGLVQPGGSLRLSCAASGRTFGDYIMGWFRQAPGKEREFVAAINWSGDN |
| | | THYADSVKGRFTISADNSKNTAYLQMNSLKPEDTAVYYCATDPPLFWGQGTLVTVSS |
| 4A-96 | 3185 | EVQLVESGGGLVQPGGSLRLSCAASGRTFGDYIMGWFRQAPGKEREIVAAINWSAGYT |
| | | PYADSVKGRFTISADNSKNTAYLQMNSLKPEDTAVYYCATDPPLFCCHFDHWGQGTL |
| | | VTVSS |
| 4A-97 | 3186 | EVQLVESGGGLVQPGGSLRLSCAASGRTFGDYIMGWFRQAPGKERESVAAINWSAGY |
| | | TAYADSVKGRFTISADNSKNTAYLQMNSLKPEDTAVYYCATAPPNTGWHFDHWGQG |
| | | TLVTVSS |
| 4A-98 | 3187 | EVQLVESGGGLVQPGGSLRLSCAASGFTWGDYTMGWFRQAPGKEREFVAAINWSGG |
| | | NTYYADSVKGRFTISADNSKNTAYLQMNSLKPEDTAVYYCAADRRGLASTRAADYD |
| | | WGQGTLVTVSS |
| 4A-99 | 3188 | EVQLVESGGGLVQPGGSLRLSCAASGIPSTLRAMGWFRQAPGKEREFVAAVSSLGPFT |
| | | RYADSVKGRFTISADNSKNTAYLQMNSLKPEDTAVYYCAAKPGWVARDPSQYNWGQ |
| | | GTLVTVSS |
| 4A-100 | 3189 | EVQLVESGGGLVQPGGSLRLSCAASGFSFDDDYVMGWFRQAPGKEREFVAAINWSGG |
| | | STYYADSVKGRFTISADNSKNTAYLQMNSLKPEDTAVYYCAADRRGLASTRAADYDW |
| | | GQGTLVTVSS |
| 4A-101 | 3190 | EVQLVESGGGLVQPGGSLRLSCAASGRTFSNAAMGWFRQAPGKEREFVARILWTGAS |
| | | RSYADSVKGRFTISADNSKNTAYLQMNSLKPEDTAVYYCATTENPNPRWGQGTLVTV |
| | | SS |
| 4A-102 | 3191 | EVQLVESGGGLVQPGGSLRLSCAASGGTFGVYHMGWFRQAPGKEREGVAAINMSGD |
| | | DSAYADSVKGRFTISADNSKNTAYLQMNSLKPEDTAVYYCAILVGPGQVEFDHWGQG |
| | | TLVTVSS |
| 4A-103 | 3192 | EVQLVESGGGLVQPGGSLRLSCAASGFTFSSYYMGWFRQAPGKEREFVARI-- |
| | | SGSTFYADSVKGRFTISADNSKNTAYLQMNSLKPEDTAVYYCAALPFVCPSGSYSDYG |
| | | DEYDWGQGTLVTVSS |
| 4A-104 | 3193 | EVQLVESGGGLVQPGGSLRLSCAASGRTFSGDFMGWFRQAPGKEREFVGRINWSGGN |
| | | TYYADSVRGLFTITADNNKNTAYLMMNLLKPEDTAVYYCPTDPPLFWGLGTLVTWSS |
| 4A-105 | 3194 | EVQLVESGGGLVQPGGSLRLSCAASGSTLRDYAMGWFRQAPGKERESVAAITWSGGS |
| | | TAYADSVKGRFTISADNSKNTAYLQMNSLKPEDTAVYYCASLLAGDRYFDYWGQGTL |
| | | VTVSS |
| 4A-106 | 3195 | EVQLVESGGGLVQPGGSLRLSCAASGFTFDDYTMGWFRQAPGKEREFVAAITDNGGS |
| | | KYYADSVKGRFTISADNSKNTAYLQMNSLKPEDTAVYYCAADRRGLASTRAADYDW |
| | | GQGTLVTVSS |
| 4A-107 | 3196 | EVQLVESGGGLVQPGGSLRLSCAASGGTFSSYGMGWFRQAPGKEREFVAAINWSGAS |
| | | TYYADSVKGRFTISADNSKNTAYLQMNSLKPEDTAVYYCARDWRDRTWGNSLDYWG |
| | | QGTLVTVSS |
| 4A-108 | 3197 | EVQLVESGGGLVQPGGSLRLSCAASGFSFDDDYVMGWFRQAPGKEREFVAAISWSED |
| | | NTYYADSVKGRFTISADNSKNTAYLQMNSLKPEDTAVYYCAADRRGLASTRAADYD |
| | | WGQGTLVTVSS |
| 4A-109 | 3198 | EVQLVESGGGLVQPGGSLRLSCAASGFSFDDDYVMGWFRQAPGKEREFVAAVSGSGD |
| | | DTYYADSVKGRFTISADNSKNTAYLQMNSLKPEDTAVYYCAADRRGLASTRAADYD |
| | | WGQGTLVTVSS |
| 4A-110 | 3199 | EVQLVESGGGLVQPGGSLRLSCAASGNIAAINVMGWFRQAPGKEREFVAAISASGRRT |
| | | DYADSVKGRFTISADNSKNTAYLQMNSLKPEDTAVYYCARRVYYYDSSGPPGVTFDI |
| | | WGQGTLVTVSS |
| 4A-111 | 3200 | EVQLVESGGGLVQPGGSLRLSCAASGIITSRYVMGWFRQAPGKEREGVAAISTGGSTIY |
| | | ADSVKGRFTISADNSKNTAYLQMNSLKPEDTAVYYCARQDSSSPYFDYWGQGTLVTV |
| | | SS |
| 4A-112 | 3201 | EVQLVESGGGLVQPGGSLRLSCAASGFSFDDDYVMGWFRQAPGKEREFVAAISNSGLS |
| | | TYYADSVKGRFTISADNSKNTAYLQMNSLKPEDTAVYYCAADRRGLASTRAADYDW |
| | | GQGTLVTVSS |
| 4A-113 | 3202 | EVQLVESGGGLVQPGGSLRLSCAASGSISSINVMGWFRQAPGKEREFVATMRWSTGST |
| | | YYADSVKGRFTISADNSKNTAYLQMNSLKPEDTAVYYCAQRVRGFFGPLRTTPSWYE |
| | | WGQGTLVTVSS |
| 4A-114 | 3203 | EVQLVESGGGLVQPGGSLRLSCAASGLTFILYRMGWFRQAPGKEREFVAAINNFGTTK |
| | | YADSVKGRFTISADNSKNTAYLQMNSLKPEDTAVYYCARTHYDFWSGYTSRTPNYFD |
| | | YWGQGTLVTVSS |
| 4A-115 | 3204 | EVQLVESGGGLVQPGGSLRLSCAASGGTFSVYHMGWFRQAPGKEREPVAAISWSGGS |
| | | TAYADSVKGRFTISADNSKNTAYLQMNSLKPEDTAVYYCAAVNTWTSPSFDSWGQGT |
| | | LVTVSS |
| 4A-116 | 3205 | EVQLVESGGGLVQPGGSLRLSCAASGRAFSTYGMGWFRQAPGKEREFVAGINWSGDT |
| | | PYYADSVKGRFTISADNSKNTAYLQMNSLKPEDTAVYYCAREVGPPPGYFDLWGQGT |
| | | LVTVSS |
| 4A-117 | 3206 | EVQLVESGGGLVQPGGSLRLSCAASGRTFSDIAMGWFRQAPGKEREFVASINWGGGNT |
| | | YYADSVKGRFTISADNSKNTAYLQMNSLKPEDTAVYYCAAKGIWDYLGRRDFGDWG |
| | | QGTLVTVSS |
| 4A-118 | 3207 | EVQLVESGGGLVQPGGSLRLSCAASGRTFSSARMGWFRQAPGKEREFVAAISWSGDNT |
| | | HYADSVKGRFTISADNSKNTAYLQMNSLKPEDTAVYYCATTENPNPRWGQGTLVTVS |
| | | S |
| 4A-119 | 3208 | EVQLVESGGGLVQPGGSLRLSCAASGFAFSSYAMGWFRQAPGKEREWVATINGDDYT |
| | | YYADSVKGRFTISADNSKNTAYLQMNSLKPEDTAVYYCVATPGGYGLWGQGTLVTVS |
| | | S |
| 4A-120 | 3209 | EVQLVESGGGLVQPGGSLRLSCAASGITFRRHDMGWFRQAPGKEREFVAAIRWSSSST |
| | | VYADSVKGRFTISADNSKNTAYLQMNSLKPEDTAVYYCAADRGVYGGRWYRTSQYT |
| | | WGQGTLVTVSS |
| 4A-121 | 3210 | EVQLVESGGGLVQPGGSLRLSCAASGTAASFNPMGWFRQAPGKEREFVAAITSGGSTN |
| | | YADSVKGRFTISADNSKNTAYLQMNSLKPEDTAVYYCAAIAYEEGVYRWDWGQGTL |
| | | VTVSS |
| 4A-122 | 3211 | EVQLVESGGGLVQPGGSLRLSCAASGNINIINYMGWFRQAPGKEREGVAAIHWNGDST |
| | | AYADSVKGRFTISADNSKNTAYLQMNSLKPEDTAVYYCASGPPYSNYFAYWGQGTLV |
| | | TVSS |
| 4A-123 | 3212 | EVQLVESGGGLVQPGGSLRLSCAASGFTFDDYAMGWFRQAPGKERESVAAISGSGGST |
| | | AYADSVKGRFTISADNSKNTAYLQMNSLKPEDTAVYYCAKIMGSGRPYFDHWGQGTL |
| | | VTVSS |
| 4A-124 | 3213 | EVQLVESGGGLVQPGGSLRLSCAASGNIFTRNVMGWFRQAPGKEREFVAAITSSGSTN |
| | | YADSVKGRFTISADNSKNTAYLQMNSLKPEDTAVYYCARPSSDLQGGVDYWGQGTLV |
| | | TVSS |
| 4A-125 | 3214 | EVQLVESGGGLVQPGGSLRLSCAASGRTFSSIAMGWFRQAPGKEREFVASINWGGGNT |
| | | IYADSVKGRFTISADNSKNTAYLQMNSLKPEDTAVYYCAAKGIWDYLGRRDFGDWGQ |
| | | GTLVTVSS |
| 4A-126 | 3215 | EVQLVESGGGLVQPGGSLRLSCAASGIPSTLRAMGWFRQAPGKEREFVAAVSSLGPFT |
| | | RYADSVKGRFTISADNSKNTAYLQMNSLKPEDTAVYYCAAKPGWVARDPSEYNWGQ |
| | | GTLVTVSS |
| 4A-127 | 3216 | EVQLVESGGGLVQPGGSLRLSCAASGFTLDDSAMGWFRQAPGKEREWVAAITNGGST |
| | | YYADSVKGRFTISADNSKNTAYLQMNSLKPEDTAVYYCARFARGSPYFDFWGQGTLV |
| | | TVSS |
| 4A-128 | 3217 | EVQLVESGGGLVQPGGSLRLSCAASGSISSFNAMGWFRQAPGKERESVAAIDWDGSTA |
| | | YADSVKGRFTISADNSKNTAYLQMNSLKPEDTAVYYCARGGGYYGSGSFEYWGQGTL |
| | | VTVSS |
| 4A-129 | 3218 | EVQLVESGGGLVQPGGSLRLSCAASGNIFSDNIIGWFRQAPGKEREMVAYYTSGGSIDY |
| | | ADSVKGRFTISADNSKNTAYLQMNSLKPEDTAVYYCARGTAVGRPPPGGMDVWGQG |
| | | TLVTVSS |
| 4A-130 | 3219 | EVQLVESGGGLVQPGGSLRLSCAASGSISSIGAMGWFRQAPGKEREGVAAISSSGSSTV |
| | | YADSVKGRFTISADNSKNTAYLQMNSLKPEDTAVYYCARVPPGQAYFDSWGQGTLVT |
| | | VSS |
| 4A-131 | 3220 | EVQLVESGGGLVQPGGSLRLSCAASGFTFDDYGMGWFRQAPGKERELVATITWSGDS |
| | | TYYADSVKGRFTISADNSKNTAYLQMNSLKPEDTAVYYCAKGGSWYYDSSGYYGRW |
| | | GQGTLVTVSS |
| 4A-132 | 3221 | EVQLVESGGGLVQPGGSLRLSCAASGRTFSNYTMGWFRQAPGKEREWVSAISWSTGST |
| | | YYADSVKGRFTISADNSKNTAYLQMNSLKPEDTAVYYCAADRYGPPWYDWGQGTLV |
| | | TVSS |
| 4A-133 | 3222 | EVQLVESGGGLVQPGGSLRLSCAASGSTNYMGWFRQAPGKEREGVAAISMSGDDTIY |
| | | ADSVKGRFTISADNSKNTAYLQMNSLKPEDTAVYYCARIGLRGRYFDLWGQGTLVTV |
| | | SS |
| 4A-134 | 3223 | EVQLVESGGGLVQPGGSLRLSCAASGGTFSSVGMGWFRQAPGKERELVAVINWSGAR |
| | | TYYADSVKGRFTISADNSKNTAYLQMNSLKPEDTAVYYCAVPWMDYNRRDWGQGTL |
| | | VTVSS |
| 4A-135 | 3224 | EVQLVESGGGLVQPGGSLRLSCAASGRIFTNTAMGWFRQAPGKEREGVAAINWSGGST |
| | | AYADSVKGRFTISADNSKNTAYLQMNSLKPEDTAVYYCARTSGSYSFDYWGQGTLVT |
| | | VSS |
| 4A-136 | 3225 | EVQLVESGGGLVQPGGSLRLSCAASGEEFSDHWMGWFRQAPGKEREFVGAIHWSGGR |
| | | TYYADSVKGRFTISADNSKNTAYLQMNSLKPEDTAVYYCAADRRGLASTRAADYDW |
| | | GQGTLVTVSS |
| 4A-137 | 3226 | EVQLVESGGGLVQPGGSLRLSCAASGRTFSSIAMGWFRQAPGKEREFVAAINWSGART |
| | | AYADSVKGRFTISADNSKNTAYLQMNSLKPEDTAVYYCAAKGIWDYLGRRDFGDWG |
| | | QGTLVTVSS |
| 4A-138 | 3227 | EVQLVESGGGLVQPGGSLRLSCAASGSTSSLRTMGWFRQAPGKEREGVAAISSRDGSTI |
| | | YADSVKGRFTISADNSKNTAYLQMNSLKPEDTAVYYCARDDSSSPYFDYWGQGTLVT |
| | | VSS |
| 4A-139 | 3228 | EVQLVESGGGLVQPGGSLRLSCAASGGGTFGSYAMGWFRQAPGKEREFVAAISIASGA |
| | | SGGTTNYADSVKGRFTISADNSKNTAYLQMNSLKPEDTAVYYCATTMNPNPRWGQGT |
| | | LVTVSS |
| 4A-140 | 3229 | EVQLVESGGGLVQPGGSLRLSCAASGRTFSNAAMGWFRQAPGKEREFVARITWNGGS |
| | | TFYADSVKGRFTISADNSKNTAYLQMNSLKPEDTAVYYCATTENPNPRWGQGTLVTV |
| | | SS |
| 4A-141 | 3230 | EVQLVESGGGLVQPGGSLRLSCAASGIILSDNAMGWFRQAPGKEREFVAAISWLGEST |
| | | YYADSVKGRFTISADNSKNTAYLQMNSLKPEDTAVYYCAADRRGLASTRAADYDWG |
| | | QGTLVTVSS |
| 4A-142 | 3231 | EVQLVESGGGLVQPGGSLRLSCAASGRTFGDYIMGWFRQAPGKERESVAAINWNGGY |
| | | TAYADSVKGRFTISADNSKNTAYLQMNSLKPEDTAVYYCATTSPNTGWHYYRWGQG |
| | | TLVTVSS |
| 4A-143 | 3232 | EVQLVESGGGLVQPGGSLRLSCAASGFNFNWYPMGWFRQAPGKERESVAAISWTGVS |
| | | TYTAYADSVKGRFTISADNSKNTAYLQMNSLKPEDTAVYYCARWGPGPAGGSPGLVG |
| | | FDYWGQGTLVTVSS |
| 4A-144 | 3233 | EVQLVESGGGLVQPGGSLRLSCAASGSIRSVSVMGWFRQAPGKEREAVAAISWSGVGT |
| | | AYADSVKGRFTISADNSKNTAYLQMNSLKPEDTAVYYCAAYQRGWGDWGQGTLVTV |
| | | SS |
| 4A-145 | 3234 | EVQLVESGGGLVQPGGSLRLSCAASGMTFRLYAMGWFRQAPGKEREFVGAINWLSES |
| | | TYYADSVKGRFTISADNSKNTAYLQMNSLKPEDTAVYYCAAKPGWVARDPSEYNWG |
| | | QGTLVTVSS |
| 4A-146 | 3235 | EVQLVESGGGLVQPGGSLRLSCAASGRTFSDDAMGWFRQAPGKEREFVAAINWSGGS |
| | | TYYADSVKGRFTISADNSKNTAYLQMNSLKPEDTAVYYCATDPPLFWGQGTMVTVSS |
| 4A-147 | 3236 | EVQLVESGGGLVQPGGSLRLSCAASGGTFSVYAMGWFRQAPGKEREGVAAISMSGDD |
| | | AAYADSVKGRFTISADNSKNTAYLQMNSLKPEDTAVYYCAKISKDDGGKPRGAFFDS |
| | | WGQGTLVTVSS |
| 4A-148 | 3237 | EVQLVESGGGLVQPGGSLRLSCAASGFALGYYAMGWFRQAPGKERESVAAISSRDGST |
| | | AYADSVKGRFTISADNSKNTAYLQMNSLKPEDTAVYYCARLATGPQAYFHHWGQGTL |
| | | VTVSS |
| 4A-149 | 3238 | EVQLVESGGGLVQPGGSLRLSCAASGFNLDDYAMGWFRQAPGKERESVAAISWDGGA |
| | | TAYADSVKGRFTISADNSKNTAYLQMNSLKPEDTAVYYCARVGRGTTAFDSWGQGTL |
| | | VTVSS |
| 4A-150 | 3239 | EVQLVESGGGLVQPGGSLRLSCAASGNTFSGGFMGWFRQAPGKEREFVASIRSGARTY |
| | | YADSVKGRFTISADNSKNTAYLQMNSLKPEDTAVYYCAQRVRGFFGPLRTTPSWYEW |
| | | GQGTLVTVSS |
| 4A-151 | 3240 | EVQLVESGGGLVQPGGSLRLSCAASGSIRSINIMGWFRQAPGKEREAVAAISWSGGSTV |
| | | YADSVKGRFTISADNSKNTAYLQMNSLKPEDTAVYYCASLLAGDRYFDYWGQGTLVT |
| | | VSS |
| 5A-1 | 3241 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCAKEDVGKPFDWGQGTLVT |
| | | VSS |
| 5A-2 | 3242 | EVQLVESGGGLVQPGGSLRLSCAASGLRFDDYAMGWFRQAPGKERELVAIKFSGGTT |
| | | DYADSVKGRFTISADNSKNTAYLQMNSLKPEDTAVYYCASWDGLIGLDAYEYDWGQ |
| | | GTLVTVSS |
| 5A-3 | 3243 | EVQLVESGGGLVQPGGSLRLSCAASGSIFSIDVMGWFRQAPGKEREFVAGISWSGDSTL |
| | | YADSVKGRFTISADNSKNTAYLQMNSLKPEDTAVYYCAAFDGYTGSDWGQGTLVTVS |
| | | S |
| 5A-4 | 3244 | EVQLVESGGGLVQPGGSLRLSCAASGFTLADYAMGWFRQAPGKEREFVAVITCSGGST |
| | | DYADSVKGRFTISADNSKNTAYLQMNSLKPEDTAVYYCAADDCYIGCGWGQGTLVTV |
| | | SS |
| 5A-5 | 3245 | EVQLVESGGGLVQPGGSLRLSCAASGRTFSSIAMGWFRQAPGKERELVAEITEGGISPS |
| | | GDNIYYADSVKGRFTISADNSKNTAYLQMNSLKPEDTAVYYCAAELHSSDYTSPGAES |
| | | DYGWGQGTLVTVSS |
| 5A-6 | 3246 | EVQLVESGGGLVQPGGSLRLSCAASGPTFSSYAMMGWFRQAPGKEREWVAAINNFGT |
| | | TKYADSVKGRFTISADNSKNTAYLQMNSLKPEDTAVYYCAASASDYGLGLELFHDEY |
| | | NWGQGTLVTVSS |
| 5A-7 | 3247 | EVQLVESGGGLVQPGGSLRLSCAASGSTGYMGWFRQAPGKEREFVAAIHSGGSTNYA |
| | | DSVKGRFTISADNSKNTAYLQMNSLKPEDTAVYYCATVATALIWGQGTLVTVSS |
| 5A-8 | 3248 | EVQLVESGGGLVQPGGSLRLSCAASGRPFSEYTMGWFRQAPGKEREFVSSIHWGGRGT |
| | | NYADSVKGRFTISADNSKNTAYLQMNSLKPEDTAVYYCAAELHSSDYTSPGAYAWGQ |
| | | GTLVTVSS |
| 5A-9 | 3249 | EVQLVESGGGLVQPGGSLRLSCAASGLTLSTYGMGWFRQAPGKEREFVAHIPRSTYSP |
| | | YYADSVKGRFTISADNSKNTAYLQMNSLKPEDTAVYYCAAIGDGAVWGQGTLVTVSS |
| 5A-10 | 3250 | EVQLVESGGGLVQPGGSLRLSCAASGFTFNNHNMGWFRQAPGKEREFVAAISSYSHTA |
| | | YADSVKGRFTISADNSKNTAYLQMNSLKPEDTAVYYCALQPFGASNYRWGQGTLVTV |
| | | SS |
| 5A-11 | 3251 | EVQLVESGGGLVQPGGSLRLSCAASGGIYRVMGWFRQAPGKERELVASTSSGGGINYA |
| | | DSVKGRFTISADNSKNTAYLQMNSLKPEDTAVYYCAAESWGRQWGQGTLVTVSS |
| 5A-12 | 3252 | EVQLVESGGGLVQPGGSLRLSCAASGYTDSNLWMGWFRQAPGKEREFVAINRSTGSTS |
| | | YADSVKGRFTISADNSKNTAYLQMNSLKPEDTAVYYCATSGSGSPNWGQGTLVTVSS |
| 5A-13 | 3253 | EVQLVESGGGLVQPGGSLRLSCAASGFTFDYYTMGWFRQAPGKEREFVAAIRSSGGLF |
| | | YADSVKGRFTISADNSKNTAYLQMNSLKPEDTAVYYCAAYLDGYSGSWGQGTLVTVS |
| | | S |
| 5A-14 | 3254 | EVQLVESGGGLVQPGGSLRLSCAASGGIFSINVMGWFRQAPGKEREWVSAIRWNGGN |
| | | TAYADSVKGRFTITADNSKNTAYLQMNSLKPEDTAVYYCAGFDGYTGSDWGQGTLVT |
| | | VSS |
| 5A-15 | 3255 | EVQLVESGGGLVQPGGSLRLSCAASGFTFDGAAMGWFRQAPGKEREFVATIRWTNST |
| | | DYADSVKGRFTISADNSKNTAYLQMNSLKPEDTAVYYCARGRYGIVERWGQGTLVTV |
| | | SS |
| 5A-16 | 3256 | EVQLVESGGGLVQPGGSLRLSCAASGRTHSIYPMGWFRQAPGKERELVAAIHSGGATV |
| | | YADSVKGRFTISADNSKNTAYLQMNSLKPEDTAVYYCAARRWIPPGPIWGQGTLVTVS |
| | | S |
| 5A-17 | 3257 | EVQLVESGGGLVQPGGSLRLSCAASGPTFSIYAMGWFRQAPGKEREFVAGIRWSDVYT |
| | | QYADSVKGRFTISADNSKNTAYLQMNSLKPEDTAVYYCALDIDYRDWGQGTLVTVSS |
| 5A-18 | 3258 | EVQLVESGGGLVQPGGSLRLSCAASGLTFDDNIHVMGWFPQAPGKEREFVAAIHWSG |
| | | GSTIYADSVKGRFTINADNSKNTAYLQMNSLKPEDTAVYYCAADVYPQDYGLGYVEG |
| | | KMYYGMDWGQGTLVTVSS |
| 5A-19 | 3259 | EVQLVESGGGLVQPGGSLRLSCAASGLTLDYYAMGWFRQAPGKEREWVASINWSGGS |
| | | TYYADSVKGRFTISADNSKNTAYLQMNSLKPEDTAVYYCAAYGSGEFDWGQGTLVTV |
| | | SS |
| 5A-20 | 3260 | EVQLVESGGGLVQPGGSLRLSCAASGRTIVPYTMGWFRQAPGKERELVAAISPSAFTEY |
| | | ADSVKGRFTISADNSKNTAYLQMNSLKPEDTAVYYCAARRWGYDWGQGTLVTVSS |
| 5A-21 | 3261 | EVQLVESGGGLVQPGGSLRLSCAASGGTFTTYHMGWFRQAPGKEREFVAHISTGGATN |
| | | YADSVKGRFTISADNSKNTAYLQMNSLKPEDTAVYYCATFPAIVTDSDYDLGNDWGQ |
| | | GTLVTVSS |
| 5A-22 | 3262 | EVQLVESGGGLVQPGGSLRLSCAASGFTFNVFAMGWFRQAPGKEREFVAAINWSDSRT |
| | | DYADSVKGRFTISADNSKNTAYLQMNSLKPEDTAVYYCASGSDNRARELSRYEYVWG |
| | | QGTLVTVSS |
| 5A-23 | 3263 | EVQLVESGGGLVQPGGSLRLSCAASGSIFSIDVMGWFRQAPGKEREFVAAISWSGESTL |
| | | YADSVKGRFTISADNSKNTAYLQMNSLKPEDTAVYYCAAFDGYSGSDWGQGTLVTVS |
| | | S |
| 5A-24 | 3264 | EVQLVESGGGLVQPGGSLRLSCAASGFTFSSYSMGWFRQAPGKEREFVAAISSYSHTA |
| | | YADSVKGRFTIIADNSKNTAYLQMNSLKPEDTAVYYCALQPFGASSYRWGQGTLVTVS |
| | | S |
| 5A-25 | 3265 | EVQLVESGGGLVQPGGSLRLSCAASGNTFSINVMGWFRQAPGKEREFVAAIHWSGDST |
| | | LYADSGKGRFTIIADNNKNTAYLQMISLKPEDTAVYYCAAFDGYSGNHWGQGTLVTV |
| | | SS |
| 5A-26 | 3266 | EVQLVESGGGLVQPGGSLRLSCAASGRTISSYIMGWFRQAPGKERELVARIYTGGDTIY |
| | | ADSVKGRFTISADNSKNTAYLQMNSLKPEDTAVYYCAARTSYNGRYDYIDDYSWGQG |
| | | TLVTVSS |
| 5A-27 | 3267 | EVQLVESGGGLVQPGGSLRLSCAASGRANSINWMGWFRQAPGKEREFVATITPGGNTN |
| | | YADSVKGRFTISADNSKNTAYLQMNSLKPEDTAVYYCAAAAGSTWYGTLYEYDWGQ |
| | | GTLVTVSS |
| 5A-28 | 3268 | EVQLVESGGGLVQPGGSLRLSCAASGGTFSVFAMGWFRQVPGKERELVAEITAGGSTY |
| | | YADSVKGRFTISADNSKNTAYLQMNSLKPEDTAVYYCAVDGPFGWGQGTLVTVSS |
| 5A-29 | 3269 | EVQLVESGGGLVQPGGSLRLSCAASGFTFDDYPMGWFRQAPGKEREGVASVLRGGYT |
| | | WYADSVKGRFTISADNSKNTAYLQMNSLKPEDTAVYYCAKDWATGLAWGQGTLVTV |
| | | SS |
| 5A-30 | 3270 | EVQLVESGGGLVQPGGSLRLSCAASGFALGYYAMGWFRQAPGKEREFVAGIRWTDAY |
| | | TEYADSVKGRFTISADNSKNTAYLQMNSLKPEDTAVYYCAADVSPSYGSRWYWGQGT |
| | | LVTVSS |
| 5A-31 | 3271 | EVQLVESGGGLVQPGGSLRLSCAASGRTLDIHVMGWFRQAPGKEREFVAVINWTGEST |
| | | LYADSVKGRFTISADNSKNTAYLQMNSLKPEDTAVYYCAAFDGYTGNYWGQGTLVT |
| | | VSS |
| 5A-32 | 3272 | EVQLVESGGGLVQPGGSLRLSCAASGFTPDNYAMGWFRQAPGKEREFVAALGWSGVT |
| | | TYHYYADSVKGRFTISADNSKNTAYLQMNSLKPEDTAVYYCASDESDAANWGQGTL |
| | | VTVSS |
| 5A-33 | 3273 | EVQLVESGGGLVQPGGSLRLSCAASGFTFDDYAMGWFRQAPGKERELVATIMWSGNT |
| | | TYYADSVRRRFIIRDNNNKNTAHLQMNSLKPEDTAVYYCATNDDDVWGQGTLVTVSS |
| 5A-34 | 3274 | EVQLVESGGGLVQPGGSLRLSCAASGRTFSRYIMGWFRQAPGKEREFVAAISWSGGDN |
| | | TYYADSVKGRFTISADNSKNTAYLQMNSLKPEDTAVYYCAAYRIVVGGTSPGDWRWG |
| | | QGTLVTVSS |
| 5A-35 | 3275 | EVQLVESGGGLVQPGGSLRLSCAASGPTFSIYAMGWFRQAPGKERELVAGISWNGGST |
| | | NYADSVKGRFTISADNSKNTAYLQMNSLKPEDTAVYYCALRRRFGGQEWGQGTLVTV |
| | | SS |
| 5A-36 | 3276 | EVQLVESGGGLVQPGGSLRLSCAASGRTFSLNAMGWFRQAPGKERELVAAISCGGGST |
| | | YADNGKGRFTIITDNSKNTAYLQMMNLKPEDTAAYYCAADNDMGYCSWGQGTLVTV |
| | | SS |
| 5A-37 | 3277 | EVQLVESGGGLVQPGGSLRLSCAASGSTFSINAMGWFRQAPGKEREFVGGISRSGATTN |
| | | YADSVKGRFTISADNSKNTAYLQMNSLKPEDTAVYYCAADGVPEYSDYASGPVWGQ |
| | | GTLVTVSS |
| 5A-38 | 3278 | EVQLVESGGGLVQPGGSLRLSCAASGRTFSMHAMGWFRQAPGKERELVASISSQGRTN |
| | | YADSVKGRFTISADNSKNTAYLQMNSLKPEDTAVYYCAAEVRNGSDYLPIDWGQGTL |
| | | VTVSS |
| 5A-39 | 3279 | EVQLVESGGGLVQPGGSLRLSCAASGVTLDLYAMGWFRQAPGKEREFVAGIRWTDAY |
| | | TEYADSVKGRFTISADNSKNTAYLQMNSLKPEDTAVYYCAVDIDYRDWGQGTLVTVS |
| | | S |
| 5A-40 | 3280 | EVQLVESGGGLVQPGGSLRLSCAASGLPFTINVMGWFRQAPGKEREFVAAIHWSGLTT |
| | | FYADSVKGLFTITEDNSKNTAHLMMNLLKPEDTAVYCCAELDGYFFAHWGQGTLVTV |
| | | SS |
| 5A-41 | 3281 | EVQLVESGGGLVQPGGSLRLSCAASGRAFSNYAMGWFRQAPGKEREFVAWINNRGTT |
| | | DYADSGSTYYADSVKGRFTISADNSKNTAYLQMNSLKPEDTAVYYCASTDDYGVDW |
| | | GQGTLVTVSS |
| 5A-42 | 3282 | EVQLVESGGGLVQPGGSLRLSCAASGFTPDDYAMGWFRQAPGKEREFVASIGYSGRSN |
| | | SYNYYADSVKGRFTISADNSKNTAYLQMNSLKPEDTAVYYCAIAHGSSTYNWGQGTL |
| | | VTVSS |
| 5A-43 | 3283 | EVQLVESGGGLVQPGGSLRLSCAASGFTLNYYGMGWFPQAPGKEREFVAAITSGGAPH |
| | | YADSVKGRFTINADNSKNTAYLQMNSLKPEDTAVYYCASAYDRGIGYDWGQGTLVT |
| | | VSS |
| 5A-44 | 3284 | EVQLVESGGGLVQPGGSLRLSCAASGLPFSTKSMGWFRQAPGKEREFVAAIHWSGLTS |
| | | YADSVKGRFTISADNSKNTAYLQMNSLKPEDTAVYYCAADRAADFFAQRDEYDWGQ |
| | | GTLVTVSS |
| 5A-45 | 3285 | EVQLVESGGGLVQPGGSLRLSCAASGRTFSINAMGWFPQAPGKERELVAAISWSGEST |
| | | QYADSVKGRFTISADNSKNTAYLQMNSLKPEDTAVYYCAAFDGGSGTQWGQGTLVT |
| | | VSS |
| 5A-46 | 3286 | EVQLVESGGGLVQPGGSLRLSCAASGEEFSDHWMGWFRQAPGKEREFVAAIHWSGDS |
| | | THRNYADSVKGRFTISADNSKNTAYLQMNSLKPEDTAVYYCATVGITLNWGQGTLVT |
| | | VSS |
| 5A-47 | 3287 | EVQLVESGGGLVQPGGSLRLSCAASGFTFGSYDMGWFRQAPGKEREFVTAINWSGAR |
| | | TAYADSVKGRFTISADNSKNTAYLQMNSLKPEDTAVYYCAARSVYSYEYNWGQGTLV |
| | | TVSS |
| 5A-48 | 3288 | EVQLVESGGGLVQPGGSLRLSCAASGLPLDLYAMGWFPPAPGKELEFVAGIRWSDAYT |
| | | EYADSVKGRFTINADNSKNPANLQMNSLKPEDTAVYYCALDIDYRHWGQGTLVTVSS |
| 5A-49 | 3289 | EVQLVESGGGLVQPGGSLRLSCAASGRTSTVNGMGWFRQAPGKEREFVASISQSGAAT |
| | | AYADSVKGRFTISADNSKNTAYLQMNSLKPEDTAVYYCAADRTYSYSSTGYYWGQGT |
| | | LVTVSS |
| 5A-50 | 3290 | EVQLVESGGGLVQPGGSLRLSCAASGFSLDYYGMGWFRQAPGKEREFVAAITSGGTPH |
| | | YADSVKGRFTISADNSKNTAYLQMNSLKPEDTAVYYCASAYNPGIGYDWGQGTLVTV |
| | | SS |
| 5A-51 | 3291 | EVQLVESGGGLVQPGGSLRLSCAASGRPNSINWMGWFRQAPGKERQFVATITPGGNTN |
| | | YADSVKGRFTISADNSKNTAYLLMNSLKPEDTAVYYCAAAAGTTWYGTLYEYDWGQ |
| | | GTLVTVSS |
| 5A-52 | 3292 | EVQLVESGGGLVQPGGSLRLSCAASGEKFSDHWMGWFRQAPGKEREFVATITFSGART |
| | | AYADSVKGRFTISADNSKNTAYLQMNSLKPEDTAVYYCAALIKPSSTDRIFEEWGQGT |
| | | LVTVSS |
| 5A-53 | 3293 | EVQLVESGGGLVQPGGSLRLSCAASGLTVVPYAMGWFRQAPGKEREFVAAIRRSAVT |
| | | NYADSVKGRFTIIADNSKNTAYLLMNSLKPEDTAVYYCAARRWGYHYWGQGTLVTV |
| | | SS |
| 5A-54 | 3294 | EVQLVESGGGLVQPGGSLRLSCAASGTTFNFNVMGWFRQAPGKERELVAVISWTGEST |
| | | LYADSVKGRFTISADNSKNTAYLQMNSLKPEDTAVYYCAAFDGYTGRDWGQGTLVT |
| | | VSS |
| 5A-55 | 3295 | EVQLVESGGGLVQPGGSLRLSCAASGIDVNRNAMGWFRQAPGKEREFVAAITWSGGW |
| | | RYYADSVKGRFTISADNSKNTAYLQMNSLKPEDTAVYYCATTFGDAGIPDQYDFGWG |
| | | QGTLVTVSS |
| 5A-56 | 3296 | EVQLVESGGGLVQPGGSLRLSCAASGRTFSSNMGWFRQAPGKEREFVARIFGGDRTLY |
| | | ADSVKGRFTISADNSKNTAYLQMNSLKPEDTAVYYCADINGDWGQGTLVTVSS |
| 5A-57 | 3297 | EVQLVESGGGLVQPGGSLRLSCAASGGTFSMGWIRWVPQAQGKELEFMGCIGWITYY |
| | | ADYAKSRFSLFTDNADNIKNPPNMEMNPQKPEDTAVYYCAPFGWGQGTLVTVSS |
| 5A-58 | 3298 | EVQLVESGGGLVQPGGSLRLSCAASGCTLDYYAMGWFRQAPGKEREFVAGIRWTDAY |
| | | TEYADSVKGRFTISADNSKNTAYLQMNSLKPEDTAVYYCAADVSPSYGGRWYWGQG |
| | | TLVTVSS |
| 5A-59 | 3299 | EVQLVESGGGLVQPGGSLRLSCAASGLTFSLYRMCWFRQAPGKEREEVSCISNIDGSTY |
| | | YADSVKGRFTISADNSKNTAYLQMNSLKPEDTAVYYCAADLLGDSDYEPSSGFGWGQ |
| | | GTLVTVSS |
| 5A-60 | 3300 | EVQLVESGGGLVQPGGSLRLSCAASGRSFSSHRMGWFRQAPGKEREFVAAIMWSGSH |
| | | RNYADSVKGRFTISADNSKNTAYLQMNSLKPEDTAVYYCAAIAYEEGVYRWDWGQG |
| | | TLVTVSS |
| 5A-61 | 3301 | EVQLVESGGGLVQPGGSLRLSCAASGRIIVPNTMGWFRQAPGKERERVTGISPSAFTEY |
| | | ADSVKGRFTISADNSKNTAYLQMNSLKPEDTAVYYCAAHGWGCHWGQGTLVTVSS |
| 5A-62 | 3302 | EVQLVESGGGLVQPGGSLRLSCAASGSIFIISMGWFRQAPGKEHEFVTGINWSGGSTTY |
| | | ADSVKGRFTINADNSKNTAYLQMNSLKPEDTAVYYCAASAIGSGALRRFEYDWGQGT |
| | | LVTVSS |
| 5A-63 | 3303 | EVQLVESGGGLVQPGGSLRLSCAASGFSLDYYDMGWFRQAPGKEREFVAALGWSGGS |
| | | TDYADSVKGRFTISADNSKNTAYLQMNSLKPEDTAVYYCAAGNGGRYGIVERWGQGT |
| | | LVTVSS |
| 5A-64 | 3304 | EVQLVESGGGLVQPGGSLRLSCAASGTSISNRVMGWFRQAPGKERELVARIYTGGDTL |
| | | YADSVKGRFTISADNSKNTAYLQMNSLKPEDTAVYYCAARKIYRSLSYYGDYDWGQG |
| | | TLVTVSS |
| 5A-65 | 3305 | EVQLVESGGGLVQPGGSLRLSCAASGNIDRLYAMGWFRQAPGKEREGVAAIDSDGST |
| | | DYADSVKGRFTISADNSKNTAYLQMNSLKPEDTAVYYCAALIDYGLGFPIEWGQGTLV |
| | | TVSS |
| 5A-66 | 3306 | EVQLVESGGGLVQPGGSLRLSCAASGNTFTINVMGWFRQAPGKEREFVAAINWNGGT |
| | | TLYADSVKGRFTISADNSKNTAYLQMNSLKPEDTAVYYCAAFDGYSGIDWGQGTLVT |
| | | VSS |
| 5A-67 | 3307 | EVQLVESGGGLVQPGGSLRLSCAASGFNVNDYAMGWFRQAPGKEREFVAGITSSVGV |
| | | TNYADSVKGRFTISADNSKNTAYLQMNSLKPEDTAVYYCAADIFFVNWGRGTLVTVSS |
| 5A-68 | 3308 | EVQLVESGGGLVQPGGSLRLSCAASGFTFDHYTMGWFRQAPGKEREFVAAISGSENVT |
| | | SYADSVKGRFTISADNSKNTAYLQMNSLKPEDTAVYYCAAEPYIPVRTMRHMTFLTW |
| | | GQGTLVTVSS |
| 6A-1 | 3309 | EVQLVESGGGLVQPGGSLRLSCAASGRTFGNYNMGWFRQAPGKEREFVATINSLGGTS |
| | | YADSVKGRFTISADNSKNTAYLQMNSLKPEDTAVYYCARVDYYMDVWGQGTLVTVS |
| | | S |
| 6A-2 | 3310 | EVQLVESGGGLVQPGGSLRLSCAASGFTMSSSWMGWFRQAPGKEREFVTVISGVGTSY |
| | | ADSVKGRFTISADNSKNTAYLQMNSLKPEDTAVYYCARGPDSSGYGFDYWGQGTLVT |
| | | VSS |
| 6A-3 | 3311 | EVQLVESGGGLVQPGGSLRLSCAASGFTFSPSWMGWFRQAPGKEREFVATINEYGGRN |
| | | YADSVKGRFTISADNSKNTAYLQMNSLKPEDTAVYYCARVDRDFDYWGQGTLVTVSS |
| 6A-4 | 3312 | EVQLVESGGGLVQPGGSLRLSCAASGFTRDYYTMGWFRQAPGKEREFVAAISRSGSLT |
| | | SYADSVKGRFTISADNSKNTAYLQMNSLKPEDTAVYYCANLAYYDSSGYYDYWGQG |
| | | TLVTVSS |
| 6A-5 | 3313 | EVQLVESGGGLVQPGGSLRLSCAASGRTFTMGWFRQAPGKEREFVASTNSAGSTNYA |
| | | DSVNGRFTISADNSKNTAYLQMNSLKPEDTAVYYCTTVDQYFDYWGQGTLVTVSS |
| 6A-6 | 3314 | EVQLVESGGGLVQPGGSLRLSCAASGTTLDYYAMGWFRQAPGKERELVAAISWSGGS |
| | | TAYADSVKGRFTISADNSKNTAYLQMNSLKPEDTAVYYCAREDYYDSSGYSWGQGTL |
| | | VTVSS |
| 6A-7 | 3315 | EVQLVESGGGLVQPGGSLRLSCAASGFTFSSYWMGWFRQAPGKEREFVATINWSGVT |
| | | AYADSVKGRFTISADNSKNTAYLQMNSLKPEDTAVYYCARADDYFDYWGQGTLVTV |
| | | SS |
| 6A-8 | 3316 | EVQLVESGGGLVQPGGSLRLSCAASGFTLSGIWMGWFLQAPGKEHEFVAIITTGGRTTY |
| | | ADSXKGRFTSSSDNSKNTAYLQMNLLKPEDTAEYYCAGYSTFGSSSAYYYYSMDVGW |
| | | GQGTLVTVSS |
| 6A-9 | 3317 | EVQLVESGGGLVQPGGSLRLSCAASGFTFDYYAMGWFRQAPGKEREFVSAIDSEGRTS |
| | | YADSVKGRFTISADNSKNTAYLQMNSLKPEDTAVYYCARWGPFDIWGQGTLVTVSS |
| 6A-10 | 3318 | EVQLVESGGGLVQPGGSLRLSCAASGSIASIHAMGWFRQAPGKEREFVAAISRSGGFGS |
| | | YADSVKGRFTISADNSKNTAYLQMNSLKPEDTAVYYCARDDKYYDSSGYPAYFQHW |
| | | GQGTLVTVSS |
| 6A-11 | 3319 | EVQLVESGGGLVQPGGSLRLSCAASGLAFNAYAMGWFRQAPGKEREEVATIGWSGAN |
| | | TYYADSVKGRFTISADNSKNTAYLQMNSLKPEDTAVYYCASDPPGWGQGTLVTVSS |
| 6A-12 | 3320 | EVQLVESGGGLVQPGGSLRLSCAASGSTYTTYSMGWFRQAPGKEREFVAAISGSENVT |
| | | SYADSVKGRFTISADNSKNTAYLQMNSLKPEDTAVYYCARVDDYMDVWGQGTLVTV |
| | | SS |
| 6A-13 | 3321 | EVQLVESGGGLVQPGGSLRLSCAASGLTFNDYAMGWFRQAPGKEREFVAHIPRSTYSP |
| | | YYADSVKGRFTISADNSKNTAYLQMNSLKPEDTAVYYCAFLVGPQGVDHGAFDVWG |
| | | QGTLVTVSS |
| 6A-14 | 3322 | EVQLVESGGGLVQPGGSLRLSCAASGITFRFKAMGWFRQAPGKEREFVAAVSWDGRN |
| | | TYYADSVKGRFTISADNSKNTAYLQMNSLKPEDTAVYYCASDYYYMDVWGQGTLVT |
| | | VSS |
| 6A-15 | 3323 | EVQLVESGGGLVQPGGSLRLSCAASGSTVLINAMGWFRQAPGKEREFVAAVRWSDDY |
| | | TYYADSVKGRFTISADNSKNTAYLQMNSLKPEDTAVYYCAKEGRAGSLDYWGQGTL |
| | | VTVSS |
| 6A-16 | 3324 | EVQLVESGGGLVQPGGSLRLSCAASGFTFDDAAMGWFRQAPGKEREFVAHISWSGGS |
| | | TYYADSVKGRFTISADNSKNTAYLQMNSLKPEDTAVYYCATFGATVTATNDAFDIWG |
| | | QGTLVTVSS |
| 6A-17 | 3325 | EVQLVESGGGLVQPGGSLRLSCAASGNTGSTGYMGWFRQAPGKEREMVAGVINDGST |
| | | VYADSVKGRFTISADNSKNTAYLQMNSLKPEDTAVYYCARLATSHQDGTGYLFDYWG |
| | | QGTLVTVSS |
| 6A-18 | 3326 | EVQLVESGGGLVQPGGSLRLSCAASGLTFRNYAMGWFRQAPGKEREFIAGMMWSGGT |
| | | TTYADSVKGRFTISADNSKNTAYLQMNSLKPEDTAVYYCAREGYYYDSSGYLNYFDY |
| | | WGQGTLVTVSS |
| 6A-19 | 3327 | EVQLVESGGGLVQPGGSLRLSCAASGSILSIAVMGWFRQAPGKEREFVAAISPSAVTTY |
| | | YADSVKGRFTISADNSKNTAYLQMNSLKPEDTAVYYCAIGYYDSSGYFDYWGQGTLV |
| | | TVSS |
| 6A-20 | 3328 | EVQLVESGGGLVQPGGSLRLSCAASGSTLPYHAMGWFRQAPGKEREFVAAITWNGAS |
| | | TSYADSVKGRFTISADNSKNTAYLQMNSLKPEDTAVYYCARDRYYDTSASYFESETW |
| | | GQGTLVTVSS |
| 6A-21 | 3329 | EVQLVESGGGLVQPGGSLRLSCAASGTLFKINAMGWFRQAPGKEREFVAAITSSGSNID |
| | | YTYYADSVKGRFTISADNSKNTAYLQMNSLKPEDTAVYYCARSNTGWYSFDYWGQG |
| | | TLVTVSS |
| 6A-22 | 3330 | EVQLVESGGGLVQPGGSLRLSCAASGRTFSEVVMGWFRQAPGKEREFVATIHSSGSTS |
| | | YADSVKGRFTISADNSKNTAYLQMNSLKPEDTAVYYCVRVTSDYSMDSWGQGTLVTV |
| | | SS |
| 6A-23 | 3331 | EVQLVESGGGLVQPGGSLRLSCAASGSIFSMNTMGWFRQAPGKEREFVALINRSGGGI |
| | | NYADSVKGRFTISADNSKNTAYLQMNSLKPEDTAVYYCVRLSSGYYDFDYWGQGTLV |
| | | TVSS |
| 6A-24 | 3332 | EVQLVESGGGLVQPGGSLRLSCAASGFTLDYYAMGWFRQAPGKEREFVAAINWSGDN |
| | | THYADSVKGRFTISADNSKNTAYLQMNSLKPEDTAVYYCARAPFYCTTTKCQDNYYY |
| | | MDVWGQGTLVTVSS |
| 6A-25 | 3333 | EVQLVESGGGLVQPGGSLRLSCAASGLTFGTYTMGWFRQAPGKEREFVAAISRFGSTY |
| | | YADSVKGRFTISADNSKNTAYLQMNSLKPEDTAVYYCARGGDYDFWSVDYMDVWG |
| | | QGTLVTVSS |
| 6A-26 | 3334 | EVQLVESGGGLVQPGGSLRLSCAASGDTFSTSWMGWFRQAPGKEREFVATINTGGGT |
| | | NYADSVKGRFTISADNSKNTAYLQMNSLKPEDTAVYYCARVTTSFDYWGQGTLVTVS |
| | | S |
| 6A-27 | 3335 | EVQLVESGGGLVQPGGSLRLSCAASGITFRFKAMGWFRQAPGKEREFVASISRSGTTYY |
| | | ADSVKGRFTISADNSKNTAYLQMNSLKPEDTAVYYCATDYSAFDMWGQGTLVTVSS |
| 6A-28 | 3336 | EVQLVESGGGLVQPGGSLRLSCAASGDTYGSYWMGWFRQAPGKEREFVATITSDDRT |
| | | NYADSVKGRFTISADNSKNTAYLQMNSLKPEDTAVYYCARVTSSLSGMDVWGQGTLV |
| | | TVSS |
| 6A-29 | 3337 | EVQLVESGGGLVQPGGSLRLSCAASGYTLKNYYAMGWFRQAPGKERXLVAAIIWTGE |
| | | STLDADSVKGRFTISADNSKNTAYLQMNSLKPEDTAVYYCAREGYYDSSGYYWGQGT |
| | | LVTVSS |
| 6A-30 | 3338 | EVQLVESGGGLVQPGGSLRLSCAASGFAFGDSWMGWFRQAPGKEREFVATINWSGVT |
| | | AYADSVKGRFTISADNSKNTAYLQMNSLKPEDTAVYYCARADGYFDYWGQGTLVTV |
| | | SS |
| 6A-31 | 3339 | EVQLVESGGGLVQPGGSLRLSCAASGDTFSANRMGWFRQAPGKEREFVASITWSSANT |
| | | YYADSVKGRFTISADNSKNTAYLQMNSLKPEDTAVYYCATFNWNDEGFDFWGQGTL |
| | | VTVSS |
| 6A-32 | 3340 | EVQLVESGGGLVQPGGSLRLSCAASGFTLDYYDMGWFRQAPGKEREFVALISWSGGST |
| | | YYADSVKGRFTISADNSKNTAYLQMNSLKPEDTAVYYCATDFYGWGTRERDAFDIWG |
| | | QGTLVTVSS |
| 6A-33 | 3341 | EVQLVESGGGLVQPGGSLRLSCAASGTFQRINHMGWFRQAPGKEREFVATINTGGQPN |
| | | YADSVKGRFTISADNSKNTAYLQMNSLKPEDTAVYYCASLIAAQDYYFDYWGQGTLV |
| | | TVSS |
| 6A-34 | 3342 | EVQLVESGGGLVQPGGSLRLSCAASGSAFRSNAMGWFRQAPGKEREFVAHISWSSKST |
| | | YYADSVKGRFTISADNSKNTAYLQMNSLKPEDTAVYYCATYCSSTSCFDYWGQGTLV |
| | | TVSS |
| 6A-35 | 3343 | EVQLVESGGGLVQPGGSLRLSCAASGFTLAYYAMGWFRQAPGKEREFVAAISMSGDD |
| | | TIYADSVKGRFTISADNSKNTAYLQMNSLKPEDTAVYYCARELGYSSTVWPWGQGTL |
| | | VTVSS |
| 6A-36 | 3344 | EVQLVESGGGLVQPGGSLRLSCAASGFDFSVSWMGWFRQAPGKEREFVTAITWSGDST |
| | | NYADSVKGRFTISADNSKNTAYLQMNSLKPEDTAVYYCASLLHTGPSGGNYFDYWGQ |
| | | GTLVTVSS |
| 6A-37 | 3345 | EVQLVESGGGLVQPGGSLRLSCAASGHTFSTSWMGWFRQAPGKEREFVATINSLGGTN |
| | | YADSVKGRFTISADNSKNTAYLQMNSLKPEDTAVYYCARVSSGDYGMDVWGQGTLV |
| | | TVSS |
| 6A-38 | 3346 | EVQLVESGGGLVQPGGSLRLSCAASGNTFSGGFMGWFRQAPGKEREFVAVISSLSSKS |
| | | YADSVKGRFTISADNSKNTAYLQMNSLKPEDTAVYYCAKVDSGYDYWGQGTLVTVSS |
| 6A-39 | 3347 | EVQLVESGGGLVQPGGSLRLSCAASGFTFSPSWMGWFRQAPGKEREFVAAISWSGGST |
| | | AYADSVKGRFTISADNSKNTAYLQMNSLKPEDTAVYYCHGLGEGDPYGDYEGYFDL |
| | | WGQGTLVTVSS |
| 6A-40 | 3348 | EVQLVESGGGLVQPGGSLRLSCAASGFTFSDYWMGWFRQAPGKERELVARVWWNGG |
| | | SAYYADSVKGRFTISADNSKNTAYLQMNSLKPEDTAVYYCAREVLRQQVVLDYWGQ |
| | | GTLVTVSS |
| 6A-41 | 3349 | EVQLVESGGGLVQPGGSLRLSCAASGFTFSTSWMGWFRQAPGKEREFVASINEYGGRN |
| | | YADSVKGRFTISADNSKNTAYLQMNSLKPEDTAVYYCAGLHYYYDSSGYNPTEYYGM |
| | | DVWGQGTLVTVSS |
| 6A-42 | 3350 | EVQLVESGGGLVQPGGSLRLSCAASGDTYGSYWMGWFRQAPGKEREFVAVITSGGST |
| | | NYADSVKGRFTISADNSKNTAYLQMNSLKPEDTAVYYCTHVQNSYYYAMDVWGQGT |
| | | LVTVSS |
| 6A-43 | 3351 | EVQLVESGGGLVQPGGSLRLSCAASGRTFSSYAMMGWFRQAPGKEREFVASVNWDAS |
| | | QINYADSVKGRFTISADNSKNTAYLQMNSLKPEDTAVYYCTTLGAVYFDSSGYHDYFD |
| | | YWGQGTLVTVSS |
| 6A-44 | 3352 | EVQLVESGGGLVQPGGSLRLSCAASGGTFGVYHMGWFRQAPGKEREFIGRITWTDGST |
| | | YYADSVKGRFTISADNSKNTAYLQMNSLKPEDTAVYYCFGLLEVYDMTFDYWGQGTL |
| | | VTVSS |
| 6A-45 | 3353 | EVQLVESGGGLVQPGGSLRLSCAASGNMFSINAMGWFRQAPGKEREFVTLISWSSGRT |
| | | SYADSVKGRFTISADNSKNTAYLQMNSLKPEDTAVYYCASLGYCSGGSCFDYWGQGT |
| | | LVTVSS |
| 6A-46 | 3354 | EVQLVESGGGLVQPGGSLRLSCAASGLTFSAMGWFRQAPGKEREFVALIRRDGSTIYA |
| | | DSVKGRFTISADNSKNTAYLQMNSLKPEDTAVYYCAALGILFGYDAFDIWGQGTLVTV |
| | | SS |
| 6A-47 | 3355 | EVQLVESGGGLVQPGGSLRLSCAASGRTFSMHAMGWFRQAPGKERELVASITYGGNIN |
| | | YADSVKGRFTISADNSKNTAYLQMNSLKPEDTAVYYCAKEGYYDSTGYRTYFQQWG |
| | | QGTLVTVSS |
| 6A-48 | 3356 | EVQLVESGGGLVQPGGSLRLSCAASGFTVSNYAMGWFRQAPGKEREFVASVNWSGGT |
| | | TSYADSVKGRFTISADNSKNTAYLQMNSLKPEDTAVYYCATTGTVTLGYWGQGTLVT |
| | | VSS |
| 6A-49 | 3357 | EVQLVESGGGLVQPGGSLRLSCAASGSTVLINAMGWFRQAPGKEREFVAAISWSPGRT |
| | | DYADSVKGRFTISADNSKNTAYLQMNSLKPEDTAVYYCARDCSGGSCYSGDYWGQG |
| | | TLVTVSS |
| 6A-50 | 3358 | EVQLVESGGGLVQPGGSLRLSCAASGFSFDRWAMGWFRQAPGKEREWVASLATGGN |
| | | TNYADSVKGRFTISADNSKNTAYLQMNSLKPEDTAVYYCARVTNYDAFDIWGQGTLV |
| | | TVSS |
| 6A-51 | 3359 | EVQLVESGGGLVQPGGSLRLSCAASGYTYSSYVMGWFRQAPGKEREFVAAISRFGSTY |
| | | YADSVKGRFTISADNSKNTAYLQMNSLKPEDTAVYYCARDSGEHFWDSGYIDHWGQG |
| | | TLVTVSS |
| 6A-52 | 3360 | EVQLVESGGGLVQPGGSLRLSCAASGDTYGSYWMGWFRQAPGKEREVVAAITSGGST |
| | | VYADSVKGRFTISADNSKNTAYLQMNSLKPEDTAVYYCARVDSRFDYWGQGTLVTVS |
| | | S |
| 6A-53 | 3361 | EVQLVESGGGLVQPGGSLRLSCAASGISINTNVMGWFRQAPGKEREFVAAISTGSVTIY |
| | | ADSVKGRFTISADNSKNTAYLQMNSLKPEDTAVYYCARVDDFGYFDLWGQGTLVTVS |
| | | S |
| 6A-54 | 3362 | EVQLVESGGGLVQPGGSLRLSCAASGFEFENHWMGWFRQAPGKEREYVAHITAGGLS |
| | | NYADSVKGRFTISADNSKNTAYLQMNSLKPEDTAVYYCGRHWGIYDSSGFSSFDYWG |
| | | QGTLVTVSS |
| 6A-55 | 3363 | EVQLVESGGGLVQPGGSLRLSCAASGFTMSSSWMGWFRQAPGKEREFVARITSGGSTG |
| | | YADSVKGRFTISADNSKNTAYLQMNSLKPEDTAVYYCASVDGYFDYWGQGTLVTVSS |
| 6A-56 | 3364 | EVQLVESGGGLVQPGGSLRLSCAASGNIFRSNMGWFRQAPGKEREFVAGITWNGDTTY |
| | | YADSVKGRFTISADNSKNTAYLQMNSLKPEDTAVYYCARALGVTYQFDYWGQGTLV |
| | | TVSS |
| 6A-57 | 3365 | EVQLVESGGGLVQPGGSLRLSCAASGLTFDDHSMGWFRQAPGKEREFVAAVPLSGNT |
| | | YYADSVKGRFTISADNSKNTAYLQMNSLKPEDTAVYYCASFSGGPADFDYWGQGTLV |
| | | TVSS |
| 6A-58 | 3366 | EVQLVESGGGLVQPGGSLRLSCAASGRAVSTYAMGWFRQAPGKEREFVAAISGSENVT |
| | | SYADSVKGRFTISADNSKNTAYLQMNSLKPEDTAVYYCLSVTGDTEDYGVFDTWGQG |
| | | TLVTVSS |
| 6A-59 | 3367 | EVQLVESGGGLVQPGGSLRLSCAASGISGSVFSRTPMGWFRQAPGKEREWVSSIYSDGS |
| | | NTYYADSVKGRFTISADNSKNTAYLQMNSLKPEDTAVYYCAHWSWELGDWFDPWGQ |
| | | GTLVTVSS |
| 6A-60 | 3368 | EVQLVESGGGLVQPGGSLRLSCAASGDTYGSYWMGWFRQAPGKEREFVATISQSGAA |
| | | TAYADSVKGRFTISADNSKNTAYLQMNSLKPEDTAVYYCAGLLRYSGTYYDAFDVWG |
| | | QGTLVTVSS |
| 6A-61 | 3369 | EVQLVESGGGLVQPGGSLRLSCAASGDTYGSYWMGWFRQAPGKEREFVAAINWSGGS |
| | | TNYADSVKGRFTITADNNKNTAYLQMNSLKPEDTAVYYCAGLGWNYMDYWGQGTL |
| | | VTVSS |
| 6A-62 | 3370 | EVQLVESGGGLVQPGGSLRLSCAASGSTFSGNWMGWFRQAPGKEREFVAVISWTGGS |
| | | TYYADSVKGRFTISADNSKNTAYLQMNSLKPEDTAVYYCATHNSLSGFDYWGQGTLV |
| | | TVSS |
| 6A-63 | 3371 | EVQLVESGGGLVQPGGSLRLSCAASGQTFNMGWFRQAPGKEREFVAAIGSGGSTSYAD |
| | | SVKGRFTISADNSKNTAYLQMNSLKPEDTAVYYCWRLGNDYFDYWGQGTLVTVSS |
| 6A-64 | 3372 | EVQLVESGGGLVQPGGSLRLSCAASGIPSIHAMGWFRQAPGKERELVAAINWSHGVTY |
| | | YADSVKGRFTISADNSKNTAYLQMNSLKPEDTAVYYCGGGYGYHFDYWGQGTLVTV |
| | | SS |
| 6A-65 | 3373 | EVQLVESGGGLVQPGGSLRLSCAASGLPFSTLHMGWFRQAPGKEREFVASLSIFGATG |
| | | YADSVKGRFTISADNSKNTAYLQMNSLKPEDTAVYYCWMYYYDSSGYYGNYYYGM |
| | | DVWGQGTLVTVSS |
| 6A-66 | 3374 | EVQLVESGGGLVQPGGSLRLSCAASGLTFSLFAMGWFRQAPGKERELVAAISSGGSTD |
| | | YADSVKGRFTISADNSKNTAYLQMNSLKPEDTAVYYCARGNTKYYYDSSGYSSAFDY |
| | | WGQGTLVTVSS |
| 6A-67 | 3375 | EVQLVESGGGLVQPGGSLRLSCAASGSFSNYAMGWFRQAPGKEREFVAAISSSGALTS |
| | | YADSVKGRFTISADNSKNTAYLQMNSLKPEDTAVYYCWIVGPGPLDGMDVWGQGTL |
| | | VTVSS |
| 6A-68 | 3376 | EVQLVESGGGLVQPGGSLRLSCAASGFTLSDRAMGWFRQAPGKEREYVAHITAGGLS |
| | | NYADSVKGRFTISADNSKNTAYLQMNSLKPEDTAVYYCVHLASQTGAGYFDLWGQG |
| | | TLVTVSS |
| 6A-69 | 3377 | EVQLVESGGGLVQPGGSLRLSCAASGGTFSSVGMGWFRQAPGKEREFVAGISRSGGTY |
| | | YADSVKGRFTISADNSKNTAYLQMNSLKPEDTAVYYCARYDFWSGYPYWGQGTLVT |
| | | VSS |
| 6A-70 | 3378 | EVQLVESGGGLVQPGGSLRLSCAASGFNLDDYADMGWFRQAPGKEREFVAAIGWGG |
| | | GSTRYADSVKGRFTISADNSKNTAYLQMNSLKPEDTAVYYCAREILWFGEFGEPNVW |
| | | GQGTLVTVSS |
| 6A-71 | 3379 | EVQLVESGGGLVQPGGSLRLSCAASGITFSNDAMGWFRQAPGKEREFVAIITSSDTNDT |
| | | TNYADSVKGRFTISADNSKNTAYLQMNSLKPEDTAVYYCARLHYYDSSGYFDYWGQ |
| | | GTLVTVSS |
| 6A-72 | 3380 | EVQLVESGGGLVQPGGSLRLSCAASGSTLSINAMGWFRQAPGKEREFVAAIDWSGGST |
| | | AYADSVKGRFTISADNSKNTAYLQMNSLKPEDTAVYYCARDSSATRTGPDYWGQGTL |
| | | VTVSS |
| 6A-73 | 3381 | EVQLVESGGGLVQPGGSLRLSCAASGHTFSGYAMGWFRQAPGKEREFVAVITREGSTY |
| | | YADSVKGRFTISADNSKNTAYLQMNSLKPEDTAVYYCARLGGEGFDYWGQGTLVTVS |
| | | S |
| 6A-74 | 3382 | EVQLVESGGGLVQPGGSLRLSCAASGFAFGDSWMGWFRQAPGKERELVAAITSGGST |
| | | DYADSVKGRFTISADNSKNTAYLQMNSLKPEDTAVYYCARGLLWFGELFGYWGQGTL |
| | | VTVSS |
| 6A-75 | 3383 | EVQLVESGGGLVQPGGSLRLSCAASGGTFSTYWMGWFRQAPGKEREFVAAISRSGGN |
| | | TYYADSVKGRFTISADNSKNTAYLQMNSLKPEDTAVYYCVRHSGTDGDSSFDYWGQG |
| | | TLVTVSS |
| 6A-76 | 3384 | EVQLVESGGGLVQPGGSLRLSCAASGLAFDFDGMGWFRQAPGKEREGVAAINSGGST |
| | | YYADSVKGRFTISADNSKNTAYLQMNSLKPEDTAVYYCARFFRAHDYWGQGTLVTVS |
| | | S |
| 6A-77 | 3385 | EVQLVESGGGLVQPGGSLRLSCAASGFTFDRSWMGWFRQAPGKEREFVAAVTEGGTT |
| | | SYADSVKGRFTISADNSKNTAYLQMNSLKPEDTAVYYCARADYDFDYWGQGTLVTVS |
| | | S |
| 6A-78 | 3386 | EVQLVESGGGLVQPGGSLRLSCAASGRTYDAMGWFRQAPGKEREFVASVTSGGYTHY |
| | | ADSVKGRFTISADNSKNTAYLQMNSLKPEDTAVYYCAKFGRKIVGATELDYWGQGTL |
| | | VTVSS |
| 6A-79 | 3387 | EVQLVESGGGLVQPGGSLRLSCAASGSISSIDYMGWFRQAPGKEREGVSWISSSDGSTY |
| | | YADSVKGRFTISADNSKNTAYLQMNSLKPEDTAVYYCARSPSFSQIYYYYYMDVWGQ |
| | | GTLVTVSS |
| 6A-80 | 3388 | EVQLVESGGGLVQPGGSLRLSCAASGGTFSFYNMGWFRQAPGKEREFVAFISGNGGTS |
| | | YADSVKGRFTISADNSKNTAYLQMNSLKPEDTAVYYCAVVAMRMVTTEGPDVLDVW |
| | | GQGTLVTVSS |
| 6A-81 | 3389 | EVQLVESGGGLVQPGGSLRLSCAASGFIGNYHAMGWFRQAPGKEREFVAAVTWSGGT |
| | | TNYADSVKGRFTISADNSKNTAYLQMNSLKPEDTAVYYCAREGYYYDSSGYPYYFDY |
| | | WGQGTLVTVSS |
| 6A-82 | 3390 | EVQLVESGGGLVQPGGSLRLSCAASGSSLDAYGMGWFRQAPGKEREFVAAISWGGGSI |
| | | YYADSVKGRFTISADNSKNTAYLQMNSLKPEDTAVYYCARLSQGMVALDYWGQGTL |
| | | VTVSS |
| 6A-83 | 3391 | EVQLVESGGGLVQPGGSLRLSCAASGSIASIHAMGWFRQAPGKEREFVAAITWSGAITS |
| | | YADSVKGRFTISADNSKNTAYLQMNSLKPEDTAVYYCAKDGGYGELHYGMEVWGQG |
| | | TLVTVSS |
| 6A-84 | 3392 | EVQLVESGGGLVQPGGSLRLSCAASGFTPDDYAMGWFRQAPGKEREFVAAINSGGSYT |
| | | YYADSVKGRFTISADNSKNTAYLQMNSLKPEDTAVYYCARDRGPWGQGTLVTVSS |
| 6A-85 | 3393 | EVQLVESGGGLVQPGGSLRLSCAASGGTFSVFAMGWFRQAPGKEREFVSAINWSGGSL |
| | | LYADSVKGRFTISADNSKNTAYLQMNSLKPEDTAVYYCALFGDFDYWGQGTLVTVSS |
| 6A-86 | 3394 | EVQLVESGGGLVQPGGSLRLSCAASGPISGINRMGWFRQAPGKEREFVAVITSNGRPSY |
| | | ADSVKGRFTISADNSKNTAYLQMNSLKPEDTAVYYCVRLSSGYFDFDYWGQGTLVTV |
| | | SS |
| 6A-87 | 3395 | EVQLVESGGGLVQPGGSLRLSCAASGTSIMVGAMGWFRQAPGKEREFVAIIRGDGRTS |
| | | YADSVKGRFTISADNSKNTAYLQMNSLKPEDTAVYYCARFAGWDAFDIWGQGTLVTV |
| | | SS |
| 6A-88 | 3396 | EVQLVESGGGLVQPGGSLRLSCAASGRTFSTHWMGWFRQAPGKEREFVAVINWSGGSI |
| | | YYADSVKGRFTISADNSKNTAYLQMNSLKPEDTAVYYCARLSSDGYNYFDFWGQGTL |
| | | VTVSS |
| 6A-89 | 3397 | EVQLVESGGGLVQPGGSLRLSCAASGTIFASAMGWFRQAPGKEHQFVAVVNWNGSST |
| | | VYADNVKGRFTIIADNSKNTAYLQMNSLKPEDTAVYYCTTVDQYFNYWGQGTLVTVS |
| | | S |
| 6A-90 | 3398 | EVQLVESGGGLVQPGGSLRLSCAASGFPFSIWPMGWFRQAPGKEREFVAAVRWSSTYY |
| | | ADSVKGRFTISADNSKNTAYLQMNSLKPEDTAVYYCATGECDGGSCSLAYWGQGTLV |
| | | TVSS |
| 6A-91 | 3399 | EVQLVESGGGLVQPGGSLRLSCAASGRTFGNYAMGWFRQAPGKEREFVASISSSGVSK |
| | | HYADSVKGRFTISADNSKNTAYLQMNSLKPEDTAVYYCVRFGSSWARDLDQWGQGT |
| | | LVTVSS |
| 6A-92 | 3400 | EVQLVESGGGLVQPGGSLRLSCAASGFLFDSYASMGWFRQAPGKEREFVATIWRRGNT |
| | | YYANYADSVKGRFTISADNSKNTAYLQMNSLKPEDTAVYYCTETGTAAWGQGTLVTV |
| | | SS |
| 6A-93 | 3401 | EVQLVESGGGLVQPGGSLRLSCAASGLPFSTKSMGWFRQAPGKEREFVAAISMSGLTS |
| | | YADSVKGRFTISADNSKNTAYLQMNSLKPEDTAVYYCLKVLGGDYEADNWFDYWGQ |
| | | GTLVTVSS |
| 6A-94 | 3402 | EVQLVESGGGLVQPGGSLRLSCAASGNIFRIETMGWFRQAPGKEREFVAGIIRSGGETL |
| | | YADSVKGRFTISADNSKNTAYLQMNSLKPEDTAVYYCARSLYYDRSGSYYFDYWGQG |
| | | TLVTVSS |
| 6A-95 | 3403 | EVQLVESGGGLVQPGGSLRLSCAASGIPSSIRAMGWFRQAPGKEREFVAVIRWTGGST |
| | | YYADSVKGRFTISADNSKNTAYLQMNSLKPEDTAVYYCARDIGYYDSSGYYNDGGFD |
| | | YWGQGTLVTVSS |
| 6A-96 | 3404 | EVQLVESGGGLVQPGGSLRLSCAASGFTLSGNWMGWFRQAPGKEREFVAIITSGGRTN |
| | | YADSVKGRFTISADNSKNTAYLQMNSLKPEDTAVYYCAGHATFGGSSSSYYYGMDV |
| | | WGQGTLVTVSS |
| 6A-97 | 3405 | EVQLVESGGGLVQPGGSLRLSCAASGFTFSSLAMGWFRQAPGKEREFVAAITWSGDIT |
| | | NYADSVKGRFTITADNSKNTAYLQMNSLKPEDTAVYYCLRLSSSGFDHWGQGTLVTV |
| | | SS |
| 6A-98 | 3406 | EVQLVESGGGLVQPGGSLRLSCAASGTFGHYAMGWFRQAPGKEREFVAAINWSSRST |
| | | VYADSVKGRFTITADNSKNTAYLQMNSLKPEDTAVYYCAKSDGLMGELRSASAFDIW |
| | | GQGTLVTVSS |
| 6A-99 | 3407 | EVQLVESGGGLVQPGGSLRLSCAASGIPFRSRTMGWFRQAPGKEREFVAGISRSGASTA |
| | | YADSVKGRFTISADNSKNTAYLQMNSLKPEDTAVYYCTHANDYGDYWGQGTLVTVS |
| | | S |
| 6A-100 | 3408 | EVQLVESGGGLVQPGGSLRLSCAASGGTFSTSWMGWFRQAPGKEREYVAHITAGGLS |
| | | NYADSVKGRFTISADNSKNTAYLQMNSLKPEDTAVYYCARLLVREDWYFDLWGQGT |
| | | LVTVSS |
| 6A-101 | 3409 | EVQLVESGGGLVQPGGSLRLSCAASGGTFSLFAMGWFRQAPGKEREFVAAISWTGDST |
| | | YYKYYADSVKGRFTISADNSKNTAYLQMNSLKPEDTAVYYCAYNNSSGEYWGQGTL |
| | | VTVSS |
| 6A-102 | 3410 | EVQLVESGGGLVQPGGSLRLSCAASGSSFSAYAMGWFRQAPGKEREFVSAIDSEGTTT |
| | | YADSVKGRFTISADNSKNTAYLQMNSLKPEDTAVYYCAGDYNFWSGFDHWGQGTLV |
| | | TVSS |
| 6A-103 | 3411 | EVQLVESGGGLVQPGGSLRLSCAASGRTSSPIAMGWFRQAPGKEREPVAVRWSDDYT |
| | | YYADSVKGRFTISADNSKNTAYLQMNSLKPEDTAVYYCAKKLGGYYAFDIWGQGTLV |
| | | TVSS |
| 6A-104 | 3412 | EVQLVESGGGLVQPGGSLRLSCAASGLTFNQYTMGWFRQAPGKEREFVASITDGGSTY |
| | | YADSVKGRFTISADNSKNTAYLQMNSLKPEDTAVYYCARDSRYMDVWGQGTLVTVS |
| | | S |
| 6A-105 | 3413 | EVQLVESGGGLVQPGGSLRLSCAASGPTFSSMGWFRQAPGKEREFVAAISWDGGATA |
| | | YADSVKGRFTISADNSKNTAYLQMNSLKPEDTAVYYCAIEIVVGGIYWGQGTLVTVSS |
| 6A-106 | 3414 | EVQLVESGGGLVQPGGSLRLSCAASGIPSTLRAMGWFRQAPGKEREFVAATSWSGGSK |
| | | YYADSVKGRFTISADNSKNTAYLQMNSLKPEDTAVYYCATDLYYMDVWGQGTLVTV |
| | | SS |
| 6A-107 | 3415 | EVQLVESGGGLVQPGGSLRLSCAASGGVGFSVTNMGWFRQAPGKEREFVAVISSSSST |
| | | NYADSVKGRFTISADNSKNTAYLQMNSLKPEDTAVYYCTTFNWNDEGFDYWGQGTL |
| | | VTVSS |
| 6A-108 | 3416 | EVQLVESGGGLVQPGGSLRLSCAASGGTFGSYGMGWFRQAPGKEREFVAAIRWSGGIT |
| | | YYADSVKGRFTISADNSKNTAYLQMNSLKPEDTAVYYCARERYWNPLPYYYYGMDV |
| | | WGQGTLVTVSS |
| 6A-109 | 3417 | EVQLVESGGGLVQPGGSLRLSCAASGGTFSTYAMGWFRQVPGKEREFVASIDWSGLTS |
| | | YADSVKGRFTISADNSKNTAYLQMNSLKPEDTAVYYCARGPFYMYCSGTKCYSTNWF |
| | | DPWGQGTLVTVSS |
| 6A-110 | 3418 | EVQLVESGGGLVQPGGSLRLSCAASGPIYAVNRMGWFRQAPGKEREFVAGIWRSGGH |
| | | RDYADSVKGRFTISADNSKNTAYLQMNSLKPEDTAVYYCARGEIDILTGYWYDYWGQ |
| | | GTLVTVSS |
| 6A-111 | 3419 | EVQLVESGGGLVQPGGSLRLSCAASGFTFSNYWMGWFRQAPGKEREFVGGISRSGVST |
| | | SYADSVKGRFTISADNSKNTAYLQMNSLKPEDTAVYYCTTLLYYYDSSGYSFDAFDIW |
| | | GQGTLVTVSS |
| 6A-112 | 3420 | EVQLVESGGGLVQPGGSLRLSCAASGGTFSAYHMGWFRQAPGKERELVTIIDNGGPTS |
| | | YADSVKGRFTISADNSKNTAYLQMNSLKPEDTAVYYCTALLYYFDNSGYNFDPFDIWG |
| | | QGTLVTGSS |
| 2A-H1 | 3421 | EVQLLESGGGLVQPGGSLRLSCAASGFTFSNYATDWVRQAPGKGLEWVSIISGSGGAT |
| | | YYADSVKGRFTISRDNSKNTLYLQMNSLRAEDTAVYYCAKGGYCSSDTCWWEYWLD |
| | | PWGQGTLVTVSS |
| 2A-H2 | 3422 | EVQLLESGGGLVQPGGSLRLSCAASGFTFSAFAMGWVRQAPGKGLEWVSAITASGDIT |
| | | YYADSVKGRFTISRDNSKNTLYLQMNSLRAEDTAVYYCARQSDGLPSPWHFDLGGQG |
| | | TLVTVSS |
| 2A-H3 | 3423 | EVQLLESGGGLVQPGGSLRLSCAASGFTFSDFAMAWVRQAPGKGLEWVSAISGSGDIT |
| | | YYADSVKGRFTISRDNSKNTLYLQMNSLRAEDTAVYYCAREADGLHSPWHFDLWGQ |
| | | GTLVTVSS |
| 2A-H4 | 3424 | EVQLLESGGGLVQPGGSLRLSCAASGFTFSRHAMNWVRQAPGKGLEWVSGISGSGDET |
| | | YYADSVKGRFTISRDNSKNTLYLQMNSLRAEDTAVYYCARDLPASYYDSSGYYWHNG |
| | | MDVWGQGTLVTVSS |
| 2A-H5 | 3425 | EVQLLESGGGLVQPGGSLRLSCAASGFTFSDFAMAWVRQAPGKGLEWVSAISGSGDIT |
| | | YYADSVKGRFTISRDNSKNTLYLQMNSLRAEDTAVYYCAREADCLPSPWYLDLWGQG |
| | | TLVTVSS |
| 2A-H6 | 3426 | EVQLLESGGGLVQPGGSLRLSCAASGFTFSDFAMAWVRQAPGKGLEWVSAISGSGDIT |
| | | YYADSVKGRFTISRDNSKNTLYLQMNSLRAEDTAVYYCAREADGLHSPWHFDLWGQ |
| | | GTLVTVSS |
| 2A-H7 | 3427 | EVQLLESGGGLVQPGGSLRLSCAASGFTFSNYPMNWVRQAPGKGLEWVSTISGSGGNT |
| | | FYADSVKGRFTISRDNSKNTLYLQMNSLRAEDTAVYYCVRHDEYSFDYWGQGTLVTV |
| | | SS |
| 2A-H8 | 3428 | EVQLLESGGGLVQPGGSLRLSCAASGFTFSDFAMAWVRQAPGKGLEWVSAITGSGDIT |
| | | YYADSVKGRFTISRDNSKNTLYLQMNSLRAEDTAVYYCAREADGLHSPWHFDLWGQ |
| | | GTLVTVSS |
| 2A-H9 | 3429 | EVQLLESGGGLVQPGGSLRLSCAASGFTFSDYPMNWVRQAPGKGLEWVSTISGSGGIT |
| | | FYADSVKGRFTISRDNSKNTLYLQMNSLRAEDTAVYYCVRHDEYSFDYWGQGTLVTV |
| | | SS |
| 2A-H10 | 3430 | EVQLLESGGGLVQPGGSLRLSCAASGFTFSDYPMNWVRQAPGKGLEWVSAISGSGDNT |
| | | YYADSVKGRFTISRDNSKNTLYLQMNSLRAEDTAVYYCVRHDEYSFDYWGQGTLVTV |
| | | SS |
| 2A-H11 | 3431 | EVQLLESGGGLVQPGGSLRLSCAASGFTFSDFAMAWVRQAPGKGLEWVSAITGTGDIT |
| | | YYADSVKGRFTISRDNSKNTLYLQMNSLRAEDTAVYYCAREADGLHSPWGQGTLVTV |
| | | SS |
| 2A-H12 | 3432 | EVQLLESGGGLVQPGGSLRLSCAASGFTFSDYPMNWVRQAPGKGLEWVSAITGSGDIT |
| | | YYADSVKGRFTISRDNSKNTLYLQMNSLRAEDTAVYYCVRHDEYSFDYWGQGTLVTV |
| | | SS |
| 2A-H13 | 3433 | EVQLLESGGGLVQPGGSLRLSCAASGFTFSDFAMAWVRQAPGKGLEWVSAISGSGDIT |
| | | YYADSVKGRFTISRDNSKNTLYLQMNSLRAEDTAVYYCAREADGLHSPWHFDLWGQ |
| | | GTLVTVSS |
| 2A-H14 | 3434 | EVQLLESGGGLVQPGGSLRLSCAASGFTFSDFAMAWVRQAPGKGLEWVSAISGSGDIT |
| | | YYADSVKGRFTISRDNSKNTLYLQMNSLRAEDTAVYYCAREADGLHSPWHFDLWGQ |
| | | GTLVTVSS |
| 2A-H15 | 3435 | EVQLLESGGGLVQPGGSLRLSCAASGFTFPRYAMSWVRQAPGKGLEWVSTISGSGSTT |
| | | YYADSVKGRFTISRDNSKNTLYLQMNSLRAEDTAVYYCARLIDAFDIWGQGTLVTVSS |
| 2A-L1 | 3436 | DIQMTQSPSSLSASVGDRVTITCRASQSIHRFLNWYQQKPGKAPKLLIYAASNLHSGVPS |
| | | RFSGSGSGTDFTLTISSLQPEDFATYYCQQSYGLPPTFGQGTKVEIK |
| 2A-L2 | 3437 | DIQMTQSPSSLSASVGDRVTITCRASQSIHISLNWYQQKPGKAPKLLIYLASPLASGVPS |
| | | RFSGSGSGTDFTLTISSLQPEDFATYYCQQSYSAPPYTFGQGTKVEIK |
| 2A-L3 | 3438 | DIQMTQSPSSLSASVGDRVTITCRASQSIHTYLNWYQQKPGKAPKLLIYAASALASGVP |
| | | SRFSGSGSGTDFTLTISSLQPEDFATYYCQQSYSAPPYTFGQGTKVEIK |
| 2A-L4 | 3439 | DIQMTQSPSSLSASVGDRVTITCRASQTINTYLNWYQQKPGKAPKLLIYSASTLQSGVPS |
| | | RFSGSGSGTDFTLTISSLQPEDFATYYCQQSYSTFTFGQGTKVEIK |
| 2A-L5 | 3440 | DIQMTQSPSSLSASVGDRVTITCRASQNIHTYLNWYQQKPGKAPKLLIYAASTFAKGVP |
| | | SRFSGSGSGTDFTLTISSLQPEDFATYYCQQSYSAPPYTFGQGTKVEIK |
| 2A-L6 | 3441 | DIQMTQSPSSLSASVGDRVTITCRASQSIDTYLNWYQQKPGKAPKLLIYAASALASGVP |
| | | SRFSGSGSGTDFTLTISSLQPEDFATYYCQQSYSAPPYTFGQGTKVEIK |
| 2A-L7 | 3442 | DIQMTQSPSSLSASVGDRVTITCRASQSIGNYLNWYQQKPGKAPKLLIYGVSSLQSGVP |
| | | SRFSGSGSGTDFTLTISSLQPEDFATYYCQQSHSAPLTFGQGTKVEIK |
| 2A-L8 | 3443 | DIQMTQSPSSLSASVGDRVTITCRASQSIDTYLNWYQQKPGKAPKLLIYAASALASGVP |
| | | SRFSGSGSGTDFTLTISSLQPEDFATYYCQQSYSAPPYTFGQGTKVEIK |
| 2A-L9 | 3444 | DIQMTQSPSSLSASVGDRVTITCRASQSIDNYLNWYQQKPGKAPKLLIYGVSALQSGVP |
| | | SRFSGSGSGTDFTLTISSLQPEDFATYYCQQSHSAPPYFFGQGTKVEIK |
| 2A-L10 | 3445 | DIQMTQSPSSLSASVGDRVTITCRASQSIDTYLNWYQQKPGKAPKLLIYGASALESGVPS |
| | | RFSGSGSGTDFTLTISSLQPEDFATYYCQQSHSAPPYFFGQGTKVEIK |
| 2A-L11 | 3446 | DIQMTQSPSSLSASVGDRVTITCRASQSIDTYLNWYQQKPGKAPKLLIYAASALASGVP |
| | | SRFSGSGSGTDFTLTISSLQPEDFATYYCQQSYSAPPYTFGQGTKVEIK |
| 2A-L12 | 3447 | DIQMTQSPSSLSASVGDRVTITCRASQSIDTYLNWYQQKPGKAPKLLIYGVSALQSGVP |
| | | SRFSGSGSGTDFTLTISSLQPEDFATYYCQQSYSAPPYFFGQGTKVEIK |
| 2A-L13 | 3448 | DIQMTQSPSSLSASVGDRVTITCRASQSIDTYLNWYQQKPGKAPKLLIYAASALASGVP |
| | | SRFSGSGSGTDFTLTISSLQPEDFATYYCQQSYSAPPYTFGQGTKVEIK |
| 2A-L14 | 3449 | DIQMTQSPSSLSASVGDRVTITCRASQSIDNYLNWYQQKPGKAPKLLIYGVSALQSGVP |
| | | SRFSGSGSGTDFTLTISSLQPEDFATYYCQQSHSAPLTFGQGTKVEIK |
| 2A-L15 | 3450 | DIQMTQSPSSLSASVGDRVTITCRASQRIGTYLNWYQQKPGKAPKLLIYAASNLEGGVP |
| | | SRFSGSGSGTDFTLTISSLQPEDFATYYCQQNYSTTWTFGQGTKVEIK |
| 2A-H16 | 3451 | EVQLLESGGGLVQPGGSLRLSCAASGFTFSSYAMSWVRQAPGKGLEWVSVISGSGGST |
| | | YYADSVKGRFTISRDNSKNTLYLQMNSLRAEDTAVYYCAREGYRDYLWYFDLWGQG |
| | | TLVTVSS |
| 2A-H17 | 3452 | EVQLLESGGGLVQPGGSLRLSCAASGFTFSNYAMSWVRQAPGKGLEWVSAISGSAGST |
| | | YYADSVKGRFTISRDNSKNTLYLQMNSLRAEDTAVYYCARVRQGLRRTWYYFDYWG |
| | | QGTLVTVSS |
| 2A-H18 | 3453 | EVQLLESGGGLVQPGGSLRLSCAASGFTFSSYAMYWVRQAPGKGLEWVSAISGSAGST |
| | | YYADSVKGRFTISRDNSKNTLYLQMNSLRAEDTAVYYCARDTNDFWSGYSIFDPWGQ |
| | | GTLVTVSS |
| 2A-H19 | 3454 | EVQLLESGGGLVQPGGSLRLSCAASGFTFSSYTMSWVRQAPGKGLEWVSVISGSGGST |
| | | YYADSVKGRFTISRDNSKNTLYLQMNSLRAEDTAVYYCAREGYRDYLWYFDLWGQG |
| | | TLVTVSS |
| 2A-H20 | 3455 | EVQLLESGGGLVQPGGSLRLSCAASGFTFSSYDMSWVRQAPGKGLEWVSVISGSGGST |
| | | YYADSVKGRFTISRDNSKNTLYLQMNSLRAEDTAVYYCAKGPLVGWYFDLWGQGTL |
| | | VTVSS |
| 2A-L16 | 3456 | DIQMTQSPSSLSASVGDRVTITCTGTSSDVGSYDLVSWYQQKPGKAPKLLIYEGNKRPS |
| | | GVPSRFSGSGSGTDFTLTISSLQPEDFATYYCCSYAGSSVVFGQGTKVEIK |
| 2A-L17 | 3457 | DIQMTQSPSSLSASVGDRVTITCTGTSSDVGSSNLVSWYQQKPGKAPKLLIYEGSKRPS |
| | | GVPSRFSGSGSGTDFTLTISSLQPEDFATYYCCSYAGSLYVFGQGTKVEIK |
| 2A-L18 | 3458 | DIQMTQSPSSLSASVGDRVTITCTGTSSDIGSYNLVSWYQQKPGKAPKLLIYEGTKRPSG |
| | | VPSRFSGSGSGTDFTLTISSLQPEDFATYYCCSYAGSRTYVFGQGTKVEIK |
| 2A-L19 | 3459 | DIQMTQSPSSLSASVGDRVTITCTGTSTDVGSYNLVSWYQQKPGKAPKLLIYEGTKRPS |
| | | GVPSRFSGSGSGTDFTLTISSLQPEDFATYYCCSYAGSYTSVVFGQGTKVEIK |
| 2A-L20 | 3460 | DIQMTQSPSSLSASVGDRVTITCTGTSSNVGSYNLVSWYQQKPGKAPKLLIYEGTKRPS |
| | | GVPSRFSGSGSGTDFTLTISSLQPEDFATYYCCSYAGSSSFVVFGQGTKVEIK |
| 3A-H1 | 3461 | EVQLLESGGGLVQPGGSLRLSCAASGFTFRSHAMSWVRQAPGKGLEWVSSISGGGAST |
| | | YYADSVKGRFTISRDNSKNTLYLQMNSLRAEDTAVYYCARVKYLTTSSGWPRPYFDN |
| | | WGQGTLVTVSS |
| 3A-H2 | 3462 | EVQLLESGGGLVQPGGSLRLSCAASGFTFSAYSMSWVRQAPGKGLEWVSAISGSGGSR |
| | | YYADSVKGRFTISRDNSKNTLYLQMNSLRAEDTAVYYCGRSKWPQANGAFDIWGQGT |
| | | LVTVSS |
| 3A-H3 | 3463 | EVQLLESGGGLVQPGGSLRLSCAASGFMFGNYAMSWVRQAPGKGLEWVAAISGSGGS |
| | | TYYADSVKGRFTISRDNSKNTLYLQMNSLRAEDTAVYYCAKDRGYSSSWYGGFDYW |
| | | GQGTLVTVSS |
| 3A-H4 | 3464 | EVQLLESGGGLVQPGGSLRLSCAASGFTFRNHAMAWVRQAPGKGLEWVSGISGSGGT |
| | | TYYGDSVKGRFTISRDNSKNTLYLQMNSLRAEDTAVYYCARGTRFLQWSLPLDVWGQ |
| | | GTLVTVSS |
| 3A-H5 | 3465 | EVQLLESGGGLVQPGGSLRLSCAASGFTIPNYAMSWVRQAPGKGLEWVSGISGAGAST |
| | | YYADSVKGRFTISRDNSKNTLYLQMNSLRAEDTAVYYCARHTWWKGAGFFDHWGQG |
| | | TLVTVSS |
| 3A-H6 | 3466 | EVQLLESGGGLVQPGGSLRLSCAASGFTFRNYAMAWVRQAPGKGLEWVSGISGSGGT |
| | | TYYGDSVKGRFTISRDNSKNTLYLQMNSLRAEDTAVYYCARGTRFLEWSLPLDVWGQ |
| | | GTLVTVSS |
| 3A-H7 | 3467 | EVQLLESGGGLVQPGGSLRLSCAASGFTIRNYAMSWVRQAPGKGLEWVSSISGGGAST |
| | | YYADSVKGRFTISRDNSKNTLYLQMNSLRAEDTAVYYCARVKYLTTSSGWPRPYFDN |
| | | WGQGTLVTVSS |
| 3A-H8 | 3468 | EVQLLESGGGLVQPGGSLRLSCAASGFTIPNYAMSWVRQAPGKGLEWVSGISGSGAST |
| | | YYADSVKGRFTISRDNSKNTLYLQMNSLRAEDTAVYYCARHTWWKGAGFFDHWGQG |
| | | TLVTVSS |
| 3A-H9 | 3469 | EVQLLESGGGLVQPGGSLRLSCAASGFTITNYAMSWVRQAPGKGLEWVSGISGSGAGT |
| | | YYADSVKGRFTISRDNSKNTLYLQMNSLRAEDTAVYYCARHAWWKGAGFFDHWGQ |
| | | GTLVTVSS |
| 3A-H10 | 3470 | EVQLLESGGGLVQPGGSLRLSCAASGFTFRSHAMSWVRQAPGKGLEWVSSISGGGAST |
| | | YYADSVKGRFTISRDNSKNTLYLQMNSLRAEDTAVYYCARVKYLTTSSGWPRPYFDN |
| | | WGQGTLVTVSS |
| 3A-H11 | 3471 | EVQLLESGGGLVQPGGSLRLSCAASGFTITNYAMSWVRQAPGKGLEWVSGISGSGAST |
| | | YYADSVKGRFTISRDNSKNTLYLQMNSLRAEDTAVYYCARHTWWKGAGFFDHWGQG |
| | | TLVTVSS |
| 3A-H12 | 3472 | EVQLLESGGGLVQPGGSLRLSCAASGFTFRSHAMNWVRQAPGKGLEWVSAISGSGGST |
| | | NYADSVKGRFTISRDNSKNTLYLQMNSLRAEDTAVYYCARGLKFLEWLPSAFDIWGQ |
| | | GTLVTVSS |
| 3A-H13 | 3473 | EVQLLESGGGLVQPGGSLRLSCAASGFTFRSHAMSWVRQAPGKGLEWVSSISGGGAST |
| | | YYADSVKGRFTISRDNSKNTLYLQMNSLRAEDTAVYYCARVKYLTTSSGWPRPYFDN |
| | | WGQGTLVTVSS |
| 3A-H14 | 3474 | EVQLLESGGGLVQPGGSLRLSCAASGFTFRSYAMSWVRQAPGKGLEWVSSISGGGAST |
| | | YYADSVKGRFTISRDNSKNTLYLQMNSLRAEDTAVYYCARVKYLTTSSGWPRPYFDN |
| | | WGQGTLVTVSS |
| 3A-H15 | 3475 | EVQLLESGGGLVQPGGSLRLSCAASGFTITNYAMSWVRQAPGKGLEWVSGISGSGAGT |
| | | YYADSVKGRFTISRDNSKNTLYLQMNSLRAEDTAVYYCARHTWWKGAGFFDHWGQG |
| | | TLVTVSS |
| 3A-L1 | 3476 | DIQMTQSPSSLSASVGDRVTITCRASQSIRKYLNWYQQKPGKAPKLLIYASSTLQRGVPS |
| | | RFSGSGSGTDFTLTISSLQPEDFATYYCQQSLSTPFTFGQGTKVEIK |
| 3A-L2 | 3477 | DIQMTQSPSSLSASVGDRVTITCRASQNIKTYLNWYQQKPGKAPKLLIYAASKLQSGVP |
| | | SRFSGSGSGTDFTLTISSLQPEDFATYYCQQSYSTSPTFGQGTKVEIK |
| 3A-L3 | 3478 | DIQMTQSPSSLSASVGDRVTITCRASQTIYSYLNWYQQKPGKAPKLLIYATSTLQGGVP |
| | | SRFSGSGSGTDFTLTISSLQPEDFATYYCQHRGTFGQGTKVEIK |
| 3A-L4 | 3479 | DIQMTQSPSSLSASVGDRVTITCRASRSIRRYLNWYQQKPGKAPKLLIYASSSLQAGVPS |
| | | RFSGSGSGTDFTLTISSLQPEDFATYYCQQSYSTLLTFGQGTKVEIK |
| 3A-L5 | 3480 | DIQMTQSPSSLSASVGDRVTITCRASQSIGKYLNWYQQKPGKAPKLLIYASSSLQSGVPS |
| | | RFSGSGSGTDFTLTISSLQPEDFATYYCQQSYSPPFTFGQGTKVEIK |
| 3A-L6 | 3481 | DIQMTQSPSSLSASVGDRVTITCRASRSISRYLNWYQQKPGKAPKLLIYAASSLQAGVPS |
| | | RFSGSGSGTDFTLTISSLQPEDFATYYCQQSYSSLLTFGQGTKVEIK |
| 3A-L7 | 3482 | DIQMTQSPSSLSASVGDRVTITCRASQSIGKYLNWYQQKPGKAPKLLIYASSTLQRGVP |
| | | SRFSGSGSGTDFTLTISSLQPEDFATYYCQQSLSPPFTFGQGTKVEIK |
| 3A-L8 | 3483 | DIQMTQSPSSLSASVGDRVTITCRASQSIGRYLNWYQQKPGKAPKLLIYASSSLQSGVPS |
| | | RFSGSGSGTDFTLTISSLQPEDFATYYCQQSYSLPRTFGQGTKVEIK |
| 3A-L9 | 3484 | DIQMTQSPSSLSASVGDRVTITCRASQSIGRYLNWYQQKPGKAPKLLIYAASSLKSGVPS |
| | | RFSGSGSGTDFTLTISSLQPEDFATYYCQQSYSLPRTFGQGTKVEIK |
| 3A-L10 | 3485 | DIQMTQSPSSLSASVGDRVTITCRASQSIGKYLNWYQQKPGKAPKLLIYASSTLQRGVP |
| | | SRFSGSGSGTDFTLTISSLQPEDFATYYCQQSLSTPFTFGQGTKVEIK |
| 3A-L11 | 3486 | DIQMTQSPSSLSASVGDRVTITCRASQSIGRYLNWYQQKPGKAPKLLIYAASSLKSGVPS |
| | | RFSGSGSGTDFTLTISSLQPEDFATYYCQQSYSLPLTFGQGTKVEIK |
| 3A-L12 | 3487 | DIQMTQSPSSLSASVGDRVTITCRTSQSINTYLNWYQQKPGKAPKLLIYGASNVQSGVP |
| | | SRFSGSGSGTDFTLTISSLQPEDFATYYCQQSYRIPRTFGQGTKVEIK |
| 3A-L13 | 3488 | DIQMTQSPSSLSASVGDRVTITCRASQSIGKYLNWYQQKPGKAPKLLIYASSTLQRGVP |
| | | SRFSGSGSGTDFTLTISSLQPEDFATYYCQQSFSPPFTFGQGTKVEIK |
| 3A-L14 | 3489 | DIQMTQSPSSLSASVGDRVTITCRASQSIGKYLNWYQQKPGKAPKLLIYASSTLQRGVP |
| | | SRFSGSGSGTDFTLTISSLQPEDFATYYCQQSFSTPFTFGQGTKVEIK |
| 3A-L15 | 3490 | DIQMTQSPSSLSASVGDRVTITCRASQSIGRYLNWYQQKPGKAPKLLIYAASSLKSGVPS |
| | | RFSGSGSGTDFTLTISSLQPEDFATYYCQQSYSLPRTFGQGTKVEIK |
| 3A-H16 | 3491 | EVQLLESGGGLVQPGGSLRLSCAASGFTFTNFAMSWVRQAPGKGLEWVSAISGRGGGT |
| | | YYADSVKGRFTISRDNSKNTLYLQMNSLRAEDTAVYYCARDAHGYYYDSSGYDDWG |
| | | QGTLVTVSS |
| 3A-H17 | 3492 | EVQLLESGGGLVQPGGSLRLSCAASGFTFRSYPMSWVRQAPGKGLEWVSTISGSGGITY |
| | | YADSVKGRFTISRDNSKNTLYLQMNSLRAEDTAVYYCAKGVYGSTVTTCHWGQGTLV |
| | | TVSS |
| 3A-H18 | 3493 | EVQLLESGGGLVQPGGSLRLSCAASGFTLTSYAMSWVRQAPGKGLEWVSAISGSGVDT |
| | | YYADSVKGRFTISRDNSKNTLYLQMNSLRAEDTAVYYCARPTNWGFDYWGQGTLVT |
| | | VSS |
| 3A-H19 | 3494 | EVQLLESGGGLVQPGGSLRLSCAASGFTFINYAMSWVRQAPGKGLEWVSTISTSGGNT |
| | | YYADSVKGRFTISRDNSKNTLYLQMNSLRAEDTAVYYCARADSNWASSAYWGQGTL |
| | | VTVSS |
| 3A-H20 | 3495 | EVQLLESGGGLVQPGGSLRLSCAASGFPFSTYAMSWVRQAPGKGLEWVSGISVSGGFT |
| | | YYADSVKGRFTISRDNSKNTLYLQMNSLRAEDTAVYYCARDPYSYGYYYYYGMDVW |
| | | GQGTLVTVSS |
| 3A-H21 | 3496 | EVQLLESGGGLVQPGGSLRLSCAASGFTFSTYAMGWVRQAPGKGLEWVSGISGGGVS |
| | | TYYADSVKGRFTISRDNSKNTLYLQMNSLRAEDTAVYYCARARNWGPSDYWGQGTL |
| | | VTVSS |
| 3A-H22 | 3497 | EVQLLESGGGLVQPGGSLRLSCAASGFIFSDYAMTWVRQAPGKGLEWVSAISGSAFYA |
| | | DSVKGRFTISRDNSKNTLYLQMNSLRAEDTAVYYCARDATYSSSWYNWFDPWGQGTL |
| | | VTVSS |
| 3A-H23 | 3498 | EVQLLESGGGLVQPGGSLRLSCAASGFTFSDYAMTWVRQAPGKGLEWVSDISGSGGST |
| | | YYADSVKGRFTISRDNSKNTLYLQMNSLRAEDTAVYYCARGTVTSFDFWGQGTLVTV |
| | | SS |
| 3A-H24 | 3499 | EVQLLESGGGLVQPGGSLRLSCAASGFTFSIYAMGWVRQAPGKGLEWVSFISGSGGST |
| | | YYADSVKGRFTISRDNSKNTLYLQMNSLRAEDTAVYYCAKDYHSASWFSAAADYWG |
| | | QGTLVTVSS |
| 3A-H25 | 3500 | EVQLLESGGGLVQPGGSLRLSCAASGFTFASYAMTWVRQAPGKGLEWVSAISESGGST |
| | | YYADSVKGRFTISRDNSKNTLYLQMNSLRAEDTAVYYCAREGQEYSSGSSYFDYWGQ |
| | | GTLVTVSS |
| 3A-H26 | 3501 | EVQLLESGGGLVQPGGSLRLSCAASGFTFSEYAMSWVRQAPGKGLEWVSAITGSGGST |
| | | YYGDSVKGRFTISRDNSKNTLYLQMNSLRAEDTAVYYCARGSQTPYCGGDCPETFDY |
| | | WGQGTLVTVSS |
| 3A-H27 | 3502 | EVQLLESGGGLVQPGGSLRLSCAASGFTFDDYAMSWVRQAPGKGLEWVSGISGGGTS |
| | | TYYADSVKGRFTISRDNSKNTLYLQMNSLRAEDTAVYYCARDLYSSGWYGFDYWGQ |
| | | GTLVTVSS |
| 3A-H28 | 3503 | EVQLLESGGGLVQPGGSLRLSCAASGFTFNNYAMNWVRQAPGKGLEWVSAISGSVGS |
| | | TYYADSVKGRFTISRDNSKNTLYLQMNSLRAEDTAVYYCARDNYDFWSGYYTNWFD |
| | | PWGQGTLVTVSS |
| 3A-H29 | 3504 | EVQLLESGGGLVQPGGSLRLSCAASGFTFTNHAMSWVRQAPGKGLEWVSAISGSGSNI |
| | | YYADSVKGRFTISRDNSKNTLYLQMNSLRAEDTAVYYCARDSLSVTMGRGVVTYYYY |
| | | GMDFWGQGTLVTVSS |
| 3A-L16 | 3505 | DIQMTQSPSSLSASVGDRVTITCRASQIIGSYLNWYQQKPGKAPKLLIYTTSNLQSGVPS |
| | | RFSGSGSGTDFTLTISSLQPEDFATYYCQQSYITPWTFGQGTKVEIK |
| 3A-L17 | 3506 | DIQMTQSPSSLSASVGDRVTITCRASQSISRYINWYQQKPGKAPKLLIYEASSLESGVPSR |
| | | FSGSGSGTDFTLTISSLQPEDFATYYCQQSHITPLTFGQGTKVEIK |
| 3A-L18 | 3507 | DIQMTQSPSSLSASVGDRVTITCRASQSIYTYLNWYQQKPGKAPKLLIYSASNLHSGVPS |
| | | RFSGSGSGTDFTLTISSLQPEDFATYYCQQSDTTPWTFGQGTKVEIK |
| 3A-L19 | 3508 | DIQMTQSPSSLSASVGDRVTITCRASQSIATYLNWYQQKPGKAPKLLIYGASSLEGGVPS |
| | | RFSGSGSGTDFTLTISSLQPEDFATYYCQQTFSSPFTFGQGTKVEIK |
| 3A-L20 | 3509 | DIQMTQSPSSLSASVGDRVTITCRASQNINTYLNWYQQKPGKAPKLLIYSASSLQSGVPS |
| | | RFSGSGSGTDFTLTISSLQPEDFATYYCQQSSLTPWTFGQGTKVEIK |
| 3A-L21 | 3510 | DIQMTQSPSSLSASVGDRVTITCRASQGIATYLNWYQQKPGKAPKLLIYYASNLQSGVP |
| | | SRFSGSGSGTDFTLTISSLQPEDFATYYCQQSYSTRFTFGQGTKVEIK |
| 3A-L22 | 3511 | DIQMTQSPSSLSASVGDRVTITCRASERISNYLNWYQQKPGKAPKLLIYTASNLESGVPS |
| | | RFSGSGSGTDFTLTISSLQPEDFATYYCQQSYTPPRTFGQGTKVEIK |
| 3A-L23 | 3512 | DIQMTQSPSSLSASVGDRVTITCRASQSISSSLNWYQQKPGKAPKLLIYAASRLQDGVPS |
| | | RFSGSGSGTDFTLTISSLQPEDFATYYCQQSYSTPRSFGQGTKVEIK |
| 3A-L24 | 3513 | DIQMTQSPSSLSASVGDRVTITCRASQSISSHLNWYQQKPGKAPKLLIYRASTLQSGVPS |
| | | RFSGSGSGTDFTLTISSLQPEDFATYYCQQTYNTPQTFGQGTKVEIK |
| 3A-L25 | 3514 | DIQMTQSPSSLSASVGDRVTITCRASQSISSYLIWYQQKPGKAPKLLIYAASRLHSGVPS |
| | | RFSGSGSGTDFTLTISSLQPEDFATYYCQQGYNTPRTFGQGTKVEIK |
| 3A-L26 | 3515 | DIQMTQSPSSLSASVGDRVTITCRASPSISTYLNWYQQKPGKAPKLLIYTASRLQTGVPS |
| | | RFSGSGSGTDFTLTISSLQPEDFATYYCQQTYSTPSSFGQGTKVEIK |
| 3A-L27 | 3516 | DIQMTQSPSSLSASVGDRVTITCRASQNIAKYLNWYQQKPGKAPKLLIYGASGLQSGVP |
| | | SRFSGSGSGTDFTLTISSLQPEDFATYYCQQSHSPPITFGQGTKVEIK |
| 3A-L28 | 3517 | DIQMTQSPSSLSASVGDRVTITCRASQSIGTYLNWYQQKPGKAPKLLIYAASNLHSGVP |
| | | SRFSGSGSGTDFTLTISSLQPEDFATYYCQESYSAPYTFGQGTKVEIK |
| 3A-L29 | 3518 | DIQMTQSPSSLSASVGDRVTITCRASQSISPYLNWYQQKPGKAPKLLIYKASSLQSGVPS |
| | | RFSGSGSGTDFTLTISSLQPEDFATYYCQQSSSTPYTFGQGTKVEIK |
| 4A-H51 | 3519 | EVQLVESGGGLVQPGGSLRLSCAASGPGTAIMGWFRQAPGKEREFVARISTSGGSTKY |
| | | ADSVKGRFTISADNSKNTAYLQMNSLKPEDTAVYYCARTTVTTPPLIWGQGTLVTVSS |
| 4A-H52 | 3520 | EVQLVESGGGLVQPGGSLRLSCAASGRSFSNSVMGWFRQAPGKEREFVARITWNGGST |
| | | YYADSVKGRFTISADNSKNTAYLQMNSLKPEDTAVYYCATTENPNPRWGQGTLVTVS |
| | | S |
| 4A-H53 | 3521 | EVQLVESGGGLVQPGGSLRLSCAASGRTFGDDTMGWFRQAPGKEREFVAAVSWSGSG |
| | | VYYADSVKGRFTITADNSKNTAYLQMNSLKPENTAVYYCATDPPLFWGQGTLVTVSS |
| 4A-H54 | 3522 | EVQLVESGGGLVQPGGSLRLSCAASGRTFSDARMGWFRQAPGKEREFVGAVSWSGGT |
| | | TVYADSVKGRFTISADNSKNTAYLQMNSLKPEDTAVYYCATTEDPYPRWGQGTLVTV |
| | | SS |
| 4A-H49 | 3523 | EVQLVESGGGLVQPGGSLRLSCAASGRTFGDYIMGWFRQAPGKERESVAAINWSAGY |
| | | TAYADSVKGRFTISADNSKNTAYLQMNSLKPEDTAVYYCARASPNTGWHFDHWGQG |
| | | TLVTVSS |
| 4A-H55 | 3524 | EVQLVESGGGLVQPGGSLRLSCAASGSGLSINAMGWFRQAPGKERESVAAISWSGGST |
| | | YTAYADSVKGRFTISADNSKNTAYLQMNSLKPEDTAVYYCAAYQAGWGDWGQGTLV |
| | | TVSS |
| 4A-H39 | 3525 | EVQLVESGGGLVQPGGSLRLSCAASGRTFSNAAMGWFRQAPGKEREFVARILWTGAS |
| | | RNYADSVKGRFTISADNSKNTAYLQMNSLKPEDTAVYYCATTENPNPRWGQGTLVTV |
| | | SS |
| 4A-H56 | 3526 | EVQLVESGGGLVQPGGSLRLSCAASGFSLDYYGMGWFRQAPGKERESVAAISWNGDF |
| | | TAYADSVKGRFTISADNSKNTAYLQMNSLKPEDTAVYYCAKRANPTGAYFDYWGQG |
| | | TLVTVSS |
| 4A-H33 | 3527 | EVQLVESGGGLVQPGGSLRLSCAASGFTFSRHDMGWFRQAPGKEREFVAGINWESGST |
| | | NYADSVKGRFTISADNSKNTAYLQMNSLKPEDTAVYYCAADRGVYGGRWYRTSQYT |
| | | WGQGTLVTVSS |
| 4A-H57 | 3528 | EVQLVESGGGLVQPGGSLRLSCAASGLTFRNYAMGWFRQAPGKEREFVAAIGSGGYT |
| | | DYADSVKGRFTISADNSKNTAYLQMNSLKPEDTAVYYCAVKPGWVARDPSQYNWGQ |
| | | GTLVTVSS |
| 4A-H25 | 3529 | EVQLVESGGGLVQPGGSLRLSCAASGGTFSRYAMGWFRQAPGKEREWVSAVDSGGST |
| | | YYADSVKGRFTISADNSKNTAYLQMNSLKPEDTAVYYCAASPSLRSAWQWGQGTLVT |
| | | VSS |
| 4A-H58 | 3530 | EVQLVESGGGLVQPGGSLRLSCAASGFTLDYYDMGWFRQAPGKEREFVAAVTWSGGS |
| | | TYYADSVKGRFTISADNSKNTAYLQMNSLKPEDTAVYYCAADRRGLASTRAADYDW |
| | | GQGTLVTVSS |
| 4A-H59 | 3531 | EVQLVESGGGLVQPGGSLRLSCAASGRTFGDYIMGWFRQAPGKEREFVAAINWSAGY |
| | | TPYADSVKGRFTISADNSKNTAYLQMNSLKPEDTAVYYCATAPPLFCWHFDLWGQGT |
| | | LVTVSS |
| 4A-H6 | 3532 | EVQLVESGGGLVQPGGSLRLSCAASGRTFGDDIMGWFRQAPGKEREFVAAIHWSAGY |
| | | TRYADSVKGRFTISADNSKNTAYLQMNSLKPEDTAVYYCATDPPLFWGHVDLWGQGT |
| | | LVTVSS |
| 4A-H61 | 3533 | EVQLVESGGGLVQPGGSLRLSCAASGRTFGDYIMGWFRQAPGKEREIVAAINWSADYT |
| | | PYADSVKGRFTISADNSKNTAYLQMNSLKPEDTAVYYCATAPPNTGWHFDHWGQGTL |
| | | VTVSS |
| 4A-H3 | 3534 | EVQLVESGGGLVQPGGSLRLSCAASGRTFGDYIMGWFRQAPGKEREIVAAINWSAGYT |
| | | AYADSVKGRFTISADNSKNTAYLQMNSLKPEDTAVYYCATATPNTGWHFDHWGQGT |
| | | LVTVSS |
| 4A-H62 | 3535 | EVQLVESGGGLVQPGGSLRLSCAASGRTFSDDTMGWFRQAPGKEREFVAAINWSGGS |
| | | TDYADSVKGRFTISADNSKNTAYLQMNSLKPEDTAVYYCATDPPLFWGQGTLVTVSS |
| 4A-H43 | 3536 | EVQLVESGGGLVQPGGSLRLSCAASGRTFGDDTMGWFRQAPGKEREFVAGINWSGGN |
| | | TYYADSVKGRFTISADNSKNTAYLQMNSLKPEDTAVYYCATDPPLFWGQGTLVTVSS |
| 4A-H5 | 3537 | EVQLVESGGGLVQPGGSLRLSCAASGRTFGDYIMGWFRQAPGKEREFVAAINWTGGY |
| | | TSYADSVKGRFTISADNSKNTAYLQMNSLKPEDTAVYYCATDPPLFWGQGTLVTVSS |
| 4A-H42 | 3538 | EVQLVESGGGLVQPGGSLRLSCAASGRTFGDDTMGWFRQAPGKERECVAAINWSGGN |
| | | TYYADSVKGRFTISADNSKNTAYLQMNSLKPEDTAVYYCATDPPLFWGQGTLVTVSS |
| 4A-H63 | 3539 | EVQLVESGGGLVQPGGSLRLSCAASGRTFSDYTMGWFRQAPGKEREFVAAINWSGGY |
| | | TYYADSVKGRFTISADNSKNTAYLQMNSLKPEDTAVYYCATDPPLFWGQGTLVTVSS |
| 4A-H6 | 3540 | EVQLVESGGGLVQPGGSLRLSCAASGRTFGDYGMGWFRQAPGKEREFVATINWSGAL |
| | | THYADSVKGRFTISADNSKNTAYLQMNSLKPEDTAVYYCATLPFYDFWSGYYTGYYY |
| | | MDVWGQGTLVTVSS |
| 4A-H40 | 3541 | EVQLVESGGGLVQPGGSLRLSCAASGRTFSDDTMGWFRQAPGKEREFLAGVTWSGSS |
| | | TFYADSVKGRFTISADNSKNTAYLQMNSLKPEDTAVYYCATDPPLFWGQGTLVTVSS |
| 4A-H21 | 3542 | EVQLVESGGGLVQPGGSLRLSCAASGRTFSDDIMGWFRQAPGKEREFVAAISWSGGNT |
| | | HYADSVKGRFTISADNSKNTAYLQMNSLKPEDTAVYYCATDPPLFWGQGTLVTVSS |
| 4A-H64 | 3543 | EVQLVESGGGLVQPGGSLRLSCAASGRTFGDYIMGWFRQAPGKERESVAAINWSAGY |
| | | TAYADSVKGRFTISADNSKNTAYLQMNSLKPEDTAVYYCATASPNTGWHFDHWGQG |
| | | TLVTVSS |
| 4A-H47 | 3544 | EVQLVESGGGLVQPGGSLRLSCAASGFTFDDDYVMGWFRQAPGKEREFVAAVSGSGD |
| | | DTYYADSVKGRFTISADNSKNTAYLQMNSLKPEDTAVYYCAADRRGLASTRAADYD |
| | | WGQGTLVTVSS |
| 4A-H65 | 3545 | EVQLVESGGGLVQPGGSLRLSCAASGRTFGDYIMGWFRQAPGKEREFVAAINWSAGY |
| | | TAYADSVKGRFTISADNSKNTAYLQMNSLKPEDTAVYYCATEPPLSCWHFDLWGQGT |
| | | LVTVSS |
| 4A-H18 | 3546 | EVQLVESGGGLVQPGGSLRLSCAASGRTFGDYIMGWFRQAPGKEREIVAAINWSGGYT |
| | | PYADSVKGRFTISADNSKNTAYLQMNSLKPEDTAVYYCATAPPNTGWHFDHWGQGTL |
| | | VTVSS |
| 4A-H66 | 3547 | EVQLVESGGGLVQPGGSLRLSCAASGRTFGDDTMGWFRQAPGKEREIVAAINWSAGY |
| | | TPYADSVKGRFTISADNSKNTAYLQMNSLKPEDTAVYYCATDPPLFCCHFDLWGQGTL |
| | | VTVSS |
| 4A-H36 | 3548 | EVQLVESGGGLVQPGGSLRLSCAASGRTFSDDTMGWFRQAPGKEREFVAAISWSGGTT |
| | | RYADSVKGRFTISADNSKNTAYLQMNSLKPEDTAVYYCATDPPLFWGQGTLVTVSS |
| 4A-H67 | 3549 | EVQLVESGGGLVQPGGSLRLSCAASGRTFSDDTMGWFRQAPGKEREFVAAINWSGDS |
| | | TYYADSVKGRFTISADNSKNTAYLQMNSLKPEDTAVYYCATDPPLFWGQGTLVTVSS |
| 4A-H16 | 3550 | EVQLVESGGGLVQPGGSLRLSCAASGRTFSDDTMGWFRQAPGKEREFVAAINWSGGT |
| | | TRYADSVKGRFTISADNSKNTAYLQMNSLKPEDTAVYYCATDPPLFWGQGTLVTVSS |
| 4A-H11 | 3551 | EVQLVESGGGLVQPGGSLRLSCAASGRTFSDDAMGWFRQAPGKEREFVAAIHWSGSST |
| | | RYADSVKGRFTISADNSKNTAYLQMNSLKPEDTAVYYCATDPPLFWGQGTLVTVSS |
| 4A-H68 | 3552 | EVQLVESGGGLVQPGGSLRLSCAASGRTFSDDTMGWFRQAPGKERELVGTINWSGGST |
| | | YYADSVKGRFTISADNSKNTAYLQMNSLKPEDTAVYYCATDPPLFWGQGTLVTVSS |
| 4A-H34 | 3553 | EVQLVESGGGLVQPGGSLRLSCAASGRTFGDYIMGWFRQAPGKEREFVAAINWSGGY |
| | | TPYADSVKGRFTISADNSKNTAYLQMNSLKPEDTAVYYCATDPPLFWGQGTLVTVSS |
| 4A-H28 | 3554 | EVQLVESGGGLVQPGGSLRLSCAASGRTFGDDTMGWFRQAPGKERELVAAINWNGGN |
| | | THYADSVKGRFTISADNSKNTAYLQMNSLKPEDTAVYYCATDPPLFWGQGTLVTVSS |
| 4A-H69 | 3555 | EVQLVESGGGLVQPGGSLRLSCAASGRTFSDDAMGWFRQAPGKEREFVAAINWSGGT |
| | | TRYADSVKGRFTISADNSKNTAYLQMNSLKPEDTAVYYCATDPPLFWGQGTLVTVSS |
| 4A-H7 | 3556 | EVQLVESGGGLVQPGGSLRLSCAASGRTFGDYIMGWFRQAPGKERESVAAINWSAGY |
| | | TPYADSVKGRFTISADNSKNTAYLQMNSLKPEDTAVYYCATDPPLFWGHVDLWGQGT |
| | | LVTVSS |
| 4A-H71 | 3557 | EVQLVESGGGLVQPGGSLRLSCAASGRTFSDDTMGWFRQAPGKEREWVASINWSGGS |
| | | TYYADSVKGRFTISADNSKNTAYLQMNSLKPEDTAVYYCATDPPLFWGQGTLVTVSS |
| 4A-H23 | 3558 | EVQLVESGGGLVQPGGSLRLSCAASGRTFSDDAMGWFRQAPGKEREFVAGISWNGGSI |
| | | YYADSVKGRFTISADNSKNTAYLQMNSLKPEDTAVYYCATDPPLFWGQGTLVTVSS |
| 4A-H9 | 3559 | EVQLVESGGGLVQPGGSLRLSCAASGFTFDDYEMGWFRQAPGKEREFVAAISWRGGT |
| | | TYYADSVKGRFTISADNSKNTAYLQMNSLKPEDTAVYYCAADRRGLASTRAGDYDW |
| | | GQGTLVTVSS |
| 4A-H72 | 3560 | EVQLVESGGGLVQPGGSLRLSCAASGRTFGDDTMGWFRQAPGKEREFVAAINWSGGY |
| | | TPYADSVKGRFTISADNSKNTAYLQMNSLKPEDTAVYYCATDPPLFWGHVDLWGQGT |
| | | LVTVSS |
| 4A-H73 | 3561 | EVQLVESGGGLVQPGGSLRLSCAASGRTFSDDAMGWFRQAPGKEREFVAAINWSGGS |
| | | TRYADSVKGRFTISADNSKNTAYLQMNSLKPEDTAVYYCATDPPLFWGQGTLVTVSS |
| 4A-H29 | 3562 | EVQLVESGGGLVQPGGSLRLSCAASGVTLDDYAMGWFRQAPGKEREFVAVINWSGGS |
| | | TDYADSVKGRFTISADNSKNTAYLQMNSLKPEDTAVYYCARGGGWVPSSTSESLNWY |
| | | FDRWGQGTLVTVSS |
| 4A-H41 | 3563 | EVQLVESGGGLVQPGGSLRLSCAASGRTFGDYIMGWFRQAPGKERESVAAINWSGGTT |
| | | PYADSVKGRFTISADNSKNTAYLQMNSLKPEDTAVYYCATDPPLFCCHVDLWGQGTL |
| | | VTVSS |
| 4A-H74 | 3564 | EVQLVESGGGLVQPGGSLRLSCAASGLTFSDDTMGWFRQAPGKEREFVAAVSWSGGN |
| | | TYYADSVKGRFTISADNSKNTAYLQMNSLKPEDTAVYYCATDPPLFWGQGTLVTVSS |
| 4A-H75 | 3565 | EVQLVESGGGLVQPGGSLRLSCAASGRTFGDDTMGWFRQAPGKEREFVAAINWTGGY |
| | | TPYADSVKGRFTISADNSKNTAYLQMNSLKPEDTAVYYCATDPPLFWGQGTLVTVSS |
| 4A-H31 | 3566 | EVQLVESGGGLVQPGGSLRLSCAASGRTFGDYIMGWFRQAPGKEREFVATINWTAGY |
| | | TYYADSVKGRFTISADNSKNTAYLQMNSLKPEDTAVYYCATDPPLFCWHFDHWGQGT |
| | | LVTVSS |
| 4A-H32 | 3567 | EVQLVESGGGLVQPGGSLRLSCAASGRTFGDDTMGWFRQAPGKEREFVAAINWSGGN |
| | | TDYADSVKGRFTISADNSKNTAYLQMNSLKPEDTAVYYCATDPPLFWGQGTLVTVSS |
| 4A-H15 | 3568 | EVQLVESGGGLVQPGGSLRLSCAASGRTFGDYTMGWFRQAPGKEREFVAAINWSGGN |
| | | TYYADSVKGRFTISADNSKNTAYLQMNSLKPEDTAVYYCATDPPLFWGQGTLVTVSS |
| 4A-H14 | 3569 | EVQLVESGGGLVQPGGSLRLSCAASGRTFSDDTMGWFRQAPGKEREFVAGINWSGNG |
| | | VYYADSVKGRFTISADNSKNTAYLQMNSLKPEDTAVYYCATDPPLFWGQGTLVTVSS |
| 4A-H76 | 3570 | EVQLVESGGGLVQPGGSLRLSCAASGRTFGDYAMGWFRQAPGKERELVAPINWSGGS |
| | | TYYADSVKGRFTISADNSKNTAYLQMNSLKPEDTAVYYCATDPPLFWGQGTLVTVSS |
| 4A-H50 | 3571 | EVQLVESGGGLVQPGGSLRLSCAASGGTFSNSGMGWFRQAPGKERELVAVVNWSGRR |
| | | TYYADSVKGRFTISADNSKNTAYLQMNSLKPEDTAVYYCAVPWMDYNRRDWGQGTL |
| | | VTVSS |
| 4A-H17 | 3572 | EVQLVESGGGLVQPGGSLRLSCAASGQLANFASYAMGWFRQAPGKEREFVAAITRSGS |
| | | STVYADSVKGRFTISADNSKNTAYLQMNSLKPEDTAVYYCATTMNPNPRWGQGTLVT |
| | | VSS |
| 4A-H37 | 3573 | EVQLVESGGGLVQPGGSLRLSCAASGRTFSDDIMGWFRQAPGKEREFVAAINWTGGST |
| | | YYADSVKGRFTISADNSKNTAYLQMNSLKPEDTAVYYCATDPPLFWGQGTLVTVSS |
| 4A-H44 | 3574 | EVQLVESGGGLVQPGGSLRLSCAASGRTFGDYIMGWFRQAPGKEREIVAAINWSAGYT |
| | | AYADSVKGRFTISADNSKNTAYLQMNSLKPEDTAVYYCATARPNTGWHFDHWGQGT |
| | | LVTVSS |
| 4A-H77 | 3575 | EVQLVESGGGLVQPGGSLRLSCAASGRTFSDDTMGWFRQAPGKEREWVGSINWSGGS |
| | | TYYADSVKGRFTISADNSKNTAYLQMNSLKPEDTAVYYCATDPPLFWGQGTLVTVSS |
| 4A-H78 | 3576 | EVQLVESGGGLVQPGGSLRLSCAASGRTFSDDTMGWFRQAPGKEREFVAGMTWSGSS |
| | | TFYADSVKGRFTISADNSKNTAYLQMNSLKPEDTAVYYCATDPPLFWGQGTLVTVSS |
| 4A-H79 | 3577 | EVQLVESGGGLVQPGGSLRLSCAASGRTFGDYIMGWFRQAPGKERECVAAINWSGDY |
| | | TDYADSVKGRFTISADNSKNTAYLQMNSLKPEDTAVYYCATDPPLFWGQGTLVTVSS |
| 4A-H8 | 3578 | EVQLVESGGGLVQPGGSLRLSCAASGRTFGDYIMGWFRQAPGKEREFVGGINWSGGY |
| | | TYYADSVKGRFTISADNSKNTAYLQMNSLKPEDTAVYYCATDPPLFWGQGTLVTVSS |
| 4A-H81 | 3579 | EVQLVESGGGLVQPGGSLRLSCAASGRTFSDDTMGWFRQAPGKEREFVAAVNWSGGS |
| | | TYYADSVKGRFTISADNSKNTAYLQMNSLKPEDTAVYYCATDPPLFWGQGTLVTVSS |
| 4A-H82 | 3580 | EVQLVESGGGLVQPGGSLRLSCAASGRTFGDYAMGWFRQAPGKEREFVAAINWSGGY |
| | | TRYADSVKGRFTISADNSKNTAYLQMNSLKPEDTAVYYCATDPPLFWGQGTLVTVSS |
| 4A-H83 | 3581 | EVQLVESGGGLVQPGGSLRLSCAASGRTFGDDTMGWFRQAPGKEREFVAAINWSGGY |
| | | TPYADSVKGRFTISADNSKNTAYLQMNSLKPEDTAVYYCATDPPLFWGQGTLVTVSS |
| 4A-H35 | 3582 | EVQLVESGGGLVQPGGSLRLSCAASGRTFGDYIMGWFRQAPGKERESVAAINWSAGY |
| | | TAYADSVKGRFTISADNSKNTAYLQMNSLKPEDTAVYYCARASPNTGWHFDRWGQG |
| | | TLVTVSS |
| 4A-H45 | 3583 | EVQLVESGGGLVQPGGSLRLSCAASGRTFGDYIMGWFRQAPGKEREFVAAINWSGGY |
| | | THYADSVKGRFTISADNSKNTAYLQMNSLKPEDTAVYYCATDPPLFWGQGTLVTVSS |
| 4A-H84 | 3584 | EVQLVESGGGLVQPGGSLRLSCAASGRTFSDDTMGWFRQAPGKEREFVAAITWSGGR |
| | | TRYADSVKGRFTISADNSKNTAYLQMNSLKPEDTAVYYCATDRPLFWGQGTLVTVSS |
| 4A-H85 | 3585 | EVQLVESGGGLVQPGGSLRLSCAASGRTFGDYIMGWFRQAPGKERESVAAINWSGGY |
| | | TAYADSVKGRFTISADNSKNTAYLQMNSLKPEDTAVYYCATASPNTGWHFDHWGQG |
| | | TLVTVSS |
| 4A-H86 | 3586 | EVQLVESGGGLVQPGGSLRLSCAASGRTFSDDTMGWFRQAPGKEREFVAAIHWSGSST |
| | | RYADSVKGRFTISADNSKNTAYLQMNSLKPEDTAVYYCATDPPLFWGQGTLVTVSS |
| 4A-H87 | 3587 | EVQLVESGGGLVQPGGSLRLSCAASGRTFSDYTMGWFRQAPGKEREWVAAINWSGGT |
| | | TYYADSVKGRFTISADNSKNTAYLQMNSLKPEDTAVYYCATDPPLFWGQGTLVTVSS |
| 4A-H88 | 3588 | EVQLVESGGGLVQPGGSLRLSCAASGRTFGDDTMGWFRQAPGKEREFVAAINWSGDN |
| | | THYADSVKGRFTISADNSKNTAYLQMNSLKPEDTAVYYCATDPPLFWGQGTLVTVSS |
| 4A-H89 | 3589 | EVQLVESGGGLVQPGGSLRLSCAASGFAFGDNWIGWFRQAPGKEREWVASISSGGTTA |
| | | YADNVKGRFTIIADNSKNTAYLQMNSLKPEDTAVYYCAHRGGWLRPWGYWGQGTLV |
| | | TVSS |
| 4A-H9 | 3590 | EVQLVESGGGLVQPGGSLRLSCAASGRTFSDDAMGWFRQAPGKEREFVGRINWSGGN |
| | | TYYADSVKGRFTISADNSKNTAYLQMNSLKPEDTAVYYCATDPPLFWGQGTLVTVSS |
| 4A-H91 | 3591 | EVQLVESGGGLVQPGGSLRLSCAASGRTFSDDTMGWFRQAPGKEREFVGGISWSGGN |
| | | TYYADSVKGRFTISADNSKNTAYLQMNSLKPEDTAVYYCATDPPLFWGQGTLVTVSS |
| 4A-H92 | 3592 | EVQLVESGGGLVQPGGSLRLSCAASGRTFSDDTMGWFRQAPGKEREFVAAINWSGGS |
| | | TYYADSVKGRFTISADNSKNTAYLQMNSLKPEDTAVYYCATDPPLFWGQGTLVTVSS |
| 4A-H46 | 3593 | EVQLVESGGGLVQPGGSLRLSCAASGRTFGDDTMGWFRQAPGKEREFVAAINWSGGY |
| | | TYYADSVKGRFTISADNSKNTAYLQMNSLKPEDTAVYYCATDPPLFWGQGTLVTVSS |
| 4A-H20 | 3594 | EVQLVESGGGLVQPGGSLRLSCAASGRTFGDYIMGWFRQAPGKEREFVAAINWSADY |
| | | TAYADSVKGRFTISADNSKNTAYLQMNSLKPEDTAVYYCATDPPLFCWHFDHWGQGT |
| | | LVTVSS |
| 4A-H93 | 3595 | EVQLVESGGGLVQPGGSLRLSCAASGRTFSDDAMGWFRQAPGKEREFVAAINWSGSST |
| | | YYADSVKGRFTISADNSKNTAYLQMNSLKPEDTAVYYCATDPPLFWGQGTLVTVSS |
| 4A-H4 | 3596 | EVQLVESGGGLVQPGGSLRLSCAASGRTFGDYIMGWFRQAPGKEREMVAAINWIAGY |
| | | TADADSVRRLFTITADNNKNTAHLMMNLLKPENTAVYYCAEPSPNTGWHFDHWGQG |
| | | TLVTVSS |
| 4A-H2 | 3597 | EVQLVESGGGLVQPGGSLRLSCAASGRTFGDDTMGWFRQAPGKEREFVAAINWSGGN |
| | | TPYADSVKGRFTISADNSKNTAYLQMNSLKPEDTAVYYCATDPPLFWGQGTLVTVSS |
| 4A-H94 | 3598 | EVQLVESGGGLVQPGGSLRLSCAASGRTFSDDTMGWFRQAPGKEREFVAAINWSGDN |
| | | THYADSVKGRFTISADNSKNTAYLQMNSLKPEDTAVYYCATDPPLFWGQGTLVTVSS |
| 4A-H95 | 3599 | EVQLVESGGGLVQPGGSLRLSCAASGRTFGDYIMGWFRQAPGKEREIVAAINWSAGYT |
| | | AYADSVKGRFTISADNSKNTAYLQMNSLKPEDTAVYYCATAPPLFCWHFDHWGQGTL |
| | | VTVSS |
| 4A-H12 | 3600 | EVQLVESGGGLVQPGGSLRLSCAASGFTFGDYVMGWFRQAPGKEREIVAAINWNAGY |
| | | TAYADSVRGLFTITADNSKNTAYLQMNSLKPEDTAVYYCAKASPNTGWHFDHWGQG |
| | | TLVTVSS |
| 4A-H30 | 3601 | EVQLVESGGGLVQPGGSLRLSCAASGRTFGDYTMGWFRQAPGKEREFVAAINWTGGY |
| | | TYYADSVKGRFTISADNSKNTAYLQMNSLKPEDTAVYYCATDPPLFWGQGTLVTVSS |
| 4A-H27 | 3602 | EVQLVESGGGLVQPGGSLRLSCAASGRTFGDYIMGWFRQAPGKEREIVAAINWSAGYT |
| | | AYADSVKGLFTITADNSKNTAYLQMNILKPEDTAVYYCARATPNTGWHFDHWGQGTL |
| | | VTVSS |
| 4A-H22 | 3603 | EVQLVESGGGLVQPGGSLRLSCAASGRTFGDYIMGWFRQAPGKEREFVAAINWSGDN |
| | | THYADSVKGRFTISADNSKNTAYLQMNSLKPEDTAVYYCATDPPLFWGQGTLVTVSS |
| 4A-H96 | 3604 | EVQLVESGGGLVQPGGSLRLSCAASGRTFGDYIMGWFRQAPGKEREIVAAINWSAGYT |
| | | PYADSVKGRFTISADNSKNTAYLQMNSLKPEDTAVYYCATDPPLFCCHFDHWGQGTL |
| | | VTVSS |
| 4A-H97 | 3605 | EVQLVESGGGLVQPGGSLRLSCAASGRTFGDYIMGWFRQAPGKERESVAAINWSAGY |
| | | TAYADSVKGRFTISADNSKNTAYLQMNSLKPEDTAVYYCATAPPNTGWHFDHWGQG |
| | | TLVTVSS |
| 4A-H98 | 3606 | EVQLVESGGGLVQPGGSLRLSCAASGFTWGDYTMGWFRQAPGKEREFVAAINWSGG |
| | | NTYYADSVKGRFTISADNSKNTAYLQMNSLKPEDTAVYYCAADRRGLASTRAADYD |
| | | WGQGTLVTVSS |
| 4A-H99 | 3607 | EVQLVESGGGLVQPGGSLRLSCAASGIPSTLRAMGWFRQAPGKEREFVAAVSSLGPFT |
| | | RYADSVKGRFTISADNSKNTAYLQMNSLKPEDTAVYYCAAKPGWVARDPSQYNWGQ |
| | | GTLVTVSS |
| 4A-H100 | 3608 | EVQLVESGGGLVQPGGSLRLSCAASGFSFDDDYVMGWFRQAPGKEREFVAAINWSGG |
| | | STYYADSVKGRFTISADNSKNTAYLQMNSLKPEDTAVYYCAADRRGLASTRAADYDW |
| | | GQGTLVTVSS |
| 4A-H101 | 3609 | EVQLVESGGGLVQPGGSLRLSCAASGRTFSNAAMGWFRQAPGKEREFVARILWTGAS |
| | | RSYADSVKGRFTISADNSKNTAYLQMNSLKPEDTAVYYCATTENPNPRWGQGTLVTV |
| | | SS |
| 4A-H102 | 3610 | EVQLVESGGGLVQPGGSLRLSCAASGGTFGVYHMGWFRQAPGKEREGVAAINMSGD |
| | | DSAYADSVKGRFTISADNSKNTAYLQMNSLKPEDTAVYYCAILVGPGQVEFDHWGQG |
| | | TLVTVSS |
| 4A-H103 | 3611 | EVQLVESGGGLVQPGGSLRLSCAASGFTFSSYYMGWFRQAPGKEREFVARI-- |
| | | SGSTFYADSVKGRFTISADNSKNTAYLQMNSLKPEDTAVYYCAALPFVCPSGSYSDYG |
| | | DEYDWGQGTLVTVSS |
| 4A-H104 | 3612 | EVQLVESGGGLVQPGGSLRLSCAASGRTFSGDFMGWFRQAPGKEREFVGRINWSGGN |
| | | TYYADSVRGLFTITADNNKNTAYLMMNLLKPEDTAVYYCPTDPPLFWGLGTLVTWSS |
| 4A-H105 | 3613 | EVQLVESGGGLVQPGGSLRLSCAASGSTLRDYAMGWFRQAPGKERESVAAITWSGGS |
| | | TAYADSVKGRFTISADNSKNTAYLQMNSLKPEDTAVYYCASLLAGDRYFDYWGQGTL |
| | | VTVSS |
| 4A-H106 | 3614 | EVQLVESGGGLVQPGGSLRLSCAASGFTFDDYTMGWFRQAPGKEREFVAAITDNGGS |
| | | KYYADSVKGRFTISADNSKNTAYLQMNSLKPEDTAVYYCAADRRGLASTRAADYDW |
| | | GQGTLVTVSS |
| 4A-H107 | 3615 | EVQLVESGGGLVQPGGSLRLSCAASGGTFSSYGMGWFRQAPGKEREFVAAINWSGAS |
| | | TYYADSVKGRFTISADNSKNTAYLQMNSLKPEDTAVYYCARDWRDRTWGNSLDYWG |
| | | QGTLVTVSS |
| 4A-H108 | 3616 | EVQLVESGGGLVQPGGSLRLSCAASGFSFDDDYVMGWFRQAPGKEREFVAAISWSED |
| | | NTYYADSVKGRFTISADNSKNTAYLQMNSLKPEDTAVYYCAADRRGLASTRAADYD |
| | | WGQGTLVTVSS |
| 4A-H109 | 3617 | EVQLVESGGGLVQPGGSLRLSCAASGFSFDDDYVMGWFRQAPGKEREFVAAVSGSGD |
| | | DTYYADSVKGRFTISADNSKNTAYLQMNSLKPEDTAVYYCAADRRGLASTRAADYD |
| | | WGQGTLVTVSS |
| 4A-H11 | 3618 | EVQLVESGGGLVQPGGSLRLSCAASGNIAAINVMGWFRQAPGKEREFVAAISASGRRT |
| | | DYADSVKGRFTISADNSKNTAYLQMNSLKPEDTAVYYCARRVYYYDSSGPPGVTFDI |
| | | WGQGTLVTVSS |
| 4A-H111 | 3619 | EVQLVESGGGLVQPGGSLRLSCAASGIITSRYVMGWFRQAPGKEREGVAAISTGGSTIY |
| | | ADSVKGRFTISADNSKNTAYLQMNSLKPEDTAVYYCARQDSSSPYFDYWGQGTLVTV |
| | | SS |
| 4A-H112 | 3620 | EVQLVESGGGLVQPGGSLRLSCAASGFSFDDDYVMGWFRQAPGKEREFVAAISNSGLS |
| | | TYYADSVKGRFTISADNSKNTAYLQMNSLKPEDTAVYYCAADRRGLASTRAADYDW |
| | | GQGTLVTVSS |
| 4A-H113 | 3621 | EVQLVESGGGLVQPGGSLRLSCAASGSISSINVMGWFRQAPGKEREFVATMRWSTGST |
| | | YYADSVKGRFTISADNSKNTAYLQMNSLKPEDTAVYYCAQRVRGFFGPLRTTPSWYE |
| | | WGQGTLVTVSS |
| 4A-H114 | 3622 | EVQLVESGGGLVQPGGSLRLSCAASGLTFILYRMGWFRQAPGKEREFVAAINNFGTTK |
| | | YADSVKGRFTISADNSKNTAYLQMNSLKPEDTAVYYCARTHYDFWSGYTSRTPNYFD |
| | | YWGQGTLVTVSS |
| 4A-H115 | 3623 | EVQLVESGGGLVQPGGSLRLSCAASGGTFSVYHMGWFRQAPGKEREPVAAISWSGGS |
| | | TAYADSVKGRFTISADNSKNTAYLQMNSLKPEDTAVYYCAAVNTWTSPSFDSWGQGT |
| | | LVTVSS |
| 4A-H116 | 3624 | EVQLVESGGGLVQPGGSLRLSCAASGRAFSTYGMGWFRQAPGKEREFVAGINWSGDT |
| | | PYYADSVKGRFTISADNSKNTAYLQMNSLKPEDTAVYYCAREVGPPPGYFDLWGQGT |
| | | LVTVSS |
| 4A-H117 | 3625 | EVQLVESGGGLVQPGGSLRLSCAASGRTFSDIAMGWFRQAPGKEREFVASINWGGGNT |
| | | YYADSVKGRFTISADNSKNTAYLQMNSLKPEDTAVYYCAAKGIWDYLGRRDFGDWG |
| | | QGTLVTVSS |
| 4A-H118 | 3626 | EVQLVESGGGLVQPGGSLRLSCAASGRTFSSARMGWFRQAPGKEREFVAAISWSGDNT |
| | | HYADSVKGRFTISADNSKNTAYLQMNSLKPEDTAVYYCATTENPNPRWGQGTLVTVS |
| | | S |
| 4A-H119 | 3627 | EVQLVESGGGLVQPGGSLRLSCAASGFAFSSYAMGWFRQAPGKEREWVATINGDDYT |
| | | YYADSVKGRFTISADNSKNTAYLQMNSLKPEDTAVYYCVATPGGYGLWGQGTLVTVS |
| | | S |
| 4A-H12 | 3628 | EVQLVESGGGLVQPGGSLRLSCAASGITFRRHDMGWFRQAPGKEREFVAAIRWSSSST |
| | | VYADSVKGRFTISADNSKNTAYLQMNSLKPEDTAVYYCAADRGVYGGRWYRTSQYT |
| | | WGQGTLVTVSS |
| 4A-H121 | 3629 | EVQLVESGGGLVQPGGSLRLSCAASGTAASFNPMGWFRQAPGKEREFVAAITSGGSTN |
| | | YADSVKGRFTISADNSKNTAYLQMNSLKPEDTAVYYCAAIAYEEGVYRWDWGQGTL |
| | | VTVSS |
| 4A-H122 | 3630 | EVQLVESGGGLVQPGGSLRLSCAASGNINIINYMGWFRQAPGKEREGVAAIHWNGDST |
| | | AYADSVKGRFTISADNSKNTAYLQMNSLKPEDTAVYYCASGPPYSNYFAYWGQGTLV |
| | | TVSS |
| 4A-H123 | 3631 | EVQLVESGGGLVQPGGSLRLSCAASGFTFDDYAMGWFRQAPGKERESVAAISGSGGST |
| | | AYADSVKGRFTISADNSKNTAYLQMNSLKPEDTAVYYCAKIMGSGRPYFDHWGQGTL |
| | | VTVSS |
| 4A-H124 | 3632 | EVQLVESGGGLVQPGGSLRLSCAASGNIFTRNVMGWFRQAPGKEREFVAAITSSGSTN |
| | | YADSVKGRFTISADNSKNTAYLQMNSLKPEDTAVYYCARPSSDLQGGVDYWGQGTLV |
| | | TVSS |
| 4A-H125 | 3633 | EVQLVESGGGLVQPGGSLRLSCAASGRTFSSIAMGWFRQAPGKEREFVASINWGGGNT |
| | | IYADSVKGRFTISADNSKNTAYLQMNSLKPEDTAVYYCAAKGIWDYLGRRDFGDWGQ |
| | | GTLVTVSS |
| 4A-H126 | 3634 | EVQLVESGGGLVQPGGSLRLSCAASGIPSTLRAMGWFRQAPGKEREFVAAVSSLGPFT |
| | | RYADSVKGRFTISADNSKNTAYLQMNSLKPEDTAVYYCAAKPGWVARDPSEYNWGQ |
| | | GTLVTVSS |
| 4A-H127 | 3635 | EVQLVESGGGLVQPGGSLRLSCAASGFTLDDSAMGWFRQAPGKEREWVAAITNGGST |
| | | YYADSVKGRFTISADNSKNTAYLQMNSLKPEDTAVYYCARFARGSPYFDFWGQGTLV |
| | | TVSS |
| 4A-H128 | 3636 | EVQLVESGGGLVQPGGSLRLSCAASGSISSFNAMGWFRQAPGKERESVAAIDWDGSTA |
| | | YADSVKGRFTISADNSKNTAYLQMNSLKPEDTAVYYCARGGGYYGSGSFEYWGQGTL |
| | | VTVSS |
| 4A-H129 | 3637 | EVQLVESGGGLVQPGGSLRLSCAASGNIFSDNIIGWFRQAPGKEREMVAYYTSGGSIDY |
| | | ADSVKGRFTISADNSKNTAYLQMNSLKPEDTAVYYCARGTAVGRPPPGGMDVWGQG |
| | | TLVTVSS |
| 4A-H13 | 3638 | EVQLVESGGGLVQPGGSLRLSCAASGSISSIGAMGWFRQAPGKEREGVAAISSSGSSTV |
| | | YADSVKGRFTISADNSKNTAYLQMNSLKPEDTAVYYCARVPPGQAYFDSWGQGTLVT |
| | | VSS |
| 4A-H131 | 3639 | EVQLVESGGGLVQPGGSLRLSCAASGFTFDDYGMGWFRQAPGKERELVATITWSGDS |
| | | TYYADSVKGRFTISADNSKNTAYLQMNSLKPEDTAVYYCAKGGSWYYDSSGYYGRW |
| | | GQGTLVTVSS |
| 4A-H132 | 3640 | EVQLVESGGGLVQPGGSLRLSCAASGRTFSNYTMGWFRQAPGKEREWVSAISWSTGST |
| | | YYADSVKGRFTISADNSKNTAYLQMNSLKPEDTAVYYCAADRYGPPWYDWGQGTLV |
| | | TVSS |
| 4A-H134 | 3641 | EVQLVESGGGLVQPGGSLRLSCAASGGTFSSVGMGWFRQAPGKERELVAVINWSGAR |
| | | TYYADSVKGRFTISADNSKNTAYLQMNSLKPEDTAVYYCAVPWMDYNRRDWGQGTL |
| | | VTVSS |
| 4A-H135 | 3642 | EVQLVESGGGLVQPGGSLRLSCAASGRIFTNTAMGWFRQAPGKEREGVAAINWSGGST |
| | | AYADSVKGRFTISADNSKNTAYLQMNSLKPEDTAVYYCARTSGSYSFDYWGQGTLVT |
| | | VSS |
| 4A-H136 | 3643 | EVQLVESGGGLVQPGGSLRLSCAASGEEFSDHWMGWFRQAPGKEREFVGAIHWSGGR |
| | | TYYADSVKGRFTISADNSKNTAYLQMNSLKPEDTAVYYCAADRRGLASTRAADYDW |
| | | GQGTLVTVSS |
| 4A-H137 | 3644 | EVQLVESGGGLVQPGGSLRLSCAASGRTFSSIAMGWFRQAPGKEREFVAAINWSGART |
| | | AYADSVKGRFTISADNSKNTAYLQMNSLKPEDTAVYYCAAKGIWDYLGRRDFGDWG |
| | | QGTLVTVSS |
| 4A-H138 | 3645 | EVQLVESGGGLVQPGGSLRLSCAASGSTSSLRTMGWFRQAPGKEREGVAAISSRDGSTI |
| | | YADSVKGRFTISADNSKNTAYLQMNSLKPEDTAVYYCARDDSSSPYFDYWGQGTLVT |
| | | VSS |
| 4A-H139 | 3646 | EVQLVESGGGLVQPGGSLRLSCAASGGGTFGSYAMGWFRQAPGKEREFVAAISIASGA |
| | | SGGTTNYADSVKGRFTISADNSKNTAYLQMNSLKPEDTAVYYCATTMNPNPRWGQGT |
| | | LVTVSS |
| 4A-H14 | 3647 | EVQLVESGGGLVQPGGSLRLSCAASGRTFSNAAMGWFRQAPGKEREFVARITWNGGS |
| | | TFYADSVKGRFTISADNSKNTAYLQMNSLKPEDTAVYYCATTENPNPRWGQGTLVTV |
| | | SS |
| 4A-H141 | 3648 | EVQLVESGGGLVQPGGSLRLSCAASGIILSDNAMGWFRQAPGKEREFVAAISWLGEST |
| | | YYADSVKGRFTISADNSKNTAYLQMNSLKPEDTAVYYCAADRRGLASTRAADYDWG |
| | | QGTLVTVSS |
| 4A-H142 | 3649 | EVQLVESGGGLVQPGGSLRLSCAASGRTFGDYIMGWFRQAPGKERESVAAINWNGGY |
| | | TAYADSVKGRFTISADNSKNTAYLQMNSLKPEDTAVYYCATTSPNTGWHYYRWGQG |
| | | TLVTVSS |
| 4A-H143 | 3650 | EVQLVESGGGLVQPGGSLRLSCAASGFNFNWYPMGWFRQAPGKERESVAAISWTGVS |
| | | TYTAYADSVKGRFTISADNSKNTAYLQMNSLKPEDTAVYYCARWGPGPAGGSPGLVG |
| | | FDYWGQGTLVTVSS |
| 4A-H144 | 3651 | EVQLVESGGGLVQPGGSLRLSCAASGSIRSVSVMGWFRQAPGKEREAVAAISWSGVGT |
| | | AYADSVKGRFTISADNSKNTAYLQMNSLKPEDTAVYYCAAYQRGWGDWGQGTLVTV |
| | | SS |
| 4A-H145 | 3652 | EVQLVESGGGLVQPGGSLRLSCAASGMTFRLYAMGWFRQAPGKEREFVGAINWLSES |
| | | TYYADSVKGRFTISADNSKNTAYLQMNSLKPEDTAVYYCAAKPGWVARDPSEYNWG |
| | | QGTLVTVSS |
| 4A-H146 | 3653 | EVQLVESGGGLVQPGGSLRLSCAASGRTFSDDAMGWFRQAPGKEREFVAAINWSGGS |
| | | TYYADSVKGRFTISADNSKNTAYLQMNSLKPEDTAVYYCATDPPLFWGQGTMVTVSS |
| 4A-H147 | 3654 | EVQLVESGGGLVQPGGSLRLSCAASGGTFSVYAMGWFRQAPGKEREGVAAISMSGDD |
| | | AAYADSVKGRFTISADNSKNTAYLQMNSLKPEDTAVYYCAKISKDDGGKPRGAFFDS |
| | | WGQGTLVTVSS |
| 4A-H148 | 3655 | EVQLVESGGGLVQPGGSLRLSCAASGFALGYYAMGWFRQAPGKERESVAAISSRDGST |
| | | AYADSVKGRFTISADNSKNTAYLQMNSLKPEDTAVYYCARLATGPQAYFHHWGQGTL |
| | | VTVSS |
| 4A-H149 | 3656 | EVQLVESGGGLVQPGGSLRLSCAASGFNLDDYAMGWFRQAPGKERESVAAISWDGGA |
| | | TAYADSVKGRFTISADNSKNTAYLQMNSLKPEDTAVYYCARVGRGTTAFDSWGQGTL |
| | | VTVSS |
| 4A-H15 | 3657 | EVQLVESGGGLVQPGGSLRLSCAASGNTFSGGFMGWFRQAPGKEREFVASIRSGARTY |
| | | YADSVKGRFTISADNSKNTAYLQMNSLKPEDTAVYYCAQRVRGFFGPLRTTPSWYEW |
| | | GQGTLVTVSS |
| 4A-H151 | 3658 | EVQLVESGGGLVQPGGSLRLSCAASGSIRSINIMGWFRQAPGKEREAVAAISWSGGSTV |
| | | YADSVKGRFTISADNSKNTAYLQMNSLKPEDTAVYYCASLLAGDRYFDYWGQGTLVT |
| | | VSS |
| 7A-1 | 3659 | EVQLVESGGGLVQPGGSLRLSCAASGFTLGDYVMGWFRQAPGKEREFVAAIHSGGST |
| | | YADSVKGRFTISADNSKNTAYLQMNSLKPEDTAVYYCAAKEYGGTRRYDRAYNWGQ |
| | | GTLVTVSS |
| 7A-2 | 3660 | EVQLVESGGGLVQPGGSLRLSCAASGGGTFGSYAMGWFRQAPGKERELVAAISSGGST |
| | | NYADSVKGRFTISADNSKNTAYLQMNSLKPEDTAVYYCARGDWRYGWGQGTLVTVS |
| | | S |
| 7A-3 | 3661 | EVQLVESGGGLVQPGGSLRLSCAASGRTYSISAMGWFRQAPGKEREFVAAISMSGDDS |
| | | AYADSVKGRFTISADNSKNTAYLQMNSLKPEDTAVYYCAAQLGYESGYSLTYDYDW |
| | | GQGTLVTVSS |
| 7A-4 | 3662 | EVQLVESGGGLVQPGGSLRLSCAASGGTFSTYPMGWFRQAPGKEREFVAAITSDGSTL |
| | | YADSVKGRFTISADNSKNTAYLQMNSLKPEDTAVYYCAATDYNKAYAREGRRYDWG |
| | | QGTLVTVSS |
| 7A-5 | 3663 | EVQLVESGGGLVQPGGSLRLSCAASGSIFRINAMGWFRQAPGKEREFVAAIHWSGSSTR |
| | | YADSVKGRFTISADNSKNTAYLQMNSLKPEDTAVYYCAAQDRRRGDYYTFDYHWGQ |
| | | GTLVTVSS |
| 7A-6 | 3664 | EVQLVESGGGLVQPGGSLRLSCAASGGTFNNYAMGWFRQAPGKERELVAAITSGGST |
| | | DYADSVKGRFTISADNSKNTAYLQMNSLKPEDTAVYYCARGDWRYGWGQGTLVTVS |
| | | S |
| 7A-7 | 3665 | EVQLVESGGGLVQPGGSLRLSCAASGTIVNINVMGWFRQAPGKEREFVAAIHWSGGLK |
| | | AYADSVKGRFTISADNSKNTAYLQMNSLKPEDTAVYYCAMNRAGIYEWGQGTLVTVS |
| | | S |
| 7A-8 | 3666 | EVQLVESGGGLVQPGGSLRLSCAASGSTFSNYAMGWFRQAPGKERELVAAITSGGSTS |
| | | YADSVKGRFTISADNSKNTAYLQMNSLKPEDTAVYYCARGDWRYGWGQGTLVTVSS |
| 7A-9 | 3667 | EVQLVESGGGLVQPGGSLRLSCAASGFSFDDYVMGWFRQAPGKEREFVAAISRSGNLK |
| | | SYADSVKGRFTISADNSKNTAYLQMNSLKPEDTAVYYCAAKEYGGTRRYDRAYNWG |
| | | QGTLVTVSS |
| 7A-10 | 3668 | EVQLVESGGGLVQPGGSLRLSCAASGSAFRSTVMGWFRQAPGKEREFVAAVIGSSGIT |
| | | DYADSVKGRFTISADNSKNTAYLQMNSLKPEDTAVYYCARGDWRYGWGQGTLVTVS |
| | | S |
| 7A-11 | 3669 | EVQLVESGGGLVQPGGSLRLSCAASGRTFSDAGMGWFRQAPGKEREFVAAISRSGNLK |
| | | AYADSVKGRFTISADNSKNTAYLQMNSLKPEDTAVYYCAVQVNGTWAWGQGTLVTV |
| | | SS |
| 7A-12 | 3670 | EVQLVESGGGLVQPGGSLRLSCAASGFTLDYYAMGWFRQAPGKERELVAAISWNGGS |
| | | TSYADSVKGRFTISADNSKNTAYLQMNSLKPEDTAVYYCARGDWRYGWGQGTLVTV |
| | | SS |
| 7A-13 | 3671 | EVQLVESGGGLVQPGGSLRLSCAASGGTFSTYVMGWFRQAPGKEREFVAAISWSGEST |
| | | LYADSVKGRFTISADNSKNTAYLQMNSLKPEDTAVYYCAADLMYGVDRRYDWGQGT |
| | | LVTVSS |
| 7A-14 | 3672 | EVQLVESGGGLVQPGGSLRLSCAASGISSSKRNMGWFRQAPGKEREFVAGISWTGGIT |
| | | YYADSVKGRFTISADNSKNTAYLQMNSLKPEDTAVYYCAIAGRGRWGQGTLVTVSS |
| 7A-15 | 3673 | EVQLVESGGGLVQPGGSLRLSCAASGRRFSAYGMGWFRQAPGKEREFVAVISRSGTLT |
| | | RYADSVKGRFTISADNSKNTAYLQMNSLKPEDTAVYYCASSGPADARNGERWHWGQ |
| | | GTLVTVSS |
| 7A-16 | 3674 | EVQLVESGGGLVQPGGSLRLSCAASGLTFSSFVMGWFRQAPGKEREFVAAISSNGGST |
| | | RYADSVKGRFTISADNSKNTAYLQMNSLKPEDTAVYYCAAKEYGGTRRYDRAYNWG |
| | | QGTLVTVSS |
| 7A-17 | 3675 | EVQLVESGGGLVQPGGSLRLSCAASGTVFSISAMGWFRQAPGKEREFVAAISMSGDDT |
| | | AYADSVKGRFTISADNSKNTAYLQMNSLKPEDTAVYYCAAQLGYESGYSLTYDYDW |
| | | GQGTLVTVSS |
| 7A-18 | 3676 | EVQLVESGGGLVQPGGSLRLSCAASGSIFSPNVMGWFRQAPGKEREFVAAITNGGSTK |
| | | YADSVKGRFTISADNSKNTAYLQMNSLKPEDTAVYYCAAQRWRGGSYEWGQGTLVT |
| | | VSS |
| 7A-19 | 3677 | EVQLVESGGGLVQPGGSLRLSCAASGIPASIRVMGWFRQAPGKEREFVAAIHWSGSSTR |
| | | YADSVKGRFTISADNSKNTAYLQMNSLKPEDTAVYYCALSRAIVPGDSEYDYRWGQG |
| | | TLVTVSS |
| 7A-20 | 3678 | EVQLVESGGGLVQPGGSLRLSCAASGRTFSMSAMGWFRQAPGKEREFVSAISWSGGST |
| | | LYADSVKGRFTISADNSKNTAYLQMNSLKPEDTAVYYCAAQLGYESGYSLTYDYDWG |
| | | QGTLVTVSS |
| 7A-21 | 3679 | EVQLVESGGGLVQPGGSLRLSCAASGRTFSNYAMGWFRQAPGKERELVAAITSGGSTD |
| | | YADSVKGRFTISADNSKNTAYLQMNSLKPEDTAVYYCARGDWRYGWGQGTLVTVSS |
| 7A-22 | 3680 | EVQLVESGGGLVQPGGSLRLSCAASGRTFSSYAMGWFRQAPGKERELVAAISTGGSTY |
| | | YADSVKGRFTISADNSKNTAYLQMNSLKPEDTAVYYCARGDWRYGWGQGTLVTVSS |
| 7A-23 | 3681 | EVQLVESGGGLVQPGGSLRLSCAASGRSFSSVGMGWFRQAPGKEREFVAVISRSGAST |
| | | AYADSVKGRFTISADNSKNTAYLQMNSLKPEDTAVYYCASAGPADARNGERWAWGQ |
| | | GTLVTVSS |
| 7A-24 | 3682 | EVQLVESGGGLVQPGGSLRLSCAASGRAFRRYTMGWFRQAPGKERELIAVINWSGDR |
| | | RYYADSVKGRFTISADNSKNTAYLQMNSLKPEDTAVYYCAATLAKGGGRWGQGTLV |
| | | TVSS |
| 7A-25 | 3683 | EVQLVESGGGLVQPGGSLRLSCAAMAWAGFARRRAKNAKWWRALPRGGPTYADSV |
| | | KGRFTISADNSKNTAYLQMNSLKPEDTAVYYCAAGGMWYGSSLYVRFDLLEDGMDW |
| | | GQGTLVTVSS |
| 7A-26 | 3684 | EVQLVESGGGLVQPGGSLRLSCAASGSISSINGMGWFRQAPGKERELVALISRSGGTTY |
| | | YADSVKGRFTISADNSKNTAYLQMNSLKPEDTAVYYCASAGPADARNGERWAWGQG |
| | | TLVTVSS |
| 7A-27 | 3685 | EVQLVESGGGLVQPGGSLRLSCAASGRTFSNNVMGWFRQAPGKERELVAAAISGGSTY |
| | | YADSVKGRFTISADNSKNTAYLQMNSLKPEDTAVYYCARGDWRYGWGQGTLVTVSS |
| 7A-28 | 3686 | EVQLVESGGGLVQPGGSLRLSCAASGRTFSISAMGWFRQAPGKEREFVAAISRSGTTM |
| | | YADSVKGRFTISADNSKNTAYLQMNSLKPEDTAVYYCAAQLGYESGYSLTYDYDWG |
| | | QGTLVTVSS |
| 7A-29 | 3687 | EVQLVESGGGLVQPGGSLRLSCAASGGTFSYYDLAAMGWFRQAPGKEREFVAAISWS |
| | | QYNTKYADSVKGRFTISADNSKNTAYLQMNSLKPEDTAVYYCAARVVVRTAHGFEDN |
| | | WGQGTLVTVSS |
| 7A-30 | 3688 | EVQLVESGGGLVQPGGSLRLSCAASGRTFNNYGMGWFRQAPGKEREFVAVISRSGSLK |
| | | AYADSVKGRFTISADNSKNTAYLQMNSLKPEDTAVYYCASDPTYGSGRWTWGQGTL |
| | | VTVSS |
| 7A-31 | 3689 | EVQLVESGGGLVQPGGSLRLNCAASGFTLDDYVMGWFRQTPGKEREFVAAISSSGALT |
| | | SYADSVKGRFTISADNSKNTAYLQMNSLKPEDAAVYYCAAKEYGGTRRYDRAYNWG |
| | | QGTLVTVSS |
| 7A-32 | 3690 | EVQLVESGGGLVQPGGSLRLSCAASGRTFNAMGWFRQAPGKEREFVAAIRWSGDMSV |
| | | YADSVKGRFTISADNSKNTAYLQMNSLKPEDTAVYYCAAQDRRRGDYYTFDYHWGQ |
| | | GTLVTVSS |
| 7A-33 | 3691 | EVQLVESGGGLVQPGGSLRLSCAASGLTFSTYAMGWFRQAPGKEREFVAAITSGGSTD |
| | | YADSVKGRFTISADNSKNTAYLQMNSLKPEDTAVYYCARGDWRYGWGQGTLVTVSS |
| 7A-34 | 3692 | EVQLVESGGGLVQPGGSLRLSCAASGSIFTINAMGWFRQAPGKEREGVAAIGSDGSTSY |
| | | ADSVKGRFTISADNSKNTAYLQMNSLKPEDTAVYYCAVVRWGADWGQGTLVTVSS |
| 7A-35 | 3693 | EVQLVESGGGLVQPGGSLRLSCAASGLTFSSYAMGWFRQAPGKERELVAAITSSSGSTP |
| | | AYADSVKGRFTISADNSKNTAYLQMNSLKPEDTAVYYCARGDWRYGWGQGTLVTVS |
| | | S |
| 7A-36 | 3694 | EVQLVESGGGLVQPGGSLRLSCAASGIPFSTRTMGWFRQAPGKEREFVAAISWSQYNT |
| | | KYADSVKGRFTISADNSKNTAYLQMNSLKPEDTAVYYCAARHWGMFSRSENDYNWG |
| | | QGTLVTVSS |
| 7A-37 | 3695 | EVQLVESGGGLVQPGGSLRLSCAASGRSRFSTYVMGWFRQAPGKEREFVAAISWSQY |
| | | NTKYADSVKGRFTISADNSKNTAYLQMNSLKPEDTAVYYCAAGNGGRNYGHSRARY |
| | | DWGQGTLVTVSS |
| 7A-38 | 3696 | EVQLVESGGGLVQPGGSLRLSCAASGLTLSSYGMGWFRQAPGKEREYVAVISRSGSLK |
| | | AYADSVKGRFTISADNSKNTAYLQMNSLKPEDTAVYYCATRADAEGWWDWGQGTLV |
| | | TVSS |
| 7A-39 | 3697 | EVQLVESGGGLVQPGGSLRLSCAASGSIFRVNVMGWFRQAPGKEREFVAAINNFGTTK |
| | | YADSVKGRFTISADNSKNTAYLQMNSLKPEDTAVYYCAADLPSRWGQGTLVTVSS |
| 7A-40 | 3698 | EVQLVESGGGLVQPGGSLRLSCAASGRTFRNYAMGWFRQAPGKERELVAAISSGGSTD |
| | | YADSVKGRFTISADNSKNTAYLQMNSLKPEDTAVYYCARGDWRYGWGQGTLVTVSS |
| 7A-41 | 3699 | EVQLVESGGGLVQPGGSLRLSCAASGRTFSSFAMGWFRQAPGKERELVAAISSGGSTN |
| | | YADSVKGRFTISADNSKNTAYLQMNSLKPEDTAVYYCARGDWRYGWGQGTLVTVSS |
| 7A-42 | 3700 | EVQLVESGGGLVQPGGSLRLSCAASGTTFRINAMGWFRQAPGKEREFVAAMNWSGGS |
| | | TKYADSVKGRFTISADNSKNTAYLQMNSLKPEDTAVYYCAAQDRRRGDYYTFDYHW |
| | | GQGTLVTVSS |
| 7A-43 | 3701 | EVQLVESGGGLVQPGGSLRLSCAASGFTLGDYVMGWFRQAPGKEREFVAAIHSGGSTL |
| | | YADSVKGRFTISADNSKNTAYLQMNSLKPEDTAVYYCAAKEYGGTRRYDRTYNWGQ |
| | | GTLVTVSS |
| 7A-44 | 3702 | EVQLVESGGGLVQPGGSLRLSCAASGFTFSRSAMGWFRQAPGKERELVAGILSSGATV |
| | | YADSVKGRFTISADNSKNTAYLQMNSLKPEDTAVYYCAKAPRDWGQGTLVTVSS |
| 7A-45 | 3703 | EVQLVESGGGLVQPGGSLRLSCAASGRTFNNYAMGWFRQAPGKERELVAAITSGGST |
| | | DYADSVKGRFTISADNSKNTAYLQMNSLKPEDTAVYYCARGDWRYGWGQGTLVTVS |
| | | S |
| 7A-46 | 3704 | EVQLVESGGGLVQPGGSLRLSCAASGFTFRSYPMGWFRQAPGKEREFVAAINNFGTTK |
| | | YADSVKGRFTISADNSKNTAYLQMNSLKPEDTAVYYCAAAKGIGVYGWGQGTLVTVS |
| | | S |
| 7A-47 | 3705 | EVQLVESGGGLVQPGGSLRLSCAASGNIFTRNVMGWFRQAPGKEREFVAAIHWNGDS |
| | | TKYADSVKGRFTISADNSKNTAYLQMNSLKPEDTAVYYCAAGSNIGGSRWRYDWGQ |
| | | GTLVTVSS |
| 7A-48 | 3706 | EVQLVESGGGLVQPGGSLRLSCAASGRTISRYTMGWFRQAPGKERELVAAIKWSGAST |
| | | VYADSVKGRFTISADNSKNTAYLQMNSLKPEDTAVYYCAAKGIWDYLGRRDFGDWG |
| | | QGTLVTVSS |
| 7A-49 | 3707 | EVQLVESGGGLVQPGGSLRLSCAASGFRFSSYGMGWFRQAPGKEREFVAIITSGGLTVY |
| | | ADSVKGRFTISADNSKNTAYLQMNSLKPEDTAVYYCAARKTFYFGTSSYPNDYAWGQ |
| | | GTLVTVSS |
| 7A-50 | 3708 | EVQLVESGGGLVQPGGSLRLSCAASGRTFDNHAMGWFRQAPGKEREGVAAIGSDGST |
| | | SYADSVKGRFTISADNSKNTAYLQMNSLKPEDTAVYYCAVVRWGVDWGQGTLVTVS |
| | | S |
| 7A-51 | 3709 | EVQLVESGGGLVQPGGSLRLSCAASGFTFSSHAMGWFRQAPGKEREFVAGISWSGEST |
| | | LTRYADSVKGRFTISADNSKNTAYLQMNSLKPEDTAVYYCADVNGDWGQGTLVTVSS |
| 7A-52 | 3710 | EVQLVESGGGLVQPGGSLRLSCAASGMTFRLYAMGWFRQAPGKEREFVAAISWSQYN |
| | | TKYADSVKGRFTISADNSKNTAYLQMNSLKPEDTAVYYCAAQLGYESGYSLTYDYDW |
| | | GQGTLVTVSS |
| 7A-53 | 3711 | EVQLVESGGGLVQPGGSLRLSCAASGGTFRKLAMGWFRQAPGKEREFVAVISWTGGS |
| | | SYYADSVKGRFTISADNSKNTAYLQMNSLKPEDTAVYYCARLTSFATWGQGTLVTVSS |
| 7A-54 | 3712 | EVQLVESGGGLVQPGGSLRLSCAASGRTFSANGMGWFRQAPGKEREFVAAISASGTLR |
| | | AYADSVKGRFTISADNSKNTAYLQMNSLKPEDTAVYYCAARSPMSPTWDWGQGTLV |
| | | TVSS |
| 7A-55 | 3713 | EVQLVESGGGLVQPGGSLRLSCAASGSAFRSTVMGWFRQAPGKEREFVAAISWTGEST |
| | | LYADSVKGRFTISADNSKNTAYLQMNSLKPEDTAVYYCATGPYRSYFARSYLWGQGT |
| | | LVTVSS |
| 7A-56 | 3714 | EVQLVESGGGLVQPGGSLRLSCAASGGTFDYSGMGWFRQAPGKEREFVAVVSQSGRT |
| | | TYYADSVKGLFTITADNSKNTAYLQMNLLKPEDTAVYYCPTATRPGEWDGGQGTLVT |
| | | VSR |
| 7A-57 | 3715 | EVQLVESGGGLVQPGGSLRLSCAASGVFGPIRAMGWFRQAPGKERELVALMGNDGST |
| | | YADSVKGRFTISADNSKNTAYLQMNSLKPEDTAVYYCAAIGWRWGQGTLVTVSS |
| 7A-58 | 3716 | EVQLVESGGGLVQPGGSLRLSCAASGFNFNWYPMGWFRQAPGKEREFVAAIRWSGGI |
| | | TYYADSVKGRFTISADNSKNTAYLQMNSLKPEDTAVYYCATGPYRSYFARSYLWGQG |
| | | TLVTVSS |
| 7A-59 | 3717 | EVQLVESGGGLVQPGGSLRLSCAASGMTFHRYVMGWFRQAPGKERELVASITTGGTP |
| | | NYADSVKGRFTIITDNNKNTAYLLMINLQPEDTAVYYCCKVPYIWGQGTLGTVGT |
| 7A-60 | 3718 | EVQLVESGGGLVQPGGSLRLSCAASGISTMGWFRQAPGKEREFVAAINNFGTTKYADS |
| | | VKGRFTISADNSKNTAYLQMNSLKPEDTAVYYCAAASQSGSGYDWGQGTLVTVSS |
| 7A-61 | 3719 | EVQLVESGGGLVQPGGSLRLSCAASGRAFNTRAMGWFRQAPGKERELVALMGNDGST |
| | | YADSVKGRFTISADNSKNTAYLQMNSLKPEDTAVYYCAAIGWRWGQGTLVTVSS |
| 7A-62 | 3720 | EVQLVESGGGLVQPGGSLRLSCAASGLTDRRYTMGWFRQAPGKEREFVAAINSGGSTL |
| | | YADSVKGRFTISADNSKNTAYLQMNSLKPEDTAVYYCAAGNGGRTYGHSRARYEWG |
| | | QGTLVTVSS |
| 7A-63 | 3721 | EVQLVESGGGLVQPGGSLRLSCAASGRTFNVMGWFRQAPGKERELVALMGNDGSTY |
| | | ADSVKGRFTISADNSKNTAYLQMNSLKPEDTAVYYCAAVRWGVDWGQGTLVTVSS |
| 7A-64 | 3722 | EVQLVESGGGLVQPGGSLRLSCAASGRAFNTRAMGWFRQAPGKERELVALMGNDGST |
| | | NYADSVKGRFTISADNSKNTAYLQMNSLKPEDTAVYYCAAIGWRWGQGTLVTVSS |
| 7A-65 | 3723 | EVQVVESGGGVVHPGGSVRMRCAASGVTVDYSGMGWFGQAPGKEREFVAVVSQSAR |
| | | TTYYADSVKGRFTISADNSKNTEYLQMNSMKPEDTAVYYCATATRPGEWDWGQGTL |
| | | VTVSS |
| 7A-66 | 3724 | EVQLVESGGGLVQPGGSLRLSCAASGRTPRLGAMGWFRQAPGKEREFVAAISRSGGLT |
| | | SYADSVKGRFTISADNSKNTAYLQMNSLKPEDTAVYYCAAQLVGSNIGGSRWRYDWG |
| | | QGTLVTVSS |
| 7A-67 | 3725 | EVQLVESGGGLVQPGGSLRLSCAASGLTFRNYAMGWFRQAPGKEREFVAAITSGGSTL |
| | | YADSVKGRFTISADNSKNTAYLQMNSLKPEDTAVYYCARGDWRYGWGHGTLVTESS |
| 8A-1 | 3726 | EVQLVESGGGLVQPGGSLRLSCAASGGRTFSDLAMGWFRQAPGKEREFVALITRSGGT |
| | | TFYADSVKGRFTISADNSKNTAYLQMNSLKPEDTAVYYCAIGRGSWGQGTLVTVSS |
| 8A-2 | 3727 | EVQLVESGGGLVQPGGSLRLSCAASGFTFGEYAMGWFRQAPGKEREFVAAVSSLGPFT |
| | | RYADSVKGRFTISADNSKNTAYLQMNSLKPEDTAVYYCAAVLDGYSGSWGQGTLVTV |
| | | SS |
| 8A-3 | 3728 | EVQLVESGGGLVQPGGSLRLSCAASGFAFSSYGMGWFRQAPGKEREFVAAISWSGVRS |
| | | GVSAIYADSVKGRFTISADNSKNTAYLQMNSLKPEDTAVYYCTTDLTGDLWYFDLWG |
| | | QGTLVTVSS |
| 8A-4 | 3729 | EVQLVESGGGLVQPGGSLRLSCAASGLTAGTYAMCWFRQAPGKEREGVACASSTDGS |
| | | TAYADSVKGRFTISADNSKNTAYLQMNSLKPEDTAVYYCAAVRTYGSATYDWGQGT |
| | | LVTVSS |
| 8A-5 | 3730 | EVQLVESGGGLVQPGGSLRLSCAASGFTLDDYVMGWFRQAPGKERELVAAVSSLGPF |
| | | TRYADSVKGRFTISADNSKNTAYLQMNSLKPEDTAVYYCAAKEYGGTRRYDRAYNW |
| | | GQGTLVTVSS |
| 8A-6 | 3731 | EVQLVESGGGLVQPGGSLRLSCAASGPTLGSYVMGWFRQAPGKEREFVAAISWSQYN |
| | | TKYADSVKGRFTISADNSKNTAYLQMNSLKPEDTAVYYCAAQRWRGGSYEWGQGTL |
| | | VTVSS |
| 8A-7 | 3732 | EVQLVESGGGLVQPGGSLRLSCAASGPTFSSYVMGWFRQAPGKEREFVAAISWSQYNT |
| | | KYADSVKGRFTISADNSKNTAYLQMNSLKPEDTAVYYCAAASRSGSGYDWGQGTLVT |
| | | VSS |
| 8A-8 | 3733 | EVQLVESGGGLVQPGGSLRLSCAASGYLYSKDCMGWFRQAPGKEREGVATICTGDGS |
| | | TAYADSVKGRFTISADNSKNTAYLQMNSLKPEDTAVYYCAVIAYEEGVYRWDWGQG |
| | | TLVTVSS |
| 8A-9 | 3734 | EVQLVESGGGLVQPGGSLRLSCAASGFTIDDYAMGWFRQAPGKEREGVAAISGSGDDT |
| | | YYADSVKGRFTISADNSKNTAYLQMNSLKPEDTAVYYCAKLPYVSGDYWGQGTLVT |
| | | VSS |
| 8A-10 | 3735 | EVQLVESGGGLVQPGGSLRLSCAASGGRFSDYGMGWFRQAPGKERELVALISRSGNLK |
| | | SYADSVKGRFTISADNSKNTAYLQMNSLKPEDTAVYYCAAKTGTSFVWGQGTLVTVS |
| | | S |
| 8A-11 | 3736 | EVQLVESGGGLVQPGGSLRLSCAASGLSFSNYAMGWFRQAPGKERELVAAITSGGSTD |
| | | YADSVKGRFTISADNSKNTAYLQMNSLKPEDTAVYYCARGDWRYGWGQGTLVTVSS |
| 8A-12 | 3737 | EVQLVESGGGLVQPGGSLRLSCAASGIPSTLRAMGWFRQAPGKEREFVALINRSGGSQF |
| | | YADSVKGRFTISADNSKNTAYLQMNSLKPEDTAVYYCAIGRGSWGQGTLVTVSS |
| 9A-1 | 3738 | EVQLVESGGGLVQPGGSLRLSCAASGRTFSRLAMGWFRQAPGKEREFVAAISRSGRST |
| | | SYADSVKGRFTISADNSKNTAYLQMNSLKPEDTAVYYCAARRSQILFTSRTDYEWGQG |
| | | TLVTVSS |
| 9A-2 | 3739 | EVQLVESGGGLVQPGGSLRLSCAASGSFSIAAMGWFRQAPGKEREFVATINYSGGGTY |
| | | YADSVKGRFTISADNSKNTAYLQMNSLKPEDTAVYYCAAVNIFDESAYAAFACYDVV |
| | | WGQGTLVTVSS |
| 9A-3 | 3740 | EVQLVESGGGLVQPGGSLRLSCAASGRTFSRYAMGWFRQAPGKEREFVAAISRSGKST |
| | | YYADSVKGRFTISADNSKNTAYLQMNSLKPEDTAVYYCAASSVFSDLRYRKNPKWGQ |
| | | GTLVTVSS |
| 9A-4 | 3741 | EVQLVESGGGLVQPGGSLRLSCAASGRTFSKYAMGWFRQAPGKEREFVSHISRDGGRT |
| | | FSSSTMGWFRQAPGKERELVALITPSSRTTYYADSVKGRFTISADNSKNTAYLQMNSLK |
| | | PEDTAVYYCAIAGRGRWGQGTLVTVSS |
| 9A-5 | 3742 | EVQLVESGGGLVQPGGSLRLSCAASGRTFRRYAMGWFRQAPGKEREFVASINWGGGN |
| | | TYYADSVKGRFTISADNSKNTAYLQMNSLKPEDTAVYYCAKTKRTGIFTTARMVDWG |
| | | QGTLVTVSS |
| 9A-6 | 3743 | EVQLVESGGGLVQPGGSLRLSCAASGRTFSRFAMGWFRQAPGKEREFVAAIRWSGGRT |
| | | VYADSVKGRFTISADNSKNTAYLQMNSLKPEDTAVYYCAIEPGTIRNWRNRVPFARGN |
| | | FGWGQGTLVTVSS |
| 9A-7 | 3744 | EVQLVESGGGLVQPGGSLRLSCAASGLGIAFSRRTAMGWFRQAPGKEREFVAAISWRG |
| | | GNTYYADSVKGRFTISADNSKNTAYLQMNSLKPEDTAVYYCAARRWIPPGPIWGQGT |
| | | LVTVSS |
| 9A-8 | 3745 | EVQLVESGGGLVQPGGSLRLSCAASGRTFRRYPMGWFRQAPGKEREFVAAISRSGGST |
| | | YYADSVKGRFTISADNSKNTAYLQMNSLKPEDTAVYYCAAKRLRSFASGGSYDWGQG |
| | | TLVTVSS |
| 9A-9 | 3746 | EVQLVESGGGLVQPGGSLRLSCAASGGTLRGYGMGWFRQAPGKEREFVASISRSGGST |
| | | YYADSVKGRFTISADNSKNTAYLQMNSLKPEDTAVYYCAARRRVTLFTSRADYDWGQ |
| | | GTLVTVSS |
| 9A-10 | 3747 | EVQLVESGGGLVQPGGSLRLSCAASGRMFSSRSMGWFRQAPGKEREFVALINRSGGSQ |
| | | FYADSVKGRFTISADNSKNTAYLQMNSLKPEDTAVYYCAARRWIPPGPIWGQGTLVTV |
| | | SS |
| 9A-11 | 3748 | EVQLVESGGGLVQPGGSLRLSCAASGRTFGRRAMGWFRQAPGKEREFVAGISRGGGT |
| | | NYADSVKGRFTISADNSKNTAYLQMNSLKPEDTAVYYCAAKGIWDYLGRRDFGDWG |
| | | QGTLVTVSS |
| 10A-1 | 3749 | EVQLVESGGGLVQPGGSLRLSCAASGLSSPPFDDFPMGWFRQAPGKEREFVSSIYSDDG |
| | | DSMYADSVKGRFTISADNSKNTAYLQMNSLKPEDTAVYYCARQTFDFWSASLGGNFW |
| | | YFDLWGQGTLVTVSS |
| 10A-2 | 3750 | EVQLVESGGGLVQPGGSLRLSCAASGGTFSSYSMGWFRQAPGKEREFVSAISWIIGSGG |
| | | TTNYADSVKGRFTISADNSKNTAYLQMNSLKPEDTAVYYCTAGAGDSWGQGTLVTVS |
| | | S |
| 10A-3 | 3751 | EVQLVESGGGLVQPGGSLRLSCAASGSIFSTRTMGWFRQAPGKEREFVASITKFGSTNY |
| | | ADSVKGRFTISADNSKNTAYLQMNSLKPEDTAVYYCTRGGGRFFDWLYLRWGQGTLV |
| | | TVSS |
| 10A-4 | 3752 | EVQLVESGGGLVQPGGSLRLSCAASGRTLWRSNMGWFRQAPGKEREFVASISSFGSTK |
| | | YADSVKGRFTISADNSKNTAYLQMNSLKPEDTAVYYCARGHGRYFDWLLFARPPDYW |
| | | GQGTLVTVSS |
| 10A-5 | 3753 | EVQLVESGGGLVQPGGSLRLSCAASGRSLGIYRMGWFRQAPGKEREFVAAITSGGRKN |
| | | YADSVKGRFTISADNSKNTAYLQMNSLKPEDTAVYYCAKRTIFGVGRWLDPWGQGTL |
| | | VTVSS |
| 10A-6 | 3754 | EVQLVESGGGLVQPGGSLRLSCAASGTTLTFRIMGWFRQAPGKEREFVPAISSTGLASY |
| | | TDSVKGRFTISADNSKNTAYLQMNSLKPEDTAVYYCSKDRAPNCFACCPNGFDVWGQ |
| | | GTLVTVSS |
| 10A-7 | 3755 | EVQLVESGGGLVQPGGSLRLSCAASGSRFSGRFNILNMGWFRQAPGKEREFVARIGYS |
| | | GQSISYADSVKGRFTISADNSKNTAYLQMNSLKPEDTAVYYCARGRFLGGTEWGQGTL |
| | | VTVSS |
| 10A-8 | 3756 | EVQLVESGGGLVQPGGSLRLSCAASGTLFKINAMGWFRQAPGKEREFVAQINRHGVTY |
| | | YADSVKGRFTISADNSKNTAYLQMNSLKPEDTAVYYCARGRTIFFGGGRYFDYWGQG |
| | | TLVTVSS |
| 10A-9 | 3757 | EVQLVESGGGLVQPGGSLRLSCAASGIPFRSRTMGWFRQAPGKEREFVAGITGSGRSQY |
| | | YADSVKGRFTISADNSKNTAYLQMNSLKPEDTAVYYCARGARIFGSVAPWRGGNYYG |
| | | MDVWGQGTLVTVSS |
| 10A-10 | 3758 | EVQLVESGGGLVQPGGSLRLSCAASGFTFSSFRMGWFRQAPGKEREFVAGISRGGSTN |
| | | YADSVKGRFTISADNSKNTAYLQMNSLKPEDTAVYYCARASGLWFRRPHVWGQGTL |
| | | VTVSS |
| 10A-11 | 3759 | EVQLVESGGGLVQPGGSLRLSCAASGRNFRRNSMGWFRQAPGKEREFVAGISWSGAR |
| | | THYADSVKGRFTISADNSKNTAYLQMNSLKPEDTAVYYCARVSRRPRSPPGYYYGMD |
| | | VWGQGTLVTVSS |
| 10A-12 | 3760 | EVQLVESGGGLVQPGGSLRLSCAASGRNLRMYRMGWFRQAPGKEREFVATIRWSDGS |
| | | TYYADSVKGRFTISADNSKNTAYLQMNSLKPEDTAVYYCTRARLRYFDWLFPTNFDY |
| | | WGQGTLVTVSS |
| 10A-13 | 3761 | EVQLVESGGGLVQPGGSLRLSCAASGGLTFSSNTMGWFRQAPGKEREFVASISSSGRTS |
| | | YADSVKGRFTISADNSKNTAYLQMNSLKPEDTAVYYCARRVRRLWFRSYFDLWGQGT |
| | | LVTVSS |
| 10A-14 | 3762 | EVQLVESGGGLVQPGGSLRLSCAASGFTLAYYAMGWFRQAPGKEREFVAAISWSGRNI |
| | | NYADSVKGRFTISADNSKNTAYLQMNSLKPEDTAVYYCARERARWFGKFSVSWGQGT |
| | | LVTVSS |
| 10A-15 | 3763 | EVQLVESGGGLVQPGGSLRLSCAASGRTFSSFPMGWFRQAPGKEREFVAAISWSGSTS |
| | | YADSVKGRFTISADNSKNTAYLQMNSLKPEDTAVYYSACGRLGFGAWGQGTLVTVSS |
| 10A-16 | 3764 | EVQLVESGGGLVQPGGSLRLSCAASGISSSKRNMGWFRQAPGKEREFVATWTSRGITT |
| | | YADSVKGRFTISADNSKNTAYLQMNSLKPEDTAVYYCARGGPPRLWGSYRRKYFDY |
| | | WGQGTLVTVSS |
| 10A-17 | 3765 | EVQLVESGGGLVQPGGSLRLSCAASGRTFSIYAMGWFRQAPGKEREFVARITRGGITKY |
| | | ADSVKGRFTISADNSKNTAYLQMNSLKPEDTAVYYCARGLGWLLGYYWGQGTLVTV |
| | | SS |
| 10A-18 | 3766 | EVQLVESGGGLVQPGGSLRLSCAASGRMYNSYSMGWFRQAPGKEREFVARISPGGTFY |
| | | ADSVKGRFTISADNSKNTAYLQMNSLKPEDTAVYYCTTSARSGWFWRYFDSWGQGTL |
| | | VTVSS |
| 10A-19 | 3767 | EVQLVESGGGLVQPGGSLRLSCAASGRTFRSYGMGWFRQAPGKEREFVASISRSGTTM |
| | | YADSVKGRFTISADNSKNTAYLQMNSLKPEDTAVYYCARRGLLQWFGAPNSWFDPW |
| | | GQGTLVTVSS |
| 10A-20 | 3768 | EVQLVESGGGLVQPGGSLRLSCAASGRTIRTMGWFRQAPGKEREFVATINSRGITNYA |
| | | DSVKGRFTISADNSKNTAYLQMNSLKPEDTAVYYCTTERDGLLWFRELFRPSWGQGTL |
| | | VTVSS |
| 10A-21 | 3769 | EVQLVESGGGLVQPGGSLRLSCAASGRSFSFNAMGWFRQAPGKEREFVARISRFGRTN |
| | | YADSVKGRFTISADNSKNTAYLQMNSLKPEDTAVYYCAKVHSYVWGGHSDYWGQGT |
| | | LVTVSS |
| 10A-22 | 3770 | EVQLVESGGGLVQPGGSLRLSCAASGRTYYAMGWFRQAPGKEREFVGAIDWSGRRIT |
| | | YADSVKGRFTISADNSKNTAYLQMNSLKPEDTAVYYCARVRFSRLGGVIGRPIDSWGQ |
| | | GTLVTVSS |
| 10A-23 | 3771 | EVQLVESGGGLVQPGGSLRLSCAASGRAFRRYTMGWFRQAPGKEREFVASITKFGSTN |
| | | YADSVKGRFTISADNSKNTAYLQMNSLKPEDTAVYYCAKDRGVLWFGELWYWGQGT |
| | | LVTVSS |
| 10A-24 | 3772 | EVQLVESGGGLVQPGGSLRLSCAASGRTFSNYRMGWFRQAPGKEREFVASINRGGSTK |
| | | YADSVKGRFTISADNSKNTAYLQMNSLKPEDTAVYYCASGKGGSATIFHLSRRPLYFD |
| | | YWGQGTLVTVSS |
| 10A-25 | 3773 | EVQLVESGGGLVQPGGSLRLSCAASGITFSPYAMGWFRQAPGKEREFVATINWSGGYT |
| | | VYADSVKGRFTISADNSKNTAYLQMNSLKPEDTAVYYCAKRKNRGPLWFGGGGWGY |
| | | WGQGTLVTVSS |
| 10A-26 | 3774 | EVQLVESGGGLVQPGGSLRLSCAASGRTFSGFTMSSTWMGWFRQAPGKEREFVAGIIT |
| | | NGSTNYADSVKGRFTISADNSKNTAYLQMNSLKPEDTAVYYCARRVAYSSFWSGLRK |
| | | HMDVWGQGTLVTVSS |
| 10A-27 | 3775 | EVQLVESGGGLVQPGGSLRLSCAASGRTFRRYSMGWFRQAPGKEREFVASITPGGNTN |
| | | YADSVKGRFTISADNSKNTAYLQMNSLKPEDTAVYYCASRRRWLTPYIFWGQGTLVT |
| | | VSS |
| 10A-28 | 3776 | EVQLVESGGGLVQPGGSLRLSCAASGSIFSIGMGWFRQAPGKEREFVARIWWRSGATY |
| | | YADSVKGRFTISADNSKNTAYLQMNSLKPEDTAVYYCAAISIFGRLKWGQGTLVTVSS |
| 10A-29 | 3777 | EVQLVESGGGLVQPGGSLRLSCAASGRTFTSYRMGWFRQAPGKEREFVAEISSSGGYT |
| | | YYADSVKGRFTISADNSKNTAYLQMNSLKPEDTAVYYCARVGPLRFLAQRPRLRPDY |
| | | WGQGTLVTVSS |
| 10A-30 | 3778 | EVQLVESGGGLVQPGGSLRLSCAASGRTFSSFRFRAMGWFRQAPGKEREFVALIFSGGS |
| | | TYYADSVKGRFTISADNSKNTAYLQMNSLKPEDTAVYYCAREWGRWLQRGSYWGQG |
| | | TLVTVSS |
| 10A-31 | 3779 | EVQLVESGGGLVQPGGSLRLSCAASGRTFGSYGMGWFRQAPGKEREFVATISIGGRTY |
| | | YADSVKGRFTISADNSKNTAYLQMNSLKPEDTAVYYCARGSGSGFMWYHGNNNYDR |
| | | WRYWGQGTLVTVSS |
| 10A-32 | 3780 | EVQLVESGGGLVQPGGSLRLSCAASGRTFRSYPMGWFRQAPGKEREFVASINRGGSTN |
| | | YADSVKGRFTISADNSKNTAYLQMNSLKPEDTAVYYCARGRYDFWSGYYRWFDPWG |
| | | QGTLVTVSS |
| 10A-33 | 3781 | EVQLVESGGGLVQPGGSLRLSCAASGRTFSRSDMGWFRQAPGKEREFVAAISWSGGST |
| | | SYADSVKGRFTISADNSKNTAYLQMNSLKPEDTAVYYCATVPPPRRFLEWLPRRLTYI |
| | | WGQGTLVTVSS |
| 10A-34 | 3782 | EVQLVESGGGLVQPGGSLRLSCAASGRTFRRYTMGWFRQAPGKEREFVASMRGSRSY |
| | | YADSVKGRFTISADNSKNTAYLQMNSLKPEDTAVYYCARMSGFPFLDYWGQGTLVTV |
| | | SS |
| 10A-35 | 3783 | EVQLVESGGGLVQPGGSLRLSCAASGSIFRLSTMGWFRQAPGKEREFVASISSFGSTYY |
| | | ADSVKGRFTISADNSKNTAYLQMNSLKPEDTAVYYCARTRGIFLWFGESFDYWGQGT |
| | | LVTVSS |
| 10A-36 | 3784 | EVQLVESGGGLVQPGGSLRLSCAASGIAFRIRTMGWFRQAPGKEREFVASITSGGSTNY |
| | | ADSVKGRFTISADNSKNTAYLQMNSLKPEDTAVYYCARGGPRFGGFRGYFDPWGQGT |
| | | LVTVSS |
| 10A-37 | 3785 | EVQLVESGGGLVQPGGSLRLSCAASGFTFTSYRMGWFRQAPGKEREFVAGISRFFGTA |
| | | YYADSVKGRFTISADNSKNTAYLQMNSLKPEDTAVYYCARVTRWFGGLDVWGQGTL |
| | | VTVSS |
| 10A-38 | 3786 | EVQLVESGGGLVQPGGSLRLSCAASGRTFSRYVMGWFRQAPGKEREFVASISRFGRTN |
| | | YADSVKGRFTISADNSKNTAYLQMNSLKPEDTAVYYCARHHGLGILWWGTMDVWGQ |
| | | GTLVTVSS |
| 10A-39 | 3787 | EVQLVESGGGLVQPGGSLRLSCAASGRTFSMGWFRQAPGKEREFVASISRFGRTNYAD |
| | | SVKGRFTISADNSKNTAYLQMNSLKPEDTAVYYCAKRSTWLPQHFDSWGQGTLVTVS |
| | | S |
| 10A-40 | 3788 | EVQLVESGGGLVQPGGSLRLSCAASGRTFSTYTMGWFRQAPGKEREFVARIWRSGGNT |
| | | YYADSVKGRFTISADNSKNTAYLQMNSLKPEDTAVYYCARGVRGVFRAYFDHWGQG |
| | | TLVTVSS |
| 10A-41 | 3789 | EVQLVESGGGLVQPGGSLRLSCAASGRNLRMYRMGWFRQAPGKEREFVALISRVGVT |
| | | SYADSVKGRFTISADNSKNTAYLQMNSLKPEDTAVYYCARGTSFFNFWSGSLGRVGFD |
| | | SWGQGTLVTVSS |
| 10A-42 | 3790 | EVQLVESGGGLVQPGGSLRLSCAASGITIRTHAMGWFRQAPGKEREFVATISRSGGNTY |
| | | YADSVKGRFTISADNSKNTAYLQMNSLKPEDTAVYYCTTAGVLRYFDWFRRPYWGQ |
| | | GTLVTVSS |
| 10A-43 | 3791 | EVQLVESGGGLVQPGGSLRLSCAASGRTFRRYHMGWFRQAPGKEREFVAAITSGGRTN |
| | | YADSVKGRFTISADNSKNTAYLQMNSLKPEDTAVYYCTTDGLRYFDWFPWASAFDIW |
| | | GQGTLVTVSS |
| 10A-44 | 3792 | EVQLVESGGGLVQPGGSLRLSCAASGRTFRRYTMGWFRQAPGKEREFVAVISWSGGST |
| | | KYADSVKGRFTISADNSKNTAYLQMNSLKPEDTAVYYCARKGRWSGMNVWGQGTLV |
| | | TVSS |
| 10A-45 | 3793 | EVQLVESGGGLVQPGGSLRLSCAASGRTFSWYPMGWFRQAPGKEREFVASISWGGAR |
| | | TYYADSVKGRFTISADNSKNTAYLQMNSLKPEDTAVYYCARSTGPRGSGRYRAHWFD |
| | | SWGQGTLVTVSS |
| 10A-46 | 3794 | EVQLVESGGGLVQPGGSLRLSCAASGRTFTSYRMGWFRQAPGKEREFVAAITWNSGRT |
| | | RYADSVKGRFTISADNSKNTAYLQMNSLKPEDTAVYYCSPSSWPFYFGAWGQGTLVT |
| | | VSS |
| 10A-47 | 3795 | EVQLVESGGGLVQPGGSLRLSCAASGRPLRRYVMGWFRQAPGKEREFVAAITNGGST |
| | | KYADSVKGRFTISADNSKNTAYLQMNSLKPEDTAVYYCARGTPWRLLWFGTLGPPPA |
| | | FDYWGQGTLVTVSS |
| 10A-48 | 3796 | EVQLVESGGGLVQPGGSLRLSCAASGRTFRRYAMGWFRQAPGKEREFVAAINRSGSTE |
| | | YADSVKGRFTISADNSKNTAYLQMNSLKPEDTAVYYCARQHQDFWTGYYTVWGQGT |
| | | LVTVSS |
| 10A-49 | 3797 | EVQLVESGGGLVQPGGSLRLSCAASGRTFRRYTMGWFRQAPGKEREFVASISRSGTTY |
| | | YADSVKGRFTISADNSKNTAYLQMNSLKPEDTAVYYCAKEGWRWLQLRGGFDYWGQ |
| | | GTLVTVSS |
| 10A-50 | 3798 | EVQLVESGGGLVQPGGSLRLSCAASGRTLSTYNMGWFRQAPGKEREFVASISRFGRTN |
| | | YADSVKGRFTISADNSKNTAYLQMNSLKPEDTAVYYCARRGKLSAAMHWFDPWGQG |
| | | TLVTVSS |
| 10A-51 | 3799 | EVQLVESGGGLVQPGGSLRLSCAASGRFFSTRVMGWFRQAPGKEREFVARIWPGGSTY |
| | | YADSVKGRFTISADNSKNTAYLQMNSLKPEDTAVYYCARDRGIFGVSRWGQGTLVTV |
| | | SS |
| 10A-52 | 3800 | EVQLVESGGGLVQPGGSLRLSCAASGRFFSICSMGWFRQAPGKEREFVAGINWRSGGS |
| | | TYYADSVKGRFTISADNSKNTAYLQMNSLKPEDTAVYYCARGSGWWEYWGQGTLVT |
| | | VSS |
| 10A-53 | 3801 | EVQLVESGGGLVQPGGSLRLSCAASGRMFSSRSNMGWFRQAPGKEREFVASISSGGTT |
| | | AYADSVKGRFTISADNSKNTAYLQMNSLKPEDTAVYYCARGFGRRFLEWLPRFDYWG |
| | | QGTLVTVSS |
| 10A-54 | 3802 | EVQLVESGGGLVQPGGSLRLSCAASGRTFSSARMGWFRQAPGKEREFVAGINMISSTK |
| | | YADSVKGRFTISADNSKNTAYLQMNSLKPEDTAVYYCAHFRRFLPRGYVDYWGQGTL |
| | | VTVSS |
| 10A-55 | 3803 | EVQLVESGGGLVQPGGSLRLSCAASGRTFRRYTMGWFRQAPGKEREFVARIAGGSTYY |
| | | ADSVKGRFTISADNSKNTAYLQMNSLKPEDTAVYYCARQQYYDFWSGYFRSGYFDLW |
| | | GQGTLVTVSS |
| 10A-56 | 3804 | EVQLVESGGGLVQPGGSLRLSCAASGHTFRNYGMGWFRQAPGKEREFVAAITSSGSTN |
| | | YADSVKGRFTISADNSKNTAYLQMNSLKPEDTAVYYCATVPPPRRFLEWLPRRLTYTW |
| | | GQGTLVTVSS |
| 10A-57 | 3805 | EVQLVESGGGLVQPGGSLRLSCAASGRTFSRYAMGWFRQAPGKEREFVASITKFGSTN |
| | | YADSVKGRFTISADNSKNTAYLQMNSLKPEDTAVYYCAKERESRFLKWRKTDWGQGT |
| | | LVTVSS |
| 10A-58 | 3806 | EVQLVESGGGLVQPGGSLRLSCAASGRNLRMYRMGWFRQAPGKEREFVASISRFGRT |
| | | NYADSVKGRFTISADNSKNTAYLQMNSLKPEDTAVYYCARHDSIGLFRHGMDVWGQ |
| | | GTLVTVSS |
| 10A-59 | 3807 | EVQLVESGGGLVQPGGSLRLSCAASGRTFRRYAMGWFRQAPGKEREFVARISSGGSTS |
| | | YADSVKGRFTISADNSKNTAYLQMNSLKPEDTAVYYCARDRGFGFWSGLRGYFDLWG |
| | | QGTLVTVSS |
| 10A-60 | 3808 | EVQLVESGGGLVQPGGSLRLSCAASGIPASMYLGWFRQAPGKEREFVAAITSGGRTSY |
| | | ADSVKGRFTISADNSKNTAYLQMNSLKPEDTAVYYCAKRKKRGPLWFGGGGWGYWG |
| | | QGTLVTVSS |
| 10A-61 | 3809 | EVQLVESGGGLVQPGGSLRLSCAASGIPFRSRTFSAYAMGWFRQAPGKEREFVAQITRG |
| | | GSTNYADSVKGRFTISADNSKNTAYLQMNSLKPEDTAVYYCARRHWFGFDYWGQGT |
| | | LVTVSS |
| 9-1 | 3810 | QVQLVESGGGVVQPGRSLRLSCAASGFTFSSYAMHWVRQAPGKGLEWVAVISYDGNH |
| | | EYYADSVKGRFTISRDNSKNTLYLQMNSLRAEDTAVYYCARGYKGYYYMDVWGQGT |
| | | LVTVSS |
| 9-2 | 3811 | QVQLVESGGGVVQPGRSLRLSCAASGFSFNNYGMHWVRQAPGKGLEWVAVISFDGSN |
| | | EYYADSVKGRFTISRDNSKNTLYLQMNSLRAEDTAVYYCAKENWLGYFDPWGQGTL |
| | | VTVSS |
| 9-3 | 3812 | QVQLVESGGGVVQPGRSLRLSCAASGFTFGTYAMHWVRQAPGKGLEWVAVVSTEGG |
| | | TTYYADSVKGRFTISRDNSKNTLYLQMNSLRAEDTAVYYCAGSYGAYFDYWGQGTLV |
| | | TVSS |
| 9-4 | 3813 | QVQLVESGGGVVQPGRSLRLSCAASGFDFSDYYMHWVRQAPGKGLEWVAVISYDGS |
| | | NKYYADSVKGRFTISRDNSKNTLYLQMNSLRAEDTAVYYCAREEPVYGMDVWGQGT |
| | | LVTVSS |
| 9-5 | 3814 | QVQLVESGGGVVQPGRSLRLSCAASGFTFGTYAMHWVRQAPGKGLEWVAVVSTEGG |
| | | TTYYADSVKGRFTISRDNSKNTLYLQMNSLRAEDTAVYYCAGSYGAYFDYWGQGTLV |
| | | TVSS |
| 9-6 | 3815 | QVQLVESGGGVVQPGRSLRLSCAASGFTFSGYAMHWVRQAPGKGLEWVAVISYDGSN |
| | | EYYADSVKGRFTISRDNSKNTLYLQMNSLRAEDTAVYYCARTNSGSYYGPFDYWGQG |
| | | TLVTVSS |
| 9-7 | 3816 | QVQLVESGGGVVQPGRSLRLSCAASGFTFSSYAMHWVRQAPGKGLEWVAVISYDGNH |
| | | EYYADSVKGRFTISRDNSKNTLYLQMNSLRAEDTAVYYCARGYKGYYYMDVWGQGT |
| | | LVTVSS |
| 9-8 | 3817 | QVQLVESGGGVVQPGRSLRLSCAASGFTFSSYAMHWVRQAPGKGLEWVAVISYDGNH |
| | | EYYADSVKGRFTISRDNSKNTLYLQMNSLRAEDTAVYYCARGYKGYYYMDVWGQGT |
| | | LVTVSS |
| 9-9 | 3818 | QVQLVESGGGVVQPGRSLRLSCAASGFIFRSYAMHWVRQAPGKGLEWVAVISYDGSS |
| | | KYYADSVKGRFTISRDNSKNTLYLQMNSLRAEDTAVYYCARPSSGSYFPPFDYWGQGT |
| | | LVTVSS |
| 9-10 | 3819 | QVQLVESGGGVVQPGRSLRLSCAASGFTFSDYGMHWVRQAPGKGLEWVAVVSYDGT |
| | | TKYYADSVKGRFTISRDNSKNTLYLQMNSLRAEDTAVYYCAKENWLGYFDPWGQGT |
| | | LVTVSS |
| 9-11 | 3820 | QVQLVESGGGVVQPGRSLRLSCAASGFTFSNFPMHWVRQAPGKGLEWVAVISYDGSL |
| | | KYYADSVKGRFTISRDNSKNTLYLQMNSLRAEDTAVYYCARYQGGYMDVWGQGTLV |
| | | TVSS |
| 9-12 | 3821 | QVQLVESGGGVVQPGRSLRLSCAASGFTFSRFAMHWVRQAPGKGLEWVAVISYDGSN |
| | | KYYADSVKGRFTISRDNSKNTLYLQMNSLRAEDTAVYYCARDTGLGFDPWGQGTLVT |
| | | VSS |
| 9-13 | 3822 | QVQLVESGGGVVQPGRSLRLSCAASGFTFNNYAMHWVRQAPGKGLEWVAVISYDGN |
| | | NKYYADSVKGRFTISRDNSKNTLYLQMNSLRAEDTAVYYCAKTMGGSYFDAFDIWGQ |
| | | GTLVTVSS |
| 9-14 | 3823 | QVQLVESGGGVVQPGRSLRLSCAASGFTFSDYTMHWVRQAPGKGLEWVAVISYEGSI |
| | | KYYADSVKGRFTISRDNSKNTLYLQMNSLRAEDTAVYYCARSSSGSYPSLVDYWGQG |
| | | TLVTVSS |
| 9-15 | 3824 | QVQLVESGGGVVQPGRSLRLSCAASGFSFSSYAMHWVRQAPGKGLEWVAVISFDGSN |
| | | EYYADSVKGRFTISRDNSKNTLYLQMNSLRAEDTAVYYCARDYWVDYFKPGGRGALL |
| | | TTSS |
| 10-1 | 3825 | QVQLVESGGGVVQPGRSLRLSCAASGFTFSRYAMHWVRQAPGKGLEWVAVISYDGTN |
| | | EYYADSVKGRFTISRDNSKNTLYLQMNSLRAEDTAVYYCARDTGLGFDPWGQGTLVT |
| | | VSS |
| 10-2 | 3826 | QVQLVESGGGVVQPGRSLRLSCAASGFTFSRYAMHWVRQAPGKGLEWVAVISYDGTN |
| | | EYYADSVKGRFTISRDNSKNTLYLQMNSLRAEDTAVYYCARDTGLGFDPWGQGTLVT |
| | | VSS |
| 10-3 | 3827 | QVQLVESGGGVVQPGRSLRLSCAASGFTFSRYAMHWVRQAPGKGLEWVAVISYDGTN |
| | | EYYADSVKGRFTISRDNSKNTLYLQMNSLRAEDTAVYYCARDTGLGFDPWGQGTLVT |
| | | VSS |
| 10-4 | 3828 | QVQLVESGGGVVQPGRSLRLSCAASGFTFSRYAMHWVRQAPGKGLEWVAVISYDGTN |
| | | EYYADSVKGRFTISRDNSKNTLYLQMNSLRAEDTAVYYCARDTGLGFDPWGQGTLVT |
| | | VSS |
| 10-5 | 3829 | QVQLVESGGGVVQPGRSLRLSCAASGFTFSRYAMHWVRQAPGKGLEWVAVISYDGTN |
| | | EYYADSVKGRFTISRDNSKNTLYLQMNSLRAEDTAVYYCARDTGLGFDPWGQGTLVT |
| | | VSS |
| 10-6 | 3830 | QVQLVESGGGVVQPGRSLRLSCAASGFTFSRYAMHWVRQAPGKGLEWVAVISYDGTN |
| | | EYYADSVKGRFTISRDNSKNTLYLQMNSLRAEDTAVYYCARDTGLGFDPWGQGTLVT |
| | | VSS |
| 11-1 | 3831 | QVQLVESGGGVVQPGRSLRLSCAASGFTFGSYGMHWVRQAPGKGLEWVAVISYDGG |
| | | DEYYADSVKGRFTISRDNSKNTLYLQMNSLRAEDTAVYYCARDISRYGYYGMDVWG |
| | | QGTLVTVSS |
| 11-2 | 3832 | QVQLVESGGGVVQPGRSLRLSCAASGFTFGTYAMHWVRQAPGKGLEWVAVVSTEGG |
| | | TTYYADSVKGRFTISRDNSKNTLYLQMNSLRAEDTAVYYCAGSYGAYFDYWGQGTLV |
| | | TVSS |
| 11-3 | 3833 | QVQLVESGGGVVQPGRSLRLSCAASGFTFSNFAMHWVRQAPGKGLEWVAVISYDGNH |
| | | EYYADSVKGRFTISRDNSKNTLYLQMNSLRAEDTAVYYCAKTNSGSYGGMFDYWGQ |
| | | GTLVTVSS |
| 11-4 | 3834 | QVQLVESGGGVVQPGRSLRLSCAASGFTFDNYAMHWVRQAPGKGLEWVAVISDDGR |
| | | NKYYADSVKGRFTISRDNSKNTLYLQMNSLRAEDTAVYYCAKDNYYDSSGYYGGGM |
| | | DVWGQGTLVTVSS |
| 11-5 | 3835 | QVQLVESGGGVVQPGRSLRLSCAASGFTFSSFAMHWVRQAPGKGLEWVAVISYDGSN |
| | | KYYADSVKGRFTISRDNSKNTLYLQMNSLRAEDTAVYYCARSRSGSYSSYFDYWGQG |
| | | TLVTVSS |
| 11-6 | 3836 | QVQLVESGGGVVQPGRSLRLSCAASGFTFGTYAMHWVRQAPGKGLEWVAVVSTEGG |
| | | TTYYADSVKGRFTISRDNSKNTLYLQMNSLRAEDTAVYYCAGEYYDSSGSSIDYWGQ |
| | | GTLVTVSS |
| 11-7 | 3837 | QVQLVESGGGVVQPGRSLRLSCAASGFTFSSYAMHWVRQAPGKGLEWVAVISYDGSN |
| | | QYYADSVKGRFTISRDNSKNTLYLQMNSLRAEDTAVYYCARAKGGGYRGAFDIWGQ |
| | | GTLVTVSS |
| 11-8 | 3838 | QVQLVESGGGVVQPGRSLRLSCAASGFTFSSYAMHWVRQAPGKGLEWVAVISYDGSN |
| | | TYYADSVKGRFTISRDNSKNTLYLQMNSLRAEDTAVYYCARPRGGSYWTYFDYWGQ |
| | | GTLVTVSS |
| 11-9 | 3839 | QVQLVESGGGVVQPGRSLRLSCAASGFTFGTYAMHWVRQAPGKGLEWVAVVSTEGG |
| | | TTYYADSVKGRFTISRDNSKNTLYLQMNSLRAEDTAVYYCAGSYGAYFDYWGQGTLV |
| | | TVSS |
| 11-10 | 3840 | QVQLVESGGGVVQPGRSLRLSCAASGFIFNNYGMHWVRQAPGKGLEWVAVISYDGSN |
| | | IYYADSVKGRFTISRDNSKNTLYLQMNSLRAEDTAVYYCARDYNDGIGSYTGAFDSW |
| | | GQGTLVTVSS |
| 11-11 | 3841 | QVQLVESGGGVVQPGRSLRLSCAASGFTFDNYAMHWVRQAPGKGLEWVAVISYDGS |
| | | NKYYADSVKGRFTISRDNSKNTLYLQMNSLRAEDTAVYYCLREGILWDVWGQGTLVT |
| | | VSS |
| 11-12 | 3842 | QVQLVESGGGVVQPGRSLRLSCAASGFTFSSQAMHWVRQAPGKGLEWVAVISYDGSN |
| | | KYYADSVKGRFTISRDNSKNTLYLQMNSLRAEDTAVYYCAKTEGGTYGGAFDIWGQG |
| | | TLVTVSS |
| 11-13 | 3843 | QVQLVESGGGVVQPGRSLRLSCAASGFSFSSYGMHWVRQAPGKGLEWVAVISYDGSD |
| | | KYYADSVKGRFTISRDNSKNTLYLQMNSLRAEDTAVYYCAKDNYYDSSGYYGGGMD |
| | | VWGQGTLVTVSS |
| 11-14 | 3844 | QVQLVESGGGVVQPGRSLRLSCAASGFTFSSYSMHWVRQAPGKGLEWVAVISYDGSH |
| | | KYYADSVKGRFTISRDNSKNTLYLQMNSLRAEDTAVYYCARDGWGYFDYWGQGTLV |
| | | TVSS |
| 11-15 | 3845 | QVQLVESGGGVVQPGRSLRLSCAASGFIFSNYGMHWVRQAPGKGLEWVAVISYDGSD |
| | | KYYADSVKGRFTISRDNSKNTLYLQMNSLRAEDTAVYYCARDDYMYGFEHWGQGTL |
| | | VTVSS |
| 11-16 | 3846 | QVQLVESGGGVVQPGRSLRLSCAASGFTFSDHYMHWVRQAPGKGLEWVAVISYDGSN |
| | | EYYADSVKGRFTISRDNSKNTLYLQMNSLRAEDTAVYYCAKDLGPAGVDYWGQGTL |
| | | VTVSS |
| 11-17 | 3847 | QVQLVESGGGVVQPGRSLRLSCAASGFIFSSYAMHWVRQAPGKGLEWVAVISYDGSN |
| | | KYYADSVKGRFTISRDNSKNTLYLQMNSLRAEDTAVYYCARSRSGSYSSWFDYWGQG |
| | | TLVTVSS |
| 11-18 | 3848 | QVQLVESGGGVVQPGRSLRLSCAASGFTFGTYAMHWVRQAPGKGLEWVAVISYDGN |
| | | NKYYADSVKGRFTISRDNSKNTLYLQMNSLRAEDTAVYYCAKTGSGSYYSWFDYWG |
| | | QGTLVTVSS |
| 11-19 | 3849 | QVQLVESGGGVVQPGRSLRLSCAASGFTFSSYAMHWVRQAPGKGLEWVAVISYDGTN |
| | | DYYADSVKGRFTISRDNSKNTLYLQMNSLRAEDTAVYYCARTRGGSYFTPFDYWGQG |
| | | TLVTVSS |
| 11-20 | 3850 | QVQLVESGGGVVQPGRSLRLSCAASGFTFDDYAMHWVRQAPGKGLEWVAVISYDGS |
| | | NKYYADSVKGRFTISRDNSKNTLYLQMNSLRAEDTAVYYCASPHSGSYWAAFDIWGQ |
| | | GTLVTVSS |
| 12-1 | 3851 | QVQLVESGGGVVQPGRSLRLSCAASGFTFSYYGMHWVRQAPGKGLEWVAVTSYDGS |
| | | NKYYADSVKGRFTISRDNSKNTLYLQMNSLRAEDTAVYYCARPQGGSYFAAFDIWGQ |
| | | GTLVTVSS |
| 12-2 | 3852 | QVQLVESGGGVVQPGRSLRLSCAASGFIFRSYAMHWVRQAPGKGLEWVAVISYDGSS |
| | | KYYADSVKGRFTISRDNSKNTLYLQMNSLRAEDTAVYYCARPSSGSYFPPFDYWGQGT |
| | | LVTVSS |
| 12-3 | 3853 | QVQLVESGGGVVQPGRSLRLSCAASGFTFSSYAMHWVRQAPGKGLEWVAVISYDGSN |
| | | QYYADSVKGRFTISRDNSKNTLYLQMNSLRAEDTAVYYCAKTRTGSYFSAFDIWGQG |
| | | TLVTVSS |
| 12-4 | 3854 | QVQLVESGGGVVQPGRSLRLSCAASGFTFSYYGMHWVRQAPGKGLEWVAVISYDGT |
| | | NDYYADSVKGRFTISRDNSKNTLYLQMNSLRAEDTAVYYCAKPHSGSYRGYFDYWG |
| | | QGTLVTVSS |
| 12-5 | 3855 | QVQLVESGGGVVQPGRSLRLSCAASGFTFSYYGMHWVRQAPGKGLEWVAVTSYDGS |
| | | NKYYSDSVKGRFTISRDNSKNTLYLQMNSLRAEDTAVYYCARPKSGSYATYFDYWGQ |
| | | GTLVTVSS |
| 12-6 | 3856 | QVQLVESGGGVVQPGRSLRLSCAASGFIFRNYAMHWVRQAPGKGLEWVAVISYDGSN |
| | | KYYADSVKGRFTISRDNSKNTLYLQMNSLRAEDTAVYYCARPRGGSYHGAFDIWGQG |
| | | TLVTVSS |
| 12-7 | 3857 | QVQLVESGGGVVQPGRSLRLSCAASGFTFSIYAMHWVRQAPGKGLEWVAVISYDGTN |
| | | EYYADSVKGRFTISRDNSKNTLYLQMNSLRAEDTAVYYCAKSRGGSYYGAFDYWGQ |
| | | GTLVTVSS |
| 12-8 | 3858 | QVQLVESGGGVVQPGRSLRLSCAASGFTFNNYVMHWVRQAPGKGLEWVAVISYDGT |
| | | NDYYADSVKGRFTISRDNSKNTLYLQMNSLRAEDTAVYYCARGESGSYWGAFDYWG |
| | | QGTLVTVSS |
| 12-9 | 3859 | QVQLVESGGGVVQPGRSLRLSCAASGFTFSSYGMHWVRQAPGKGLEWVAVISYDGTT |
| | | EYYADSVKGRFTISRDNSKNTLYLQMNSLRAEDTAVYYCARPSSGSYLGFFDYWGQG |
| | | TLVTVSS |
| 12-10 | 3860 | QVQLVESGGGVVQPGRSLRLSCAASGFIFRSYAMHWVRQAPGKGLEWVAVISYDGSIK |
| | | YYADSVKGRFTISRDNSKNTLYLQMNSLRAEDTAVYYCARTRGGSYYGAFDYWGQG |
| | | TLVTVSS |
| 12-11 | 3861 | QVQLVESGGGVVQPGRSLRLSCAASGFSFGGYGMHWVRQAPGKGLEWVAVISYDGS |
| | | NEYYADSVKGRFTISRDNSKNTLYLQMNSLRAEDTAVYYCAKSYSGSYSSYFDYWGQ |
| | | GTLVTVSS |
| 12-12 | 3862 | QVQLVESGGGVVQPGRSLRLSCAASGFAFSSHAMHWVRQAPGKGLEWVAVISYDGSN |
| | | KYYADSEKGRFTISRDNSKNTLYLQMNSLRAEDTAVYYCAKAYSGSYMGYFDYWGQ |
| | | GTLVTVSS |
| 12-13 | 3863 | QVQLVESGGGVVQPGRSLRLSCAASGFSFSTYGMHWVRQAPGKGLEWVAVISYDGSN |
| | | KYYADSVKGRFTISRDNSKNTLYLQMNSLRAEDTAVYYCARPLSGSYWSWFDPWGQ |
| | | GTLVTVSS |
| 12-14 | 3864 | QVQLVESGGGVVQPGRSLRLSCAASGFTFSSYSMHWVRQAPGKGLEWVAVISYDGSN |
| | | KYYADSVKGRFTISRDNSKNTLYLQMNSLRAEDTAVYYCARGKGGGYYSSFDFWGQ |
| | | GTLVTVSS |
| 12-15 | 3865 | QVQLVESGGGVVQPGRSLRLSCAASGFSFGGYGMHWVRQAPGKGLEWVAVISYDGS |
| | | NKYYADSVKGRFTISRDNSKNTLYLQMNSLRAEDTAVYYCARPYSGSYISWFDYWGQ |
| | | GTLVTVSS |
| 12-16 | 3866 | QVQLVESGGGVVQPGRSLRLSCAASGFIFRSYAMHWVRQAPGKGLEWVAVISYDGSS |
| | | KYYADSVKGRFTISRDNSKNTLYLQMNSLRAEDTAVYYCARTLGGSYFAAFDIWGQG |
| | | TLVTVSS |
| 12-17 | 3867 | QVQLVESGGGVVQPGRSLRLSCAASGFTFGSYGMHWVRQAPGKGLEWVAVISYDGN |
| | | HEYYADSVKGRFTISRDNSKNTLYLQMNSLRAEDTAVYYCARPHSGSYTAYFDYWGQ |
| | | GTLVTVSS |
| 12-18 | 3868 | QVQLVESGGGVVQPGRSLRLSCAASGFTFSSYAMHWVRQAPGKGLEWVAVISYDGSN |
| | | QYYADSVKGRFTISRDNSKNTLYLQMNSLRAEDTAVYYCARGYGGSYSYFDYWGQG |
| | | TLVTVSS |
| 12-19 | 3869 | QVQLVESGGGVVQPGRSLRLSCAASGFAFSSYAMHWVRQAPGKGLEWVAVISYDGTY |
| | | EYYADSVKGRFTISRDNSKNTLYLQMNSLRAEDTAVYYCARSLGGSYFSGMDVWGQG |
| | | TLVTVSS |
| 12-20 | 3870 | QVQLVESGGGVVQPGRSLRLSCAASGFSFGGYGMHWVRQAPGKGLEWVAVISYDGS |
| | | NKYYADSVKGRFTISRDNSKNTLYLQMNSLRAEDTAVYYCARSKGGSYYGPFDYWG |
| | | QGTLVTVSS |
| 12-21 | 3871 | QVQLVESGGGVVQPGRSLRLSCAASGFSFGGYGMHWVRQAPGKGLEWVAVISYDGS |
| | | NKYYADSVKGRFTISRDNSKNTLYLQMNSLRAEDTAVYYCARPKGGNYWNAFDIWG |
| | | QGTLVTVSS |
| 12-22 | 3872 | QVQLVESGGGVVQPGRSLRLSCAASGFTFSSYGMHWVRQAPGKGLEWVAVISYDGNH |
| | | EYYADSVKGRFTISRDNSKNTLYLQMNSLRAEDTAVYYCARPKSGSYVSYFDYWGQG |
| | | TLVTVSS |
| 12-23 | 3873 | QVQLVESGGGVVQPGRSLRLSCAASGFIFSSYAMHWVRQAPGKGLEWVAVISYDGSN |
| | | KYYADSVKGRFTISRDNSKNTLYLQMNSLRAEDTAVYYCARPRGGNYLNYFDYWGQ |
| | | GTLVTVSS |
| 12-24 | 3874 | QVQLVESGGGVVQPGRSLRLSCAASGFTFSNFPMHWVRQAPGKGLEWVAVISYDGNN |
| | | KYYADSVKGRFTISRDNSKNTLYLQMNSLRAEDTAVYYCAKDHGDHYFDYWGQGTL |
| | | VTVSS |
| 12-25 | 3875 | QVQLVESGGGVVQPGRSLRLSCAASGFTFSSYAMHWVRQAPGKGLEWVAVISYDGSN |
| | | QYYADSVKGRFTISRDNSKNTLYLQMNSLRAEDTAVYYCARDKGGSYYGPFDYWGQ |
| | | GTLVTVSS |
| 12-26 | 3876 | QVQLVESGGGVVQPGRSLRLSCAASGFTFSNYAMHWVRQAPGKGLEWVAVISYDGSN |
| | | EYYADSEKGRFTISRDNSKNTLYLQMNSLRAEDTAVYYCAKSGSGSYFSPFDYWGQGT |
| | | LVTVSS |
| 12-27 | 3877 | QVQLVESGGGVVQPGRSLRLSCAASGFSFGGYGMHWVRQAPGKGLEWVAVISYDGST |
| | | KYYADSVKGRFTISRDNSKNTLYLQMNSLRAEDTAVYYCARPRGGSYKDAFDIWGQG |
| | | TLVTVSS |
| 12-28 | 3878 | QVQLVESGGGVVQPGRSLRLSCAASGFTFSSYAMHWVRQAPGKGLEWVAVISYDGTN |
| | | EYYADSEKGRFTISRDNSKNTLYLQMNSLRAEDTAVYYCARAHGGSYFSGMDVWGQ |
| | | GTLVTVSS |
| 12-29 | 3879 | QVQLVESGGGVVQPGRSLRLSCAASGFSFSNYGMHWVRQAPGKGLEWVAVISYDGN |
| | | NKYYADSVKGRFTISRDNSKNTLYLQMNSLRAEDTAVYYCARSKGGSYYGPFDDWG |
| | | QGTLVTVSS |
| 12-30 | 3880 | QVQLVESGGGVVQPGRSLRLSCAASGFTFSGYAMHWVRQAPGKGLEWVAVISYDGSN |
| | | KYYADSVKGRFTISRDNSKNTLYLQMNSLRAEDTAVYYCARSRGGSYYAPFDYWGQG |
| | | TLVTVSS |
| 12-31 | 3881 | QVQLVESGGGVVQPGRSLRLSCAASGFTFSYYTMHWVRQAPGKGLEWVAVTSYDGS |
| | | NKYYADSVKGRFTISRDNSKNTLYLQMNSLRAEDTAVYYCARPLGGSYFAAFDIWGQ |
| | | GTLVTVSS |
| 12-32 | 3882 | QVQLVESGGGVVQPGRSLRLSCAASGFTFGTYAMHWVRQAPGKGLEWVAVISYDGN |
| | | NKYYADSVKGRFTISRDNSKNTLYLQMNSLRAEDTAVYYCAKTMSGSYFSAFDIWGQ |
| | | GTLVTVSS |
| 12-33 | 3883 | QVQLVESGGGVVQPGRSLRLSCAASGFTFSSYAMHWVRQAPGKGLEWVAVISYDGSN |
| | | QYYADSVKGRFTISRDNSKNTLYLQMNSLRAEDTAVYYCARPHGGNYFDWFDPWGQ |
| | | GTLVTVSS |
| 12-34 | 3884 | QVQLVESGGGVVQPGRSLRLSCAASGFIFRSYAMHWVRQAPGKGLEWVAVISYDGSS |
| | | KYYADSVKGRFTISRDNSKNTLYLQMNSLRAEDTAVYYCARPSGGSYFDPFDYWGQG |
| | | TLVTVSS |
| 12-35 | 3885 | QVQLVESGGGVVQPGRSLRLSCAASGFTFSSSSMHWVRQAPGKGLEWVAVISYDGSN |
| | | KYYADSVKGRFTISRDNSKNTLYLQMNSLRAEDTAVYYCAKVDSGSYVGYFDYWGQ |
| | | GTLVTVSS |
| 12-36 | 3886 | QVQLVESGGGVVQPGRSLRLSCAASGFSFNNYGMHWVRQAPGKGLEWVAVISYDGS |
| | | NDYYADSVKGRFTISRDNSKNTLYLQMNSLRAEDTAVYYCARPNSGSYSNYFDYWGQ |
| | | GTLVTVSS |
| 12-37 | 3887 | QVQLVESGGGVVQPGRSLRFSCAGTGFTFSSYAMHWVRQAPGKGLEWVAVISYDGSN |
| | | QYYADSVKGRFTISRDNSKNTLYLQMNSLRAEDTAVYYCARSRSGSYLAYFDYWGQG |
| | | TLVTVSS |
| 12-38 | 3888 | QVQLVESGGGVVQPGRSLRLSCAASGFTFSSYAMHWVRQAPGKGLEWVAVISYDGSN |
| | | QYYADSVKGRFTISRDNSKNTLYLQMNSLRAEDTAVYYCARAAGGSYSSWFDPWGQ |
| | | GTLVTVSS |
| 12-39 | 3889 | QVQLVESGGGVVQPGRSLRLSCAASGFTFSSYAMHWVRQAPGKGLEWVAVISYDGNH |
| | | EYYADSVKGRFTISRDNSKNTLYLQMNSLRAEDTAVYYCARAHSGSYFSHFDYWGQG |
| | | TLVTVSS |
| 12-40 | 3890 | QVQLVESGGGVVQPGRSLRLSCAASGFTFSSYAMHWVRQAPGKGLEWVAVISYDGSN |
| | | TYYADSVKGRFTISRDNSKNTLYLQMNSLRAEDTAVYYCARPTSGSYFSWFDPWGQG |
| | | TLVTVSS |
| 12-41 | 3891 | QVQLVESGGGVVQPGRSLRLSCAASGFIFSSYAMHWVRQAPGKGLEWVAVISYDGSN |
| | | KYYADSVKGRFTISRDNSKNTLYLQMNSLRAEDTAVYYCARPNSGSYWGPFDYWGQ |
| | | GTLVTVSS |
| 12-42 | 3892 | QVQLVESGGGVVQPGRSLRLSCAASGFTFGSYGMHWVRQAPGKGLEWVAVISYDGSH |
| | | KYYADSVKGRFTISRDNSKNTLYLQMNSLRAEDTAVYYCARALGGNYYYFDYWGQG |
| | | TLVTVSS |
| 12-43 | 3893 | QVQLVESGGGVVQPGRSLRLSCAASGFIFSSYGMHWVRQAPGKGLEWVAVISYDGSN |
| | | EYYADSVKGRFTISRDNSKNTLYLQMNSLRAEDTAVYYCARPRSGSYLSAFDYWGQG |
| | | TLVTVSS |
| 13-1 | 3894 | QVQLVESGGGVVQPGRSLRLSCAASGFTFSSYSMHWVRQAPGKGLEWVAVISYDGRN |
| | | QYYADSVKGRFTISRDNSKNTLYLQMNSLRAEDTAVYYCAKGYGGNYYYMDGWGQ |
| | | GTLVTVSS |
| 13-2 | 3895 | QVQLVESGGGVVQPGRSLRLSCAASGFTFSSYAMHWVRQAPGKGLEWVAVISYDGNN |
| | | KYYADSVKGRFTISRDNSKNTLYLQMNSLRAEDTAVYYCARTYGGSYYSAFDYWGQ |
| | | GTLVTVSS |
| 13-3 | 3896 | QVQLVESGGGVVQPGRSLRLSCAASGFSFNNHAMHWVRQAPGKGLEWVAVISYDGS |
| | | DKYYADSVKGRFTISRDNSKNTLYLQMNSLRAEDTAVYYCARNLLRGYGMDVWGQG |
| | | TLVTVSS |
| 13-4 | 3897 | QVQLVESGGGVVQPGRSLRLSCAASGFAFDDYAMHWVRQAPGKGLEWVAVISYDGS |
| | | NKYYADSVKGRFTISRDNSKNTLYLQMNSLRAEDTAVYYCATLGYGDYPDYWGQGT |
| | | LVTVSS |
| 13-5 | 3898 | QVQLVESGGGVVQPGRSLRLSCAASGFIFRSYAMHWVRQAPGKGLEWVAVISYDGSS |
| | | KYYADSVKGRFTISRDNSKNTLYLQMNSLRAEDTAVYYCARPLGGGYQDAFDIWGQG |
| | | TLVTVSS |
| 1N-1 | 3899 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCAKEDVGKPFYWGQGTLVT |
| | | VSS |
| 1N-2 | 3900 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCAKEDVGKPFFWGQGTLVTV |
| | | SS |
| 1N-3 | 3901 | EVQLVESGGGLVQPGGSLRLSCAASGGTFSSIGMGWFRQAPGKEREFVAAISWDGGST |
| | | AYADSVKGRFTISADNSKNTAYLQMNSLKPEDTAVYYCAKEDVGKPFDWGQGTLVT |
| | | VSS |
| 1N-4 | 3902 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCAKEDVGKPFCWGQGTLVTV |
| | | SS |
| 1N-5 | 3903 | EVQLVESGGGLVQPGGSLRLSCAASGGTFSSIGMGWFRQAPGKEREFVAAISWNGGAT |
| | | AYADSVKGRFTISADNSKNTAYLQMNSLKPEDTAVYYCAKEDVGKPFDWGQGTLVT |
| | | VSS |
| 1N-6 | 3904 | EVQLVESGGGLVQPGGSLRLSCAASGGTFSSIGMGWFRQAPGKEREFVAAISWDGGRT |
| | | AYADSVKGRFTISADNSKNTAYLQMNSLKPEDTAVYYCAKEDVGKPFDWGQGTLVT |
| | | VSS |
| 1N-7 | 3905 | EVQLVESGGGLVQPGGSLRLSCAASGGTFSSIGMGWFRQAPGKEREFVAAISWSGGAT |
| | | AYADSVKGRFTISADNSKNTAYLQMNSLKPEDTAVYYCAKEDVGKPFDWGQGTLVT |
| | | VSS |
| 1N-8 | 3906 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCAKEDVGKPLDWGQGTLVT |
| | | VSS |
| 1N-9 | 3907 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCAKEDVGPPFDWGQGTLVTV |
| | | SS |
| 1N-10 | 3908 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCAKEDVGKPFHWGQGTLVT |
| | | VSS |
| 1N-11 | 3909 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCAKEDVGPPFYWGQGTLVTV |
| | | SS |
| 1N-12 | 3910 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCWKEDVGKPGDWGQGTLVT |
| | | VSS |
| 1N-13 | 3911 | EVQLVESGGGLVQPGGSLRLSCAASGGTFSSIGMGWFRQAPGKEREFVAAISWDRGAT |
| | | AYADSVKGRFTISADNSKNTAYLQMNSLKPEDTAVYYCAKEDVGKPFCWGQGTLVTV |
| | | SS |
| 1N-14 | 3912 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCRKEDVGKPFFWGQGTLVTV |
| | | SS |
| 1N-15 | 3913 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCYKEDVGKPFYWGQGTLVT |
| | | VSS |
| 1N-16 | 3914 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCHKEDVGKPFYWGQGTLVT |
| | | VSS |
| 1N-17 | 3915 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCFKEDVGKPFFWGQGTLVTV |
| | | SS |
| 1N-18 | 3916 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCYKEDVGKPFFWGQGTLVTV |
| | | SS |
| 1N-19 | 3917 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCFKEDVGKPFWWGQGTLVT |
| | | VSS |
| 1N-20 | 3918 | EVQLVESGGGLVQPGGSLRLSCAASGGTFSSIGMGWFRQAPGKEREFVAAISWDGGAT |
| | | AYADSVKGRFTISADNSKNTAYLQMNSLKPEDTAVYYCWKEDVGKPFDEGQGTLVTV |
| | | SS |
| 1N-21 | 3919 | EVQLVESGGGLVQPGGSLRLSCAASGFTFSGSWMGWFRQAPGKEREFVATINEYGGR |
| | | NYADSVKGRFTISADNSKNTAYLQMNSLKPEDTAVYYCARVDRDFDYWGQGTLVTV |
| | | SS |
| 1N-22 | 3920 | EVQLVESGGGLVQPGGSLRLSCAASGFWFSPSWMGWFRQAPGKEREFVATINEYGGR |
| | | NYADSVKGRFTISADNSKNTAYLQMNSLKPEDTAVYYCARVDRDFDYWGQGTLVTV |
| | | SS |
| 1N-23 | 3921 | EVQLVESGGGLVQPGGSLRLSCAASGFTFSPSWMGWFRQAPGKEREFVATINEYGGRN |
| | | YADSVKGRFTISADNSKNTAYLQMNSLKPEDTAVYYCARVTRDFDYWGQGTLVTVSS |
| 1N-24 | 3922 | EVQLVESGGGLVQPGGSLRLSCAASGFTFSPSWMGWFRQAPGKEREFVATINEYGGRN |
| | | YADSVKGRFTISADNSKNTAYLQMNSLKPEDTAVYYCARVWRDFDYWGQGTLVTVS |
| | | S |
| 1N-25 | 3923 | EVQLVESGGGLVQPGGSLRLSCAASGFTFSPSWMGWFRQAPGKEREFVATINSYGGRN |
| | | YADSVKGRFTISADNSKNTAYLQMNSLKPEDTAVYYCARVDRDFDYWGQGTLVTVSS |
| 1N-26 | 3924 | EVQLVESGGGLVQPGGSLRLSCAASGFTFSPSWMGWFRQAPGKEREFVAFINEYGGRN |
| | | YADSVKGRFTISADNSKNTAYLQMNSLKPEDTAVYYCARVDRDFDYWGQGTLVTVSS |
| 1N-27 | 3925 | EVQLVESGGGLVQPGGSLRLSCAASGFTFSPSWMGWFRQAPGKEREFVATINPYGGRN |
| | | YADSVKGRFTISADNSKNTAYLQMNSLKPEDTAVYYCARVDRDFDYWGQGTLVTVSS |
| 1N-28 | 3926 | EVQLVESGGGLVQPGGSLRLSCAASGTTFSPSWMGWFRQAPGKEREFVATINEYGGRN |
| | | YADSVKGRFTISADNSKNTAYLQMNSLKPEDTAVYYCARVDRDFDYWGQGTLVTVSS |
| 1N-29 | 3927 | EVQLVESGGGLVQPGGSLRLSCAASGFTFSPSWFGWFRQAPGKEREFVATINEYGGRN |
| | | YADSVKGRFTISADNSKNTAYLQMNSLKPEDTAVYYCARVDRDFDYWGQGTLVTVSS |
| 1N-30 | 3928 | EVQLVESGGGLVQPGGSLRLSCAASGFTFSPSWMGWFRQAPGKEREFVATINEYGGRN |
| | | YADSVKGRFTISADNSKNTAYLQMNSLKPEDTAVYYCARVYRDFDYWGQGTLVTVSS |
| 1N-31 | 3929 | EVQLVESGGGLVQPGGSLRLSCAASGFTFSPSWMGWFRQAPGKEREFVAWINPYGGR |
| | | NYADSVKGRFTISADNSKNTAYLQMNSLKPEDTAVYYCARVDRDFDYWGQGTLVTV |
| | | SS |
| 1N-32 | 3930 | EVQLVESGGGLVQPGGSLRLSCAASGFWFSPSWMGWFRQAPGKEREFVATINEYGGR |
| | | NYADSVKGRFTISADNSKNTAYLQMNSLKPEDTAVYYCARVTRDFDYWGQGTLVTVS |
| | | S |
| 1N-33 | 3931 | EVQLVESGGGLVQPGGSLRLSCAASGFFFYPSWMGWFRQAPGKEREFVATINEYGGRN |
| | | YADSVKGRFTISADNSKNTAYLQMNSLKPEDTAVYYCARVDRDFDYWGQGTLVTVSS |
| 1N-34 | 3932 | EVQLVESGGGLVQPGGSLRLSCAASGFFFHPSWMGWFRQAPGKEREFVATINEYGGRN |
| | | YADSVKGRFTISADNSKNTAYLQMNSLKPEDTAVYYCARVDRDFDYWGQGTLVTVSS |
| 1N-35 | 3933 | EVQLVESGGGLVQPGGSLRLSCAASGFWFSPSWMGWFRQAPGKEREFVATINEYGGR |
| | | NYADSVKGRFTISADNSKNTAYLQMNSLKPEDTAVYYCARVSRDFDYWGQGTLVTVS |
| | | S |
| 1N-36 | 3934 | EVQLVESGGGLVQPGGSLRLSCAASGFWFSPSWMGWFRQAPGKEREFVATINEYGGR |
| | | NYADSVKGRFTISADNSKNTAYLQMNSLKPEDTAVYYCARVDGDFDYWGQGTLVTV |
| | | SS |
| 1N-37 | 3935 | EVQLVESGGGLVQPGGSLRLSCAASGFWFSPSWMGWFRQAPGKEREFVATINNYGGR |
| | | NYADSVKGRFTISADNSKNTAYLQMNSLKPEDTAVYYCARVDRDFDYWGQGTLVTV |
| | | SS |
| 1N-38 | 3936 | EVQLVESGGGLVQPGGSLRLSCAASGFCFSPSWMGWFRQAPGKEREFVATINEYGGRN |
| | | YADSVKGRFTISADNSKNTAYLQMNSLKPEDTAVYYCARVTRDFDYWGQGTLVTVSS |
| 1N-39 | 3937 | EVQLVESGGGLVQPGGSLRLSCAASGWFFSPSWMGWFRQAPGKEREFVATINEYGGR |
| | | NYADSVKGRFTISADNSKNTAYLQMNSLKPEDTAVYYCARVDRDFDYWGQGTLVTV |
| | | SS |
| 1N-40 | 3938 | EVQLVESGGGLVQPGGSLRLSCAASGFWFSPSWMGWFRQAPGKEREFVATINEYGGR |
| | | NYADSVKGRFTISADNSKNTAYLQMNSLKPEDTAVYYCARVYRDFDYWGQGTLVTV |
| | | SS |
| 1N-41 | 3939 | EVQLVESGGGLVQPGGSLRLSCAASGFTFSPSWMGWFRQAPGKEREFVSTINEYGGRN |
| | | YADSVKGRFTISADNSKNTAYLQMNSLKPEDTAVYYCARVTRDFDYWGQGTLVTVSS |
| 1N-42 | 3940 | EVQLVESGGGLVQPGGSLRLSCAASGFTFYPSWMGWFRQAPGKEREFVATINPYGGRN |
| | | YADSVKGRFTISADNSKNTAYLQMNSLKPEDTAVYYCARVDRDFDYWGQGTLVTVSS |
| 1N-43 | 3941 | EVQLVESGGGLVQPGGSLRLSCAASGFWFSPSWMGWFRQAPGKEREFVATINEYGGR |
| | | NYADSVKGRFTISADNSKNTAYLQMNSLKPEDTAVYYCARVDYDFDYWGQGTLVTV |
| | | SS |
| 1N-44 | 3942 | EVQLVESGGGLVQPGGSLRLSCAASGFWFEPSWMGWFRQAPGKEREFVATINEYGGR |
| | | NYADSVKGRFTISADNSKNTAYLQMNSLKPEDTAVYYCARVDRDFDYWGQGTLVTV |
| | | SS |
| 1N-45 | 3943 | EVQLVESGGGLVQPGGSLRLSCAASGFLFSPSWMGWFRQAPGKEREFVATINERGGRN |
| | | YADSVKGRFTISADNSKNTAYLQMNSLKPEDTAVYYCARVDRDFDYWGQGTLVTVSS |
| 1N-46 | 3944 | EVQLVESGGGLVQPGGSLRLSCAASGQTFVMGWFRQAPGKEREFVAAIGSGGSTSYAD |
| | | SVKGRFTISADNSKNTAYLQMNSLKPEDTAVYYCWRLGNDYFDYWGQGTLVTVSS |
| 1N-47 | 3945 | EVQLVESGGGLVQPGGSLRLSCAASGQTFMNIGWFRQAPGKEREFVAAIGSGGSTSYA |
| | | DSVKGRFTISADNSKNTAYLQMNSLKPEDTAVYYCWRLGNDYFDYWGQGTLVTVSS |
| 1N-48 | 3946 | EVQLVESGGGLVQPGGSLRLSCAASGQTFFMGWFRQAPGKEREFVAAIGSGGSTSYAD |
| | | SVKGRFTISADNSKNTAYLQMNSLKPEDTAVYYCWRLGNDYFDYWGQGTLVTVSS |
| 1N-49 | 3947 | EVQLVESGGGLVQPGGSLRLSCAASGQTFLMGWFRQAPGKEREFVAAIGSGGSTSYAD |
| | | SVKGRFTISADNSKNTAYLQMNSLKPEDTAVYYCWRLGNDYFDYWGQGTLVTVSS |
| 1N-50 | 3948 | EVQLVESGGGLVQPGGSLRLSCAASGQTFNMGWFRQAPGKEREFVAAIGSGGSTSYAD |
| | | SVKGRFTISADNSKNTAYLQMNSLKPEDTAVYYCWRYGNDYFDYWGQGTLVTVSS |
| 1N-51 | 3949 | EVQLVESGGGLVQPGGSLRLSCAASGQTFNMGWFRQAPGKEREFVAAIGSGGSTSYAD |
| | | SVKGRFTISADNSKNTAYLQMNSLKPEDTAVYYCWRFGNDYFDYWGQGTLVTVSS |
| 1N-52 | 3950 | EVQLVESGGGLVQPGGSLRLSCAASGQYFNMGWFRQAPGKEREFVAAIGSGGSTSYA |
| | | DSVKGRFTISADNSKNTAYLQMNSLKPEDTAVYYCWRLGNDYFDYWGQGTLVTVSS |
| 1N-53 | 3951 | EVQLVESGGGLVQPGGSLRLSCAASGQTFNMGWFRQAPGKEREFVADIGSGGSTSYAD |
| | | SVKGRFTISADNSKNTAYLQMNSLKPEDTAVYYCWRLGNDYFDYWGQGTLVTVSS |
| 1N-54 | 3952 | EVQLVESGGGLVQPGGSLRLSCAASGQTYNMGWFRQAPGKEREFVAAIGSGGSTSYA |
| | | DSVKGRFTISADNSKNTAYLQMNSLKPEDTAVYYCWRLGNDYFDYWGQGTLVTVSS |
| 1N-55 | 3953 | EVQLVESGGGLVQPGGSLRLSCAASGQFFNMGWFRQAPGKEREFVAAIGSGGSTSYAD |
| | | SVKGRFTISADNSKNTAYLQMNSLKPEDTAVYYCWRLGNDYFDYWGQGTLVTVSS |
| 1N-56 | 3954 | EVQLVESGGGLVQPGGSLRLSCAASGQTFIMGWFRQAPGKEREFVAAGGSGGSTSYAD |
| | | SVKGRFTISADNSKNTAYLQMNSLKPEDTAVYYCWRLGNDYFDYWGQGTLVTVSS |
| 1N-57 | 3955 | EVQLVESGGGLVQPGGSLRLSCAASGQTQPMGWFRQAPGKEREFVAAIGSGGSTSYAD |
| | | SVKGRFTISADNSKNTAYLQMNSLKPEDTAVYYCWRLGNDYFDYWGQGTLVTVSS |
| 1N-58 | 3956 | EVQLVESGGGLVQPGGSLRLSCAASGQTFIMGWFRQAPGKEREFVAAIGSGGSTSYAD |
| | | SVKGRFTISADNSKNTAYLQMNSLKPEDTAVYYCWREGNDYFDYWGQGTLVTVSS |
| 1N-59 | 3957 | EVQLVESGGGLVQPGGSLRLSCAASGQTFTMGWFRQAPGKEREFVAAIGSGGSTSYAD |
| | | SVKGRFTISADNSKNTAYLQMNSLKPEDTAVYYCWREGNDYFDYWGQGTLVTVSS |
| 1N-60 | 3958 | EVQLVESGGGLVQPGGSLRLSCAASGQTFTMGWFRQAPGKEREFVAAIGSGGWTSYA |
| | | DSVKGRFTISADNSKNTAYLQMNSLKPEDTAVYYCWRLGNDYFDYWGQGTLVTVSS |
| 1N-61 | 3959 | EVQLVESGGGLVQPGGSLRLSCAASGQTFTMGWFRQAPGKEREFVAAIGSGGSTSYAD |
| | | SVKGRFTISADNSKNTAYLQMNSLKPEDTAVYYCWRGGNDYFDYWGQGTLVTVSS |
| 1N-62 | 3960 | EVQLVESGGGLVQPGGSLRLSCAASGQTFTMGWFRQAPGKEREFVAAGGSGGSTSYA |
| | | DSVKGRFTISADNSKNTAYLQMNSLKPEDTAVYYCWRLGNDYFDYWGQGTLVTVSS |
| 1N-63 | 3961 | EVQLVESGGGLVQPGGSLRLSCAASGQTFFFGWFRQAPGKEREFVAAIGSGGSTSYAD |
| | | SVKGRFTISADNSKNTAYLQMNSLKPEDTAVYYCWRLGNDYFDYWGQGTLVTVSS |
| 1N-64 | 3962 | EVQLVESGGGLVQPGGSLRLSCAASGQTFTMGWFRQAPGKEREFVAPIGSGGSTSYAD |
| | | SVKGRFTISADNSKNTAYLQMNSLKPEDTAVYYCWRLGNDYFDYWGQGTLVTVSS |
| 1N-65 | 3963 | EVQLVESGGGLVQPGGSLRLSCAASGQTFMNIGWFRQAPGKEREFVAAIGSGGSTSYA |
| | | DSVKGRFTISADNSKNTAYLQMNSLKPEDTAVYYCWRYGNDYFDYWGQGTLVTVSS |
| 1N-66 | 3964 | EVQLVESGGGLVQPGGSLRLSCAASGQTFVMGWFRQAPGKEREFVAAIGSGGSTSYAD |
| | | SVKGRFTISADNSKNTAYLQMNSLKPEDTAVYYCWRYGNDYFDYWGQGTLVTVSS |
| 1N-67 | 3965 | EVQLVESGGGLVQPGGSLRLSCAASGQTFMNIGWFRQAPGKEREFVGAIGSGGSTSYA |
| | | DSVKGRFTISADNSKNTAYLQMNSLKPEDTAVYYCWRLGNDYFDYWGQGTLVTVSS |
| 1N-68 | 3966 | EVQLVESGGGLVQPGGSLRLSCAASGQTFTMGWFRQAPGKEREFVAAIGSGGSTSYAD |
| | | SVKGRFTISADNSKNTAYLQMNSLKPEDTAVYYCWRLTNDYFDYWGQGTLVTVSS |
| 1N-69 | 3967 | EVQLVESGGGLVQPGGSLRLSCAASGQTFFVGWFRQAPGKEREFVAAIGSGGSTSYAD |
| | | SVKGRFTISADNSKNTAYLQMNSLKPEDTAVYYCWRLGNDYFDYWGQGTLVTVSS |
| 1N-70 | 3968 | EVQLVESGGGLVQPGGSLRLSCAASGQTFVFGWFRQAPGKEREFVAAIGSGGSTSYAD |
| | | SVKGRFTISADNSKNTAYLQMNSLKPEDTAVYYCWRLGNDYFDYWGQGTLVTVSS |
| 1N-71 | 3969 | EVQLVESGGGLVQPGGSLRLSCAASGQTFIMGWFRQAPGKEREFVAAGGSGGSPSYAD |
| | | SVKGRFTISADNSKNTAYLQMNSLKPEDTAVYYCWRYGNDYFDYWGQGTLVTVSS |
| 1N-72 | 3970 | EVQLVESGGGLVQPGGSLRLSCAASGQTFIMGWFRQAPGKEREFVAAGGSGGSTSYAD |
| | | SVKGRFTISADNSKNTAYLQMNSLKPEDTAVYYCWRYGNDYFDYWGQGTLVTVSS |
| 1N-73 | 3971 | EVQLVESGGGLVQPGGSLRLSCAASGQTFIMGWFRQAPGKEREFVAAGGSGGSPSYAD |
| | | SVKGRFTISADNSKNTAYLQMNSLKPEDTAVYYCWRLGNDYFDYWGQGTLVTVSS |
| 1N-74 | 3972 | EVQLVESGGGLVQPGGSLRLSCAASGQTFIMGWFRQAPGKEREFVGAGGSGGSTSYAD |
| | | SVKGRFTISADNSKNTAYLQMNSLKPEDTAVYYCWRLGNDYFDYWGQGTLVTVSS |
| 1N-75 | 3973 | EVQLVESGGGLVQPGGSLRLSCAASGQTQPMGWFRQAPGKEREFVAAIGSGGSTSYAD |
| | | SVKGRFTISADNSKNTAYLQMNSLKPEDTAVYYCWRYGNDYFDYWGQGTLVTVSS |
| 1N-76 | 3974 | EVQLVESGGGLVQPGGSLRLSCAASGQTFIMGWFRQAPGKEREFVAAGGSGGSTSPAD |
| | | SVKGRFTISADNSKNTAYLQMNSLKPEDTAVYYCWRLGNDYFDYWGQGTLVTVSS |
| 1N-77 | 3975 | EVQLVESGGGLVQPGGSLRLSCAASGQTFIMGWFRQAPGKEREFVAAGGSGGSTSYAD |
| | | SVKGRFTISADNSKNTAYLQMNSLKPEDTAVYYCWRFGNDYFDYWGQGTLVTVSS |
| 1N-78 | 3976 | EVQLVESGGGLVQPGGSLRLSCAASGQTFIMGWFRQAPGKEREFVAAGGSGGSTSYAD |
| | | SVKGRFTISADNSKNTAYLQMNSLKPEDTAVYYCPRLGNDYFDYWGQGTLVTVSS |
| 1N-79 | 3977 | EVQLVESGGGLVQPGGSLRLSCAASGQTFIMGWFRQAPGKEREFVAAGGSGGSTSYAD |
| | | SVKGRFTISADNSKNTAYLQMNSLKPEDTAVYYCWRTGNDYFDYWGQGTLVTVSS |
| 1N-80 | 3978 | EVQLVESGGGLVQPGGSLRLSCAASGQTQPMGWFRQAPGKEREFVAAIGSGGSTSYAD |
| | | SVKGRFTISADNSKNTAYLQMNSLKPEDTAVYYCWRHGNDYFDYWGQGTLVTVSS |
| 1N-81 | 3979 | EVQLVESGGGLVQPGGSLRLSCAASGSIFRSNAMGWFRQAPGKEREWVATIGSDGTTI |
| | | YADSVKGRFTISADNSKNTAYLQMNSLKPEDTAVYYCAGDYDFWSGFDHWGQGTLV |
| | | TVSS |
| 1N-82 | 3980 | EVQLVESGGGLVQPGGSLRLSCAASGFDFSVSWMGWFRQAPGKEREFVASTNSAGST |
| | | YADSVKGRFTISADNSKNTAYLQMNSLKPEDTAVYYCARHYYDSSDYYPHYYYYGM |
| | | DVWGQGTLVTVSS |
| 1N-83 | 3981 | EVQLVESGGGLVQPGGSLRLSCAASGYTYSSNWMGWFRQAPGKEREFVSAIDSEGRTS |
| | | YADSVKGRFTISADNSKNTAYLQMNSLKPEDTAVYYCARHMGYYDSGTYFDYFDYW |
| | | GQGTLVTVSS |
| 1N-84 | 3982 | EVQLVESGGGLVQPGGSLRLSCAASGGTFSFYGMGWFRQAPGKEREFVATISWSGGD |
| | | GRSYYADSVKGRFTISADNSKNTAYLQMNSLKPEDTAVYYCTTVDQYFDYWGQGTLV |
| | | TVSS |
| 1N-85 | 3983 | EVQLVESGGGLVQPGGSLRLSCAASGSIFRSNAMGWFRQAPGKEREWVATIGSDGTTI |
| | | YADSVKGRFTISADNSKNTAYLQMNSLKPEDTAVYYCARYDFWSGYPYWGQGTLVT |
| | | VSS |
| 1N-86 | 3984 | EVQLVESGGGLVQPGGSLRLSCAASGDIFSINAMGWFRQAPGKEHEFVASISGSDKITN |
| | | YADSVKGRFTISADNSKNTAYLQMNSLKPEDTAVYYCAKFAVYDYWSGTSFDYWGQ |
| | | GTLVTVSS |
| 1N-87 | 3985 | EVQLVESGGGLVQPGGSLRLSCAASGFTFDRSWMGWFRQAPGKEREFVAAISGSGGST |
| | | NYADSVKGRFTISADNSKNTAYLQMNSLKPEDTAVYYCTSLVGLTAGFADYWGQGTL |
| | | VTVSS |
| 1N-88 | 3986 | EVQLVESGGGLVQPGGSLRLSCAASDSTFSIDVMGWFRQAPGKEREFVAAISWSAGST |
| | | LYADSVKGRFTISADNSKNTAYLQMNSLKPEDTAVYYCAAFDGYTGSDWGQGTLVTV |
| | | SS |
| 1N-89 | 3987 | EVQLVESGGGLVQPGGSLRLSCAASGDTFSWYAMGWFRQAPGKEREFVAVISWSGAY |
| | | TEYADSVKGRFTISADNSKNTAYLQMNSLKPEDTAVYYCAGDYDFWSGFDHWGQGT |
| | | LVTVSS |
| 1N-90 | 3988 | EVQLVESGGGLVQPGGSLRLSCAASGEEFSDHWMGWFRQAPGKEREFVGTINSGGDT |
| | | NYADSVKGRFTISADNSKNTAYLQMNSLKPEDTAVYYCARADNYFDYWGQGTLVTV |
| | | SS |
| 1N-91 | 3989 | EVQLVESGGGLVQPGGSLRLSCAASGSTFRINVMGWFRQAPGKEREFVAATSWSGGTT |
| | | VYADSVKGRFTISADNSKNTAYLQMNSLKPEDTAVYYCAAFDGYTGSDWGQGTLVT |
| | | VSS |
| 1N-92 | 3990 | EVQLVESGGGLVQPGGSLRLSCAASGGTFSTYGMGWFRQAPGKEREFVAAISWGGGS |
| | | DTLYADSVKGRFTISADNSKNTAYLQMNSLKPEDTAVYYCARDYGDYYYFDYWGQG |
| | | TLVTVSS |
| 1N-93 | 3991 | EVQLVESGGGLVQPGGSLRLSCAASGFTFDRSWMGWFRQAPGKEREFVTVITWSGGST |
| | | YYADSVKGRFTISADNSKNTAYLQMNSLKPEDTAVYYCAKGPSSGYAFDIWGQGTLV |
| | | TVSS |
| 1N-94 | 3992 | EVQLVESGGGLVQPGGSLRLSCAASGSIFETNTMGWFRQAPGKERELVASITSGGSTVY |
| | | ADSVKGRFTISADNSKNTAYLQMNSLKPEDTAVYYCTTVDQYFDYWGQGTLVTVSS |
| 1N-95 | 3993 | EVQLVESGGGLVQPGGSLRLSCAASGFTDGIDAMGWFRQAPGKESEWVSAISWNGSN |
| | | TYYADSVKGRFTISADNSKNTAYLQMNSLKPEDTAVYYCTTVDQYFDYWGQGTLVTV |
| | | SS |
| 1N-96 | 3994 | EVQLVESGGGLVQPGGSLRLSCAASGNTFSINVMGWFRQAPGKERELVAAISWSGAST |
| | | IYADSVKGRFTISADNSKNTAYLQMNSLKPEDTAVYYCAAFDGYSGSDWGQGTLVTV |
| | | SS |
| 1N-97 | 3995 | EVQLVESGGGLVQPGGSLRLSCAASGSDVWFNVMGWFRQAPGKERELVATITRALNT |
| | | AYADSVKGRFTISADNSKNTAYLQMNSLKPEDTAVYYCTTFNWNDEGFDYWGQGTL |
| | | VTVSS |
| 1N-98 | 3996 | EVQLVESGGGLVQPGGSLRLSCAASGSTFSVNVMGWFRQAPGKERELVAAISWSGAST |
| | | IYADSVKGRFTISADNSKNTAYLQMNSLKPEDTAVYYCAAFDGYTGSDWGQGTLVTV |
| | | SS |
| 1N-99 | 3997 | EVQLVESGGGLVQPGGSLRLSCAASGVTLDDYAMGWFRQAPGKEREWVSEITSGGYT |
| | | YADSVKGRFTISADNSKNTAYLQMNSLKPEDTAVYYCVRSNMAGFDHWGQGTLVTV |
| | | SS |
| 1N-100 | 3998 | EVQLVESGGGLVQPGGSLRLSCAASGDTYGSYWMGWFRQAPGKEREGISLITSDDGST |
| | | YYADSVKGRFTISADNSKNTAYLQMNSLKPEDTAVYYCAAFDGYSGSDWGQGTLVTV |
| | | SS |

TABLE 13
Variable Domain Light Chain Sequences

| | SEQ | |
|---|---|---|
| Variant | ID NO | Sequence |
| 1-1 | 3999 | QSALTQPASVSGSPGQSITISCTGTSSDVGSNNLVSWYQQHPGKAPKLMIYEGDKRPSG |
| | | VSNRFSGSKSGNTASLTISGLQAEDEADYYCCSYATGFYVFGGGTKLTVL |
| 1-2 | 4000 | QSALTQPASVSGSPGQSITISCTGTSSVGGYNLVSWYQQHPGKAPKLMIYEGSKRPSGV |
| | | SNRFSGSKSGNTASLTISGLQAEDEADYYCCSYAGSYTLAVFGGGTKLTVL |
| 1-3 | 4001 | QSALTQPASVSGSPGQSITISCTGTSSNVGSYNLVSWYQQHPGKAPKLMIYEGTKRPSG |
| | | VSNRFSGSKSGNTASLTISGLQAEDEADYYCCSYAGSSTFKAYVFGGGTKLTVL |
| 1-4 | 4002 | QSALTQPASVSGSPGQSITISCTGTSSDVGGYNLVSWYQQHPGKAPKLMIYEGTKRPSG |
| | | VSNRFSGSKSGNTASLTISGLQAEDEADYYCCSYAGSSTHYVFGGGTKLTVL |
| 1-5 | 4003 | QSALTQPASVSGSPGQSITISCTGTSSDVGSYHLVSWYQQHPGKAPKLMIYEGTKRPSG |
| | | VSNRFSGSKSGNTASLTISGLQAEDEADYYCCSYAGSYTFGVVFGGGTKLTVL |
| 1-6 | 4004 | QSALTQPASVSGSPGQSITISCTGTSSDVGSNNLVSWYQQHPGKAPKLMIYEGGKRPSG |
| | | VSNRFSGSKSGNTASLTISGLQAEDEADYYCCSYSGRYTYVFGGGTKLTVL |
| 1-7 | 4005 | QSALTQPASVSGSPGQSITISCTGTSSDVGNYNLVSWYQQHPGKAPKLMIYEGTKRPSG |
| | | VSNRFSGSKSGNTASLTISGLQAEDEADYYCCSYAGSYTFAVFGGGTKLTVL |
| 1-8 | 4006 | QSALTQPASVSGSPGQSITISCTGTSSDIGSYNLVSWYQQHPGKAPKLMIYEASRPSGV |
| | | SNRFSGSKSGNTASLTISGLQAEDEADYYCCSYAGSGIFYVFGGGTKLTVL |
| 1-9 | 4007 | QSALTQPASVSGSPGQSITISCTGTGSDVGYNLVSWYQQHPGKAPKLMIYEVSKRPSGV |
| | | SNRFSGSKSGNTASLTISGLQAEDEADYYCCSYAGSSTFEVFGGGTKLTVL |
| 1-10 | 4008 | QSALTQPASVSGSPGQSITISCTGTSSDVGDYNLVSWYQQHPGKAPKLMIYEGGKRPSG |
| | | VSNRFSGSKSGNTASLTISGLQAEDEADYYCCSYAGSSTNVVFGGGTKLTVL |
| 1-11 | 4009 | QSALTQPASVSGSPGQSITISCTGTSSDVGTYNLVSWYQQHPGKAPKLMIYEGYKRPSG |
| | | VSNRFSGSKSGNTASLTISGLQAEDEADYYCCSYAGNLWLFGGGTKLTVL |
| 1-12 | 4010 | QSALTQPASVSGSPGQSITISCTGTSSDVGHYNLVSWYQQHPGKAPKLMIYEGGKRPSG |
| | | VSNRFSGSKSGNTASLTISGLQAEDEADYYCCSYAGRDTYVAFGGGTKLTVL |
| 1-13 | 4011 | QSALTQPASVSGSPGQSITISCTGTSSDVGRYNLVSWYQQHPGKAPKLMIYEGTKRPSG |
| | | VSNRFSGSKSGNTASLTISGLQAEDEADYYCCSYAGSRTVVFGGGTKLTVL |
| 1-14 | 4012 | QSALTQPASVSGSPGQSITISCTGASSDVGSYNLVSWYQQHPGKAPKLMIYEGTKRPSG |
| | | VSNRFSGSKSGNTASLTISGLQAEDEADYYCCSYAGSSGVFGGGTKLTVL |
| 1-15 | 4013 | QSALTQPASVSGSPGQSITISCTGTSTDVGSYNLVSWYQQHPGKAPKLMIYEGFKRPSG |
| | | VSNRFSGSKSGNTASLTISGLQAEDEADYYCCSYAGSYTLGVFGGGTKLTVL |
| 1-16 | 4014 | QSALTQPASVSGSPGQSITISCTGTTSDVGSYNLVSWYQQHPGKAPKLMIYEGTKRPSG |
| | | VSNRFSGSKSGNTASLTISGLQAKDEADYYCSYTSSRTGVFGGGTKLTVL |
| 1-17 | 4015 | QSALTQPASVSGSPGQSITISCTATSSDVGSYNLVSWYQQHPGKAPKLMIYEGTKRPSG |
| | | VSNRFSGSKSGNTASLTISGLQAEDEADYYCCSYAGSWVFGGGTKLTVL |
| 1-18 | 4016 | QSALTQPASVSGSPGQSITISCTGTSSDVGSNNLVSWYQQHPGKAPKLMIYEGSKWPSG |
| | | VSNRFSGSKSGNTASLTISGLQAEDEADYYCCSFAGSSTDVVFGGGTKLTVL |
| 1-19 | 4017 | QSALTQPASVSGSPGQSITISCTGASSDVGSYNLVSWYQQHPGKAPKLMIYEGFKRPSG |
| | | VSNRFSGSKSGNTASLTISGLQAEDEADYYCCSYAGSHTYVFGGGTKLTVL |
| 1-20 | 4018 | QSALTQPASVSGSPGQSITISCTGTSSDVGSYYLVSWYQQHPGKAPKLMIYEGFKRPSG |
| | | VSNRFSGSKSGNTASLTISGLQAEDEADYYCCSYAGSLYVFGGGTKLTVL |
| 1-21 | 4019 | QSALTQPASVSGSPGQSITISCTGTSSDVGSYSLVSWYQQHPGKAPKLMIYEGDKRPSG |
| | | VSNRFSGSKSGNTASLTISGLQAEDEADYYCCSYAGSRRVFGGGTKLTVL |
| 1-22 | 4020 | QSALTQPASVSGSPGQSITISCTGSSSDVGSYNLVSWYQQHPGKAPKLMIYEGTKRPSG |
| | | VSNRFSGSKSGNTASLTISGLQAEDEADYYCCSYAGSSNWVFGGGTKLTVL |
| 1-23 | 4021 | QSALTQPASVSGSPGQSITISCTGTSSDVGYYNLVSWYQQHPGKAPKLMIYEGGKRPSG |
| | | VSNRFSGSKSGNTASLTISGLQAEDEADYYCCSYAGSSTPYVVFGGGTKLTVL |
| 1-24 | 4022 | QSALTQPASVSGSPGQSITISCTGTSSDVGSNNLVSWYQQHPGKAPKLMIYEGSKWPSG |
| | | VSNRFSGSKSGNTASLTISGLQAEDEADYYCCSFAGSSTDVVFGGGTKLTVL |
| 1-25 | 4023 | QSALTQPASVSGSPGQSITISCTGTSSDVGSSNLVSWYQQHPGKAPKLMIYEGDKRPSG |
| | | VSNRFSGSKSGNTASLTISGLQAEDEADYYCCSYAGSYGVVFGGGTKLTVL |
| 1-26 | 4024 | QSALTQPASVSGSPGQSITISCTGTSSDIGSYNLVSWYQQHPGKAPKLMIYEGFKRPSG |
| | | VSNRFSGSKSGNTASLTISGLQAEDEADYYCCSYAGSYSVVFGGGTKLTVL |
| 1-27 | 4025 | QSALTQPASVSGSPGQSITISCTGTSSDVGAYNLVSWYQQHPGKAPKLMIHEGNKRPSG |
| | | VSNRFSGSKSGNTASLTISGLQAEDEADYYCCSYAGDSFPYVFGGGTKLTVL |
| 1-28 | 4026 | QSALTQPASVSGSPGQSITISCTGTSRDVGSYNLVSWYQQHPGKAPKLMIYEASKRPSG |
| | | VSNRFSGSKSGNTASLTISGLQAEDEADYYCCSYAGSYTLYVFGGGTKLTVL |
| 1-29 | 4027 | QSALTQPASVSGSPGQSITISCTGTSSDVGHYNLVSWYQQHPGKAPKLMIYEGGKRPSG |
| | | VSNRFSGSKSGNTASLTISGLQAEDEADYYCCSYAGSSIYVFGGGTKLTVL |
| 1-30 | 4028 | QSALTQPASVSGSPGQSITISCTGTSSDVGNYNLVSWYQQHPGKAPKLMIYEGTKRPSG |
| | | VSNRFSGSKSGNTASLTISGLQAEDEADYYCCSYAGTVVFGGGTKLTVL |
| 1-31 | 4029 | QSALTQPASVSGSPGQSITISCTGTSSDVGKYNLVSWYQQHPGKAPKLMIYEGSQRPSG |
| | | VSNRFSGSKSGNTASLTISGLQAEDEADYYCCSYAGSSTVFGGGTKLTVL |
| 1-32 | 4030 | QSALTQPASVSGSPGQSITISCTGTSSDVGSNNLVSWYQQHPGKAPKLMIYEGDKRPSG |
| | | VSNRFSGSKSGNTASLTISGLQAEDEADYYCCSYTGSYTVVFGGGTKLTVL |
| 1-33 | 4031 | QSALTQPASVSGSPGQSITISCTGTSSDVGDYNLVSWYQQHPGKAPKLMIYEGGKRPSG |
| | | VSNRFSGSKSGNTASLTISGLQAEDEADYYCCSYAGSSTNVVFGGGTKLTVL |
| 1-34 | 4032 | QSALTQPASVSGSPGQSITISCTGTSSDVGKYNLVSWYQQHPGKAPKLMIYEASKRPSG |
| | | VSNRFSGSKSGNTASLTISGLQAEDEADYYCYSYAGSYTLGVFGGGTKLTVL |
| 1-35 | 4033 | QSALTQPASVSGSPGQSITISCTGTSSDVGSYNHVSWYQQHPGKAPKLMIYEGGKRPSG |
| | | VSNRFSGSKSGNTASLTISGLQAEDEADYYCCSYAGTTTPFVFGGGTKLTVL |
| 1-36 | 4034 | QSALTQPASVSGSPGQSITISCTGTSSDVGKYNLVSWYQQHPGKAPKLMIYETRKRPSG |
| | | VSNRFSGSKSGNTASLTISGLQAEDEADYYCCSYAGSSTVVFGGGTKLTVL |
| 2A-1 | 4035 | DIQMTQSPSSLSASVGDRVTITCRASQSIHRFLNWYQQKPGKAPKLLIYAASNLHSGVP |
| | | SRFSGSGSGTDFTLTISSLQPEDFATYYCQQSYGLPP-TFGQGTKVEIK |
| 2A-10 | 4036 | DIQMTQSPSSLSASVGDRVTITCRASQSIHISLNWYQQKPGKAPKLLIYLASPLASGVP |
| | | SRFSGSGSGTDFTLTISSLQPEDFATYYCQQSYSAPPYTFGQGTKVEIK |
| 2A-5 | 4037 | DIQMTQSPSSLSASVGDRVTITCRASQSIHTYLNWYQQKPGKAPKLLIYAASALASGVP |
| | | SRFSGSGSGTDFTLTISSLQPEDFATYYCQQSYSAPPYTFGQGTKVEIK |
| 2A-2 | 4038 | DIQMTQSPSSLSASVGDRVTITCRASQTINTYLNWYQQKPGKAPKLLIYSASTLQSGVP |
| | | SRFSGSGSGTDFTLTISSLQPEDFATYYCQQSYSTFTFGQGTKVEIK |
| 2A-4 | 4039 | DIQMTQSPSSLSASVGDRVTITCRASQSIDTYLNWYQQKPGKAPKLLIYAASALASGVP |
| | | SRFSGSGSGTDFTLTISSLQPEDFATYYCQQSYSAPPYTFGQGTKVEIK |
| 2A-6 | 4040 | DIQMTQSPSSLSASVGDRVTITCRASQSIDTYLNWYQQKPGKAPKLLIYAASALASGVP |
| | | SRFSGSGSGTDFTLTISSLQPEDFATYYCQQSYSAPPYTFGQGTKVEIK |
| 2A-11 | 4041 | DIQMTQSPSSLSASVGDRVTITCRASQSIGNYLNWYQQKPGKAPKLLIYGVSSLQSGVP |
| | | SRFSGSGSGTDFTLTISSLQPEDFATYYCQQSHSAPLTFGQGTKVEIK |
| 2A-12 | 4042 | DIQMTQSPSSLSASVGDRVTITCRASQSIDNYLNWYQQKPGKAPKLLIYGVSALQSGVP |
| | | SRFSGSGSGTDFTLTISSLQPEDFATYYCQQSHSAPPYFFGQGTKVEIK |
| 2A-13 | 4043 | DIQMTQSPSSLSASVGDRVTITCRASQSIDTYLNWYQQKPGKAPKLLIYGASALESGVP |
| | | SRFSGSGSGTDFTLTISSLQPEDFATYYCQQSHSAPPYFFGQGTKVEIK |
| 2A-14 | 4044 | DIQMTQSPSSLSASVGDRVTITCRASQSIDTYLNWYQQKPGKAPKLLIYGVSALQSGVP |
| | | SRFSGSGSGTDFTLTISSLQPEDFATYYCQQSYSAPPYFFGQGTKVEIK |
| 2A-7 | 4045 | DIQMTQSPSSLSASVGDRVTITCRASQSIDTYLNWYQQKPGKAPKLLIYAASALASGVP |
| | | SRFSGSGSGTDFTLTISSLQPEDFATYYCQQSYSAPPYTFGQGTKVEIK |
| 2A-8 | 4046 | DIQMTQSPSSLSASVGDRVTITCRASQSIDTYLNWYQQKPGKAPKLLIYAASALASGVP |
| | | SRFSGSGSGTDFTLTISSLQPEDFATYYCQQSYSAPPYTFGQGTKVEIK |
| 2A-15 | 4047 | DIQMTQSPSSLSASVGDRVTITCRASQSIDNYLNWYQQKPGKAPKLLIYGVSALQSGVP |
| | | SRFSGSGSGTDFTLTISSLQPEDFATYYCQQSHSAPLTFGQGTKVEIK |
| 2A-9 | 4048 | DIQMTQSPSSLSASVGDRVTITCRASQRIGTYLNWYQQKPGKAPKLLIYAASNLEGGVP |
| | | SRFSGSGSGTDFTLTISSLQPEDFATYYCQQNYSTTWTFGQGTKVEIK |
| 2A-21 | 4049 | DIQMTQSPSSLSASVGDRVTITCRASQSIHTYLNWYQQKPGKAPKLLIYAASALASGVP |
| | | SRFSGSGSGTDFTLTISSLQPEDFATYYCQQSYSAPPYTFGQGTKVEIK |
| 2A-22 | 4050 | DIQMTQSPSSLSASVGDRVTITCRASQSIHTYLNWYQQKPGKAPKLLIYAASALASGVP |
| | | SRFSGSGSGTDFTLTISSLQPEDFATYYCQQSYSAPPYTFGQGTKVEIK |
| 2A-23 | 4051 | DIQMTQSPSSLSASVGDRVTITCRASQTINTFLNWYQQKPGKAPKLLIYSASTLQSGVP |
| | | SRFSGSGSGTDFTLTISSLQPEDFATYYCQQSYSTFTFGGGTKVEIK |
| 2A-24 | 4052 | DIQMTQSPSSLSASVGDRVTITCRASQTIRTYLNWYRQKPGKAPKLLIYDASTLQRGVP |
| | | SRFSGSGSGTDFTLTISSLQPEDFATYYCQQSYRTPPWTFGGGTKVEIK |
| 2A-25 | 4053 | DIQMTQSPSSLSASVGDRVTITCRSSQSISSYLNWYQQKPGEAPKLLIYGASRLRSGVP |
| | | SRFSGSGSGTDFTLTISSLQPEDFATYYCQQGYSAPWTFGGGTKVEIK |
| 2A-26 | 4054 | DIQMTQSPSSLSASVGDRVTITCRASQSISGSLNWYQQKPGKAPKLLIYAESRLHSGVP |
| | | SRFSGSGSGTDFTLTISSLQPEDFATYYCQQSYSPPQTFGGGTKVEIK |
| 2A-27 | 4055 | DIQMTQSPSSLSASVGDRVTITCRASRSISTYLNWYQQKPGKAPKLLIYAASNLQGGVP |
| | | SRISGSGSGTDFTLTISSLQPEDFATYYCQQSHSIPRTFGGGTKVEIK |
| 2A-28 | 4056 | DIQMTQSPSSLSASVGDRVTITCRASQSIHTYLNWYQQKPGKAPKLLIYAASALASGVP |
| | | SRFSGSGSGTDFTLTISSLQPEDFATYYCQQSYSAPPYTFGQGTKVEIK |
| 3A-10 | 4057 | DIQMTQSPSSLSASVGDRVTITCRASQSIRKYLNWYQQKPGKAPKLLIYASSTLQRGVP |
| | | SRFSGSGSGTDFTLTISSLQPEDFATYYCQQSLSTPFTFGGGTKVEIK |
| 3A-4 | 4058 | DIQMTQSPSSLSASVGDRVTITCRASRSIRRYLNWYQQKPGKAPKLLIYASSSLQAGVP |
| | | SRFSGSGSGTDFTLTISSLQPEDFATYYCQQSYSTLLTFGQGTKVEIK |
| 3A-7 | 4059 | DIQMTQSPSSLSASVGDRVTITCRASQSIGRYLNWYQQKPGKAPKLLIYASSSLQSGVP |
| | | SRFSGSGSGTDFTLTISSLQPEDFATYYCQQSYSLPRTFGQGTKVEIK |
| 3A-1 | 4060 | DIQMTQSPSSLSASVGDRVTITCRASQTIYSYLNWYQQKPGKAPKLLIYATSTLQGGVP |
| | | SRFSGSGSGTDFTLTISSLQPEDFATYYCQHRGTFGQGTKVEIK |
| 3A-5 | 4061 | DIQMTQSPSSLSASVGDRVTITCRASQSIGRYLNWYQQKPGKAPKLLIYAASSLKSGVP |
| | | SRFSGSGSGTDFTLTISSLQPEDFATYYCQQSYSLPRTFGQGTKVEIK |
| 3A-6 | 4062 | DIQMTQSPSSLSASVGDRVTITCRASQSIGKYLNWYQQKPGKAPKLLIYASSSLQSGVP |
| | | SRFSGSGSGTDFTLTISSLQPEDFATYYCQQSYSPPFTFGQGTKVEIK |
| 3A-15 | 4063 | DIQMTQSPSSLSASVGDRVTITCRASQNIKTYLNWYQQKPGKAPKLLIYAASKLQSGVP |
| | | SRFSGSGSGTDFTLTISSLQPEDFATYYCQQSYSTSPTFGQGTKVEIK |
| 3A-3 | 4064 | DIQMTQSPSSLSASVGDRVTITCRASRSISRYLNWYQQKPGKAPKLLIYAASSLQAGVP |
| | | SRFSGSGSGTDFTLTISSLQPEDFATYYCQQSYSSLLTFGQGTKVEIK |
| 3A-11 | 4065 | DIQMTQSPSSLSASVGDRVTITCRASQSIGKYLNWYQQKPGKAPKLLIYASSTLQRGVP |
| | | SRFSGSGSGTDFTLTISSLQPEDFATYYCQQSLSPPFTFGQGTKVEIK |
| 3A-8 | 4066 | DIQMTQSPSSLSASVGDRVTITCRASQSIGRYLNWYQQKPGKAPKLLIYAASSLKSGVP |
| | | SRFSGSGSGTDFTLTISSLQPEDFATYYCQQSYSLPLTFGQGTKVEIK |
| 3A-2 | 4067 | DIQMTQSPSSLSASVGDRVTITCRTSQSINTYLNWYQQKPGKAPKLLIYGASNVQSGVP |
| | | SRFSGSGSGTDFTLTISSLQPEDFATYYCQQSYRIPRTFGQGTKVEIK |
| 3A-12 | 4068 | DIQMTQSPSSLSASVGDRVTITCRASQSIGKYLNWYQQKPGKAPKLLIYASSTLQRGVP |
| | | SRFSGSGSGTDFTLTISSLQPEDFATYYCQQSLSTPFTFGQGTKVEIK |
| 3A-14 | 4069 | DIQMTQSPSSLSASVGDRVTITCRASQSIGKYLNWYQQKPGKAPKLLIYASSTLQRGVP |
| | | SRFSGSGSGTDFTLTISSLQPEDFATYYCQQSFSTPFTFGQGTKVEIK |
| 3A-9 | 4070 | DIQMTQSPSSLSASVGDRVTITCRASQSIGRYLNWYQQKPGKAPKLLIYAASSLKSGVP |
| | | SRFSGSGSGTDFTLTISSLQPEDFATYYCQQSYSLPRTFGQGTKVEIK |
| 3A-13 | 4071 | DIQMTQSPSSLSASVGDRVTITCRASQSIGKYLNWYQQKPGKAPKLLIYASSTLQRGVP |
| | | SRFSGSGSGTDFTLTISSLQPEDFATYYCQQSFSPPFTFGQGTKVEIK |
| 3A-16 | 4072 | DIQMTQSPSSLSASVGDRVTITCRASQIIGSYLNWYQQKPGKAPKLLIYTTSNLQSGVP |
| | | SRFSGSGSGTDFTLTISSLQPEDFATYYCQQSYITPWTFGQGTKVEIK |
| 3A-17 | 4073 | DIQMTQSPSSLSASVGDRVTITCRASQSISRYINWYQQKPGKAPKLLIYEASSLESGVP |
| | | SRFSGSGSGTDFTLTISSLQPEDFATYYCQQSHITPLTFGQGTKVEIK |
| 3A-18 | 4074 | DIQMTQSPSSLSASVGDRVTITCRASQSIYTYLNWYQQKPGKAPKLLIYSASNLHSGVP |
| | | SRFSGSGSGTDFTLTISSLQPEDFATYYCQQSDTTPWTFGQGTKVEIK |
| 3A-19 | 4075 | DIQMTQSPSSLSASVGDRVTITCRASQSIATYLNWYQQKPGKAPKLLIYGASSLEGGVP |
| | | SRFSGSGSGTDFTLTISSLQPEDFATYYCQQTFSSPFTFGQGTKVEIK |
| 3A-2 | 4076 | DIQMTQSPSSLSASVGDRVTITCRASQNINTYLNWYQQKPGKAPKLLIYSASSLQSGVP |
| | | SRFSGSGSGTDFTLTISSLQPEDFATYYCQQSSLTPWTFGQGTKVEIK |
| 3A-21 | 4077 | DIQMTQSPSSLSASVGDRVTITCRASQGIATYLNWYQQKPGKAPKLLIYYASNLQSGVP |
| | | SRFSGSGSGTDFTLTISSLQPEDFATYYCQQSYSTRFTFGQGTKVEIK |
| 3A-22 | 4078 | DIQMTQSPSSLSASVGDRVTITCRASERISNYLNWYQQKPGKAPKLLIYTASNLESGVP |
| | | SRFSGSGSGTDFTLTISSLQPEDFATYYCQQSYTPPRTFGQGTKVEIK |
| 3A-23 | 4079 | DIQMTQSPSSLSASVGDRVTITCRASQSISSSLNWYQQKPGKAPKLLIYAASRLQDGVP |
| | | SRFSGSGSGTDFTLTISSLQPEDFATYYCQQSYSTPRSFGQGTKVEIK |
| 3A-24 | 4080 | DIQMTQSPSSLSASVGDRVTITCRASQSISSHLNWYQQKPGKAPKLLTYRASTLQSGVP |
| | | SRFSGSGSGTDFTLTISSLQPEDFATYYCQQTYNTPQTFGQGTKVEIK |
| 3A-25 | 4081 | DIQMTQSPSSLSASVGDRVTITCRASQSISSYLIWYQQKPGKAPKLLIYAASRLHSGVP |
| | | SRFSGSGSGTDFTLTISSLQPEDFATYYCQQGYNTPRTFGQGTKVEIK |
| 3A-26 | 4082 | DIQMTQSPSSLSASVGDRVTITCRASPSISTYLNWYQQKPGKAPKLLIYTASRLQTGVP |
| | | SRFSGSGSGTDFTLTISSLQPEDFATYYCQQTYSTPSSFGQGTKVEIK |
| 3A-27 | 4083 | DIQMTQSPSSLSASVGDRVTITCRASQNIAKYLNWYQQKPGKAPKLLIYGASGLQSGVP |
| | | SRFSGSGSGTDFTLTISSLQPEDFATYYCQQSHSPPITFGQGTKVEIK |
| 3A-28 | 4084 | DIQMTQSPSSLSASVGDRVTITCRASQSIGTYLNWYQQKPGKAPKLLIYAASNLHSGVP |
| | | SRFSGSGSGTDFTLTISSLQPEDFATYYCQESYSAPYTFGQGTKVEIK |
| 3A-29 | 4085 | DIQMTQSPSSLSASVGDRVTITCRASQSISPYLNWYQQKPGKAPKLLIYKASSLQSGVP |
| | | SRFSGSGSGTDFTLTISSLQPEDFATYYCQQSSSTPYTFGQGTKVEIK |
| 9-1 | 4086 | EIVLTQSPATLSLSPGERATLSCRASQGVSNYLAWYQQKPGQAPRLLIYDASNRATGIP |
| | | ARFSGSGSGTDFTLTISSLEPEDFAVYYCQQRYSWVTFGGGTKVEIK |
| 9-2 | 4087 | EIVLTQSPATLSLSPGERATLSCRASQSVSSSLAWYQQKPGQAPRLLIYDASNRATGIP |
| | | ARFSGSGSGTDFTLTISSLEPEDFAVYYCQQRINWPRSFGGGTKVEIK |
| 9-3 | 4088 | EIVLTQSPATLSLSPGERATLSCRASQSVNSYLAWYQQKPGQAPRLLIYDVSNRATGIP |
| | | ARFSGSGSGTDFTLTISSLEPEDFAVYYCQQFSNWPTFGGGTKVEIK |
| 9-4 | 4089 | EIVLTQSPATLSLSPGERATLSCRASQSVGTSLAWYQQKPGQAPRLLIYGASNRATGIP |
| | | ARFSGSGSGTDFTLTISSLEPEDFAVYYCQQRSNWQPFGGGTKVEIK |
| 9-5 | 4090 | EIVLTQSPATLSLSPGERATLSCRATQYVNSYLAWYQQKPRQAPRLIIYDVSNRATGIP |
| | | ARFSGSGSGTDFTLTISSLEPEDFAVYYCQQFSNWPTFGGGTKVEIK |
| 9-6 | 4091 | EIVLTQSPATLSLSPGERATLSCRASQSVGTSLAWYQQKPGQAPRLLIYGASNRATGIP |
| | | ARFSGSGSGTDFTLTISSLEPEDFAVYYCQLRSNWYTFGGGTKVEIK |
| 9-7 | 4092 | EIVLTQSPATLSLSPGERATLSCRASQGVSNYLAWYQQKPGQAPRLLIYDASNRATGIP |
| | | ARFSGSGSGTDFTLSISSLEPEDFAVYYCQQRYSWVTFGGGTKVEIK |
| 9-8 | 4093 | EIVLTQSPATLSLSPGERATLSCRASQGVSNYLAWYQQKPGQAPRLLIYDASNRATGIP |
| | | ARFSGSGSGTDFTLTISSLEPEDFAVYYCQQRYSWVTFGGGTKVEIK |
| 9-9 | 4094 | EIVLTQSPATLSLSPGERATLSCRASQSVDSRLAWYQQKPGQAPRLLIYDTSNRATGIP |
| | | ARFSGSGSGTDFTLTISSLEPEDFAVYYCQQRSTWPPVFGGGTKVEIK |
| 9-10 | 4095 | EIVLTQSPATLSLSPGERATLSCRASQSVRHHLAWYQQKPGQAPRLLIYDASNRATGIP |
| | | ARFSGSGSGTDFTLTISSLEPEDFAVYYCQQRTDWPRAFGGGTKVEIK |
| 9-11 | 4096 | EIVLTQSPATLSLSPGERATLSCRASQSVGNFLAWYQQKPGQAPRLLIYDASNRATGIP |
| | | ARFSGSGSGTDFTLTISSLEPEDFAVYYCQQSSTWPLTFGGGTKVEIK |
| 9-12 | 4097 | EIVLTQSPATLSLSPGERATLSCRASESISTYLAWYQQKPGQAPRLLIYDASNRATGIP |
| | | ARFSGSGSGTDFTLTISSLEPEDFAVYYCQQRSGLITFGGGTKVEIK |
| 9-13 | 4098 | EIVLTQSPATLSLSPGERATLSCRASQSVGDFLAWYQQKPGQAPRLLIYDTSNRATGIP |
| | | ARFSGSGSGTDFTLTISSLEPEDFAVYYCQQRSNLTFGGGTKVEIK |
| 9-14 | 4099 | DIQMTQSPSSLSASVGDRVTITCRASQTIRNSLNWYQQKPGKAPKLLIYASSSLQSGVP |
| | | SRFSGSGSGTDFTLTISSLQPEDFATYYCQQTHSIPKTFGQGTKVEIK |
| 9-15 | 4100 | EIVLTQSPATLSLSPGERATLSCRASQSVSSSLAWYQQKPGQAPRLLIYDASNRATGIP |
| | | ARFSGSGSGTDFTLTISSLEPEDFAVYYCQQRINWPRSFGGGTKVEIK |
| 10-1 | 4101 | EIVLTQSPATLSLSPGERATLSCRASQDVSTYLAWYQQKPGQAPRLLIYDASNRATGIP |
| | | ARFSGSGSGTDFTLTISSLEPEDFAVYYCQQRRDWPQTFGGGTKVEIK |
| 10-2 | 4102 | EIVLTQSPATLSLSPGERATLSCRASQDVSTYLAWYQQKPGQAPRLLIYDASNRATGIP |
| | | ARFSGSGSGTDFTPFITNFEPEDFAVYYCQQRRDWPQTFGGGTKVEIK |
| 10-3 | 4103 | EIVLTQSPATLSLSPGERATLSCRASQDVSTYLAWYQQKPGQAPRLLIYDASNRATGIP |
| | | ARFSGSGSGTDFTPFITNFEPEDFAVYYCQQRRDWPQTFGGGTKVEIK |
| 10-4 | 4104 | EIVLTQSPATLSLSPGERATLSCRASQDVSTYLAWYQQKPGQAPRLLIYDASNRATGIP |
| | | ARFSGSGSGTDFTLFITNFEPEDFAVYYCQQRRDWPQTFGGGTKVEIK |
| 10-5 | 4105 | EIVLTQSPATLSLSPGERATLSCRASQDVSTYLAWYQQKPGQAPRLLIYDASNRATGIP |
| | | ARFSGSGSGTDFTLTISSFEPEDFAVYYCQQRRDWPQTFGGGTKVEIK |
| 10-6 | 4106 | EIVLTQSPATLSLSPGERATLSCRASQDVSTYLAWYQQKPGQAPRLLIYDASNRATGIP |
| | | ARFSGSGSGTDFTPFITNFEHEDFAVYYCQQRRDWPQTFGGGTKVEIK |
| 11-1 | 4107 | EIVLTQSPATLSLSPGERATLSCRASQSLGSFLAWYQQKPGQAPRLLIYDASNRATGIP |
| | | ARFSGSGSGTDFTLTISSLEPEDFAVYYCQQRALWPRLTFGGGTKVEIK |
| 11-2 | 4108 | EIVLTQSPATLSLSPGERATLSCRASQSVNSYLAWYQQKPGQAPRLLIYDVSNRATGIP |
| | | ARFSGSGSGTDFTLTISSLEPEDFAVYYCQQFSNWPTFGGGTKVEIK |
| 11-3 | 4109 | EIVLTQSPATLSLSPGERATLSCRASQNIGNHLAWYQQKPGQAPRLLIYDASNRATGIP |
| | | ARFSGSGSGTDFTLTISSLEPEDFAVYYCQQRDNGPPEGTFGGGTKVEIK |
| 11-4 | 4110 | EIVLTQSPATLSLSPGERATLSCRASQSVGSYLAWYQQKPGQAPRLLIYDAVNRATGIP |
| | | ARFSGSGSGTDFTLTISSLEPEDFAVYYCQQRFTWPTTFGGGTKVEIK |
| 11-5 | 4111 | EIVLTQSPATLSLSPGERATLSCRASQSITDYLAWYQQKPGQAPRLLIYDASNRATGIP |
| | | ARFSGSGSGTDFTLTISSLEPEDFAVYYCHQRNNWPPTFGGGTKVEIK |
| 11-6 | 4112 | EIVLTQSPATLSLSPGERATLSCRASQSVDSSLAWYQQKPGQAPRLLIYDASNRATGIP |
| | | ARFSGSGSGTDFTLTISSLEPEDFAVYYCQQQSNWPGTFGGGTKVEIK |
| 11-7 | 4113 | EIVLTQSPATLSLSPGERATLSCRASQSIGSYLAWYQQKPGQAPRLLIYDGSNRATGIP |
| | | ARFSGSGSGTDFTLTISSLEPEDFAVYYCQQRTNWPLFSFGGGTKVEIK |
| 11-8 | 4114 | EIVLTQSPATLSLSPGERATLSCRASQTVTNYLAWYQQKPGQAPRLLIYDTSNRATGIP |
| | | ARFSGSGSGTDFTLTISSLEPEDFAVYYCQHRDDWPPTFGGGTKVEIK |
| 11-9 | 4115 | EIVLTQSPATLSLSPGERATLSCRASQSVSYYLAWYQQKPGQAPRLLIYDSSNRATGIP |
| | | ARFSGSGSGTDFTLTISSLEPEDFAVYYCQQRSNWQGNFGGGTKVEIK |
| 11-10 | 4116 | EIVLTQSPATLSLSPGERATLSCRASQSVSTSLAWYQQKPGQAPRLLIYDATNRATGIP |
| | | ARFSGSGSGTDFTLTISSLEPEDFAVYYCQQHYSWPLTFGGGTKVEIK |
| 11-11 | 4117 | EIVLTQSPATLSLSPGERATLSCRASHNINNFLAWYQQKPGQAPRLLIYDTSNRATGIP |
| | | ARFSGSGSGTDFTLTISSLEPEDFAVYYCQQGRNWPPSSFGGGTKVEIK |
| 11-12 | 4118 | EIVLTQSPATLSLSPGERATLSCRASQSVGTSLAWYQQKPGQAPRLLIYGASNRATGIP |
| | | ARFSGSGSGTDFTLTISSLEPEDFAVYYCQERSNWPDTFGGGTKVEIK |
| 11-13 | 4119 | EIVLTQSPATLSLSPGERATLSCRASQSVSSQLAWYQQKPGQAPRLLMYDTSNRATGIP |
| | | ARFSGSGSGTDFTLTISSLEPEDFAVYYCQQRYNWPSTFGGGTKVEIK |
| 11-14 | 4120 | EIVLTQSPATLSLSPGERATLSCRASQSVDSRLAWYQQKPGQAPRLLIYDASNRATGIP |
| | | ARFSGSGSGTDFTLTISSLEPEDFAVYYCQQRTNLPPSITFGGGTKAKLK |
| 11-15 | 4121 | EMVVPQSPPTVSLSPGERATLSCRASQSVGSYLAWYQQKPGQAPRLLIYDAVNRATGIP |
| | | ARFSGSGSGTDFTLTISSLEPEDFAVYYCQQRSDSITFGGGTKVEIK |
| 11-16 | 4122 | EIVLTQSPATLSLSPGERATLSCRASQSLGRYLAWYQQKPGQAPRLLIYDSSNRATGIP |
| | | ARFSGSGSGTDFTLTISSLEPEDFAVYYCQQYGDWPETFGGGTKVEIK |
| 11-17 | 4123 | EIVLTQSPATLSLSPGERATLSCRASQNIGSHLAWYQQKPGQAPRLLIYDVSNRATGIP |
| | | ARFSGSGSGTDFTLTISSLEPEDFAVYYCQQRDYWPPYTFGGGTKVEIK |
| 11-18 | 4124 | EIVLTQSPATLSLSPGERATLSCRASQSLTSYLAWYQQKPGQAPRLLIYDASNRATGIP |
| | | ARFSGSGSGTDFTLTISSLEPEDFAVYYCQQRHYWPPITFGGGTKVEIK |
| 11-19 | 4125 | EIVLTQSPATLSLSPGERATLSCRASQSIGSYLAWYQQKPGQAPRLLIYDASNRATGIP |
| | | ARFSGSGSGTDFTLTISSLEPEDFAVYYCQQRDSWPHTFGGGTKVEIK |
| 11-20 | 4126 | EIVLTQSPATLSLSPGERATLSCRASQSVGSYLAWYQQKPGQAPRLLIYDAVNRATGIP |
| | | ARFSGSGSGTDFTLTISSLEPEDFAVYYCQQRSLWPFGGGTKVEIK |
| 12-1 | 4127 | EIVLTQSPATLSLSPGERATLSCRASQSVSSHLAWYQQKPGQAPRLLIYDVSNRATGIP |
| | | ARFSGSGSGTDFTLTISSLEPEDFAVYYCQQRDTFTFGGGTKVEIK |
| 12-2 | 4128 | EIVLTQSPATLSLSPGERATLSCRASQSVDSRLAWYQQKPGQAPRLLIYDTSNRATGIP |
| | | ARFSGSGSGTDFTLTISSLEPEDFAVYYCQQRSTWPPVFGGGTKVEIK |
| 12-3 | 4129 | EIVLTQSPATLSLSPGERATLSCRASQSVGDFLAWYQQKPGQAPRLLIYDTSNRATGIP |
| | | ARFSGSGSGTDFTLTISSLEPEDFAVYYCQYRSNFTFGGGTKVEIK |
| 12-4 | 4130 | EIVLTQSPATLSLSPGERATLSCRASQSVGSHLAWYQQKPGQAPRLLIYDASNRATGIP |
| | | SRFSGSGSGTDFTLTISSLEPEDFAVYYCQQISNWPLTFGGGTKVEIK |
| 12-5 | 4131 | EIVLTQSPATLSLSPGERATLSCRASQNVGQSLAWYQQKPGQAPRLLIYDASNRATGIP |
| | | ARFSGSGSGTDFTLTISSLEPEDFAVYYCQQRENWPPTFGGGTKVEIK |
| 12-6 | 4132 | EIVLTQSPATLSLSPGERATLSCRASQSLGNYLAWYQQKPGQAPRLLIYDSSNRATGIP |
| | | ARFSGSGSGTDFTLTISSLEPEDFAVYYCQQRNWPYTFGGGTKVEIK |
| 12-7 | 4133 | EIVLTQSPATLSLSPGERATLSCRASQSLGNYLAWYQQKPGQAPRLLIYDSSNRATGIP |
| | | ARFSGSGSGTDFTLTISSLEPEDFAVYYCQQRTDWPPSFGGGTKVEIK |
| 12-8 | 4134 | EIVLTQSPATLSLSPGERATLSCRASQNIGNHLAWYQQKPGQAPRLLIYDVSNRATGIP |
| | | ARFSGSGSGTDFTLTISSLEPEDFAVYYCQQRKSWPPFTFGGGTKVEIK |
| 12-9 | 4135 | EIVLTQSPATLSLSPGERATLSCRASQSVSTSLAWYQQKPGQAPRLLIYDATNRATGIP |
| | | ARFSGSGSGTDFTLTISSLEPEDFAVYYCQRRTDWPPTFGGGTKVEIK |
| 12-10 | 4136 | EIVLTQSPATLSLSPGERATLSCRASQSVNSDLAWYQQKPGQAPRLLIYDASNRATGIP |
| | | ARFSGSGSGTDFTLTISSLEPEDFAVYYCQQRTDWPPATFGGGTKVEIK |
| 12-11 | 4137 | EIVLTQSPATLSLSPGERATLSCRASQSVGSYLAWYQQKPGQAPRLLIYDAVNRATGIP |
| | | ARFSGSGSGTDFTLTISSLEPEDFAVYYCQQRFTWPTTFGGGTKVEIK |
| 12-12 | 4138 | EIVLTQSPATLSLSPGERATLSCRASQSVSSSLAWYQQKPGQAPRLLIYDASNRATGIP |
| | | ARFSGSGSGTDFTLTISSLEPEDFAVYYCQHRDDWPPTFGGGTKVEIK |
| 12-13 | 4139 | EIVLTQSPATLSLSPGERATLSCRASQSVGSYLAWYQQKPGQAPRLLIYDAVNRATGIP |
| | | ARFSGSGSGTDFTLTISSLEPEDFAVYYCQQRNSWPPATFGGGTKVEIK |
| 12-14 | 4140 | EIVLTQSPATLSLSPGERATLSCRASQSVGSYLAWYQQKPGQAPRLLIYDAVNRATGIP |
| | | ARFSGSGSGTDFTLTISSLEPEDFAVYYCQQVSNWPLTFGGGTKVEIK |
| 12-15 | 4141 | EIVLTQSPATLSLSPGERATLSCRASQSVSSHLAWYQQKPGQAPRLLIYDVSNRATGIP |
| | | ARFSGSGSGTDFTLTISSLEPEDFAVYYCQVRSDWPPLTFGGGTKVEIK |
| 12-16 | 4142 | EIVLTQSPATLSLSPGERATLSCRASQSLDSYLAWYQQKPGQAPRLLIYDVSNRATGIP |
| | | ARFSGSGSGTDFTLTISSLEPEDFAVYYCQQRRGWPPVTFGGGTKVEIK |
| 12-17 | 4143 | EIVLTQSPATLSLSPGERATLSCRASQSVSKFLAWYQQKPGQAPRLLIYDASNRATGIP |
| | | ARFSGSGSGTDFTLTISSLEPEDFAVYYCHQHSDWPLTFGGGTKVEIK |
| 12-18 | 4144 | EIVLTQSPATLSLSPGERATLSCRASQSIGGSLAWYQQKPGQAPRLLIYDASNRATGIP |
| | | ARFSGSGSGTDFTLTISSLEPEDFAVYYCQQRYSYFTFGGGTKVEIK |
| 12-19 | 4145 | EIVLTQSPATLSLSPGERATLSCRASQSISRYLAWYQQKPGQAPRLLIYDVSNRATGIP |
| | | ARFSGSGSGTDFTLTISSLEPEDFAVYYCQQSSNWPLFTFGGGTKVEIK |
| 12-20 | 4146 | EIVLTQSPATLSLSPGERATLSCRASQSLGNYLAWYQQKPGQAPRLLIYDSSNRATGIP |
| | | ARFSGSGSGTDFTLTISSLEPEDFAVYYCQQRNTWPGVTFGGGTKVEIK |
| 12-21 | 4147 | EIVLTQSPATLSLSPGERATLSCRASQSVNSDLAWYQQKPGQAPRLLIYDASNRATGIP |
| | | ARFSGSGSGTDFTLTISSLEPEDFAVYYCQERSLFGGGTKVEIK |
| 12-22 | 4148 | EIVLTQSPATLSLSPGERATLSCRASQSVRHHLAWYQQKPGQAPRLLIYDASNRATGIP |
| | | ARFSGSGSGTDFTLTISSLEPEDFAVYYCQERSDWPITFGGGTKVEIK |
| 12-23 | 4149 | EIVLTQSPATLSLSPGERATLSCRASQSVDSRLAWYQQKPGQAPRLLIYDASNRATGIP |
| | | ARFSGSGSGTDFTLTISSLEPEDFAVYYCQQRSTWPPVFGGGTKVEIK |
| 12-24 | 4150 | EIVLTQSPATLSLSPGERATLSCRASQSFGDSLAWYQQKPGQAPRLLIYDASNRATGIP |
| | | ARFSGSGSGTDFTLTISSLEPEDFAVYYCQQRSIPITFGGGTKVEIK |
| 12-25 | 4151 | EIVLTQSPATLSLSPGERATLSCRASQSVNSYLAWYQQKPGQAPRLLIYDVSNRATGIP |
| | | ARFSGSGSGTDFTLTISSLEPEDFAVYYCQERGNWPPFTFGGGTKVEIK |
| 12-26 | 4152 | EIVLTQSPATLSLSPGERATLSCRASQSVSTSLAWYQQKPGQAPRLLIYDISNRATGIP |
| | | ARFSGSGSGTDFTLTISSLEPEDFAVYYCQQRRSGLTFGGGTKVEIK |
| 12-27 | 4153 | EIVLTQSPATLSLSPGERATLSCRASDTVSSYLAWYQQKPGQAPRLLIYDTSNRATGIP |
| | | ARFSGSGSGTDFTLTISSLEPEDFAVYYCQQRASWPLSFGGGTKVEIK |
| 12-28 | 4154 | EIVLTQSPATLSLSPGERATLSCRASQSVRHHLAWYQQKPGQAPRLLIYDASNRATGIP |
| | | ARFSGSGSGTDFTLTISSLEPEDFAVYYCQQSGSWPLTFGGGTKVEIK |
| 12-29 | 4155 | EIVLTQSPATLSLSPGERATLSCRASQIISSYLAWYQQKPGQAPRLLIYDTSNRATGIP |
| | | ARFSGSGSGTDFTLTISSLEPEDFAVYYCQVRSNWPPLTFGGGTKVEIK |
| 12-30 | 4156 | EIVLTQSPATLSLSPGERATLSCRASHNIGTYLAWYQQKPGQAPRLLIYDVSNRATGIP |
| | | ARFSGSGSGTDFTLTISSLEPEDFAVYYCQQRADWPQTFGGGTKVEIK |
| 12-31 | 4157 | EIVLTQSPATLSLSPGERATLSCRASQSIGSYLAWYQQKPGQAPRLLIYDVSNRATGIP |
| | | ARFSGSGSGTDFTLTISSLEPEDFAVYYCQQRDSFTFGGGTKVEIK |
| 12-32 | 4158 | EIVLTQSPATLSLSPGERATLSCRASQSIGSYLAWYQQKPGQAPRLLIYDVSNRATGIP |
| | | ARFSGSGSGTDFTLTISSLEPEDFAVYYCQQRDSFTFGGGTKVEIK |
| 12-33 | 4159 | EIVLTQSPATLSLSPGERATLSCRASQDVSTYLAWYQQKPGQAPRLLIYDASNRATGIP |
| | | ARFSGSGSGTDFTLTISSLEPEDFAVYYCQQRAYWPGTFGGGTKVEIK |
| 12-34 | 4160 | EIVLTQSPATLSLSPGERATLSCRASQSVGNFLAWYQQKPGQAPRLLIYDASNRATGIP |
| | | ARFSGSGSGTDFTLTISSLEPEDFAVYYCQHRRLLTFGGGTKVEIK |
| 12-35 | 4161 | EIVLTQSPATLSLSPGERATLSCRASQRVSSYLAWYQQKPGQAPRLLIYDAFNRATGIP |
| | | ARFSGSGSGTDFTLTISSLEPEDFAVYYCQQRQDWPLTFGGGTKVEIK |
| 12-36 | 4162 | EIVLTQSPATLSLSPGERATLSCRASQGISTYLAWYQQKPGQAPRLLIYDASNRATGIP |
| | | ARFSGSGSGTDFTLTISSLEPEDFAVYYCQQRRRWPPTFGGGTKVEIK |
| 12-37 | 4163 | EIELTQSPATLSLSPGERATLSCRASESVSESLAWYQQKPGQAPRLLIYDASNRATGIP |
| | | ARFSGSGSGTDFTLTISSLEPEDFAVYYCQQRTHGVTFGGGTKVEIK |
| 12-38 | 4164 | EIVLTQSPATLSLSPGERATLSCRASQSVSTSLAWYQQKPGQAPRLLIYDATNRATGIP |
| | | ARFSGSGSGTDFTLTISSLEPEDFAVYYCQQRQKWPLTFGGGTKVEIK |
| 12-39 | 4165 | EIVLTQSPATLSLSPGERATLSCRASESISTYLAWYQQKPGQAPRLLIYDASNRATGIP |
| | | ARFSGSGSGTDFTLTISSLEPEDLAVYYCQQRRNSLTFGGGTKVEIK |
| 12-40 | 4166 | EIVLTQSPATLSLSPGERATLSCRASQSVNSDLAWYQQKPGQAPRLLIYDASNRATGIP |
| | | ARFSGSGSGTDFTLTISSLEPEDFAVYYCQQRSTWSPLTFGGGTKVEIK |
| 12-41 | 4167 | EIVLTQSPATLSLSPGERATLSCRASQNVGQSLAWYQQKPGQAPRLLIYDASNRATGIP |
| | | ARFSGSGSGTDFTLTISSLEPEDFAVYYCQLRTNWPPVTFGGGTKVEIK |
| 12-42 | 4168 | EIVLTQSPATLSLSPGERATLSCRASQSVDSRLAWYQQKPGQAPRLLIYDTSNRATGIP |
| | | ARFSGSGSGTDFTLTISSLEPEDFAVYYCQQRSSNWTFGGGTKVEIK |
| 12-43 | 4169 | EIVLTQSPATLSLSPGERATLSCRASQSVGKSLAWYQQKPGQAPRLLIYDTSNRATGIP |
| | | ARFSGSGSGTDFTLTISSLEPEDFAVYYCQQRGSFPLTFGGGTKVEIK |
| 13-1 | 4170 | EIVLTQSPATLSLSPGERATLSCRASQSVGDFLAWYQQKPGQAPRLLIYDTSNRATGIP |
| | | ARFSGSGSGTDFTLTISSLEPEDFAVYYCQQRSIRGTFGGGTKVEIK |
| 13-2 | 4171 | EIVLTQSPATLSLSPGERATLSCRASDTVSSYLAWYQQKPGQAPRLLIYDTSNRATGIP |
| | | ARFSGSGSGTDFTLTISSLEPEDFAVYYCQQRGGWPPAFGGGTKVEIK |
| 13-3 | 4172 | DIQMTQSPSSLSASVGDRVTITCRASQSIGDYLNWYQQKPGKAPKLLIYEASSLQSGVP |
| | | SRFSGSGSGTDFTLTISSLQPEDFATYYCLHTYLPPYSFGQGTKVEIK |
| 13-4 | 4173 | DIQMTQSPSSLSASVGDRVTITCRASQSITRYLNWYQQKPGKAPKLLIYAASSLQSGVP |
| | | SRFSGSGSGTDFTLTISSLQPEDFATYYCQQTYNFPHTFGQGTKVEIK |
| 13-5 | 4174 | EIVLTQSPATLSLSPGERATLSCRASQSIGSYLAWYQQKPGQAPRLLIYDVSNRATGIP |
| | | ARFSGSGSGTDFTLTISSFEPEDFAVYYCQQRHHWPPVTFGGGTKVEIK |

TABLE 14
Antibody Sequences

| | SEQ | |
|---|---|---|
| Antibody | ID NO | Sequence |
| Antibody 1 | 4175 | EVQLVESGGGLVQPGGSLRLSCAASGSTFSIN |
| | | AMGWFRQAPGKEREFVAGITSSGGYTNYADSV |
| | | KGRFTISADNSKNTAYLQMNSLKPEDTAVYYC |
| | | AADGVPEYSDYASGPVWGQGTLVTVSSGGGGS |
| | | GGGGSASEVQLVESGGGLVQPGGSLRLSCAAS |
| | | GFTFSPSWMGWFRQAPGKEREFVATINEYGGR |
| | | NYADSVKGRFTISADNSKNTAYLQMNSLKPED |
| | | TAVYYCARVDRDFDYWGQGTLVTVSSGGGGSE |
| | | PKSSDKTHTCPPCPAPELLGGPSVFLFPPKPK |
| | | DTLMISRTPEVTCVVVDVSHEDPEVKFNWYVD |
| | | GVEVHNAKTKPREEQYNSTYRVVSVLTVLHQD |
| | | WLNGKEYKCKVSNKALPAPIEKTISKAKGQPR |
| | | EPQVYTLPPSREEMTKNQVSLTCLVKGFYPSD |
| | | IAVEWESNGQPENNYKTTPPVLDSDGSFFLYS |
| | | KLTVDKSRWQQGNVFSCSVMHEALHNHYTQKS |
| | | LSLSPG |
| Antibody 2 | 4176 | EVQLVESGGGLVQPGGSLRLSCAASGFTFSPS |
| | | WMGWFRQAPGKEREFVATINEYGGRNYADSVK |
| | | GRFTISADNSKNTAYLQMNSLKPEDTAVYYCA |
| | | RVDRDFDYWGQGTLVTVSSGGGGSEPKSSDKT |
| | | HTCPPCPAPELLGGPSVFLFPPKPKDTLMISR |
| | | TPEVTCVVVDVSHEDPEVKFNWYVDGVEVHNA |
| | | KTKPREEQYNSTYRVVSVLTVLHQDWLNGKEY |
| | | KCKVSNKALPAPIEKTISKAKGQPREPQVYTL |
| | | PPSREEMTKNQVSLTCLVKGFYPSDIAVEWES |
| | | NGQPENNYKTTPPVLSDGSFFLYSKLTVDKSR |
| | | WQQGNVFSCSVMHEALHNHYTQKSLSLSPGGG |
| | | GGSGGGGSASEVQLVESGGGLVQPGGSLRLSC |
| | | AASGSTFSINAMGWFRQAPGKEREFVAGITSS |
| | | GGYTNYADSVKGRFTISADNSKNTAYLQMNSL |
| | | KPEDTAVYYCAADGVPEYSDYASGPVWGQGTL |
| | | VTVSS |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A device for detecting a virus in a sample comprising:
a) a sample application pad for receiving the sample; and
b) a membrane substrate comprising a first test line, the first test line comprising an immobilized antibody or antibody fragment, wherein the immobilized antibody or antibody fragment comprises a predetermined number of variants within a complementarity determining region (CDR) relative to a reference antibody or antibody fragment, and wherein the immobilized antibody or antibody fragment comprises at least a 2.5× higher binding affinity than a binding affinity of the reference antibody or antibody fragment.

2. The device of claim 1, wherein the device is a lateral flow immunoassay.

3-6. (canceled)

7. The device of claim 1, wherein the immobilized antibody comprises an amino acid sequence comprising at least about 80% sequence identity to any one of SEQ ID NOs: 1-4212.

8-17. (canceled)

18. The device of claim 1, wherein the CDR comprises at least one variant relative to the reference antibody or antibody fragment.

19. The device of claim 1, wherein the CDR is a CDR1, CDR2, and CDR3 on a heavy chain.

20. The device of claim 1, wherein the CDR is a CDR1, CDR2, and CDR3 on a light chain.

21. The device of claim 1, wherein the immobilized antibody comprises an EC50 of less than about 5 nM.

22. (canceled)

23. The device of claim 1, wherein the immobilized antibody comprises a binding affinity of less than about 100 nM.

24-29. (canceled)

30. The device of claim 1, wherein the sample comprises saliva, blood, semen, vaginal fluid, or urine.

31. The device of claim 1, wherein the sample comprises saliva.

32. The device of claim 1, wherein the membrane substrate further comprises at least one control line.

33. The device of claim 1, wherein the device further comprises a backing.

34. The device of claim 1, wherein the device further comprises a wicking pad.

35. The device of claim 1, wherein the device comprises a sensitivity of at least about 70% for detecting the virus.

36. The device of claim 1, wherein the device detects viral titers in a range of about 10³ to about 10⁴ viral particles.

37. The device of claim 1, wherein the device comprises a specificity of at least about 70% for detecting the virus as compared to another virus.

38. The device of claim 1, wherein the device is specific for detecting SARS-CoV-2.

39. (canceled)

40. A method of detecting a virus, the method comprising:
a) contacting a sample comprising the virus with a device of claim 1; and
b) detecting the virus if the first test line undergoes a color change.

41. The method of claim 40, wherein the method detects the virus in at most about 20 minutes.

42. (canceled)

43. A kit comprising:
a) a device of claim 1, and
b) instructions for use thereof.
